(19) United States
(12) Patent Application Publication
(10) Pub. No.: US 2022/0305136 A1
(43) Pub. Date: Sep. 29, 2022

(54) ANTI-CTLA4 CONJUGATES

(71) Applicant: Ascendis Pharma Oncology Division A/S, Hellerup (DK)

(72) Inventors: Thomas KNAPPE, Heidelberg (DE); Sebastian STARK, Heidelberg (DE); Burkhardt LAUFER, Heidelberg (DE); Sarah NOWAK, Heidelberg (DE); Roman KITYK, Heidelberg (DE); Nicolas ZYDZIAK, Heidelberg (DE)

(21) Appl. No.: 17/596,828

(22) PCT Filed: Jun. 19, 2020
(86) PCT No.: PCT/EP2020/067157

§ 371 Date: Dec. 20, 2021

(30) Foreign Application Priority Data
Jun. 21, 2019 (EP) 19181807.9
Jan. 3, 2020 (EP) 20150235.8

(51) Int. Cl. A61K 47/68; A61K 47/61; A61K 47/60; A61K 47/69; A61P 35/00

(52) U.S. Cl. CPC A61K 47/6851; A61K 47/6849; A61K 47/61; A61K 47/60; A61K 47/6903; A61P 35/00

(57) ABSTRACT

The present invention relates to an anti-CTLA4 conjugate or a pharmaceutically acceptable salt thereof, wherein said conjugate comprises a plurality of anti-CTLA4 moieties -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety; and related aspects.

The present invention relates to an anti-CTLA4 conjugate or a pharmaceutically acceptable salt thereof, wherein said conjugate comprises a plurality of anti-CTLA4 moieties -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety; and related aspects.

Recent advances in the treatment of cancer include therapy with immune modulating drugs which work by activating immune responses within a patient against that patient's cancer cells. Treatment with agents such as antibodies which target and block inhibitory checkpoint receptors on lymphocytes, such as T cells, increases the activation of those lymphocytes in vitro and in vivo and have demonstrated clinical efficacy in treating cancer leading to their approval for multiple cancer indications. However, a significant drawback to systemic treatment with inhibitory checkpoint receptor blocking drugs is the induction of systemic off-tumor immune activation which can lead to undesired and dose limiting side effects. This is particularly true for agents which block Cytotoxic T Lymphocyte Antigen 4 (CTLA4), an inhibitory receptor transiently expressed on most activated T cells and constitutively expressed on regulatory T cells.

CTLA4 is an inhibitory receptor on activated T cells and is expressed at high levels on regulatory T cells. CTLA4 functions by multiple mechanisms which include binding with high affinity to co-stimulatory ligands B7.1 (CD80) and B7.2 (CD86), effectively sequestering these ligands and blocking them from activating T cells by inhibiting their interaction with costimulatory receptor CD28. CTLA4 is also proposed to receive signals from B7.1 and B7.2 in a way that enhances the suppressive functions of regulatory T cells toward other immune cells.

Mice deficient in CTLA4 display potent autoimmune phenotypes (Chambers et al. Immunity. 1997) including lethal lymphoproliferation and multi organ tissue destruction (Tivol E A et al. Immunity. 1995). Blockade of CTLA4 increases T cell responses in vitro (Walunas et al. Immunity. 1994) and in vivo (Kearney et al. J. Immunol. 1995) and enhances anti-tumor immunity (Leach D R et al. Science. 1996) (van Elsas et al. J Exp Med. 1999). Unfortunately, systemic blockade of CTLA4 also enhances the development of autoimmune conditions such as diabetes (Luhder et al. J Exp Med. 1998), myasthenia gravis (Wang H B et al. J Immunol. 2001), experimental autoimmune encephalomyelitis (Perrin P J, et al. J Immunol. 1996), and autoimmune gastritis (Takahashi T et al. J Exp Med. 2000). Not surprisingly, human studies have identified associations between human CTLA4 gene polymorphisms and increased risks of autoimmune diabetes and thyroid disease (Ueda H et al. Nature. 2003).

Ipilimumab is an anti-CTLA-4 antibody on a human IgG1 isotype which was first approved to treat melanoma. Mechanistically, Ipilimumab is capable of interfering with (blocking) the interaction between CTLA4 and its ligands B7.1 and B7.2. The Constant Fragment (Fc) domain of Ipilimumab, as an IgG1 isotype, can also interact with Fcγ Receptors. In mouse models, Fcγ Receptor engagement is critical for the function of anti-CTLA4 analogs mAbs and results in depletion of regulatory T cells in the tumor. However, depletion of regulatory T cells in human tumors has not been clearly demonstrated after Ipilimumab treatment so the role of the Fc of Ipilimumab in patients is less clear (Sharma A et al. Clin Cancer Res. 2019). It remains possible that the major mechanism of action of Ipilimumab in human patients is blocking the interaction of CTLA4 with B7.1 and B7.2 with a minimal role of the Fc domain. Tremelimumab is another example of an anti-CTLA4 antagonist (blocking) antibody.

A significant challenge in treatments targeting CTLA4 is that virtually all activated T cells or regulatory T cells express CTLA4 throughout the body (not just in the tumor) and so systemic blockade will result in systemic T cell activation in a manner that is not tumor specific and allows for the development of immune related adverse events (irAEs) which are dose limiting and potentially life threatening. While Ipilimumab treatment can demonstrate significant clinical benefit in a subset of patients, its utility is limited by a number of undesirable and dangerous systemic irAEs including dermatitis, colitis, thyroiditis, hypophysitis, and hepatitis (Cheng et al J Gastroenterology and Hepatology 2015). Close monitoring for irAEs in patients being treated with ipilimumab is essential as early treatment is critical to reduce the risk of sequelae, which may be life-threatening (Weber et al JCO 2012). Hepatic injury and colitis are significant concerns because they can develop with little warning and may potentially be severe. Guidelines for treating Ipilimumab induced irAEs involve cessation of Ipilimumab treatment and often include initiation of immune suppressive agents such as high dose steroids (Beck et al. JCO 2006, Weber et al JCO 2012). While Ipilimumab was first tested and approved at a systemically administered 3 mpk dose level, current clinical trials have lowered the systemically administered dose to 1 mpk to reduce irAEs. Unfortunately, this likely comes at a cost to overall efficacy potential as previous studies demonstrated that higher doses of Ipilimumab yield better Overall Survival in melanoma patients (Ascierto et al. Lancet Oncology 2017).

Clearly, blockade of CTLA4 can robustly augment immune function, which can have beneficial and efficacious consequences for treating patients with cancer. However, systemic blockade of CTLA4 results in a number of dangerous irAEs which limit the dosing and potential efficacy of CTLA4 treatment.

In summary, there is a need for a more efficacious treatment.

It is an object of the present invention to at least partially overcome the above-described shortcomings.

This objective is achieved with an anti-CTLA4 conjugate or a pharmaceutically acceptable salt thereof, wherein said conjugate comprises a plurality of anti-CTLA4 moieties -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety.

It was surprisingly found that the anti-CTLA4 conjugates of the present invention allow for a local treatment with agents which block CTLA4 at or near the site of a tumor to enhance local immune responses against that tumor while limiting systemic levels of anti-CTLA4 drug and blockade to allow the benefits of blocking CTLA4 near the tumor site while avoiding the perils of robust systemic CTLA4 blockade.

Within the present invention the terms are used having the meaning as follows.

As used herein the terms “anti-CTLA4 drug” and “anti-CTLA4 moiety” refer to a drug or drug moiety, respectively, which binds to CTLA4 and which may block the interaction with its ligands B7.1 and B7.2 (CD80 and CD86). In certain embodiments such anti-CTLA4 drug or anti-CTLA4 moiety may be selected from the group consisting of antibodies, antibody fragments, affibodies, affilins, affimers, affitins, alphamabs, alphabodies, anticalins, avimers, DARPins, Fynomers®, Kunitz domain peptides, monobodies, nanoCLAMPs, cyclic peptides, small molecules and nanobodies.

As used herein the term “anti-tumor activity” means the ability to reduce the speed of tumor growth by at least 20%, such as by at least 25%, by at least 30%, by at least 35%, by at least 40%, by at least 45%, or by at least 50%; the ability to inhibit tumors from growing larger, i.e. tumor growth inhibition or tumor stasis; or the ability to cause a reduction in the size of a tumor, i.e. tumor regression. Anti-tumor activity may be determined by comparing the mean relative tumor volumes between control and treatment conditions. Relative volumes of individual tumors (individual RTVs) for day “x” may be calculated by dividing the absolute individual tumor volume on day “x” (Tₓ) following treatment initiation by the absolute individual tumor volume of the same tumor on the day treatment started (To) multiplied by 100:

$${RTV}_{x} [ \% ] = \frac{{T}_{x}}{{T}_{0}} \times 100$$

Anti-tumor activity may be observed between 7 to 21 days following treatment initiation.

Tumor size, reported in mm³, may be measured physically by measuring the length (L) measured in mm and width (W) measured in mm of the tumors, which may include injected and non-inject tumors. Tumor volume can be determined by methods such as ultrasound imaging, magnetic resonance imaging, computed tomography scanning, or approximated by using the equation V=½×(L×W²), with V being the tumor volume. Tumor burden, i.e. the total number of cancer cells in an individuum, can also be measured in the case of an experimental tumor model that expresses a reporter, such as luciferase enzyme or a fluorescent protein or another measurable protein or enzyme, by measuring the reporter element, i.e. luminescence or fluorescence, or the expressed reporter protein or enzyme product as a measure of the total number of tumor cells present and total tumor size. The latter reporter models can be useful for tumors that are not readily measurable on the surface of the animals (i.e. orthotopic tumors). It is understood that in general the term “animal” also covers human and in certain embodiments means mouse, rat, non-human primate or human. In certain embodiments “animal” means human.

As used herein the term “therapeutic dose” “therapeutically effective dose” refers to a dose that upon administration to a patient results in anti-tumor activity at 7 to 21 days post administration. As experiments with human subjects are strictly regulated, it may not be feasible to test for anti-tumor activity in humans. Accordingly, said anti-tumor activity is in certain embodiments measured in animals, such as in mouse, rat or non-human primates. In certain embodiments anti-tumor activity is measured in mouse. In certain embodiments anti-tumor activity is measured in rat. In certain embodiments anti-tumor activity is measured in non-human primates. In certain embodiments anti-tumor activity is measured in human. Even though anti-tumor activity is to be measured at 7 to 21 days post administration this, however, does not exclude anti-tumor activity prior to 7 days or later than 21 days post administration.

As used herein the term “essentially the same anti-tumor activity” refers to the anti-tumor activity observed between two different treatments, wherein one treatment does not vary by more than 30%, such as no more than 25% or no more than 20%, compared to a reference treatment.

The effects of treating with anti-CTLA4 as a single therapy or in combination with other therapies can be measured by increases in T cell activation.

As used herein the term “local anti-CTLA4-induced T cell activation” refers to effects of an anti-CTLA4 conjugate that are restricted to an area near the site of administration of the anti-CTLA4 conjugate and/or the draining lymph node(s) closest to the injection site. The specific size of the area near the site of administration will depend on the amount of anti-CTLA4 administered, the diffusion rate within the tissue, the time at which the signal is measured following injection, and the rate of drug uptake by neighboring cells, but would typically be detectable within a distance of 2 times the radius (r) from the injection site in any direction, wherein r is the distance in centimeters (cm) calculated from the volume (V) of anti-CTLA4 conjugate injected in cubic centimeters (cm³) following the equation V=(4/3)×πr³. For example, if 0.5 cm³ anti-CTLA4 conjugate is injected into a given tissue, a sample of tissue weighing at least 0.025 g taken within 0.98 cm in any direction of the injection site would display a local anti-CTLA4-induced T cell activation signal. Within a volume of 2 times r, tissue samples are to be taken for determining the presence of local anti-CTLA4-induced T cell activation markers. However, this does not mean that said T cell activation markers outside a volume of 2 times r may not be upregulated. In general, effects of anti-CTLA4 intensity decreases with increasing distance from the administration site. However, the person skilled in the art understands that providing an outer boundary of such localized anti-CTLA4 effects is not feasible, because the extend of these effects depends on various factors, such as for example tumor type. In any way, the person skilled in the art will easily be able to distinguish between local and systemic anti-CTLA4-induced effects where systemic effects would be measured in peripheral secondary lymphoid tissues such as the blood or spleen or in lymph nodes which are contralateral to or do not drain from the injection site.

In general, systemic concentrations may be measured in plasma or serum. In certain embodiments systemic concentrations are measured in plasma. In certain embodiments systemic concentrations are measured in serum.

As used herein the term “local” or “locally” refers to a volume of tissue within a distance of 2 times the radius (r) from an injection site in any direction, wherein r is the distance in centimeters (cm) calculated from the volume (V) of anti-CTLA4 conjugate injected in cubic centimeters (cm³) following the spheroid equation V=(4/3)×πr³. For example, if 0.5 cm³ anti-CTLA4 conjugate is injected into a given tissue, a sample of tissue weighing at least 0.025 g taken within 0.98 cm in any direction of the injection site is referred to as a local sample.

As used herein the term “pattern recognition receptor agonist” (“PRRA”) refers to a molecule that binds to and activates one or more immune cell-associated receptor that recognizes pathogen-associated molecular patterns (PAMPs) or damage-associated molecular patterns (DAMPs), leading to immune cell activation and/or pathogen- or damage-induced inflammatory responses. Pattern recognition receptors (PRRs) are typically expressed by cells of the innate immune system such as monocytes, macrophages, dendritic cells (DCs), neutrophils, and epithelial cells, as well as cells of the adaptive immune system.

As used herein the terms “cytotoxic agent” and “chemotherapeutic agent” are used synonymously and refer to compounds that are toxic to cells, which prevent cellular replication or growth, leading to cellular destruction/death. Examples of cytotoxic agents include chemotherapeutic agents and toxins, such as small molecule toxins or enzymatically active toxins of bacterial, fungal, plant or animal origin, including synthetic analogues and derivatives thereof.

As used herein the terms “immune checkpoint inhibitor” and “immune checkpoint antagonist” are used synonymously and refer to compounds that interfere with the function of, or inhibit binding of ligands that induce signaling through, cell-membrane expressed receptors that inhibit immune cell function upon receptor activation. Such compounds may for example be biologics, such as antibodies, nanobodies, probodies, anticalins or cyclic peptides, or small molecule inhibitors.

As used herein the term “immune agonist” refers to compounds that directly or indirectly activate cell-membrane expressed receptors that stimulate immune cell function upon receptor activation.

As used herein the terms “multi-specific” and “multi-specific drugs” refer to compounds that simultaneously bind to two or more different antigens and can mediate antagonistic, agonistic, or specific antigen binding activity in a target-dependent manner.

As used herein the term “antibody-drug conjugate” (ADC) refers to compounds typically consisting of an antibody linked to a biologically active cytotoxic payload, radiotherapy, or other drug designed to deliver cytotoxic agents to the tumor environment. ADCs are particularly effective for reducing tumor burden without significant systemic toxicity and may act to improve the effectiveness of the immune response induced by checkpoint inhibitor antibodies.

As used herein the term “radionuclides” refers to radioactive isotopes that emit ionizing radiation leading to cellular destruction/death. Radionuclides conjugated to tumor targeting carriers are referred to as “targeted radionuclide therapeutics”.

As used here in the term “DNA damage repair inhibitor” refers to a drug that targets DNA damage repair elements, such as for example CHK1, CHK2, ATM, ATR and PARP. Certain cancers are more susceptive to targeting these pathways due to existing mutations, such as BRCA1 mutated patients to PARP inhibitors due to the concept of synthetic lethality.

As used herein, the term “tumor metabolism inhibitor” refers to a compound that interferes with the function of one or more enzymes expressed in the tumor environment that produce metabolic intermediates that may inhibit immune cell function.

As used herein the term “protein kinase inhibitor” refers to compounds that inhibit the activity of one or more protein kinases. Protein kinases are enzymes that phosphorylate proteins, which in turn can modulate protein function. It is understood that a protein kinase inhibitor may target more than one kinase and any classification for protein kinase inhibitors used herein refers to the main or most characterized target.

As used herein the term “protein kinase inhibitor” refers to compounds that inhibit the activity of one or more protein kinases. Protein kinases are enzymes that phosphorylate proteins, which in turn can modulate protein function. It is understood that a protein kinase inhibitor may target more than one kinase and any classification for protein kinase inhibitors used herein refers to the main or most characterized target.

As used herein the term “chemokine receptor and chemoattractant receptor agonist” refers to compounds that activate chemokine or chemoattractant receptors, a subset of G-protein coupled receptors or G-protein coupled-like receptors that are expressed on a wide variety of cells and are primarily involved in controlling cell motility (chemotaxis or chemokinesis). These receptors may also participate in non-cell migratory processes, such as angiogenesis, cell maturation or inflammation.

As used herein the term “cytokine receptor agonist” refers to soluble proteins which control immune cell activation and proliferation. Cytokines include for example interferons, interleukins, lymphokines, and tumor necrosis factor.

As used herein the term “death receptor agonist” refers to a molecule which is capable of inducing pro-apoptotic signaling through one or more of the death receptors, such as DR4 (TRAIL-R1) or DR5 (TRAIL-R2). The death receptor agonist may be selected from the group consisting of antibodies, death ligands, cytokines, death receptor agonist expressing vectors, peptides, small molecule agonists, cells (such as for example stem cells) expressing the death receptor agonist, and drugs inducing the expression of death ligands.

As used herein the term “intra-tissue administration” refers to a type of administration, for example local injection, of a drug into a tissue of interest such as intra-tumoral, intra-muscular, subdermal or subcutaneous injections or injection into or adjacent to a normal or diseased tissue or organ. In certain embodiments intra-tissue administration is intravenous administration.

As used herein, the term “intra-tumoral administration” refers to a mode of administration, in which the drug is administered directly into tumor tissue. The term “intra-tumoral administration” also refers to administration pre- or post-resection into or onto the tumor bed. When tumor boundary is not well defined, it is also understood that intra-tumoral administration includes administration to tissue adjacent to the tumor cells (“peri-tumoral administration”). Exemplary tumors for intra-tumoral administration are solid tumors and lymphomas. Administration may occur via injection.

As used herein the term “systemic administration” means intravenous administration, such as via intravenous injection or infusion.

As used herein, the term “water-insoluble” refers to the property of a compound of which less than 1 g can be dissolved in one liter of water at 20° C. to form a homogeneous solution. Accordingly, the term “water-soluble” refers to the property of a compound of which 1 g or more can be dissolved in one liter of water at 20° C. to form a homogeneous solution.

As used herein, the term “a π-electron-pair-donating heteroaromatic N-comprising moiety” refers to the moiety which after cleavage of the linkage between -D and -L¹- results in a drug D-H and wherein the drug moiety -D and analogously the corresponding D-H comprises at least one, such as one, two, three, four, five, six, seven, eight, nine or ten heteroaromatic nitrogen atoms that donate a π-electron pair to the aromatic π-system. Examples of chemical structures comprising such heteroaromatic nitrogens that donate a π-electron pair to the aromatic

π-system include, but are not limited to, pyrrole, pyrazole, imidazole, isoindazole, indole, indazole, purine, tetrazole, triazole and carbazole. For example, in the imidazole ring below the heteroaromatic nitrogen which donates a π-electron pair to the aromatic π-system is marked with “#”.

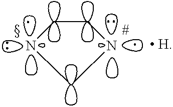

The π-electron-pair-donating heteroaromatic nitrogen atoms do not comprise heteroaromatic nitrogen atoms which only donate one electron (i.e. not a pair of π-electrons) to the aromatic π-system, such as for example the nitrogen that is marked with “§” in the abovementioned imidazole ring structure. The drug D-H may exist in one or more tautomeric forms, such as with one hydrogen atom moving between at least two heteroaromatic nitrogen atoms. In all such cases, the linker moiety is covalently and reversibly attached at a heteroaromatic nitrogen that donates a π-electron pair to the aromatic π-system.

As used herein, the term “drug” refers to a substance used in the treatment, cure, prevention or diagnosis of a disease or used to otherwise enhance physical or mental well-being of a patient. In certain embodiments such substance is used in the treatment of a disease. If a drug is conjugated to another moiety, the moiety of the resulting product that originated from the drug is referred to as “drug moiety”.

Any reference to a biologic drug herein, i.e. to a drug manufactured in, extracted from, or semisynthesized from biological sources such as a protein drug, also covers biosimilar versions of said drug.

As used herein the term “prodrug” refers to a drug moiety reversibly and covalently connected to a specialized protective group through a reversible prodrug linker moiety which is a linker moiety comprising a reversible linkage with the drug moiety and wherein the specialized protective group alters or eliminates undesirable properties in the parent molecule. This also includes the enhancement of desirable properties in the drug and the suppression of undesirable properties. The specialized non-toxic protective group may also be referred to as “carrier”. A prodrug releases the reversibly and covalently bound drug moiety in the form of its corresponding drug. In other words, a prodrug is a conjugate comprising a drug moiety, which is covalently and reversibly conjugated to a carrier moiety via a reversible linker moiety, which covalent and reversible conjugation of the carrier to the reversible linker moiety is either directly or through a spacer. The reversible linker may also be referred to as “reversible prodrug linker”. Such conjugate may release the formerly conjugated drug moiety in the form of a free drug, in which case the reversible linker or reversible prodrug linker is a traceless linker.

As used herein, the term “free form” of a drug means the drug in its unmodified, pharmacologically active form.

As used herein the term “spacer” or “linker” refers to a moiety that connects at least two other moieties with each other.

As used herein, the term “reversible”, “reversibly”, “degradable” or “degradably” with regard to the attachment of a first moiety to a second moiety means that the linkage that connects said first and second moiety is cleavable under physiological conditions, which physiological conditions are aqueous buffer at pH 7.4 and 37° C., with a half-life ranging from at least 7 days, such as at least 14 days, at least 21 days, at least 25 days, at least 40 days, at least 50 days, at least 100 days or at least 180 days. Such cleavage is in certain embodiments non-enzymatically, i.e. independent of enzymatic activity. Accordingly, the term “stable” with regard to the attachment of a first moiety to a second moiety means that the linkage that connects said first and second moiety exhibits a half-life of more than 12 months under physiological conditions.

As used herein, the term “reagent” means a chemical compound, which comprises at least one functional group for reaction with the functional group of another chemical compound or drug. It is understood that a drug comprising a functional group is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety, such as an anti-CTLA4 moiety, is released from a reversible linkage as a drug, such as an anti-CTLA4 drug.

It is understood that if the chemical structure of a group of atoms is provided and if this group of atoms is attached to two moieties or is interrupting a moiety, said sequence or chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety

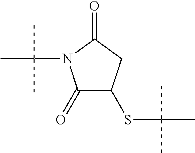

can be attached to two moieties or can interrupt a moiety either as

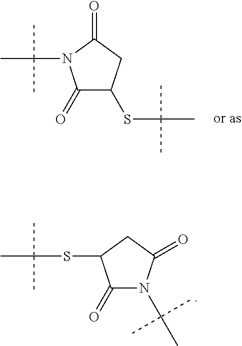

The term “substituted” as used herein means that one or more —H atom(s) of a molecule or moiety are replaced by a different atom or a group of atoms, which are referred to as “substituent”.

As used herein, the term “substituent” in certain embodiments refers to a moiety selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3}), —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

—R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein the term “crosslinker” refers to a moiety that is a connection between different elements of a hydrogel, such as between two or more backbone moieties or between two or more hyaluronic acid strands.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity. In certain embodiments the hydrogel is insoluble due to the presence of covalent chemical crosslinks.

As used herein the term “continuous gel” refers to a hydrogel in a flexible shape, i.e. a shape that is not pre-formed, but adjusts its shape to fit its surrounding. Upon administration, such as via injection, such continuous gel may in certain embodiments fragment into smaller sized particles. In certain embodiments such continuous gel does not fragment upon administration, such as via injection, and remains essentially the same volume, but may temporarily or permanently change its shape as required to pass through a needle, for example.

As used herein the term “about” in combination with a numerical value is used to indicate a range ranging from and including the numerical value plus and minus no more than 25% of said numerical value, such as no more than plus and minus 20% of said numerical value or such as no more than plus and minus 10% of said numerical value. For example, the phrase “about 200” is used to mean a range ranging from and including 200+/−25%, i.e. ranging from and including 150 to 250; such as 200+/−20%, i.e. ranging from and including 160 to 240; such as ranging from and including 200+/−10%, i.e. ranging from and including 180 to 220. It is understood that a percentage given as “about 50%” does not mean “50%+/−25%”, i.e. ranging from and including 25 to 75%, but “about 50%” means ranging from and including 37.5 to 62.5%, i.e. plus and minus 25% of the numerical value which is 50.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes, for example, alternating copolymers in which monomers of different types alternate, periodic copolymers, in which monomers of different types are arranged in a repeating sequence; statistical copolymers, in which monomers of different types are arranged randomly; block copolymers, in which blocks of different homopolymers consisting of only one type of monomers are linked by a covalent bond; and gradient copolymers, in which the composition of different monomers changes gradually along a polymer chain. In certain embodiments a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, it preferably has a molecular weight of at most 1000 kDa, such as at most 750 kDa, such as at most 500 kDa, such as at most 300 kDa, such as at most 200 kDa, such as at most 100 kDa. It is understood that a polymer may also comprise one or more other moieties, such as, for example, one or more functional groups.

The term “polymer” also relates to a peptide or protein, even though the side chains of individual amino acid residues may be different. It is understood that for covalently crosslinked polymers, such as hydrogels, no meaningful molecular weight ranges can be provided.

As used herein, the term “polymeric” refers to a reagent or a moiety comprising one or more polymers or polymer moieties. A polymeric reagent or moiety may optionally also comprise one or more other moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

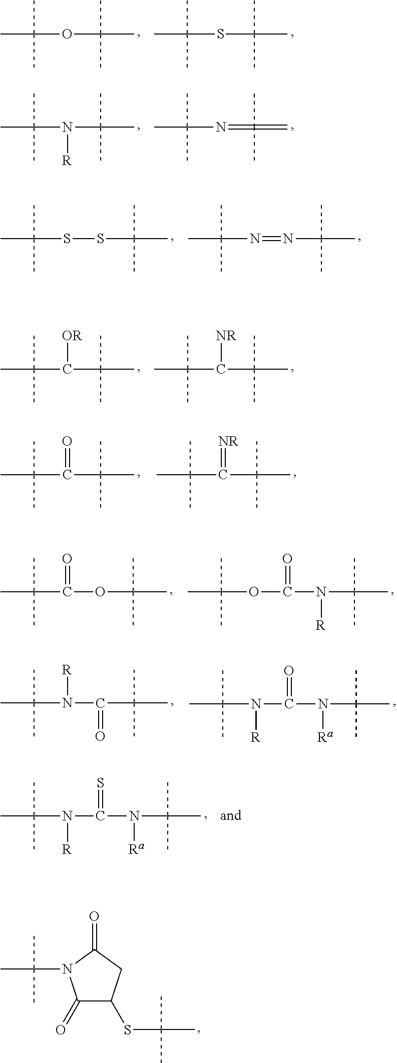

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−25%, such as x+/−20% or such as x+/−10%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. Such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, or such as at least 95% (w/w) PEG. The remaining weight percentage of the PEG-based moiety or reagent may be other moieties, such as those selected from the group consisting of.
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group consisting of

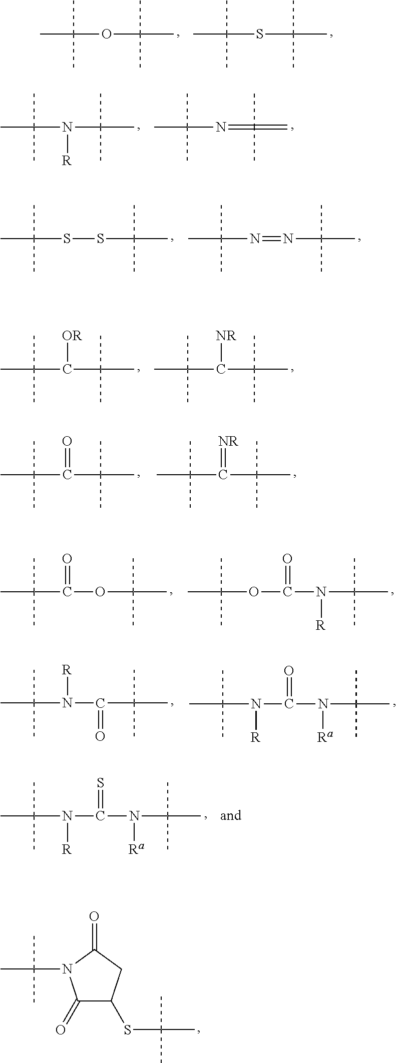

wherein
dashed lines indicate attachment to the remainder of the moiety or reagent, and —R and —R^{a} are independently of each other selected from the group consisting of —H, and C₁₋₆ alkyl; and
which moieties and linkages are optionally further substituted.

The terms “poly(alkylene glycol)-based”, “poly(propylene glycol)-based” and “hyaluronic acid-based” are used accordingly.

The term “interrupted” means that a moiety is inserted between two carbon atoms or—if the insertion is at one of the moiety's ends—between a carbon or heteroatom and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which may be selected from the group consisting of

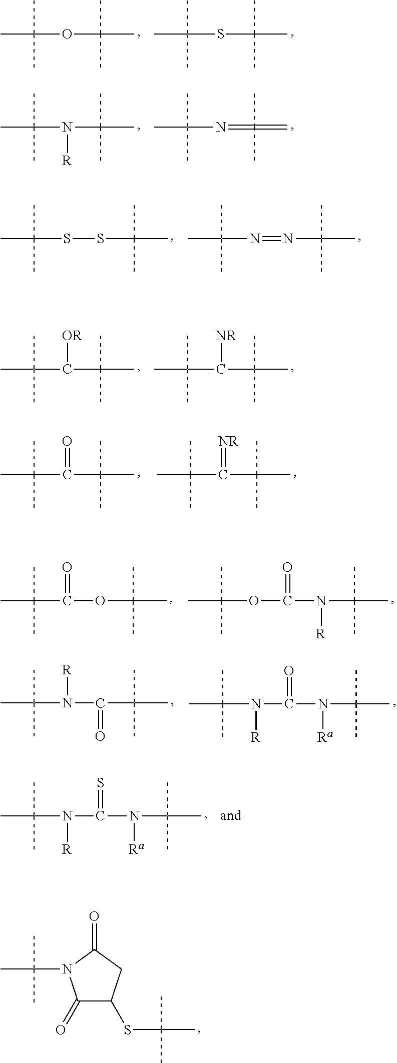

wherein
dashed lines indicate attachment to the remainder of the moiety or reagent; and
—R and —R^{a} are independently of each other selected from the group consisting of —H and C₁₋₆ alkyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In one embodiment a 8- to 30-membered carbopolycyclyl means a cyclic moiety of two, three, four or five rings. In another embodiment a 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including=N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent.

Similarly, the term “8- to 30-membered heteropolycyclyl” or “8- to 30-membered heteropolycycle” means a heterocyclic moiety of more than two rings with 8 to 30 ring atoms, such as of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including=N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

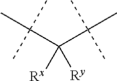

means that R^{x} and R^{y} form the following structure:

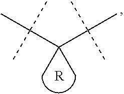

wherein R is a C₃₋₁₀ cycloalkyl or a 3- to 10-membered heterocyclyl.

It is also understood that the phrase “the pair R^{x}/R^{y} is joint together with the atoms to which they are attached to form a ring A” in relation with a moiety of the structure

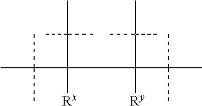

means that R^{x} and R^{y} form the following structure:

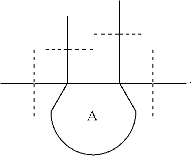

As used herein, “halogen” means fluoro, chloro, bromo or iodo. In certain embodiments halogen is fluoro or chloro.

It is also understood that the phrase “—R¹ and an adjacent —R² form a carbon-carbon double bond provided that n is selected from the group consisting of 1, 2, 3 and 4” in relation with a moiety of the structure:

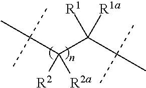

means that for example when n is 1, —R¹ and the adjacent —R² form the following structure:

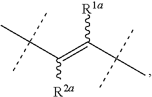

and if for example, n is 2, —R¹ and the adjacent —R² form the following structure:

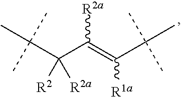

wherein the wavy bond means that —R^{1a} and —R^{2a} may be either on the same side of the double bond, i.e. in cis configuration, or on opposite sides of the double bond, i.e. in trans configuration and wherein the term “adjacent” means that —R¹ and —R² are attached to carbon atoms that are next to each other.

It is also understood that the phrase “two adjacent —R² form a carbon-carbon double bond provided that n is selected from the group consisting of 2, 3 and 4” in relation with a moiety of the structure:

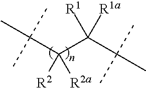

means that for example when n is 2, two adjacent —R² form the following structure:

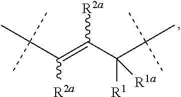

wherein the wavy bond means that each —R^{2a} may be either on the same side of the double bond, i.e. in cis configuration, or on opposite sides of the double bond, i.e. in trans configuration and wherein the term “adjacent” means that two —R² are attached to carbon atoms that are next to each other.

It is understood that the “N” in the phrase “π-electron-pair-donating heteroaromatic N” refers to nitrogen.

It is understood that “N⁺” in the phrases “an electron-donating heteroaromatic N⁺-comprising moiety” and “attachment to the N⁺ of -D⁺” refers to a positively charged nitrogen atom.

As used herein the term “alkali metal ion” refers to Na⁺, K⁺, Li⁺, Rb⁺ and Cs⁺. In certain embodiments “alkali metal ion” refers to Na⁺, K⁺ and L⁺.

As used herein the term “alkaline earth metal ion” refers to Mg²⁺, Ca²⁺, Sr²⁺ and Ba²⁺. In certain embodiments an alkaline earth metal ion is Mg²⁺ or Ca²⁺.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the compounds of the present invention comprise one or more acidic or basic groups, the invention also comprises their corresponding pharmaceutically or toxicologically acceptable salts, in particular their pharmaceutically utilizable salts. Thus, the compounds of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine, amino acids, and quarternary ammonium salts, like tetrabutylammonium or cetyl trimethylammonium. Compounds of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid, and other acids known to the person skilled in the art. For the person skilled in the art further methods are known for converting the basic group into a cation like the alkylation of an amine group resulting in a positively-charge ammonium group and an appropriate counterion of the salt. If the compounds of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe), the FDA (US) or any other national regulatory agency for use in animals, such as for use in humans.

As used herein, the term “excipient” refers to a diluent, adjuvant, or vehicle with which the therapeutic, such as a drug or the anti-CTLA4 conjugate of the present invention, is administered. Such pharmaceutical excipient may be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, may also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or may contain detergents, like Tween, poloxamers, poloxamines, CHAPS, Igepal, or amino acids like, for example, glycine, lysine, or histidine.

These pharmaceutical compositions may take the form of solutions, suspensions, emulsions, tablets, pills, capsules, powders, sustained-release formulations and the like. The pharmaceutical composition may be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions may contain a therapeutically effective amount of the drug, such as the anti-CTLA4 conjugate of the present invention, together with a suitable amount of excipient so as to provide the form for proper administration to the patient. The formulation should suit the mode of administration.

The term “peptide” as used herein refers to a chain of at least 2 and up to and including 50 amino acid monomer moieties, which may also be referred to as “amino acid residues”, linked by peptide (amide) linkages, which may be linear, branched or cyclic. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein the term “small molecule drug” refers to drugs that are organic compounds with a molecular weight of no more than 1 kDa, such as up to 900 kDa.

As used herein the term “biologics” or “biopharmaceutical” refers to any pharmaceutical drug manufactured in, extracted from, or semi-synthesized from biological sources. Different from totally synthesized pharmaceuticals, they may include vaccines, blood, blood components, allergenics, somatic cells, gene therapies, tissues, recombinant therapeutic protein, and living cells used in cell therapy. Biologics may be composed of sugars, proteins, or nucleic acids or complex combinations of these substances, or may be living cells or tissues. They or their precursors or components are isolated from living sources, such as from human, animal, plant, fungal or microbial sources.

In general, the terms “comprise” or “comprising” also encompasses “consist of” or “consisting of”.

It is understood that each moiety -D is covalently conjugated via at least one moiety -L¹-L²- to a polymeric moiety Z.

In certain embodiments -D is selected from the group consisting of wild-type F, anti-CTLA4 antibodies, Fc enhanced for effector function/FcγR binding anti-CTLA4 antibodies, anti-CTLA4 antibodies conditionally active in tumor microenvironment, anti-CTLA4 small molecules, CTLA4 antagonist fusion proteins, anti-CTLA4 anticalins, anti-CTLA4 nanobodies and anti-CTLA4 multispecific biologics based on antibodies, scFVs or other formats. In certain embodiments -D is a wild-type F, anti-CTLA4 antibody. In certain embodiments -D is a Fc enhanced for effector function/FcγR binding anti-CTLA4 antibody. In certain embodiments -D is an anti-CTLA4 antibody conditionally active in tumor microenvironment. In certain embodiments -D is an anti-CTLA4 small molecule. In certain embodiments -D is a CTLA4 antagonist fusion protein. In certain embodiments -D is an anti-CTLA4 anticalin. In certain embodiments -D is an anti-CTLA4 nanobody. In certain embodiments -D is an anti-CTLA4 multispecific biologic based on an antibody, scFV or other format. In certain embodiments -D is an anti-CTLA4 multispecific biologic based on an antibody. In certain embodiments -D is an anti-CTLA4 multispecific based on a scFV.

Exemplary wild-type Fe anti-CTLA4 antibodies are selected from the group consisting of ipilimumab, tremelimumab, MK-1308, CBT509 (also known as APL-509), ONC392, IBI310, CG0161, BCD145, ADU1604, AGEN1884 and CS1002. In certain embodiments -D is ipilimumab. In certain embodiments -D is tremelimumab.

Exemplary Fc enhanced for effector function/FcγR binding anti-CTLA4 antibodies are selected from the group consisting of AGEN1181 and anti-CTLA-4 SIFbody.

Exemplary anti-CTLA4 antibodies conditionally active in tumor microenvironment are selected from the group consisting of BMS-986249 and BA3071.

An exemplary anti-CTLA4 small molecule is BPI-002.

An exemplary CTLA4 antagonist fusion protein is FPT155.

An exemplary anti CTLA4 anticalin is PRS010.

Exemplary anti-CTLA4 multispecific biologics are selected from the group consisting of TE1254, XmAb22841, XmAb20717, MEDI5752, MGD019, ALPN-202, ATOR-1015 and ATOR-1144.

In certain embodiments all moieties -D of an anti-CTLA4 conjugate are identical. It is understood that this does not exclude the occurrence of changes in the chemical structure of individual anti-CTLA4 moieties due to, for example, molecular rearrangements or degradation, as may for example occur during storage. In certain embodiments the anti-CTLA4 conjugate comprises more than one type of -D, i.e. two or more different types of -D, such as two different types of -D, three different types of -D, four different types of -D or five different types of -D.

If the anti-CTLA4 conjugate of the present comprises more than one type of -D, all -D may be connected to the same type of -L¹- or may be connected to different types of -L¹-, i.e. a first type of -D may be connected to a first type of -L¹-, a second type of -D may be connected to a second type of -L¹- and so on. Using different types of -L¹- may in certain embodiments allow different release kinetics for different types of -D, such as for example a faster release for a first type of -D, a medium release for a second type of -D and a slow release for a third type of -D. Likewise, two different types of -D may be connected to the same type of -L¹-, allowing for release of both types of -D with the same release kinetics. Accordingly, in certain embodiments the conjugates of the present invention comprise one type of -L¹-. In certain embodiments the conjugates of the present invention comprise two types of -L¹-. In certain embodiments the conjugates of the present invention comprise three types of -L¹-. In certain embodiments the conjugates of the present invention comprise four types of -L¹-. In certain embodiments the conjugates of the present invention comprise five types of -L¹-.

In certain embodiments the conjugates of the present invention comprise one type of -D and one type of -L¹-. In certain embodiments the conjugates of the present invention comprise two types of -D and two types of -L¹-. In certain embodiments the conjugates of the present invention comprise three types of -D and three types of -L¹-. In certain embodiments the conjugates of the present invention comprise four types of -D and four types of -L¹-. -. In certain embodiments the conjugates of the present invention comprise two types of -D and one type of -L¹-. In certain embodiments the conjugates of the present invention comprise three types of -D and one or two types of -L¹-.

In certain embodiments at least 10% of all moieties -D of the anti-CTLA4 conjugate are ipilimumab, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the anti-CTLA4 conjugate are ipilimumab.

In certain embodiments at least 10% of all moieties -D of the anti-CTLA4 conjugate are tremelimumab, such as at least 20% of all moieties -D, such as at least 30% of all moieties -D, such as at least 40% of all moieties -D, such as at least 50% of all moieties -D, such as at least 60% of all moieties -D, such as at least 70% of all moieties -D, such as at least 80% of all moieties -D, such as at least 90% of all moieties -D. In certain embodiments all moieties -D of the anti-CTLA4 conjugate are tremelimumab.

In certain embodiments the anti-CTLA4 conjugate comprises in addition to the at least one moiety -D in the form of an anti-CTLA4 moiety one or more drug moieties -D of at least one different class of drugs, i.e. some of the moieties -D of the anti-CTLA4 conjugate are anti-CTLA4 moieties as described above and in addition the anti-CTLA4 conjugate comprises moieties -D that are from one or more different classes of drugs or—in other words—are non-anti-CTLA4 moieties.

In certain embodiments these moieties -D in the form of a different class of drugs are selected from the group consisting of cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones or any combination thereof. In certain embodiments these moieties -D in the form of a different class of drugs are selected from the group consisting of cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones or any combination thereof.

In certain embodiments these moieties -D in the form of a different class of drugs are selected from the group consisting of cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones or any combination thereof. In certain embodiments these moieties -D in the form of a different class of drugs are selected from the group consisting of cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones.

In certain embodiments the one or more non-anti-CTLA4 moieties -D are cytotoxic/chemotherapeutic agents. In certain embodiments the one or more non-anti-CTLA4 moieties -D are immune checkpoint inhibitors or antagonists. In certain embodiments the one or more non-anti-CTLA4 moieties -D are multi-specific drugs. In certain embodiments the one or more non-anti-CTLA4 moieties -D are antibody-drug conjugates (ADC). In certain embodiments the one or more non-anti-CTLA4 moieties -D are targeted radionuclide therapeutics. In certain embodiments the one or more non-anti-CTLA4 moieties -D are DNA damage repair inhibitors. In certain embodiments the one or more non-anti-CTLA4 moieties -D are tumor metabolism inhibitors. In certain embodiments the one or more non-anti-CTLA4 moieties -D are pattern recognition receptor agonists. In certain embodiments the one or more non-anti-CTLA4 moieties -D are protein kinase inhibitors. In certain embodiments the one or more non-anti-CTLA4 moieties -D are chemokines and chemoattractant receptor agonists. In certain embodiments the one or more non-anti-CTLA4 moieties -D are chemokines or chemokine receptor antagonists. In certain embodiments the one or more non-anti-CTLA4 moieties -D are cytokine receptor agonists. In certain embodiments the one or more non-anti-CTLA4 moieties -D are death receptor agonists. In certain embodiments the one or more non-anti-CTLA4 moieties -D are CD47 antagonists. In certain embodiments the one or more non-anti-CTLA4 moieties -D are SIRPα antagonists. In certain embodiments the one or more non-anti-CTLA4 moieties -D are oncolytic drugs. In certain embodiments the one or more non-anti-CTLA4 moieties -D are signal converter proteins. In certain embodiments the one or more non-anti-CTLA4 moieties -D are epigenetic modifiers. In certain embodiments the one or more non-anti-CTLA4 moieties -D are tumor peptides or tumor vaccines. In certain embodiments the one or more non-anti-CTLA4 moieties -D are heat shock protein (HSP) inhibitors. In certain embodiments the one or more non-anti-CTLA4 moieties -D are proteolytic enzymes. In certain embodiments the one or more non-anti-CTLA4 moieties -D are ubiquitin and proteasome inhibitors. In certain embodiments the one or more non-anti-CTLA4 moieties -D are adhesion molecule antagonists. In certain embodiments the one or more non-anti-CTLA4 moieties -D are hormones including hormone peptides and synthetic hormones.

Examples for cytotoxic or chemotherapeutic agent are alkylating agents, anti-metabolites, anti-microtubule agents, topoisomerase inhibitors, cytotoxic antibiotics, auristatins, enediynes, lexitropsins, duocarmycins, cyclopropylpyrroloindoles, puromycin, dolastatins, maytansine derivatives, alkylsufonates, triazenes and piperazine.

Example for an alkylating agent are nitrogen mustards, such as mechlorethamine, cyclophosphamide, melphalan, chlorambucil, ifosfamide and busulfan; nitrosoureas, such as N-nitroso-N-methylurea, carmustine, lomustine, semustine, fotemustine and streptozotocin; tetrazines, such as dacarbazine, mitozolomide and temozolomide; ethylenimines, such as altretamine; aziridines, such as thiotepa, mitomycin and diaziquone; cisplatin and derivatives, such as cisplatin, carboplatin, oxaliplatin; and non-classical alkylating agents, such as procarbazine and hexamethylmelamine.

Examples for an anti-metabolite are anti-folates, such as methotrexate and pemetrexed; fluoropyrimidines, such as fluorouracil and capecitabine; deoxynucleoside analogues, such as cytarabine, gemcitabine, decitabine, azacytidine, fludarabine, nelarabine, cladribine, clofarabine and pentostatin; and thiopurines, such as thioguanine and mercaptopurine.

Examples for an anti-microtubule agent are Vinca alkaloids, such as vincristine, vinblastine, vinorelbine, vindesine and vinflunine; taxanes, such as paclitaxel and docetaxel; podophyllotoxins and derivatives, such as podophyllotoxin, etoposide and teniposide; stilbenoid phenol and derivatives, such as zybrestat (CA4P); and BNC105.

Examples for a topoisomerase inhibitor are topoisomerase I inhibitors, such as irinotecan, topotecan and camptothecin; and topoisomerase II inhibitors, such as etoposide, doxorubicin, mitoxantrone, teniposide, novobiocin, merbarone and aclarubicin.

Examples for a cytotoxic antibiotic are anthracyclines, such as doxorubicin, daunorubicin, epirubicin and idarubicin; pirarubicin, aclarubicin, bleomycin, mitomycin C, mitoxantrone, actinomycin, dactinomycin, adriamycin, mithramycin and tirapazamine.

Examples for an auristatin are monomethyl auristatin E (MMAE) and monomethyl auristatin F (MMAF).

Examples for an enediyne are neocarzinostatin, lidamycin (C-1027), calicheamicins, esperamicins, dynemicins and golfomycin A.

Examples for a maytansine derivative are ansamitocin, mertansine (emtansine, DM1) and ravtansine (soravtansine, DM4).

Examples for an immune checkpoint inhibitor or antagonist are inhibitors of CTLA-4 (cytotoxic T-lymphocyte-associated protein 4), such as ipilimumab, tremelimumab, MK-1308, FPT155, PRS010, BMS-986249, BPI-002, CBT509, JS007, ONC392, TE1254, IB1310, BR02001, CG0161, KN044, PBI5D3H5, BCD145, ADU1604, AGEN1884, AGEN1181, CS1002 and CP675206; inhibitors of PD-1 (programmed death 1), such as pembrolizumab, nivolumab, pidilizumab, AMP-224, BMS-936559, cemiplimab and PDR001; inhibitors of PD-L1 (programmed cell death protein 1), such as MDX-1105, MED14736, atezolizumab, avelumab, BMS-936559 and durvalumab; inhibitors of PD-L2 (programmed death-ligand 2); inhibitors of KIR (killer-cell immunoglobulin-like receptor), such as lirlumab (IPH2102) and IPH2101; inhibitors of B7-H3, such as MGA271; inhibitors of B7-H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; and inhibitors of PVRIG, such as COM701.

In certain embodiments said one or more further drug is an inhibitor of PD-1. In certain embodiments said one or more further drug is an inhibitor of PD-L1.

Examples for an immune agonist are CD27, such as recombinant CD70, such as HERA-CD27L, and varlilumab (CDX-1127); agonists of CD28, such as recombinant CD80, recombinant CD86, TGN1412 and FPT155; agonists of CD40, such as recombinant CD40L, CP-870,893, dacetuzumab (SGN-40), Chi Lob 7/4, ADC-1013 and CDX1140; agonists of 4-1BB (CD137), such as recombinant 4-1BBL, urelumab, utomilumab and ATOR-1017; agonists of OX40, such as recombinant OX40L, MEDI0562, GSK3174998, MOXR0916 and PF-04548600; agonists of GITR, such as recombinant GITRL, TRX518, MEDI1873, INCAGN01876, MK-1248, MK-4166, GWN323 and BMS-986156; and agonists of ICOS, such as recombinant ICOSL, JTX-2011 and GSK3359609.

Examples for a multi-specific drug are biologics and small molecule immune checkpoint inhibitors. Examples for biologics are multi-specific immune checkpoint inhibitors, such as CD137/HER2 lipocalin, PD1/LAG3, FS118, XmAb22841 and XmAb20717; and multi-specific immune agonists. Such multi-specific immune agonists may be selected from the group consisting of Ig superfamily agonists, such as ALPN-202; TNF superfamily agonists, such as ATOR-1015, ATOR-1144, ALG.APV-527, lipocalin/PRS-343, PRS344/ONC0055, FAP-CD40 DARPin, MP0310 DARPin, FAP-0X40 DARPin, EGFR-CD40 DARPin, EGFR41BB/CD137 DARPin, EGFR-0X40/DARFPin, HER2-CD40 DARPin, HER2-41BB/CD137 DARPin, HER2-0X40 DARPin, FIBRONECTIN ED-B-CD40 DARPin, FIBRONECTIN ED-B-41BB/CD137 and FIBRONECTIN ED-B-0X40 DARPin; CD3 multispecific agonists, such as blinatumomab, solitomab, MEDI-565, ertumaxomab, anti-HER2/CD3 1Fab-immunoblobulin G TDB, GBR1302, MGD009, MGD007, EGFRBi, EGFR-CD Probody, RG7802, PF-06863135, PF-06671008, MOR209/ES414, AMG212/BAY2010112 and CD3-5T4; and CD16 multispecific agonists, such as 1633 BiKE, 161533 TriKE, OXS-3550, OXS-C3550, AFM13 and AFM24.

An example for a small molecule immune checkpoint inhibitor is CA-327 (TIM3/PD-L1 antagonist).

Examples for an antibody-drug conjugate are ADCs targeting hematopoietic cancers, such as gemtuzumab ozogamicin, brentuximab vedotin, inotuzumab ozogamicin, SAR3419, BT062, SGN-CD19A, IMGN529, MDX-1203, polatuzumab vedotin (RG7596), pinatuzumab vedotin (RG7593), RG7598, milatuzumab-doxorubicin and OXS-1550; and ADCs targeting solid tumor antigens, such as trastuzumab emtansine, glembatumomab vedotin, SAR56658, AMG-172, AMG-595, BAY-94-9343, BIIB015, vorsetuzumab mafodotin (SGN-75), ABT-414, ASG-5ME, enfortumab vedotin (ASG-22ME), ASG-16M8F, IMGN853, indusatumab vedotin (MLN-0264), vadortuzumab vedotin (RG7450), sofituzumab vedotin (RG7458), lifastuzumab vedotin (RG7599), RG7600, DEDN6526A (RG7636), PSMA TTC, 1095 from Progenics Pharmaceuticals, lorvotuzumab mertansine, lorvotuzumab emtansine, IMMU-130, sacituzumab govitecan (IMMU-132), PF-06263507 and MEDI0641.

Examples for radionuclides are β-emitters, such as ¹⁷⁷Lutetium, ¹⁶⁶Holmium, ¹⁸⁶Rhenium, ¹⁸⁸Rhenium, ⁶⁷Copper, ¹⁴⁹Promethium, ¹⁹⁹Gold, ⁷⁷Bromine, ¹⁵³Samarium, ¹⁰⁵Rhodium, ⁸⁹Strontium, ⁹⁰Yttrium, ¹³¹Iodine; α-emitters, such as ²¹³Bismuth, ²²³Radium, ²²⁵Actinium, ²¹¹Astatine; and Auger electron-emitters, such as ⁷⁷Bromine, ¹¹¹Indium, ¹²³Iodine and ¹²⁵Iodine.

Examples for targeted radionuclide therapeutics are zevalin (⁹⁰Y-ibritumomab tiuxetan), bexxar (¹³¹I-tositumomab), oncolym (¹³¹I-Lym 1), lymphocide (⁹⁰Y-epratuzumab), cotara (¹³¹I-chTNT-1/B), labetuzumab (⁹⁰Y or ¹³¹I-CEA), theragyn (⁹⁰Y-pemtumomab), licartin (¹³¹I-metuximab), radretumab (¹³¹I-L19) PAM4 (⁹⁰Y-clivatuzumab tetraxetan), xofigo (²²³Ra dichloride), lutathera (¹⁷⁷Lu-DOTA-Tyr³-Octreotate) and ¹³¹I-MIBG.

Examples for a DNA damage repair inhibitor are poly (ADP-ribose) polymerase (PARP) inhibitors, such as olaparib, rucaparib, niraparib, veliparib, CEP 9722 and E7016; CHK1/CHK2 dual inhibitors, such as AZD7762, V158411, CBP501 and XL844; CHK1 selective inhibitors, such as PF477736, MK8776/SCH900776, CCT244747, CCT245737, LY2603618, LY2606368/prexasertib, AB-IsoG, ARRY575, AZD7762, CBP93872, ESPO1, GDC0425, SAR020106, SRA737, V158411 and VER250840; CHK2 inhibitors, such as CCT241533 and PV1019; ATM inhibitors, such as AZD0156, AZD1390, KU55933, M3541 and SX-RDS1; ATR inhibitors, such as AZD6738, BAY1895344, M4344 and M6620 (VX-970); and DNA-PK inhibitors, such as M3814.

Examples for a tumor metabolism inhibitor are inhibitors of the adenosine pathway, inhibitors of the tryptophan metabolism and inhibitors of the arginine pathway.

Examples for an inhibitor of the adenosine pathway are inhibitors of A2AR (adenosine A2A receptor), such as ATL-444, istradefylline (KW-6002), MSX-3, preladenant (SCH-420,814), SCH-58261, SCH412,348, SCH-442,416, ST-1535, caffeine, VER-6623, VER-6947, VER-7835, vipadenant (BIIB-014), ZM-241,385, PBF-509 and V81444; inhibitors of CD73, such as IPH53 and SRF373; and inhibitors of CD39, such as IPH52.

Examples for an inhibitor of the tryptophane metabolism are inhibitors of IDO, such as indoximod (NLG8189), epacadostat, navoximod, BMS-986205 and MK-7162; inhibitors of TDO, such as 680C91; and IDO/TDO dual inhibitors.

Examples for inhibitors of the arginine pathway are inhibitors of arginase, such as INCB001158.

Examples for a pattern recognition agonist are Toll-like receptor agonists, NOD-like receptors, RIG-I-like receptors, cytosolic DNA sensors, STING, and aryl hydrocarbon receptors (AhR).

Examples for Toll-like receptor agonists are agonists of TLR1/2, such as peptidoglycans, lipoproteins, Pam3CSK4, Amplivant, SLP-AMPLIVANT, HESPECTA, ISA101 and ISA201; agonists of TLR2, such as LAM-MS, LPS-PG, LTA-BS, LTA-SA, PGN-BS, PGN-EB, PGN-EK, PGN-SA, CL429, FSL-1, Pam2CSK4, Pam3CSK4, zymosan, CBLB612, SV-283, ISA204, SMP105, heat killed Listeria monocytogenes; agonists of TLR3, such as poly(A:U), poly(I:C) (poly-ICLC), rintatolimod, apoxxim, IPH3102, poly-ICR, PRV300, RGCL2, RGIC.1, Riboxxim (RGC100, RGIC100), Riboxxol (RGIC50) and Riboxxon; agonists of TLR4, such as lipopolysaccharides (LPS), neoceptin-3, glucopyranosyl lipid adjuvant (GLA), GLA-SE, G100, GLA-AF, clinical center reference endotoxin (CCRE), monophosphoryl lipid A, grass MATA MPL, PEPA10, ONT-10 (PET-Lipid A, oncothyreon), G-305, ALD046, CRX527, CRX675 (RC527, RC590), GSK1795091, OM197MPAC, OM294DP and SAR439794; agonists of TLR2/4, such as lipid A, OM174 and PGN007; agonists of TLR5, such as flagellin, entolimod, mobilan, protectan CBLB501; agonists of TLR6/2, such as diacylated lipoproteins, diacylated lipopeptides, FSL-1, MALP-2 and CBLB613; agonists of TLR7, such as CL264, CL307, imiquimod (R837), TMX-101, TMX-201, TMX-202, TMX-302, gardiquimod, 5-27609, 851, UC-IV150, 852A (3M-001, PF-04878691), loxoribine, polyuridylic acid, GSK2245035, GS-9620, R06864018 (ANA773, RG7795), R07020531, isatoribine, AN0331, ANA245, ANA971, ANA975, DSP0509, DSP3025 (AZD8848), GS986, MBS2, MBS5, RG7863 (R06870868), sotirimod, SZU101 and TQA3334; agonists of TLR8, such as ssPolyUridine, ssRNA40, TL8-506, XG-1-236, VTX-2337 (motolimod), VTX-1463, TMX-302, VTX-763, DN1508052 and GS9688; agonists of TLR7/8, such as CL075, CL097, poly(dT), resiquimod (R-848, VML600, S28463), MED19197 (3M-052), NKTR262, DV1001, IM04200, IPH3201 and VTX1463; agonists of TLR9, such as CpG DNA, CpG ODN, lefitolimod (MGN1703), SD-101, QbG10, CYT003, CYT003-QbG10, DUK-CpG-001, CpG-7909 (PF-3512676), GNKG168, EMD 1201081, IMO-2125, IMO-2055, CpG10104, AZD1419, ASTO08, IM02134, MGN1706, IRS 954, 1018 ISS, actilon (CPG10101), ATP00001, AVE0675, AVE7279, CMP001, DIMS0001, DIMS9022, DIMS9054, DIMS9059, DV230, DV281, EnanDIM, heplisav (V270), kappaproct (DIMS0150), NJP834, NP1503, SAR21609 and tolamba; and agonists of TLR7/9, such as DV1179.

In certain embodiments the agonist of TLR7/8 is a conjugate as described in PCT/EP2020/050093. In particular the agonist of TLR7/8 is in certain embodiments of formula (I)

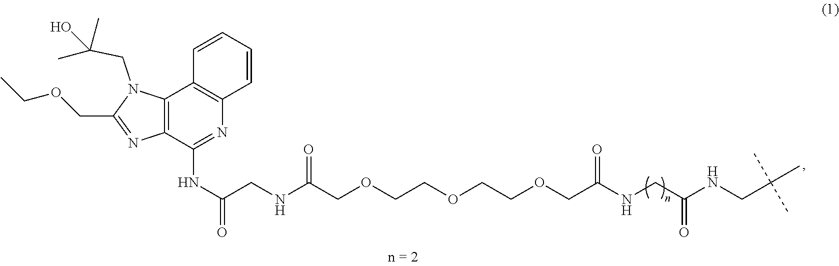

wherein the dashed line indicates attachment to a PEG hydrogel. It is understood that a plurality of the moieties of formula (I) are conjugated to said hydrogel.

Examples for CpG ODN are ODN 1585, ODN 2216, ODN 2336, ODN 1668, ODN 1826, ODN 2006, ODN 2007, ODN BW006, ODN D-SL01, ODN 2395, ODN M362 and ODN D-SL03.

Examples for NOD-like receptors are agonists of NOD1, such as C12-iE-DAP, C14-Tri-LAN-Gly, iE-DAP, iE-Lys, and Tri-DAP; and agonists of NOD2, such as L18-MDP, MDP, M-TriLYS, murabutide and N-glycolyl-MDP.

Examples for RIG-I-like receptors are 3p-hpRNA, 5′ppp-dsRNA, 5′ppp RNA (M8), 5′OH RNA with kink (CBS-13-BPS), 5′PPP SLR, KIN100, KIN 101, KIN1000, KIN1400, KIN1408, KIN1409, KIN1148, KIN131A, poly(dA:dT), SB9200, RGT100 and hiltonol.

Examples for cytosolic DNA sensors are cGAS agonists, dsDNA-EC, G3-YSD, HSV-60, ISD, ODN TTAGGG (A151), poly(dG:dC) and VACV-70.

Examples for STING are MK-1454, ADU-S100 (MIW815), 2′3′-cGAMP, 3′3′-cGAMP, c-di-AMP, c-di-GMP, cAIMP (CL592), cAIMP difluor (CL614), cAIM(PS)₂ difluor (Rp/Sp) (CL656), 2′2′-cGAMP, 2′3′-cGAM(PS)2 (Rp/Sp), 3′3′-cGAM fluorinated, c-di-AMP fluorinated, 2′3′-c-di-AMP, 2′3′-c-di-AM(PS)2 (Rp,Rp), c-di-GMP fluorinated, 2′3′-c-di-GMP, c-di-IMP, c-di-UMP and DMXAA (vadimezan, ASA404).

Examples for an aryl hydrocarbon receptor (AhR) are of FICZ, ITE and L-kynurenine.

Examples for a protein kinase inhibitor are receptor tyrosine kinase inhibitors, intracellular kinase inhibitors, cyclin dependent kinase inhibitors, phosphoinositide-3-kinase inhibitors, mitogen-activated protein kinase inhibitors, inhibitors of nuclear factor kappa-β kinase (IKK), and Wee-1 inhibitors.

Examples for receptor tyrosine kinase inhibitors are EGF receptor inhibitors, such as afatinib, cetuximab, erlotinib, gefitinib, pertuzumab and margetuximab; VEGF receptor inhibitors, such as axitinib, lenvatinib, pegaptanib and linifanib (ABT-869); C-KIT Receptor inhibitors, such as CDX0158 (KTN0158); ERBB2 (HER2) inhibiors, such as herceptin (trastuzumab); ERBB3 receptor inhibitors, such as CDX3379 (MED13379, KTN3379) and AZD8931 (sapitinib); FGF receptor inhibitors, such as erdafitinib; AXL receptor inhibitors, such as BGB324 (BGB 324, R428, R428, bemcentinib) and SLC391; and MET receptor inhibitors, such as CGEN241.

Examples for intracellular kinase inhibitors are Bruton's tyrosine kinase (BTK) inhibitors, such as ibrutinib, acalabrutinib, GS-4059, spebrutinib, BGB-3111, HM71224, zanubrutinib, ARQ531, BI-BTKT and vecabrutinib; spleen tyrosine kinase inhibitors, such as fostamatinib; Bcr-Abl tyrosine kinase inhibitors, such as imatinib and nilotinib; Janus kinase inhibitors, such as ruxolitinib, tofacitinib and fedratinib; and multi-specific tyrosine kinase inhibitors, such as bosutinib, crizotinib, cabozantinib, dasatinib, entrectinib, lapatinib, mubritinib, pazopanib, sorafenib, sunitinib, SU6656 and vandetanib.

Examples for cyclin dependent kinase inhibitors are ribociclib, palbociclib, abemaciclib, trilaciclib, purvalanol A, olomucine II and MK-7965.

Examples for phophoinositide-3-kinase inhibitors are IP1549, GDc-0326, pictilisib, serabelisib, IC-87114, AMG319, seletalisib, idealisib and CUDC907.

Examples for mitogen-activated protein kinase inhibitors are Ras/farnesyl transferase inhibitors, such as tipirafinib and LB42708; Raf inhibitors, such as regorafenib, encorafenib, vemurafenib, dabrafenib, sorafenib, PLX-4720, GDC-0879, AZ628, lifirafenib, PLX7904 and RO5126766; MEK inhibitors, such as cobimetinib, trametinib, binimetinib, selumetinib, pimasertib, refametinib and PD0325901; ERK inhibitors, such as MK-8353, GDC-0994, ulixertinib and SCH772984.

Examples for inhibitors of nuclear factor kappa-β kinase (IKK) are BPI-003 and AS602868.

An example of a Wee-1 inhibitor is adavosertib.

Examples for a chemokine receptor and chemoattractant receptor agonist are CXC chemokine receptors, CC chemokine receptors, C chemokine receptors, CX3C chemokine receptors and chemoattractant receptors.

Examples for a CXC chemokine receptor are CXCR1 agonists, such as recombinant CXCL8 and recombinant CXCL6; CXCR2 agonists, such as recombinant CXCL8, recombinant CXCL1, recombinant CXCL2, recombinant CXCL3, recombinant CXCL5, recombinant CXCL6, MGTA 145 and SB251353; CXCR3 agonists, such as recombinant CXCL9, recombinant CXCL10, recombinant CXCL11 and recombinant CXCL4; CXCR4 agonists, such as recombinant CXCL12, ATI2341, CTCE0214, CTCE0324 and NNZ4921; CXCR5 agonists, such as recombinant CXCL13; CXCR6 agonists, such as recombinant CXCL16; and CXCL7 agonists, such as recombinant CXCL11.

Examples for a CC chemokine receptor are CCR1 agonists, such as recombinant CCL3, ECI301, recombinant CCL4, recombinant CCL5, recombinant CCL6, recombinant CCL8, recombinant CCL9/10, recombinant CCL14, recombinant CCL15, recombinant CCL16, recombinant CCL23, PB103, PB105 and MPIF1; CCR2 agonists, such as recombinant CCL2, recombinant CCL8, recombinant CCL16, PB103 and PB105; CCR3 agonists, such as recombinant CCL11, recombinant CCL26, recombinant CCL7, recombinant CCL13, recombinant CCL15, recombinant CCL24, recombinant CCL5, recombinant CCL28 and recombinant CCL18; CCR4 agonists, such as recombinant CCL3, ECI301, recombinant CCL5, recombinant CCL17 and recombinant CCL22; CCR5 agonists, such as recombinant CCL3, ECI301, recombinant CCL5, recombinant CCL8, recombinant CCL11, recombinant CCL13, recombinant CCL14, recombinant CCL16, PB103 and PB105; CCR6 agonists, such as recombinant CCL20; CCR7 agonists, such as recombinant CCL19 and recombinant CCL21; CCR8 agonists, such as recombinant CCL1, recombinant CCL16, PB103 and PB105; CCR9 agonists, such as recombinant CCL25; CCR10 agonists, such as recombinant CCL27 and recombinant CCL28; and CCR11 agonists, such as recombinant CCL19, recombinant CCL21 and recombinant CCL25.

Examples for C chemokine receptors are XCR1 agonist, such as recombinant XCL1 or recombinant XCL2.

Examples for CX3C chemokine receptors are CX3CR1 agonist, such as recombinant CX3CL1.

Examples for chemoattractant receptors are formyl peptide receptor agonists, such as N-formyl peptides, N-formylmethionine-leucyl-phenylalanine, enfuvirtide, T21/DP107, annexin A1, Ac2-26 and Ac9-25; C5a receptor agonists; and chemokine-like receptor 1 agonists, such as chemerin.

Examples for chemokine antagonists are inhibitors of CXCL chemokines, such as UNBS5162; inhibitors of CXCL8, such as BMS986253 and PA620; inhibitors of CXCL10, such as TM110, eldelumab and NI0801; inhibitors of CXCL12, such as NOX-A12 and JVS100; inhibitors of CXCL13, such as VX5; inhibitors of CCL2, such as PA508, ABN912, AF2838, BN83250, BN83470, C243, CGEN54, CNT0888, NOXE36, VT224 and SSR150106; inhibitors of CCL5, such as HGS1025 and NI0701; inhibitors of CCL2/CCL5, such as BKTP46; inhibitors of CCL5/FMLP receptor, such as RAP160; inhibitors of CCL11, such as bertilimumab and RAP701; inhibitors of CCL5/CXCL4, such as CT2008 and CT2009; inhibitors of CCL20, such as GSK3050002; and inhibitors of CX3CL1, such as quetmolimab.

Examples for chemokine receptor antagonists are inhibitors of CXCR1, such as repertaxin, CCX832, FX68 and KB03; inhibitors of CXCR2, such as AZD5069, AZD5122, AZD8309, GSK1325756, GSK1325756H, PS291822, SB332235 and SB656933; inhibitors of CXCR1/CXCR2, such as DF1970, DF2156A, DF2162, DF2755A, reparixin, SX576, SX682, PACG31P, AZD4721 and PA401; inhibitors of CXCR3; inhibitors of CXCR4, such as BL8040; inhibitors of CXCR4/E-selectin, such as GMI1359; inhibitors of CXCR6, such as CCX5224; inhibitors of CCR1, such as AZD4818, BAY865047, BMS817399, CCX354, CCX634, CCX9588, CP481715, MLN3701, MLN3897, PS031291, PS375179 and PS386113; inhibitors of CCR2, such as AZD2423, BL2030, BMS741672, CCX140, CCX598, CCX872, CCX915, CNTX6970, INCB3284, INCB3344, INCB8696, JNJ17166864, JNJ27141491, MK0812, OPLCCL2LPM, PF4136309, serocion, STIB0201, STIB0211, STIB0221, STIB0232, STIB0234, TAK202, TPI526; inhibitors of CCR2/CCR5, such as PF04634817, RAP103 and TBR652; inhibitors of CCR2/CCR5/CCR8, such as RAP310; inhibitors of CCR3, such as ASM8, AXP1275, BMS639623, CM101, DPC168, GW766994, GW824575, MT0814, OPLCCL11LPM and QAP642; inhibitors of CCR4, such as AT008, AZD2098, CCX6239, FLX193, FLX475, GBV3019, GSK2239633, IC487892 and poteligeo; inhibitors of CCR5, such as 5P12-RANTES, AZD5672, AZD8566, CMPD167, ESN196, GSK706769, GW873140, HGS004, INCB15050, INCB9471, L872, microbicide, PF232798, PRO140, RAP101, SAR113244, SCH350634, SCH351125, SCH417690, selzentry, TAK779, TBR220, TD0232 and VX286; inhibitors of CCR5/CXCR4, such as AMD887, ND401 and SP01A; inhibitors of CCR6, such as CCX507, CCX9664 and STIB100X; inhibitors of CCR6, such as CCX025, CCX507, CCX807, eut22, MLN3126, POL7085, traficet-EN; inhibitors of CXCR3, such as AMG487, AT010, STIA120X; inhibitors of CXCR4, such as AD114, AD214, ALX0651, ALX40-4C, AMD070, AT007, AT009, BKT170, BMS936564, celixafor, CTCE9908, GBV4086, GSK812397, KRH2731, KRH3140, LY2510924, LY2624587, mozobil, OPLCXCL12LPM, PF06747143, POL6326, Q122, revixil, TG0054, USL311, X4P001 and X4P002; and inhibitors of CXCR7, such as CCX650 and CCX662.

Examples for a cytokine receptor agonist are mRNAs, DNAs or plasmids encoding the genes for IL-2, IL-15, IL-7, IL-10, IL-12, IL-21, IFNα 1-17, IFNβ, IFNγ, IL-18, IL-27, TNFα, GM-CSF, FLT3L and TRAIL and recombinant proteins, such as agonists of IL-2/IL-15 β/γ receptors, agonists of IL-10 receptor, agonists of IL-12 receptor, agonists of IL-18 receptor, agonists of IL-21 receptor, agonists of IL-7 receptor, agonists of IFNα/β receptor, agonists of IFN γ receptor, agonists of FLT3 receptor and agonists of TNFα receptor.

Examples for agonists of IL-2/IL-15 β/γ receptor are recombinant IL-2, recombinant IL-15, ALKS4230, ALT803, APN301, MDNA109, NKTR214, RG7461, RG7813, AM0015, NIZ985, NKTR255, RTX-212, SO-C101, XmAb24306, L19-IL2, THOR-707 and PB101.

In certain embodiments an agonist of IL-2 is as described in WO2019/185705A1, which is herewith incorporated by reference in its entirety. In particular the agonist of IL-2 is in certain embodiments a conjugate comprising an IL-2 protein of SEQ ID NO:1 PTSSSTKKTQ LQLEHLLLDL QMILNGINNY KNPKLTCMLT FKFYMPKKAT ELKHLQCLEE ELKPLEEVLN LAQSKNFHLR PRDLISNINV IVLELKGSET TFMCEYADET ATIVEFLNRW ITFSQSIIST LT,

wherein the sulfur of the cysteine at position 37 of SEQ ID NO:1 is conjugated to a moiety of formula (2)

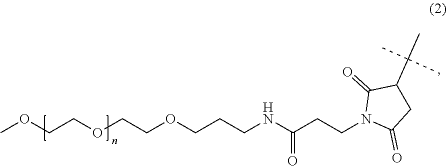

wherein the dashed line indicates attachment to said sulfur, and
n is about 113 or about 226;
   and wherein the nitrogen of the amine of the side chain of any one of the lysine residues, i.e. one of the lysine residues selected from the group consisting of the lysine residues at position 7, 8, 31, 34, 42, 47, 48, 53, 63, 75 and 96 of SEQ ID NO:1, is conjugated to a moiety of formula (3)

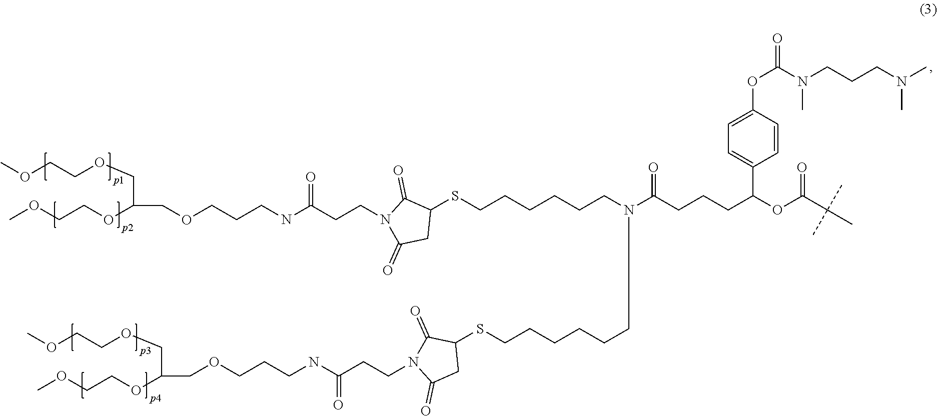

wherein the dashed line indicates attachment to said nitrogen of the side chain of said lysine residue; and
p1, p2, p3 and p4 are independently an integer ranging from 200 to 250.

In certain embodiments the sequence of the IL-2 protein varies by at least one amino acid from the sequence of SEQ ID NO:1, such as by one amino acid, by two amino acids, by three amino acids, by four amino acids or by five amino acids.

In certain embodiments the sequence of the agonist of IL-2 is of SEQ ID NO:3:

APTSSSTKKTQLQLEHLLLDLQMILNGINNYKNPK
LTCMLTFKFYMPKKATELKHLQCLEEELKPLEEVL
NLAQSKNFHLRPRDLISNINVIVLELKGSETTFMC
EYADETATIVEFLNRWITFSQSIISTLT

Accordingly, the agonist of IL-2 is in certain embodiments a conjugate comprising an IL-2 protein of SEQ ID NO:3

APTSSSTKKTQLQLEHLLLDLQMILNGINNYKNPK
LTCMLTFKFYMPKKATELKHLQCLEEELKPLEEVL
NLAQSKNFHLRPRDLISNINVIVLELKGSETTFMC
EYADETATIVEFLNRWITFSQSIISTLT,

wherein the sulfur of the cysteine at position 38 of SEQ ID NO:3 is conjugated to a moiety of formula (2)

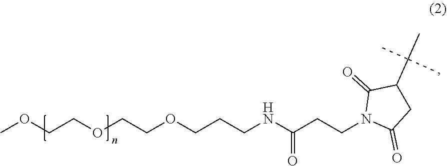

wherein the dashed line indicates attachment to said sulfur, and
n is about 113 or about 226;
   and wherein the nitrogen of the amine of the side chain of any one of the lysine residues, i.e. one of the lysine residues selected from the group consisting of the lysine residues at position 8, 9, 32, 35, 43, 48, 49, 54, 64, 76 and 97 of SEQ ID NO:3, is conjugated to a moiety of formula (3)

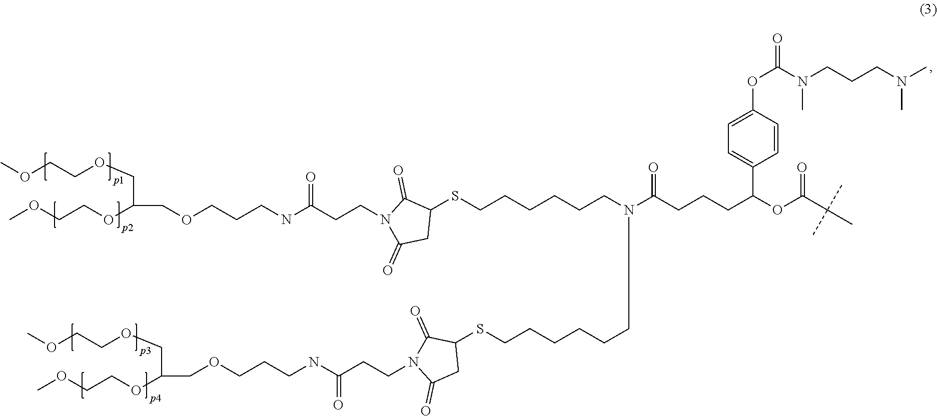

wherein the dashed line indicates attachment to said nitrogen of the side chain of said lysine residue; and
p1, p2, p3 and p4 are independently an integer ranging from 200 to 250.

In certain embodiments n of formula (2) is 113. In certain embodiments n of formula (2) is 226.

In certain embodiments p1, p2, p3 and p4 are independently an integer ranging from 220 to 240. In certain embodiments p1, p2, p3 and p4 are the same integer.

Examples for agonists of IL-10 receptor are AG011, dekavil, EG10, IL10Nanocap, Ilodecakin, AM0010, tenovil and VT310 VIRON.

Examples for agonists of IL-12 receptor are AM0012, AS1409, dodekin, HemaMax, LipoVIL12, MSB0010360N and NHS-IL12.

An example for an agonist of IL-18 receptor is SB485232.

An example for an agonist of IL-21 receptor is BMS982470 (denenicokin).

Examples for agonists of IL-7 receptor are CYT107, CYT99007 and GX-I7.

Examples for agonist of TNFα receptor are L19-TNFα, aurimune, beromun, BreMel/TNFα, fibromun, refnot and TNFPEG20.

Examples for death receptor agonists are TRAILR1/DR4 agonists, such as AMG951 (dulanermin), APG350, APG880, HGSETR1 (mapatumumab) and SL231; and TRAILR2/DR5 agonists, such as AMG655, DS8273, HGSETR2 (lexatumumab), HGSTR2J, IDD004/GEN1029, INBRX109, LBY135, MEDI3039, PR095780, RG7386 and TAS266.

Examples for CD47 antagonists are ALX148, CC-90002, Hu5F9G4, SRF231, TI061, TTI-621, TTI-622, A0176, IB1188, IMC002 and LYN00301.

An example for a SIRPα antagonist is FST89.

Examples for oncolytic drugs are CAVATAK, BCG, mobilan, TG4010, Pexa-Vec (JX-594), JX-900, JX-929 and JX-970.

Examples for signal converter proteins are Fn14-TRAIL (KAHR101), CTLA4-FasL (KAHR102), PD1-41BBL (DSP 105), PD1-CD70 (DSP 106) and SIRPα-41BBL (DSP 107).

Examples for epigenetic modifiers are DNA methyltransferase inhibitors, lysine-specific demethylase 1 inhibitors, Zeste homolog 2 inhibitors, bromodomain and extra-terminal motif (BET) protein inhibitors such as GSK525762, and histone deacetylase (HDAC) inhibitors such as beleodaq, SNDX275 and CKD-M808.

Examples for tumor peptides/vaccines are NY-ESO, WTT, MART-1, IO102 and PF-06753512.

Examples for heat shock protein (HSP) inhibitors are inhibitors of HSP90, such as PF-04929113 (SNX-5422).

Examples of proteolytic enzymes are recombinant hyaluronidase, such as rHuPH20 and PEGPH20.

Examples for ubiquitin and proteasome inhibitors are ubiquitin-specific protease (USP) inhibitors, such as P005091; 20S proteasome inhibitors, such as bortezimib, carfilzomib, ixazomib, oprozomib, delanzomib and celastrol; and immunoproteasome inhibitors, such as ONX-0914.

Examples for adhesion molecule antagonists are 02-integrin antagonists, such as imprime PGG; and selectin antagonists.

Examples for hormones are hormone receptor agonists and hormone receptor antagonists.

Examples for a hormone receptor agonist are somatostatin receptor agonists, such as somatostatin, lanreotide, octreotide, FX125L, FX141L and FX87L.

Examples for hormone receptor antagonists are anti-androgens, anti-estrogens and anti-progestogens. Examples for anti-androgens are steroidal antiandrogens, such as cyproterone acetate, megestrol acetate, chlormadinone acetate, spironolactone, oxendolone and osaterone acetate; nonsteroidal anti-androgens, such as flutamide, bicalutamide, nilutamide, topilutamide, enzalutamide and apalutamide; androgen synthesis inhibitors, such as ketoconazole, abiraterone acetate, seviteronel, aminoglutethimide, finasteride, dutasteride, epristeride and alfatradiol. Examples for anti-estrogens are selective estrogen receptor modulators (SERMs), such as tamoxifen, clomifene, Fareston and raloxifene; ER silent antagonists and selective estrogen receptor degrader (SERD), such as fulvestrant; aromatase inhibitors, such as anastrozole, letrozole, exemestane, vorozole, formestane and fadrozole; and anti-gonadotropins, such as testosterone, progestogens and GnRH analogues. Examples for anti-progestogens are mifepristone, lilopristone and onapristone.

In certain embodiments such cytotoxic or chemotherapeutic agents are selected from the group consisting of alkylating agents, anti-metabolites, anti-microtubule agents, topoisomerase inhibitors, cytotoxic antibiotics, auristatins, enediynes, lexitropsins, duocarmycins, cyclopropylpyrroloindoles, puromycin, dolastatins, maytansine derivatives, alkylsufonates, triazenes and piperazine.

The alkylating agent is in certain embodiments selected from the group consisting of nitrogen mustards, such as mechlorethamine, cyclophosphamide, melphalan, chlorambucil, ifosfamide and busulfan; nitrosoureas, such as N-nitroso-N-methylurea, carmustine, lomustine, semustine, fotemustine and streptozotocin; tetrazines, such as dacarbazine, mitozolomide and temozolomide; ethylenimines, such as altretamine; aziridines, such as thiotepa, mitomycin and diaziquone; cisplatin and derivatives, such as cisplatin, carboplatin, oxaliplatin; and non-classical alkylating agents, such as procarbazine and hexamethylmelamine.

The anti-metabolite is in certain embodiments selected from the group consisting of anti-folates, such as methotrexate and pemetrexed; fluoropyrimidines, such as fluorouracil and capecitabine; deoxynucleoside analogues, such as cytarabine, gemcitabine, decitabine, azacytidine, fludarabine, nelarabine, cladribine, clofarabine and pentostatin; and thiopurines, such as thioguanine and mercaptopurine.

The anti-microtubule agent is in certain embodiments selected from the group consisting of Vinca alkaloids, such as vincristine, vinblastine, vinorelbine, vindesine and vinflunine; taxanes, such as paclitaxel and docetaxel; podophyllotoxins and derivatives, such as podophyllotoxin, etoposide and teniposide; stilbenoid phenol and derivatives, such as zybrestat (CA4P); and BNC105.

The topoisomerase inhibitor is in certain embodiments selected from the group consisting of topoisomerase I inhibitors, such as irinotecan, topotecan and camptothecin; and topoisomerase II inhibitors, such as etoposide, doxorubicin, mitoxantrone, teniposide, novobiocin, merbarone and aclarubicin.

The cytotoxic antibiotic is in certain embodiments selected from the group consisting of anthracyclines, such as doxorubicin, daunorubicin, epirubicin and idarubicin; pirarubicin, aclarubicin, bleomycin, mitomycin C, mitoxantrone, actinomycin, dactinomycin, adriamycin, mithramycin and tirapazamine.

The auristatin is in certain embodiments selected from the group consisting of monomethyl auristatin E (MMAE) and monomethyl auristatin F (MMAF).

The enediyne is in certain embodiments selected from the group consisting of neocarzinostatin, lidamycin (C-1027), calicheamicins, esperamicins, dynemicins and golfomycin A.

The maytansine derivative is in certain embodiments selected from the group consisting of ansamitocin, mertansine (emtansine, DM1) and ravtansine (soravtansine, DM4).

The immune checkpoint inhibitor or antagonist is in certain embodiments selected from the group consisting of inhibitors of CTLA-4 (cytotoxic T-lymphocyte-associated protein 4), such as ipilimumab, tremelimumab, MK-1308, FPT155, PRS010, BMS-986249, BPI-002, CBT509, JS007, ONC392, TE1254, IB1310, BR02001, CG0161, KN044, PBI5D3H5, BCD145, ADU1604, AGEN1884, AGEN1181, CS1002 and CP675206; inhibitors of PD-1 (programmed death 1), such as pembrolizumab, nivolumab, pidilizumab, AMP-224, BMS-936559, cemiplimab and PDR001; inhibitors of PD-L1 (programmed cell death protein 1), such as MDX-1105, MEDI4736, atezolizumab, avelumab, BMS-936559 and durvalumab, inhibitors of PD-L2 (programmed death-ligand 2); inhibitors of KIR (killer-cell immunoglobulin-like receptor), such as lirlumab (IPH2102) and IPH2101; inhibitors of B7-H3, such as MGA271; inhibitors of B7-H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, B1754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; and inhibitors of PVRIG, such as COM701.

The immune agonist is in certain embodiments selected from the group consisting of agonists of CD27, such as recombinant CD70, such as HERA-CD27L, and varlilumab (CDX-1127); agonists of CD28, such as recombinant CD80, recombinant CD86, TGN1412 and FPT155; agonists of CD40, such as recombinant CD40L, CP-870,893, dacetuzumab (SGN-40), Chi Lob 7/4, ADC-1013 and CDX1140; agonists of 4-1BB (CD137), such as recombinant 4-1BBL, urelumab, utomilumab and ATOR-1017; agonists of OX40, such as recombinant OX40L, MEDI0562, GSK3174998, MOXR0916 and PF-04548600; agonists of GITR, such as recombinant GITRL, TRX518, MEDI1873, INCAGN01876, MK-1248, MK-4166, GWN323 and BMS-986156; and agonists of ICOS, such as recombinant ICOSL, JTX-2011 and GSK3359609.

The multi-specific drug is in certain embodiments selected from the group consisting of biologics and small molecule immune checkpoint inhibitors. Examples for biologics are multi-specific immune checkpoint inhibitors, such as CD137/HER2 lipocalin, PD1/LAG3, FS118, XmAb22841 and XmAb20717; and multi-specific immune agonists. Such multi-specific immune agonists may be selected from the group consisting of Ig superfamily agonists, such as ALPN-202; TNF superfamily agonists, such as ATOR-1015, ATOR-1144, ALG.APV-527, lipocalin/PRS-343, PRS344/ONC0055, FAP-CD40 DARPin, MP0310 DARPin, FAP-0X40 DARPin, EGFR-CD40 DARPin, EGFR41BB/CD137 DARPin, EGFR-0X40/DARFPin, HER2-CD40 DARPin, HER2-41BB/CD137 DARPin, HER2-0X40 DARPin, FIBRONECTIN ED-B-CD40 DARPin, FIBRONECTIN ED-B-41BB/CD137 and FIBRONECTIN ED-B-0X40 DARPin; CD3 multispecific agonists, such as blinatumomab, solitomab, MEDI-565, ertumaxomab, anti-HER2/CD3 1Fab-immunoblobulin G TDB, GBR 1302, MGD009, MGD007, EGFRBi, EGFR-CD Probody, RG7802, PF-06863135, PF-06671008, MOR209/ES414, AMG212/BAY2010112 and CD3-5T4; and CD16 multispecific agonists, such as 1633 BiKE, 161533 TriKE, OXS-3550, OXS-C3550, AFM13 and AFM24.

Such immune checkpoint inhibitor or antagonist is in certain embodiments selected from the group consisting of inhibitors of CTLA-4 (cytotoxic T-lymphocyte-associated protein 4), such as ipilimumab, tremelimumab, MK-1308, FPT155, PRS010, BMS-986249, BPI-002, CBT509, JS007, ONC392, TE1254, IBI310, BR02001, CG0161, KN044, PBI5D3H5, BCD145, ADU1604, AGEN1884, AGEN1181, CS1002 and CP675206; inhibitors of PD-1 (programmed death 1), such as pembrolizumab, nivolumab, pidilizumab, AMP-224, BMS-936559, cemiplimab and PDR001; inhibitors of PD-L1 (programmed cell death protein 1), such as MDX-1105, MED14736, atezolizumab, avelumab, BMS-936559 and durvalumab; inhibitors of PD-L2 (programmed death-ligand 2); inhibitors of KIR (killer-cell immunoglobulin-like receptor), such as lirlumab (IPH2102) and IPH2101; inhibitors of B7-H3, such as MGA271; inhibitors of B7-H4, such as FPA150; inhibitors of BTLA (B- and T-lymphocyte attenuator); inhibitors of LAG3 (lymphocyte-activation gene 3), such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 and XmAb22841; inhibitors of TIM-3 (T-cell immunoglobulin and mucin-domain containing-3), such as LY3321367, MBG453, and TSR-022; inhibitors of VISTA (V-domain Ig suppressor of T cell activation), such as JNJ-61610588; inhibitors of ILT2/LILRB1 (Ig-like transcript 2/leukocyte Ig-like receptor 1); inhibitor of ILT3/LILRB4 (Ig-like transcript 3/leukocyte Ig-like receptor 4); inhibitors of ILT4/LILRB2 (Ig-like transcript 4/leukocyte Ig-like receptor 2), such as MK-4830; inhibitors of TIGIT (T cell immunoreceptor with Ig and ITIM domains), such as MK-7684, PTZ-201, RG6058 and COM902; inhibitors of NKG2A, such as IPH-2201; and inhibitors of PVRIG, such as COM701.

In certain embodiments said one or more further drug is an inhibitor of PD-1. In certain embodiments said one or more further drug is an inhibitor of PD-L1.

A moiety -L¹- is conjugated to -D via a functional group of -D, which functional group is in certain embodiments selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, aziridine, amide, imide, imine, urea, amidine, guanidine, sulfonamide, phosphonamide, phosphoramide, hydrazide and selenol. In certain embodiments -L¹- is conjugated to -D via a functional group of -D selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, amidine and aziridine. In certain embodiments -L¹- is conjugated to -D via a functional group of -D selected from the group consisting of hydroxyl, primary amine, secondary amine, amidine and carboxylic acid.

In certain embodiments -L¹- is conjugated to -D via a hydroxyl group of -D. In certain embodiments -L¹- is conjugated to -D via a primary amine group of -D. In certain embodiments -L¹- is conjugated to -D via a secondary amine group of -D. In certain embodiments -L¹- is conjugated to -D via a carboxylic acid group of -D. In certain embodiments -L¹- is conjugated to -D via an amidine group of -D.

The moiety -L¹- may be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide, acylguanidine, acylamidine, carbonate, phosphate, sulfate, urea, hydrazide, thioester, thiophosphate, thiosulfate, sulfonamide, sulfoamidine, sulfaguanidine, phosphoramide, phosphoamidine, phosphoguanidine, phosphonamide, phosphonamidine, phosphonguanidine, phosphonate, borate and imide. In certain embodiments -L¹- is connected to -D through a linkage selected from the group consisting of amide, ester, carbonate, carbamate, acetal, aminal, imine, oxime, hydrazone, disulfide, acylamidine and acylguanidine. In certain embodiments -L¹- is connected to -D through a linkage selected from the group consisting of amide, ester, carbonate, acylamide and carbamate. It is understood that some of these linkages may not be reversible per se, but that in the present invention neighboring groups present in -L¹- render these linkages reversible.

In certain embodiments -L¹- is connected to -D through an ester linkage. In certain embodiments -L¹- is connected to -D through a carbonate linkage. In certain embodiments -L¹- is connected to -D through an acylamidine linkage. In certain embodiments -L¹- is connected to -D through a carbamate linkage. In certain embodiments -L¹- is connected to -D through an amide linkage.

The moiety -L¹- is a linker moiety from which -D is released in its free form, i.e. usually in the form of D-H or D-OH. Such moieties are also referred to as “prodrug linkers” or “reversible prodrug linkers” and are known in the art, such as for example the reversible linker moieties disclosed in WO 2005/099768 A2, WO 2006/136586 A2, WO 2011/089216 A1, WO 2013/024053 A1, WO 2011/012722 A1, WO 2011/089214 A1, WO 2011/089215 A1, WO 2013/024052 A1 and WO 2013/160340 A1, which are incorporated by reference herewith.

In certain embodiments the moiety -L¹- is as disclosed in WO 2009/095479 A2. Accordingly, in certain embodiments the moiety -L¹- is of formula (I):

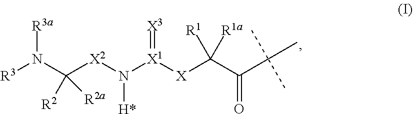

wherein the dashed line indicates the attachment to a nitrogen, hydroxyl or thiol of -D;
—X— is selected from the group consisting of —C(R⁴R^{4a})—, —N(R⁴)—, —O—, —C(R⁴R^{4a})—C(R⁵R^{5a})—, —C(R⁵R^{5a})—C(R⁴R^{4a})—, —C(R⁴R^{4a})—N(R⁶)—, —N(R⁶)—C(R⁴R^{4a})—, —C(R⁴R^{4a})—O—, —O—C(R⁴R^{4a})—, and —C(R⁷R^{7a})—, X¹ is selected from the group consisting of C and S(O);
—X²— is selected from the group consisting of —C(R⁸R^{8a})— and —C(R⁸R^{8a})—C(R⁹R^{9a})—;
═X³ is selected from the group consisting of ═O, ═S, and ═N—CN;
—R¹, —R^{1a}, —R², —R^{2a}, —R⁴, —R^{4a}, —R⁵, —R^{5a}, —R⁶, —R⁸, —R^{8a}, —R⁹ and —R^{9a} are independently selected from the group consisting of —H and C₁₋₆ alkyl;
—R³ and —R^{3a} are independently selected from the group consisting of —H and C₁₋₆ alkyl, provided that in case one or both of —R³ and —R^{3a} are other than —H they are connected to N to which they are attached through an sp³-hybridized carbon atom;
—R⁷ is selected from the group consisting of —N(R¹⁰R^{10a}) and —NR¹⁰—(C═O)—R¹¹;
—R^{7a}, —R¹⁰, —R^{10a} and —R¹¹ are independently selected from the group consisting of —H and C₁₋₆ alkyl;
alternatively, one or more of the pairs —R^{1a}/—R^{4a}, —R^{1a}/—R^{5a}, —R^{1a}/—R^{7a}, —R^{4a}/—R^{5a} and —R^{8a}/—R^{9a} form a chemical bond;
alternatively, one or more of the pairs —R¹/—R^{1a}, —R²/—R^{2a}, —R⁴/—R^{4a}, —R⁵/—R^{5a}, —R⁵/—R^{5a} and —R⁹/—R^{9a} are joined together with the atom to which they are attached to form a C₃₋₁₀ cycloalkyl or 3- to 10-membered heterocyclyl;
alternatively, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹ and —R²/—R³ are joined together with the atoms to which they are attached to form a ring A;
alternatively, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²- and wherein -L¹- is optionally further substituted, provided that the hydrogen marked with the asterisk in formula (I) is not replaced by -L²- or a substituent.

The optional further substituents of -L¹- of formula (I) are as described elsewhere herein.

In certain embodiments -L¹- of formula (I) is not further substituted.

It is understood that if —R³/—R^{3a} of formula (I) are joined together with the nitrogen atom to which they are attached to form a 3- to 10-membered heterocycle, only such 3- to 10-membered heterocycles may be formed in which the atoms directly attached to the nitrogen are sp³-hybridized carbon atoms. In other words, such 3- to 10-membered heterocycle formed by —R³/—R^{3a} together with the nitrogen atom to which they are attached has the following structure:

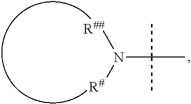

wherein
the dashed line indicates attachment to the rest of -L¹-;
the ring comprises 3 to 10 atoms comprising at least one nitrogen; and R⁴ and R⁴⁴ represent an sp³-hybridized carbon atom.

It is also understood that the 3- to 10-membered heterocycle may be further substituted.

Exemplary embodiments of suitable 3- to 10-membered heterocycles formed by —R³/—R^{3a} of formula (I) together with the nitrogen atom to which they are attached are the following:

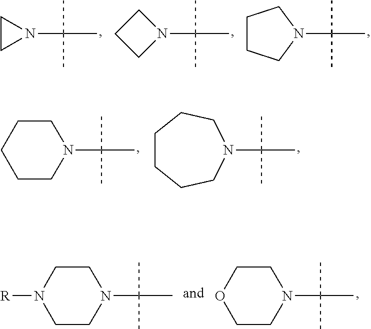

wherein
dashed lines indicate attachment to the rest of the molecule; and
—R is selected from the group consisting of —H and C₁₋₆ alkyl.

-L¹- of formula (I) may optionally be further substituted. In general, any substituent may be used as far as the cleavage principle is not affected, i.e. the hydrogen marked with the asterisk in formula (I) is not replaced and the nitrogen of the moiety

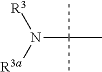

of formula (I) remains part of a primary, secondary or tertiary amine, i.e. —R³ and —R^{3a} are independently of each other —H or are connected to —N< through a sp³-hybridized carbon atom.

In certain embodiments —X— of formula (I) is —C(R⁴R^{4a})—. In certain embodiments —X— of formula (I) is —N(R⁴). In certain embodiments —X— of formula (I) is —O—. In certain embodiments —X— of formula (I) is C(R⁴R^{4a})—C(R⁵R^{5a})—. In certain embodiments —X— of formula (I) is —C(R⁵R^{5a})—C(R⁴R^{4a})—. In certain embodiments —X— of formula (I) is —C(R⁴R^{4a})—N(R⁶)—. In certain embodiments —X— of formula (I) is —N(R⁶)—C(R⁴R^{4a})—. In certain embodiments —X— of formula (I) is —C(R⁴R^{4a})—O—. In certain embodiments —X— of formula (I) is —O—C(R⁴R^{4a})—In certain embodiments —X— of formula (I) is —O—C(R⁴R^{4a})—. In certain embodiments —X— of formula (I) is —C(R⁷R^{7a})—.

In certain embodiments X¹ of formula (I) is C. In certain embodiments X¹ of formula (I) is S(O).

In certain embodiments —X²— of formula (I) is —C(R⁸R^{8a})—. In certain embodiments —X²— of formula (I) is —C(R⁸R^{8a})—.

In certain embodiments ═X³ of formula (I) is ═O. In certain embodiments ═X³ of formula (I) is ═S. In certain embodiments ═X³ of formula (I) is ═N—CN.

In certain embodiments —R¹ of formula (I) is —H. In certain embodiments —R¹ of formula (I) is methyl. In certain embodiments —R¹ of formula (I) is ethyl. In certain embodiments —R^{1a} of formula (I) is —H. In certain embodiments —R^{1a} of formula (I) is methyl. In certain embodiments —R^{1a} of formula (I) is ethyl. In certain embodiments —R² of formula (I) is —H. In certain embodiments —R² of formula (I) is methyl. In certain embodiments —R² of formula (I) is ethyl. In certain embodiments —R^{2a} of formula (I) is —H. In certain embodiments —R^{2a} of formula (I) is methyl. In certain embodiments —R^{2a} of formula (I) is ethyl. In certain embodiments —R³ of formula (I) is —H. In certain embodiments —R³ of formula (I) is methyl. In certain embodiments —R³ of formula (I) is ethyl. In certain embodiments —R^{3a} of formula (I) is —H. In certain embodiments —R^{3a} of formula (I) is methyl. In certain embodiments —R^{3a} of formula (I) is ethyl. In certain embodiments —R⁴ of formula (I) is —H. In certain embodiments —R⁴ of formula (I) is methyl. In certain embodiments —R⁴ of formula (I) is ethyl. In certain embodiments —R^{4a} of formula (I) is —H. In certain embodiments —R^{4a} of formula (I) is methyl. In certain embodiments —R^{4a} of formula (I) is ethyl. In certain embodiments —R⁵ of formula (I) is —H. In certain embodiments —R⁵ of formula (I) is methyl. In certain embodiments —R⁵ of formula (I) is ethyl. In certain embodiments —R^{5a} of formula (I) is —H. In certain embodiments —R^{5a} of formula (I) is methyl. In certain embodiments —R^{5a} of formula (I) is ethyl. In certain embodiments —R⁶ of formula (I) is —H. In certain embodiments —R⁶ of formula (I) is methyl. In certain embodiments —R⁶ of formula (I) is ethyl. In certain embodiments —R⁷ of formula (I) is —N(R¹⁰R^{10a}). In certain embodiments —R⁷ of formula (I) is —NR¹⁰—(C═O)—R¹¹. In certain embodiments —R^{7a} of formula (I) is —H. In certain embodiments —R^{7a} of formula (I) is methyl. In certain embodiments —R^{7a} of formula (I) is ethyl. In certain embodiments —R⁸ of formula (I) is —H. In certain embodiments —R⁸ of formula (I) is methyl. In certain embodiments —R⁸ of formula (I) is ethyl. In certain embodiments —R^{8a} of formula (I) is —H. In certain embodiments —R^{8a} of formula (I) is methyl. In certain embodiments —R^{8a} of formula (I) is ethyl. In certain embodiments —R⁹ of formula (I) is —H. In certain embodiments —R⁹ of formula (I) is methyl. In certain embodiments —R⁹ of formula (I) is ethyl. In certain embodiments —R^{9a} of formula (I) is —H. In certain embodiments —R^{9a} of formula (I) is methyl. In certain embodiments —R^{9a} of formula (I) is ethyl. In certain embodiments —R¹⁰ of formula (I) is —H. In certain embodiments —R¹⁰ of formula (I) is methyl. In certain embodiments —R¹⁰ of formula (I) is ethyl. In certain embodiments —R^{10a} of formula (I) is —H. In certain embodiments —R^{10a} of formula (I) is methyl. In certain embodiments —R^{10a} of formula (I) is ethyl. In certain embodiments —R¹¹ of formula (I) is —H. In certain embodiments —R¹¹ of formula (I) is methyl. In certain embodiments —R¹¹ of formula (I) is ethyl.

In certain embodiments —R¹ of formula (I) is —H, which —H is substituted with -L²-. In certain embodiments —R^{1a} of formula (I) is —H, which —H is substituted with -L²-. In certain embodiments —R² of formula (I) is —H, which —H is substituted with -L²-. In certain embodiments —R^{2a} of formula (I) is —H, which —H is substituted with -L²-. In certain embodiments —R³ of formula (I) is —H, which —H is substituted with -L²-. In certain embodiments —R^{3a} of formula (I) is —H, which —H is substituted with -L²-. In certain embodiments —R⁴ of formula (I) is —H, which —H is substituted with -L²-. In certain embodiments —R⁵ of formula (I) is —H, which —H is substituted with -L²-. In certain embodiments —R^{5a} of formula (I) is —H, which —H is substituted with -L²-. In certain embodiments —R⁶ of formula (I) is —H, which —H is substituted with -L²-. In certain embodiments —R⁷ of formula (I) is —H, which —H is substituted with -L²-. In certain embodiments —R^{7a} of formula (I) is —H, which —H is substituted with -L²-. In certain embodiments —R⁸ of formula (I) is —H, which —H is substituted with -L²-. In certain embodiments —R^{8a} of formula (I) is —H, which —H is substituted with -L²-. In certain embodiments —R⁹ of formula (I) is —H, which —H is substituted with -L²-. In certain embodiments —R^{9a} of formula (I) is —H, which —H is substituted with -L²-. In certain embodiments —R¹⁰ of formula (I) is —H, which —H is substituted with -L²-. In certain embodiments —R¹¹ of formula (I) is —H, which —H is substituted with -L²-.

Another moiety -L¹- is disclosed in WO 2016/020373 A1. Accordingly, in certain embodiments the moiety -L¹- is of formula (II):

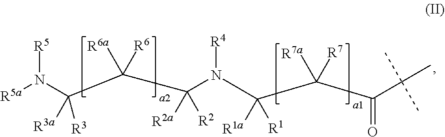

wherein
the dashed line indicates attachment to a primary or secondary amine or hydroxyl of -D by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C≡N, —C(═NR^{B})R^{8a}, —CR⁸(═CR^{8a}R^{8b}), —C≡CR^{B} and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joint together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²- and wherein -L¹- is optionally further substituted.

The optional further substituents of -L¹- of formula (II) are as described elsewhere herein.

In certain embodiments -L¹- of formula (II) is not further substituted.

Additional embodiments for -L¹- are disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

Further embodiments for -L¹- are disclosed in U.S. Pat. No. 8,946,405B2 and U.S. Pat. No. 8,754,190B2, which are herewith incorporated by reference in their entirety. Accordingly, in certain embodiments -L¹- is of formula (III):

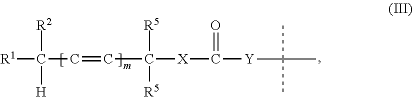

wherein
the dashed line indicates attachment to -D through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring; and
wherein -L¹- is substituted with -L²- and wherein -L¹- is optionally further substituted.

Only in the context of formula (III) the terms used have the following meaning:

The term “alkyl” as used herein includes linear, branched or cyclic saturated hydrocarbon groups of 1 to 8 carbon atoms, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

Another embodiment for -L¹- is disclosed in WO2013/036857A1, which is herewith incorporated by reference in its entirety. Accordingly, in certain embodiments -L¹- is of formula (IV):

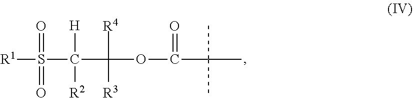

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR %;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl; and
wherein -L¹- is substituted with -L²- and wherein -L¹- is optionally further substituted.

Only in the context of formula (IV) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, preferably 6-10 carbons, including groups such as phenyl, naphthyl, and anthracene “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketne; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

A further embodiment for -L¹- is disclosed in U.S. Pat. No. 7,585,837B2, which is herewith incorporated by reference in its entirety. Accordingly, in certain embodiments -L¹- is of formula (V):

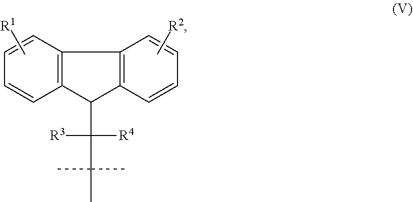

wherein
the dashed line indicates attachment to -D through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl; and
wherein -L¹- is substituted with -L²- and wherein -L¹- is optionally further substituted.

Suitable substituents for formulas (V) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (V) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (V) is not further substituted.

In certain embodiments -L¹- is as disclosed in WO2002/089789A1, which is herewith incorporated by reference in its entirety. Accordingly, in certain embodiments -L¹- is of formula (VI):

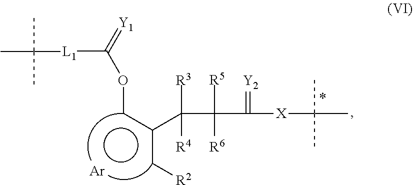

wherein
the dashed line indicates attachment to -D through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VI) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1; and
wherein -L¹- is substituted with -L²- and wherein -L¹- is optionally further substituted.

Only in the context of formula (VI) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substtued cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moeities such as methoxy; and phenoxy includes moieties such as 3-nitrophenoxy. Halo-shall be understood to include fluoro, chloro, iodo and bromo.

In certain embodiments -L¹- of formula (VI) is not further substituted.

In certain embodiments -L¹- comprises a substructure of formula (VII)

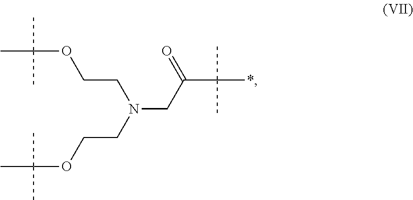

wherein
the dashed line marked with the asterisk indicates attachment to a nitrogen of -D by forming an amide bond;
the unmarked dashed lines indicate attachment to the remainder of -L¹-; and
wherein -L¹- is substituted with -L²- and wherein -L¹- is optionally further substituted.

The optional further substituents of -L¹- of formula (VII) are as described elsewhere herein.

In certain embodiments -L¹- of formula (VII) is not further substituted.

In certain embodiments -L¹- comprises a substructure of formula (VIII)

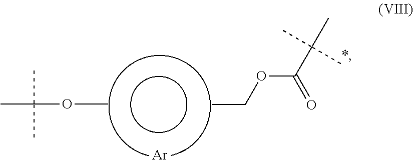

wherein
the dashed line marked with the asterisk indicates attachment to a nitrogen of -D by forming a carbamate bond;
the unmarked dashed lines indicate attachment to the remainder of -L¹-; and
wherein -L¹- is substituted with -L²- and wherein -L¹- is optionally further substituted.

The optional further substituents of -L¹- of formula (VIII) are as described above.

In certain embodiments -L¹- of formula (VIII) is not further substituted.

In one embodiment -L¹- is of formula (VIII-a):

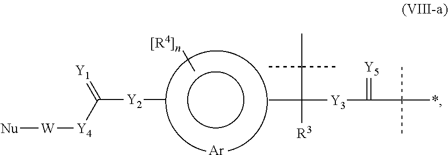

wherein

the dashed line marked with the asterisk indicates attachment to a nitrogen of -D and the unmarked dashed line indicates attachment to -L²-;

n is 0, 1, 2, 3, or 4;

═Y₁, ═Y₅ are independently of each other selected from the group consisting of ═O and ═S;

—Y₂— is selected from the group consisting of —O— and —S—;

—Y₃— is selected from the group consisting of —O— and —S—;

—Y₄— is selected from the group consisting of —O—, —NR⁵— and —C(R⁶R^{6a})—;

—R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;

—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;

—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—; —Nu is a nucleophile selected from the group consisting of —N(R⁷R^{7a}), —N(R⁷OH), —N(R⁷)—N(R^{7a}R^{7b}), —S(R⁷), —COOH,

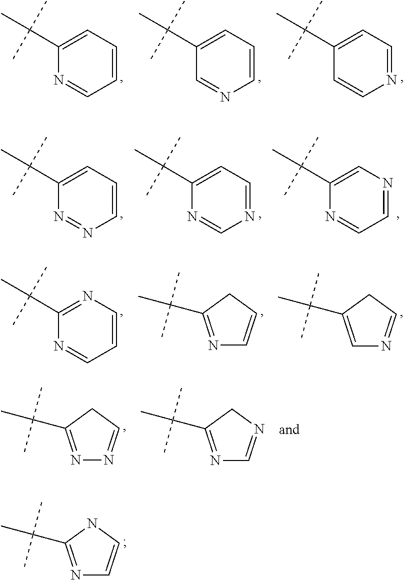

—Ar— is selected from the group consisting of

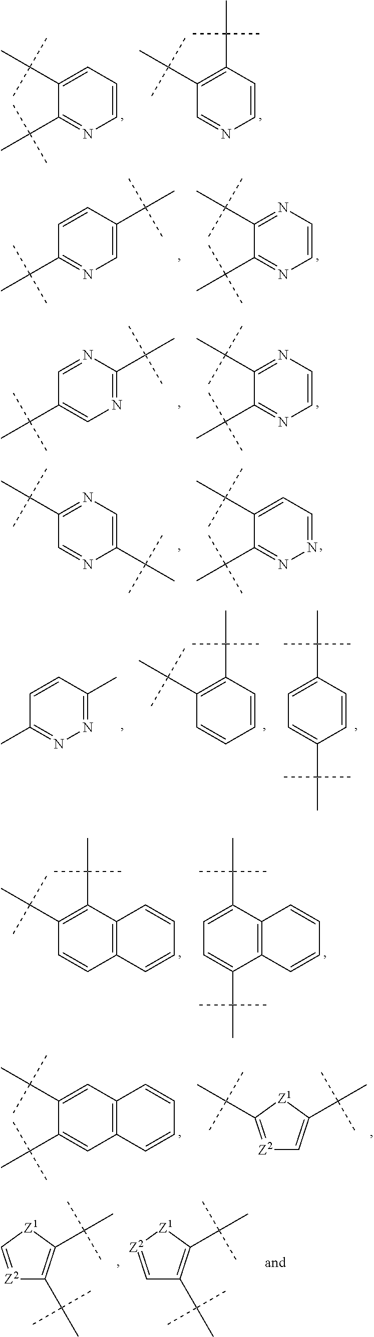

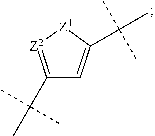

wherein
dashed lines indicate attachment to the remainder of -L¹-,
—Z¹— is selected from the group consisting of —O—, —S— and —N(R⁷)—, and
—Z²— is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein -L¹- is optionally further substituted.

In one embodiment -L¹- of formula (VIII-a) is not further substituted.

In another embodiment -L¹- is of formula (VIII-b):

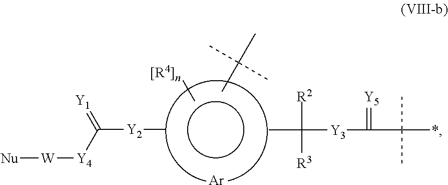

wherein

the dashed line marked with the asterisk indicates attachment to a nitrogen of -D and the unmarked dashed line indicates attachment to -L²-;

n is 0, 1, 2, 3, or 4;

═Y₁, ═Y₅ are independently of each other selected from the group consisting of ═O and ═S;

—Y₂— is selected from the group consisting of —O— and —S—;

—Y₃— is selected from the group consisting of —O— and —S—;

—Y₄— is selected from the group consisting of —O—, —NR⁵— and —C(R⁶R^{6a})—;

—R², —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;

—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;

—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—; —Nu is a nucleophile selected from the group consisting of —N(R⁷R^{7a}), —N(R⁷OH), —N(R⁷)—N(R^{7a}R^{7b}), —S(R⁷), —COOH,

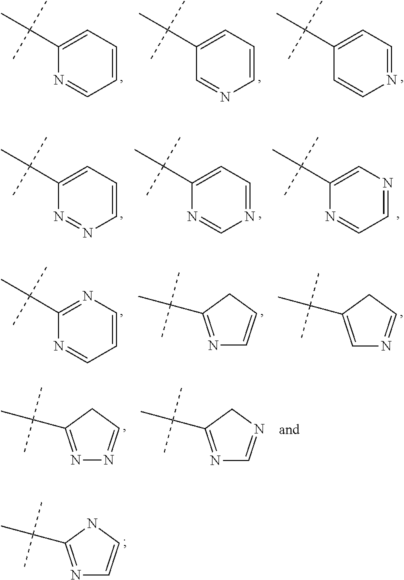

—Ar— is selected from the group consisting of

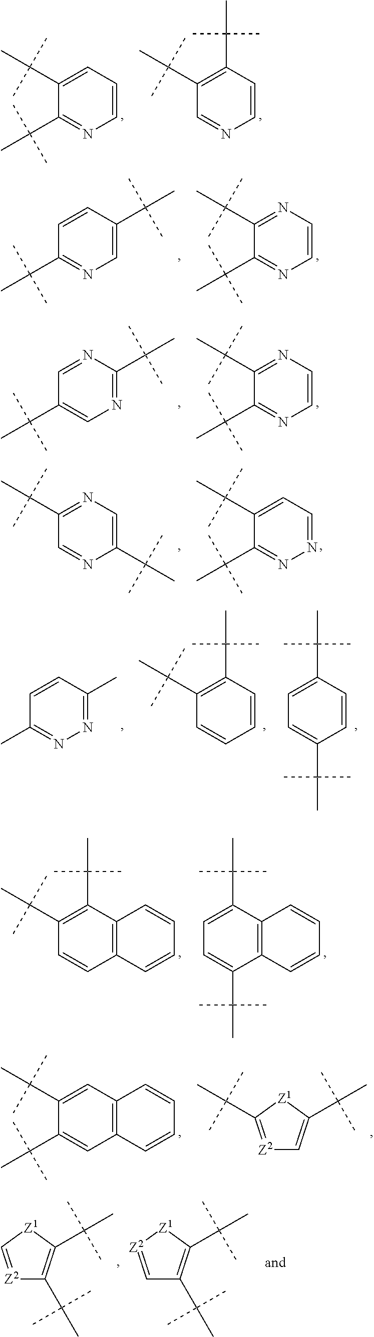

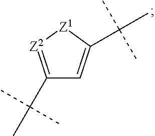

wherein
dashed lines indicate attachment to the remainder of -L¹-,
—Z¹— is selected from the group consisting of —O—, —S— and —N(R⁷)—, and
—Z²— is —N(R⁷)—; and
—R⁷, —R^{7a}, —R^{7b} are independently of each other selected from the group consisting of —H, C₁₋₆ alkyl, C₂₋₆ alkenyl and C₂₋₆ alkynyl;
wherein -L¹- is optionally further substituted.

In one embodiment -L¹- of formula (VIII-b) is not further substituted.

In certain embodiments -L¹- is of formula (IXi)

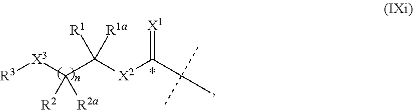

wherein
the dashed line indicates the attachment to the π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²— is selected from the group consisting of —O—, —S—, —N(R⁵)— and —C(R⁶)(R^{6a});
—X³— is selected from the group consisting of

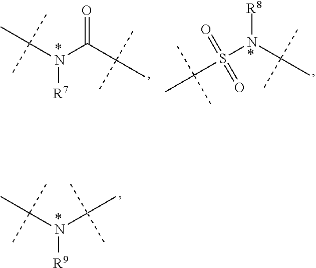

—C(R¹⁰)(R^{10a})—, —C(R¹¹)(R^{11a})—C(R¹²)(R^{12a})—, —O— and —C(O)—; —R¹, —R^{1a}, —R⁶, —R^{6a}, —R¹⁰, —R^{10a}, —R¹¹, —R^{1a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
   wherein each T is independently optionally substituted with one or more —R¹³, which are the same or different;
   wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
   wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent —R², —R⁶/—R^{6a}, —R¹⁰/—R^{10a}, —R¹¹/—R^{11a}, —R¹²/—R^{12a} and —R³/—R⁹ are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R²/—R⁵, —R³/—R^{6a}, —R⁴/—R⁵, —R⁴/—R⁶, —R⁵/—R¹⁰, —R⁶/—R¹⁰ and —R¹¹/—R¹² are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that if —X²— is —N(R⁵)—, —X³— is selected from the group consisting of

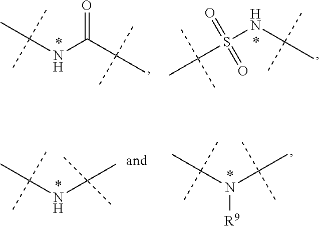

and the distance between the nitrogen atom marked with an asterisk and the carbon atom marked with an asterisk in formula (IXi) is 5, 6 or 7 atoms and if present the carbon-carbon double bond formed between —R¹ and —R² or two adjacent —R² is in a cis configuration; and
wherein -L¹- is substituted with -L²- and wherein -L¹- is optionally further substituted.

In certain embodiments -L¹- is of formula (IX)

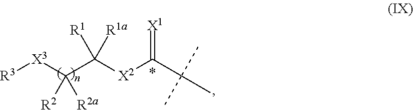

wherein
the dashed line indicates the attachment to a π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²— is selected from the group consisting of —O—, —S—, —N(R⁵)— and —C(R⁶)(R^{6a})—;
—X³— is selected from the group consisting of

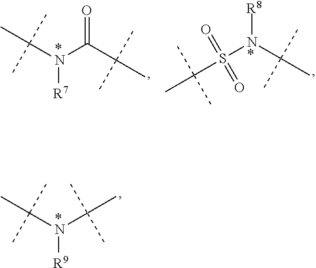

—C(R¹⁰)(R^{10a})—, —C(R¹¹)(R^{11a})—C(R¹²)(R^{12a})—, —O— and —C(O)—;
—R¹, —R^{1a}, —R⁶, —R^{6a}, —R¹⁰, —R^{10a}, —R¹¹, —R^{1a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
   wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
   wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent R², —R⁶/—R^{6a}, —R¹⁰/—R^{10a}, —R¹¹/—R^{11a} and —R¹²/—R^{12a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R³/—R^{6a}, —R⁴/—R⁵, —R⁴/—R⁶, —R⁵/—R¹⁰, and —R⁶/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that if —X²— is —N(R⁵)—, —X³— is selected from the group consisting of

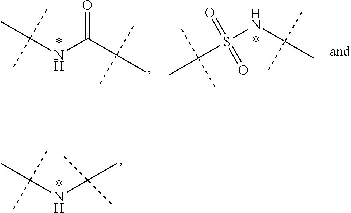

and the distance between the nitrogen atom marked with an asterisk and the carbon atom marked with an asterisk in formula (IX) is 5, 6 or 7 atoms and if present the carbon-carbon double bond formed between —R¹ and —R² or two adjacent —R² is in a cis configuration; and
wherein -L¹- is substituted with -L²- and wherein -L¹- is optionally further substituted.

It is understood that two adjacent —R² in formula (IXi) or (IX) can only exist if n is at least 2.

It is understood that the expression “distance between the nitrogen atom marked with an asterisk and the carbon atom marked with an asterisk” refers to the total number of atoms in the shortest distance between the nitrogen and carbon atoms marked with the asterisk and also includes the nitrogen and carbon atoms marked with the asterisk. For example, in the structure below, n is 1 and the distance between the nitrogen marked with an asterisk and the carbon marked with an asterisk is 5:

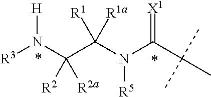

and in the structure below, n is 2, —R¹ and —R^{1a} form a cyclohexyl and the distance between the nitrogen marked with an asterisk and the carbon marked with an asterisk is 6:

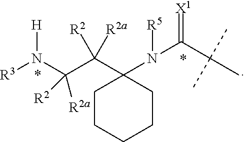

The optional further substituents of -L¹- of formula (IXi) or (IX) are as described elsewhere herein.

In certain embodiments -L¹- of formula (IXi) or (IX) is not further substituted.

In certain embodiments ═X¹ of formula (IXi) or (IX) is ═O. In certain embodiments ═X¹ of formula (IXi) or (IX) is ═S. In certain embodiments ═X¹ of formula (IXi) or (IX) is ═N(R⁴).

In certain embodiments —X²— of formula (IXi) or (IX) is —O—. In certain embodiments —X²— of formula (IXi) or (IX) is —S—. In certain embodiments —X²— of formula (IXi) or (IX) is —N(R⁵)—.

In certain embodiments, —X²— of formula (IXi) or (IX) is —C(R⁶)(R^{6a})—.

In certain embodiments —X³— of formula (IM) or (IX) is

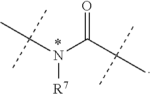

In certain embodiments —X³— of formula (IXi) or (IX) is

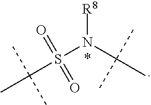

In certain embodiments —X³— of formula (IXi) or (IX) is

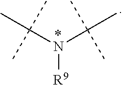

In certain embodiments —X³— of formula (IXi) or (IX) is —C(R¹⁰)(R^{10a})—. In certain embodiments —X³— of formula (IXi) or (IX) is —C(R¹¹)(R^{11a})—C(R¹²)(R^{12a})—. In certain embodiments —X³— of formula (IXi) or (IX) is —O—. In certain embodiments —X³— of formula (IXi) or (IX) is —C(O)—.

In certain embodiments —X²— of formula (IXi) or (IX) is —N(R⁵)—, —X³— is

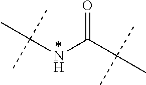

and the distance between the nitrogen atom marked with an asterisk and the carbon atom marked with an asterisk in formula (IXi) or (IX) is 5 atoms.

In certain embodiments —X²— of formula (IXi) or (IX) is —N(R⁵)—, —X³— is

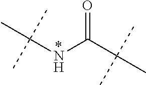

and the distance between the nitrogen atom marked with an asterisk and the carbon atom marked with an asterisk in formula (IXi) or (IX) is 6 atoms.

In certain embodiments —X²— of formula (IXi) or (IX) is —N(R⁵)—, —X³— is

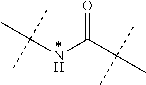

and the distance between the nitrogen atom marked with an asterisk and the carbon atom marked with an asterisk in formula (IXi) or (IX) is 7 atoms.

In certain embodiments —X²— of formula (IXi) or (IX) is —N(R⁵)—, —X³— is

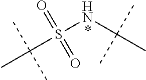

and the distance between the nitrogen atom marked with an asterisk and the carbon atom marked with an asterisk in formula (IXi) or (IX) is 5 atoms.

In certain embodiments —X²— of formula (IXi) or (IX) is —N(R⁵)—, —X³— is

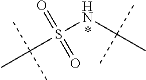

and the distance between the nitrogen atom marked with an asterisk and the carbon atom marked with an asterisk in formula (IXi) or (IX) is 6 atoms.

In certain embodiments —X²— of formula (IXi) or (IX) is —N(R⁵)—, —X³— is

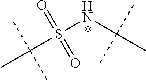

and the distance between the nitrogen atom marked with an asterisk and the carbon atom marked with an asterisk in formula (IXi) or (IX) is 7 atoms.

In certain embodiments —X²— of formula (IXi) or (IX) is —N(R⁵)—, —X³— is

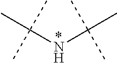

and the distance between the nitrogen atom marked with an asterisk and the carbon atom marked with an asterisk in formula (IXi) or (IX) is 5 atoms.

In certain embodiments —X²— of formula (IXi) or (IX) is —N(R⁵)—, —X³— is

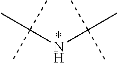

and the distance between the nitrogen atom marked with an asterisk and the carbon atom marked with an asterisk in formula (IXi) or (IX) is 6 atoms.

In certain embodiments —X²— of formula (IXi) or (IX) is —N(R⁵)—, —X³— is

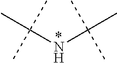

and the distance between the nitrogen atom marked with an asterisk and the carbon atom marked with an asterisk in formula (IXi) or (IX) is 7 atoms.

In certain embodiments, —X²— of formula (IXi) is —N(R⁵)—, —X³— is

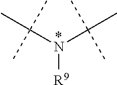

and the distance between the nitrogen atom marked with an asterisk and the carbon atom marked with an asterisk in formula (I) is 5 atoms.

In certain embodiments, —X²— of formula (IXi) is —N(R⁵)—, —X³— is

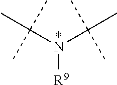

and the distance between the nitrogen atom marked with an asterisk and the carbon atom marked with an asterisk in formula (I) is 6 atoms.

In certain embodiments, —X²— of formula (IXi) is —N(R⁵)—, —X³— is

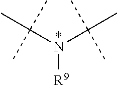

and the distance between the nitrogen atom marked with an asterisk and the carbon atom marked with an asterisk in formula (I) is 7 atoms.

In certain embodiments ═X¹ of formula (IXi) or (IX) is ═O, —X²— of formula (IXi) or (IX) is —C(R⁶)(R^{6a})—, —X³— of formula (IXi) or (IX) is

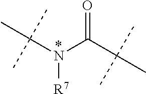

and —R³ of formula (IXi) or (IX) does not comprise an amine.

In certain embodiments —R¹, —R^{1a}, —R⁶, —R¹⁰, —R^{10a}, —R¹¹, —R¹¹, —R¹², —R^{12a} and each of —R² and —R^{2a} of formula (IXi) or (IX) are independently selected from the group consisting of —H, —C(O)OH, halogen, —CN, —OH, C₁₋₆ alkyl, C₂₋₆ alkenyl and C₂₋₆ alkynyl.

In certain embodiments —R¹ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹ of formula (IXi) or (IX) is selected from the group consisting of —H, —C(O)OH, —OH and C₁₋₆ alkyl. In certain embodiments —R¹ of formula (IXi) or (IX) is —H. In certain embodiments —R¹ of formula (IXi) or (IX) is —C(O)OH. In certain embodiments —R¹ of formula (IXi) or (IX) is halogen. In certain embodiments —R¹ of formula (IXi) or (IX) is —F. In certain embodiments —R¹ of formula (IXi) or (IX) is —CN. In certain embodiments —R¹ of formula (IXi) or (IX) is —OH. In certain embodiments —R¹ of formula (IXi) or (IX) is C₁₋₆ alkyl. In certain embodiments —R¹ of formula (IXi) or (IX) is C₂₋₆ alkenyl.

In certain embodiments —R¹ of formula (IXi) or (IX) is C₂₋₆ alkynyl. In certain embodiments —R¹ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In this case it is understood that —R¹/—R^{1a} may optionally be joined together with the atom to which they are attached to form a C₃₋₁₀ cycloalkyl and that one or more of the pairs —R¹/—R², —R¹/—R⁵, —R¹/—R⁶, —R¹/—R⁹ and —R¹/—R¹⁰ may optionally be joined together with the atoms to which they are attached to form a ring -A-, wherein -A- is used as defined for formula (IXi) or (IX).

In certain embodiments —R^{1a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{1a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{1a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{1a} of formula (IXi) or (IX) is selected from the group consisting of —H, —C(O)OH, —OH and C₁₋₆ alkyl. In certain embodiments —R^{1a} of formula (IXi) or (IX) is —H. In certain embodiments —R^{1a} of formula (IXi) or (IX) is —C(O)OH. In certain embodiments, —R^{1a} of formula (IXi) or (IX) is halogen. In certain embodiments —R^{1a} of formula (IXi) or (IX) is —F. In certain embodiments —R^{1a} of formula (IXi) or (IX) is —CN. In certain embodiments —R^{1a} of formula (IXi) or (IX) is —OH. In certain embodiments —R^{1a} of formula (IXi) or (IX) is C₁₋₆ alkyl. In certain embodiments —R^{1a} of formula (IXi) or (IX) is C₂₋₆ alkenyl. In certain embodiments —R^{1a} of formula (IXi) or (IX) is C₂₋₆ alkynyl. In certain embodiments —R^{1a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R⁶ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R⁶ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R⁶ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R⁶ of formula (IXi) or (IX) is selected from the group consisting of —H, —C(O)OH, —OH and C₁₋₆ alkyl. In certain embodiments —R⁶ of formula (IXi) or (IX) is —H. In certain embodiments —R⁶ of formula (IXi) or (IX) is —C(O)OH. In certain embodiments —R⁶ of formula (IXi) or (IX) is halogen. In certain embodiments —R⁶ of formula (IXi) or (IX) is —F. In certain embodiments —R⁶ of formula (IXi) or (IX) is —CN. In certain embodiments —R⁶ of formula (IXi) or (IX) is —OH. In certain embodiments —R⁶ of formula (IXi) or (IX) is C₁₋₆ alkyl. In certain embodiments —R⁶ of formula (IXi) or (IX) is C₂₋₆ alkenyl. In certain embodiments —R⁶ of formula (IXi) or (IX) is C₂₋₆ alkynyl. In certain embodiments —R⁶ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{6a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{6a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{6a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{6a} of formula (IXi) or (IX) is selected from the group consisting of —H, —C(O)OH, —OH and C₁₋₆ alkyl. In certain embodiments —R^{6a} of formula (IXi) or (IX) is —H. In certain embodiments —R^{6a} of formula (IXi) or (IX) is —C(O)OH. In certain embodiments, —R^{6a} of formula (IXi) or (IX) is halogen. In certain embodiments —R^{6a} of formula (IXi) or (IX) is —F. In certain embodiments —R^{6a} of formula (IXi) or (IX) is —CN. In certain embodiments —R^{6a} of formula (IXi) or (IX) is —OH. In certain embodiments —R^{6a} of formula (IXi) or (IX) is C₁₋₆ alkyl. In certain embodiments —R^{6a} of formula (IXi) or (IX) is C₂₋₆ alkenyl. In certain embodiments —R^{6a} of formula (IXi) or (IX) is C₂₋₆ alkynyl. In certain embodiments —R^{6a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹⁰ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹⁰ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹⁰ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹⁰ of formula (IXi) or (IX) is selected from the group consisting of —H, —C(O)OH, —OH and C₁₋₆ alkyl. In certain embodiments —R¹⁰ of formula (IXi) or (IX) is —H. In certain embodiments —R¹⁰ of formula (IXi) or (IX) is —C(O)OH. In certain embodiments —R¹⁰ of formula (IXi) or (IX) is halogen. In certain embodiments —R¹⁰ of formula (IXi) or (IX) is —F. In certain embodiments —R¹⁰ of formula (IXi) or (IX) is —CN. In certain embodiments —R¹⁰ of formula (IXi) or (IX) is —OH. In certain embodiments —R¹⁰ of formula (IXi) or (IX) is C₁₋₆ alkyl. In certain embodiments —R¹⁰ of formula (IXi) or (IX) is C₂₋₆ alkenyl. In certain embodiments —R¹⁰ of formula (IXi) or (IX) is C₂₋₆ alkynyl. In certain embodiments —R¹⁰ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹¹ of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹¹ of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹¹ of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹¹ of formula (IXi) or (IX) is selected from the group consisting of —H, —C(O)OH, —OH and C₁₋₆ alkyl. In certain embodiments —R¹¹ of formula (IXi) or (IX) is —H. In certain embodiments —R¹¹ of formula (IXi) or (IX) is —C(O)OH. In certain embodiments —R¹¹ of formula (IXi) or (IX) is halogen. In certain embodiments —R¹¹ of formula (IXi) or (IX) is —F. In certain embodiments —R¹¹ of formula (IXi) or (IX) is —CN. In certain embodiments —R¹¹ of formula (IXi) or (IX) is —OH. In certain embodiments —R¹¹ of formula (IXi) or (IX) is C₁₋₆ alkyl. In certain embodiments —R¹¹ of formula (IX) is C₂₋₆ alkenyl. In certain embodiments —R¹¹ of formula (IXi) or (IX) is C₂₋₆ alkynyl. In certain embodiments —R¹¹ of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{11a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{11a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{11a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{11a} of formula (IXi) or (IX) is selected from the group consisting of —H, —C(O)OH, —OH and C₁₋₆ alkyl. In certain embodiments —R^{11a} of formula (IXi) or (IX) is —H. In certain embodiments —R^{11a} of formula (IXi) or (IX) is —C(O)OH. In certain embodiments —R^{11a} of formula (IXi) or (IX) is halogen. In certain embodiments —R^{11a} of formula (IXi) or (IX) is —F. In certain embodiments —R^{11a} of formula (IXi) or (IX) is —CN. In certain embodiments —R^{11a} of formula (IXi) or (IX) is —OH. In certain embodiments —R^{11a} of formula (IXi) or (IX) is C₁₋₆ alkyl. In certain embodiments —R^{11a} of formula (IXi) or (IX) is C₂₋₆ alkenyl. In certain embodiments —R^{11a} of formula (IXi) or (IX) is C₂₋₆ alkynyl. In certain embodiments —R^{11a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹² of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R¹² of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R¹² of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R¹² of formula (IXi) or (IX) is selected from the group consisting of —H, —C(O)OH, —OH and C₁₋₆ alkyl. In certain embodiments —R¹² of formula (IXi) or (IX) is —H. In certain embodiments —R¹² of formula (IXi) or (IX) is —C(O)OH. In certain embodiments —R¹² of formula (IXi) or (IX) is halogen. In certain embodiments —R¹² of formula (IXi) or (IX) is —F. In certain embodiments —R¹² of formula (IXi) or (IX) is —CN. In certain embodiments —R¹² of formula (IXi) or (IX) is —OH. In certain embodiments —R¹² of formula (IXi) or (IX) is C₁₋₆ alkyl. In certain embodiments —R¹² of formula (IXi) or (IX) is C₂₋₆ alkenyl. In certain embodiments —R¹² of formula (IXi) or (IX) is C₂₋₆ alkynyl. In certain embodiments —R¹² of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{12a} of formula (IXi) or (IX) is selected from the group consisting of —H, —C(O)OH, halogen, —CN, —OH, C₁₋₆ alkyl, C₂₋₆ alkenyl and C₂₋₆ alkynyl. In certain embodiments —R^{12a} of formula (IXi) or (IX) is selected from the group consisting of —H, —C(O)OH, —CN, —OH, C₁₋₆ alkyl, C₂₋₆ alkenyl and C₂₋₆ alkynyl. In certain embodiments —R^{12a} of formula (IXi) or (IX) is selected from the group consisting of —H, —C(O)OH, halogen, —OH, C₁₋₆ alkyl, C₂₋₆ alkenyl and C₂₋₆ alkynyl. In certain embodiments —R^{12a} of formula (IXi) or (IX) is selected from the group consisting of —H, —C(O)OH, —OH and C₁₋₆ alkyl. In certain embodiments —R^{12a} of formula (IXi) or (IX) is —H. In certain embodiments —R^{12a} of formula (IXi) or (IX) is —C(O)OH. In certain embodiments —R^{12a} of formula (IXi) or (IX) is halogen. In certain embodiments —R^{12a} of formula (IXi) or (IX) is —F. In certain embodiments —R^{12a} of formula (IXi) or (IX) is —CN. In certain embodiments —R^{12a} of formula (IXi) or (IX) is —OH. In certain embodiments —R^{12a} of formula (IXi) or (IX) is C₁₋₆ alkyl. In certain embodiments —R^{12a} of formula (IXi) or (IX) is C₂₋₆ alkenyl. In certain embodiments —R^{12a} of formula (IXi) or (IX) is C₂₋₆ alkynyl. In certain embodiments —R^{12a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments each of —R² of formula (IXi) or (IX) is independently selected from the group consisting of —H, —C(O)OH, halogen, —CN, —OH, C₁₋₆ alkyl, C₂₋₆ alkenyl and C₂₋₆ alkynyl. In certain embodiments each of —R² of formula (IXi) or (IX) is independently selected from the group consisting of —H, —C(O)OH, —CN, —OH, C₁₋₆ alkyl, C₂₋₆ alkenyl and C₂₋₆ alkynyl. In certain embodiments each of —R² of formula (IXi) or (IX) is independently selected from the group consisting of —H, —C(O)OH, halogen, —OH, C₁₋₆ alkyl, C₂₋₆ alkenyl and C₂₋₆ alkynyl. In certain embodiments each of —R² of formula (IXi) or (IX) is independently selected from the group consisting of —H, —C(O)OH, —OH and C₁₋₆ alkyl. In certain embodiments each of —R² of formula (IXi) or (IX) is —H. In certain embodiments each of —R² of formula (IXi) or (IX) is —C(O)OH. In certain embodiments each of —R² of formula (IXi) or (IX) is halogen. In certain embodiments each of —R² of formula (IXi) or (IX) is —F. In certain embodiments each of —R² of formula (IXi) or (IX) is —CN. In certain embodiments each of —R² of formula (IXi) or (IX) is —OH. In certain embodiments each of —R² of formula (IXi) or (IX) is C₁₋₆ alkyl. In certain embodiments each of —R² of formula (IXi) or (IX) is C₂₋₆ alkenyl.

In certain embodiments each of —R² of formula (IXi) or (IX) is C₂₋₆ alkynyl. In certain embodiments each of —R² of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a ring -A-, wherein -A- is used as defined in formula (IX) or (IXi).

In certain embodiments each of —R^{2a} of formula (IXi) or (IX) is independently selected from the group consisting of —H, —C(O)OH, halogen, —CN, —OH, C₁₋₆ alkyl, C₂₋₆ alkenyl and C₂₋₆ alkynyl. In certain embodiments each of —R^{2a} of formula (IXi) or (IX) is independently selected from the group consisting of —H, —C(O)OH, —CN, —OH, C₁₋₆ alkyl, C₂₋₆ alkenyl and C₂₋₆ alkynyl. In certain embodiments each of —R^{2a} of formula (IXi) or (IX) is independently selected from the group consisting of —H, —C(O)OH, halogen, —OH, C₁₋₆ alkyl, C₂₋₆ alkenyl and C₂₋₆ alkynyl. In certain embodiments each of —R^{2a} of formula (IXi) or (IX) is independently selected from the group consisting of —H, —C(O)OH, —OH and C₁₋₆ alkyl. In certain embodiments each of —R^{2a} of formula (IXi) or (IX) is —H. In certain embodiments each of —R^{2a} of formula (IXi) or (IX) is —C(O)OH. In certain embodiments each of —R^{2a} of formula (IXi) or (IX) is halogen. In certain embodiments each of —R^{2a} of formula (IXi) or (IX) is —F. In certain embodiments each of —R^{2a} of formula (IXi) or (IX) is —CN. In certain embodiments each of —R^{2a} of formula (IXi) or (IX) is —OH. In certain embodiments each of —R^{2a} of formula (IXi) or (IX) is C₁₋₆ alkyl. In certain embodiments each of —R^{2a} of formula (IXi) or (IX) is C₂₋₆ alkenyl. In certain embodiments each of —R^{2a} of formula (IXi) or (IX) is C₂₋₆ alkynyl. In certain embodiments each of —R^{2a} of formula (IXi) or (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R³, —R⁴, —R⁵, —R⁷, —R⁸ and —R⁹ of formula (IXi) or (IX) are independently selected from the group consisting of —H, -T, —CN, C₁₋₆ alkyl, C₂₋₆ alkenyl and C₂₋₆ alkynyl. In certain embodiments —R³, —R⁴, —R⁵, —R⁷, —R⁸ and —R⁹ of formula (IXi) or (IX) are independently selected from the group consisting of —H, -T, —CN, C₁₋₆ alkyl and C₂₋₆ alkenyl. In certain embodiments —R³, —R⁴, —R⁵, —R⁷, —R⁸ and —R⁹ of formula (IXi) or (IX) are independently selected from the group consisting of —H, -T, —CN and C₁₋₆ alkyl. In certain embodiments —R³, —R⁴, —R⁵, —R⁷, —R⁸ and —R⁹ of formula (IXi) or (IX) are independently selected from the group consisting of —H, -T and C₁₋₆ alkyl. In certain embodiments —R³, —R⁴, —R⁵, —R⁷, —R⁸ and —R⁹ of formula (IXi) or (IX) are independently selected from the group consisting of —H and C₁₋₆ alkyl.

In certain embodiments —R³ of formula (IXi) or (IX) is selected from the group consisting of —H, -T, —CN, C₁₋₆ alkyl, C₂₋₆ alkenyl and C₂₋₆ alkynyl. In certain embodiments —R³ of formula (IXi) or (IX) is —H. In certain embodiments —R³ of formula (IXi) or (IX) is -T. In certain embodiments —R³ of formula (IXi) or (IX) is —CN. In certain embodiments —R³ of formula (IXi) or (IX) is C₁₋₆ alkyl. In certain embodiments —R³ of formula (IXi) or (IX) is C₂₋₆ alkenyl. In certain embodiments —R³ of formula (IXi) or (IX) is C₂₋₆ alkynyl.

In certain embodiments —R⁴ of formula (IXi) or (IX) is selected from the group consisting of —H, -T, —CN, C₁₋₆ alkyl, C₂₋₆ alkenyl and C₂₋₆ alkynyl. In certain embodiments —R⁴ of formula (IXi) or (IX) is —H. In certain embodiments —R⁴ of formula (IXi) or (IX) is -T. In certain embodiments —R⁴ of formula (IXi) or (IX) is —CN. In certain embodiments —R⁴ of formula (IXi) or (IX) is C₁₋₆ alkyl. In certain embodiments —R⁴ of formula (IXi) or (IX) is C₂₋₆ alkenyl. In certain embodiments —R⁴ of formula (IXi) or (IX) is C₂₋₆ alkynyl.

In certain embodiments —R⁵ of formula (IXi) or (IX) is selected from the group consisting of —H, -T, —CN, C₁₋₆ alkyl, C₂₋₆ alkenyl and C₂₋₆ alkynyl. In certain embodiments —R⁵ of formula (IXi) or (IX) is —H. In certain embodiments —R⁵ of formula (IXi) or (IX) is -T. In certain embodiments —R⁵ of formula (IXi) or (IX) is —CN. In certain embodiments —R⁵ of formula (IXi) or (IX) is C₁₋₆ alkyl. In certain embodiments —R⁵ of formula (IXi) or (IX) is C₂₋₆ alkenyl. In certain embodiments —R⁵ of formula (IXi) or (IX) is C₂₋₆ alkynyl.

In certain embodiments —R⁷ of formula (IXi) or (IX) is selected from the group consisting of —H, -T, —CN, C₁₋₆ alkyl, C₂₋₆ alkenyl and C₂₋₆ alkynyl. In certain embodiments —R⁷ of formula (IXi) or (IX) is —H. In certain embodiments —R⁷ of formula (IXi) or (IX) is -T. In certain embodiments —R⁷ of formula (IXi) or (IX) is —CN. In certain embodiments —R⁷ of formula (IXi) or (IX) is C₁₋₆ alkyl. In certain embodiments —R⁷ of formula (IXi) or (IX) is C₂₋₆ alkenyl. In certain embodiments —R⁷ of formula (IXi) or (IX) is C₂₋₆ alkynyl.

In certain embodiments —R⁸ of formula (IXi) or (IX) is selected from the group consisting of —H, -T, —CN, C₁₋₆ alkyl, C₂₋₆ alkenyl and C₂₋₆ alkynyl. In certain embodiments —R⁸ of formula (IXi) or (IX) is —H. In certain embodiments —R⁸ of formula (IXi) or (IX) is -T. In certain embodiments —R⁸ of formula (IXi) or (IX) is —CN. In certain embodiments —R⁸ of formula (IXi) or (IX) is C₁₋₆ alkyl. In certain embodiments —R⁸ of formula (IXi) or (IX) is C₂₋₆ alkenyl. In certain embodiments —R⁸ of formula (IXi) or (IX) is C₂₋₆ alkynyl.

In certain embodiments —R⁹ of formula (IXi) or (IX) is selected from the group consisting of —H, -T, —CN, C₁₋₆ alkyl, C₂₋₆ alkenyl and C₂₋₆ alkynyl. In certain embodiments —R⁹ of formula (IXi) or (IX) is —H. In certain embodiments —R⁹ of formula (IXi) or (IX) is -T. In certain embodiments —R⁹ of formula (IXi) or (IX) is —CN. In certain embodiments —R⁹ of formula (IXi) or (IX) is C₁₋₆ alkyl. In certain embodiments —R⁹ of formula (IXi) or (IX) is C₂₋₆ alkenyl. In certain embodiments —R⁹ of formula (IXi) or (IX) is C₂₋₆ alkynyl.

In certain embodiments T of formula (IXi) or (IX) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments T of formula (IXi) or (IX) is phenyl. In certain embodiments T of formula (IXi) or (IX) is naphthyl. In certain embodiments T of formula (IXi) or (IX) is indenyl. In certain embodiments T of formula (IXi) or (IX) is indanyl. In certain embodiments T of formula (IXi) or (IX) is tetralinyl. In certain embodiments T of formula (IXi) or (IX) is C₃₋₁₀ cycloalkyl. In certain embodiments T of formula (IXi) or (IX) is 3- to 10-membered heterocyclyl. In certain embodiments T of formula (IXi) or (IX) is 8- to 11-membered heterobicyclyl.

In certain embodiments T of formula (IXi) or (IX) is substituted with one or more —R¹³ of formula (IXi) or (IX), which are the same or different.

In certain embodiments T of formula (IXi) or (IX) is substituted with one —R¹³ of formula (IXi) or (IX).

In certain embodiments T of formula (IXi) or (IX) is not substituted with —R¹³ of formula (IXi) or (IX).

In certain embodiments —R¹³ of formula (IXi) or (IX) is selected from the group consisting of —H, —NO₂, —OCH₃, —CN, —N(R¹⁴)(R^{14a}), —OH, —C(O)OH and C₁₋₆ alkyl.

In certain embodiments —R¹³ of formula (IXi) or (IX) is —H. In certain embodiments —R¹³ of formula (IXi) or (IX) is —NO₂. In certain embodiments —R¹³ of formula (IXi) or (IX) is —OCH₃.

In certain embodiments —R¹³ of formula (IX) is —CN. In certain embodiments —R¹³ of formula (IXi) or (IX) is —N(R¹⁴)(R^{14a}). In certain embodiments —R¹³ of formula (IXi) or (IX) is —OH. In certain embodiments —R¹³ of formula (IXi) or (IX) is —C(O)OH. In certain embodiments —R¹³ of formula (IXi) or (IX) is C₁₋₆ alkyl.

In certain embodiments —R¹⁴ and —R^{14a} of formula (IXi) or (IX) are independently selected from the group consisting of —H and C₁₋₆ alkyl. In certain embodiments —R¹⁴ of formula (IXi) or (IX) is —H. In certain embodiments —R¹⁴ of formula (IXi) or (IX) is C₁₋₆ alkyl. In certain embodiments —R^{14a} of formula (IXi) or (IX) is —H. In certain embodiments —R^{14a} of formula (IXi) or (IX) is C₁₋₆ alkyl.

In certain embodiments, —R³/—R⁹ of formula (IXi) are joined with the nitrogen atom to which they are attached to form a 3- to 10-membered heterocyclyl or an 8- to 11-membered heterobicyclyl. In certain embodiments, —R³/—R⁹ of formula (IXi) are joined with the nitrogen atom to which they are attached to form a 3- to 10-membered heterocyclyl or an 8- to 11-membered heterobicyclyl, wherein the attachment of the 3- to 10-membered heterocyclyl or 8- to 11-membered heterobicyclyl to the rest of the linker moiety of formula (IXi) takes place via a sp³-hybridized nitrogen.

In certain embodiments, —R³/—R⁹ of formula (IXi) are joined with the nitrogen atom to which they are attached to form a ring selected from the group consisting of aziridine, azetidine, pyrroline, imidazoline, pyrazoline, 4-thiazoline, pyrrolidine, imidazolidine, pyrazolidine, oxazolidine, isoxazolidine, thiazolidine, isothiazolidine, thiadiazolidine, piperazine, piperidine, morpholine, triazolidine, tetrazolidine, diazepane, homopiperazine, indoline, benzimidazoline, dihydroquinazoline, dihydroquinoline, tetrahydroquinoline, decahydroquinoline, decahydroisoquinoline, tetrahydroisoquinoline and dihydroisoquinoline. Each hydrogen atom of such rings may be replaced by a substituent as defined above.

In certain embodiments n of formula (IXi) or (IX) is selected from the group consisting of 0, 1, 2 and 3. In certain embodiments n of formula (IXi) or (IX) is selected from the group consisting of 0, 1 and 2. In certain embodiments n of formula (IXi) or (IX) is selected from the group consisting of 0 and 1. In certain embodiments n of formula (IXi) or (IX) is 0. In certain embodiments n of formula (IXi) or (IX) is 1. In certain embodiments n of formula (IXi) or (IX) is 2. In certain embodiments n of formula (IXi) or (IX) is 3. In certain embodiments n of formula (IXi) or (IX) is 4.

In certain embodiments -L¹- of formula (IXi) or (IX) is connected to -D through a linkage selected from the group consisting of amide, carbamate, dithiocarbamate, 0-thiocarbamate, S-thiocarbamate, urea, thiourea, thioamide, amidine and guanidine. It is understood that some of these linkages may not be reversible per se, but that in the present invention neighboring groups present in -L¹-, such as for example amide, primary amine, secondary amine and tertiary amine, render these linkages reversible.

In certain embodiments -L¹- of formula (IXi) or (IX) is conjugated to -D through an amide linkage, i.e. ═X¹ is ═O and —X²— is —C(R⁶)(R^{6a})—.

In certain embodiments -L¹- of formula (IXi) or (IX) is conjugated to -D through a carbamate linkage, i.e. ═X¹ is ═O and —X²— is —O—.

In certain embodiments -L¹- of formula (IXi) or (IX) is conjugated to -D through a dithiocarbamate linkage, i.e. ═X¹ is ═S and —X²— is —S—.

In certain embodiments -L¹- of formula (IXi) or (IX) is conjugated to -D through an 0-thiocarbamate linkage, i.e. ═X¹ is ═S and —X²— is —O—.

In certain embodiments -L¹- of formula (IXi) or (IX) is conjugated to -D through a S-thiocarbamate linkage, i.e. ═X¹ is ═O and —X²— is —S—.

In certain embodiments -L¹- of formula (IXi) or (IX) is conjugated to -D through a urea linkage, i.e. ═X¹ is ═O and —X²— is —N(R⁵)—.

In certain embodiments -L¹- of formula (IXi) or (IX) is conjugated to -D through a thiourea linkage, i.e. ═X¹ is ═S and —X²— is —N(R⁵)—.

In certain embodiments -L¹- of formula (IXi) or (IX) is conjugated to -D through a thioamide linkage, i.e. ═X¹ is ═S and —X²— is —C(R⁶)(R^{6a})—.

In certain embodiments -L¹- of formula (IXi) or (IX) is conjugated to -D through an amidine linkage, i.e. ═X¹ is ═N(R⁴) and —X²— is —C(R⁶)(R^{6a})—.

In certain embodiments -L¹- of formula (IXi) or (IX) is conjugated to -D through a guanidine linkage, i.e. ═X¹ is ═N(R⁴) and —X²— is —N(R⁵)—.

In certain embodiments -L¹- is of formula (IX′):

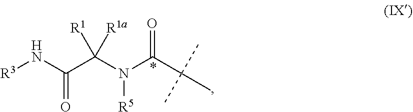

wherein the dashed line indicates the attachment to a π-electron-pair-donating heteroaromatic N of -D;
—R¹, —R^{1a}, —R³ and —R⁵ are used as defined in formula (IXi) or (IX);
optionally, the pair —R¹/—R^{1a} is joined together with the atom to which they are attached to form a C₃₋₁₀ cycloalkyl, 3- to 10-membered heterocyclyl or an 8- to 11-membered heterobicyclyl; and
optionally, the pair —R¹/—R⁵ is joined together with the atoms to which they are attached to form a 3- to 10-membered heterocyclyl or 8- to 11-membered heterobicyclyl.

In certain embodiments, —R¹ and —R^{1a} of formula (IX′) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that the pair —R¹/—R⁵ may optionally be joined together with the atoms to which they are attached to form a 3- to 10-membered heterocyclyl or 8- to 11-membered heterobicyclyl.

In certain embodiments —R¹ and —R^{1a} of formula (IX′) are both —H. In certain embodiments —R¹ of formula (IX′) is —H and —R^{1a} of formula (IX′) is C₁₋₆ alkyl. In certain embodiments, —R¹ of formula (I′) is —H and —R^{1a} of formula (I′) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments —R³ of formula (IX′) is C₁₋₆ alkyl. In certain embodiments —R³ is T. In certain embodiments —R³ of formula (IX′) is C₃₋₁₀ cycloalkyl, such as C₅- or C₆-cycloalkyl.

In certain embodiments —R⁵ of formula (IX′) is methyl. In certain embodiments —R⁵ of formula (IX′) is ethyl.

In certain embodiments, —R⁵ of formula (IX′) is —CH₃, —R¹ and —R^{1a} of formula (IX′) are —H and —R³ of formula (IX′) is —H which is replaced by one -L²-Z moiety.

In certain embodiments, —R⁵ of formula (IX′) is —CH₃, —R¹ of formula (IX′) is —H and —R^{1a} of formula (IX′) is —CH₃ and —R³ of formula (IX′) is —H which is replaced by one -L²-Z moiety.

In certain embodiments, —R⁵ of formula (IX′) is ethyl, —R¹ and —R^{1a} of formula (IX′) are —H and —R³ of formula (IX′) is —H which is replaced by one -L²-Z moiety.

In certain embodiments -L¹- is of formula (IX″):

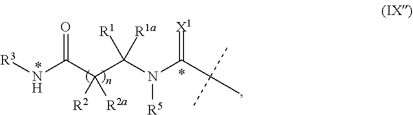

wherein the dashed line indicates the attachment to the π-electron-pair-donating heteroaromatic N of -D;
═X¹, —R¹, —R^{1a}, —R², —R^{2a}, —R³, and —R⁵ and n are used as defined in formula (IXi) or (IX);
optionally, one or more of the pairs —R/—R^{1a}, —R²/—R^{2a}, two adjacent —R² are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R²/—R⁵ and —R⁴/—R⁵ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
and wherein the distance between the nitrogen atom marked with an asterisk and the carbon atom marked with an asterisk in formula (IX″) is 5, 6 or 7 atoms and if present the carbon-carbon double bond formed between —R¹ and —R² or two adjacent —R² is in a cis configuration.

In certain embodiments, n of formula (IX″) is 0. In certain embodiments, n of formula (IX″) is 1. In certain embodiments, n of formula (IX″) is 2.

In certain embodiments, —R¹ and —R^{1a} of formula (IX″) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that —R¹/—R^{1a} may optionally be joined together with the atom to which they are attached to form a C₃₋₁₀ cycloalkyl and that one or more of the pairs —R¹/—R² and —R¹/—R⁵ may optionally be joined together with the atoms to which they are attached to form a ring -A-, wherein -A- is used as defined for formula (IXi) or (IX).

In certain embodiments, —R² and —R^{2a} of formula (IX″) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a ring -A-, wherein -A- is used as defined in formula (IXi) or (IX).

In certain embodiments, ═X¹ of formula (IX″) is ═O.

In certain embodiments, —R¹ and —R^{1a} of formula (IX″) are both —H.

In certain embodiments, —R¹ of formula (IX″) is —H and —R^{1a} of formula (IX″) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments, —R³ of formula (IX″) is C₁₋₆ alkyl.

In certain embodiments, —R⁵ of formula (IX″) is —H. In certain embodiments, —R⁵ of formula (IX″) is methyl. In certain embodiments, —R⁵ of formula (IX″) is ethyl.

In certain embodiments, —R⁷ of formula (IX″) is hydrogen. In certain embodiments, —R⁷ of formula (IX″) is methyl. In certain embodiments, —R⁷ of formula (IX″) is ethyl.

In certain embodiments -L¹- is of formula (IX′″):

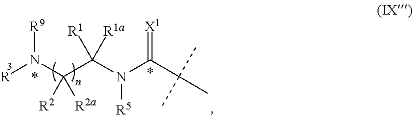

wherein the dashed line indicates the attachment to the π-electron-pair-donating heteroaromatic N of -D;
═X¹, —R¹, —R^{1a}, —R², —R^{2a}, —R³, —R⁵, —R⁹ and n are used as defined in formula (IXi) or (IX);
optionally, one or more of the pairs —R/—R^{1a}, —R²/—R^{2a}, two adjacent —R² and —R³/—R⁹ are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R²/—R⁵ and —R⁴/—R⁵ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, and 3;
optionally, two adjacent —R² form a carbon-carbon double bond provided that n is selected from the group consisting of 2, and 3;
and wherein the distance between the nitrogen atom marked with an asterisk and the carbon atom marked with an asterisk in formula (IX′″) is 5, 6 or 7 atoms and if present the carbon-carbon double bond formed between —R¹ and —R² or two adjacent —R² is in a cis configuration.

In certain embodiments, n of formula (IX′″) is 1. In certain embodiments, n of formula (IX′″) is 2. In certain embodiments, n of formula (IX′″) is 3.

In certain embodiments, —R¹ and —R^{1a} of formula (IX′″) are independently selected from the group consisting of —H and C₁₋₆ alkyl. In certain embodiments, —R¹ and —R^{1a} of formula (IX′″) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that -R¹/—R^{1a} may optionally be joined together with the atom to which they are attached to form a C₃₋₁₀ cycloalkyl and that one or more of the pairs —R¹/—R⁵, —R¹/—R⁹ and —R¹/—R¹⁰ may optionally be joined together with the atoms to which they are attached to form a ring -A-, wherein -A- is used as defined for formula (IXi) or (IX).

In certain embodiments, —R¹ and —R^{1a} of formula (IX′″) are both —H.

In certain embodiments, —R² and —R^{2a} of formula (IX′″) are independently selected from the group consisting of —H and C₁₋₆ alkyl. In certain embodiments, —R² and —R^{2a} of formula (IX′″) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In this case it is understood that one or more of the pairs —R²/—R^{2a} and two adjacent —R² may optionally be joined with the atom to which they are attached to form a C₃₋₁₀ cycloalkyl and that the pair —R²/—R⁵ may optionally be joined together with the atoms to which they are attached to form a 3- to 10-membered heterocyclyl or 8- to 11-membered heterobicyclyl.

In certain embodiments, —R² and —R^{2a} of formula (IX′″) are both —H.

In certain embodiments, —R³ of formula (IX′″) is H. In certain embodiments, —R³ of formula (IX′″) is methyl.

In certain embodiments, —R⁵ of formula (IX′″) is H. In certain embodiments, —R⁵ of formula (IX′″) is methyl.

In certain embodiments -L¹- is of formula (X)

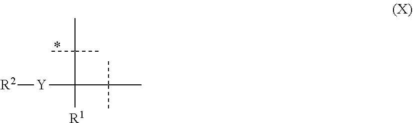

wherein
the dashed line marked with an asterisk indicates the attachment to -L²-;
the unmarked dashed line indicates the attachment to a π-electron-pair-donating heteroaromatic N of -D;
—Y— is selected from the group consisting of —N(R³)—, —O— and —S—;

—R¹, —R² and —R³ are independently selected from the group consisting of —H, -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴, which are the same or different; and
wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T is independently optionally substituted with one or more —R⁴, which are the same or different;
   wherein —R⁴, —R⁵ and —R^{5a} are independently selected from the group consisting of —H and C₁₋₆ alkyl; wherein C₁₋₆ alkyl is optionally substituted with one or more halogen, which are the same or different; and
wherein -L¹- is substituted with -L²- and wherein -L¹- is optionally further substituted.

The optional further substituents of -L¹- of formula (X) are as described elsewhere herein.

In certain embodiments -L¹- of formula (X) is not further substituted.

In certain embodiments —Y— of formula (X) is —N(R³)—.

In certain embodiments —Y— of formula (X) is —O—.

In certain embodiments —Y— of formula (X) is —S—.

In certain embodiments —R¹, —R² and —R³ of formula (X) are independently selected from the group consisting of —H, -T, C₁₋₆ alkyl, C₂₋₆ alkenyl and C₂₋₆ alkynyl.

In certain embodiments —R¹ of formula (X) is independently selected from the group consisting of —H, -T, C₁₋₆ alkyl, C₂₋₆ alkenyl and C₂₋₆ alkynyl. In certain embodiments —R¹ of formula (X) is —H. In certain embodiments —R¹ of formula (X) is -T. In certain embodiments —R¹ of formula (X) is C₁₋₆ alkyl. In certain embodiments —R¹ of formula (X) is C₂₋₆ alkenyl. In certain embodiments —R¹ of formula (X) is C₂₋₆ alkynyl.

In certain embodiments —R² of formula (X) is independently selected from the group consisting of —H, -T, C₁₋₆ alkyl, C₂₋₆ alkenyl and C₂₋₆ alkynyl. In certain embodiments —R² of formula (X) is —H. In certain embodiments —R² of formula (X) is -T. In certain embodiments —R² of formula (X) is C₁₋₆ alkyl. In certain embodiments —R² of formula (X) is C₂₋₆ alkenyl. In certain embodiments —R² of formula (X) is C₂₋₆ alkynyl.

In certain embodiments —R³ of formula (X) is independently selected from the group consisting of —H, -T, C₁₋₆ alkyl, C₂₋₆ alkenyl and C₂₋₆ alkynyl. In certain embodiments —R³ of formula (X) is —H. In certain embodiments —R³ of formula (X) is -T. In certain embodiments —R³ of formula (X) is C₁₋₆ alkyl. In certain embodiments —R³ of formula (X) is C₂₋₆ alkenyl. In certain embodiments —R³ of formula (X) is C₂₋₆ alkynyl.

In certain embodiments T of formula (X) is selected from the group consisting of phenyl, naphthyl, indenyl, indanyl, tetralinyl, C₃₋₁₀ cycloalkyl, 3- to 10-membered heterocyclyl and 8- to 11- heterobicyclyl. In certain embodiments T of formula (X) is phenyl. In certain embodiments T of formula (X) is naphthyl. In certain embodiments T of formula (X) is indenyl. In certain embodiments T of formula (X) is indanyl. In certain embodiments T of formula (X) is tetralinyl. In certain embodiments T of formula (X) is C₃₋₁₀ cycloalkyl. In certain embodiments T of formula (X) is 3- to 10-membered heterocyclyl. In certain embodiments T of formula (X) is 8- to 11-heterobicyclyl.

In certain embodiments T of formula (X) is substituted with one or more —R⁴ of formula (X).

In certain embodiments T of formula (X) is substituted with one —R⁴ of formula (X).

In certain embodiments T of formula (X) is not substituted with —R⁴ of formula (X).

In certain embodiments —R⁴, —R⁵ and —R^{5a} of formula (X) are independently selected from the group consisting of —H and C₁₋₆ alkyl.

In certain embodiments —R⁴ of formula (X) is selected from the group consisting of —H and C₁₋₆ alkyl. In certain embodiments —R⁴ of formula (X) is —H. In certain embodiments —R⁴ of formula (X) is C₁₋₆ alkyl.

In certain embodiments —R⁵ of formula (X) is selected from the group consisting of —H and C₁₋₆ alkyl. In certain embodiments —R⁵ of formula (X) is —H. In certain embodiments —R⁵ of formula (X) is C₁₋₆ alkyl.

In certain embodiments —R^{5a} of formula (X) is selected from the group consisting of —H and C₁₋₆ alkyl. In certain embodiments —R^{5a} of formula (X) is —H. In certain embodiments —R^{5a} of formula (X) is C₁₋₆ alkyl.

In certain embodiments -L¹- of formula (X) is connected to -D through a heminal linkage.

In certain embodiments -L¹- of formula (X) is connected to -D through an aminal linkage.

In certain embodiments -L¹- of formula (X) is connected to -D through a hemithioaminal linkage.

In certain embodiments, —Y— of formula (X) is —O— and —R² is C₁₋₆ alkyl. In certain embodiments, —Y— of formula (X) is —O— and —R² is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments, —Y— of formula (X) is —O— and —R² of formula (X) is methyl. In certain embodiments, —Y— of formula (X) is —O— and —R² of formula (X) is ethyl.

In certain embodiments, —Y— of formula (X) is —O— and —R² of formula (X) is C₁₋₆ alkyl, wherein C₁₋₆ alkyl is interrupted by —C(O)—.

In certain embodiments, —Y— of formula (X) is —N(R³)— and —R² of formula (X) is C₁₋₆ alkyl, wherein C₁₋₆ alkyl is interrupted by —C(O)O— and —R³ is as defined in formula (X).

In certain embodiments, —Y— is —N(R³)— and —R² is C₁₋₆ alkyl, wherein C₁₋₆ alkyl is interrupted by —C(O)O— and —R³ is selected from the group consisting of —H, methyl, ethyl and propyl.

In certain embodiments, -L¹- is of formula (Xi)

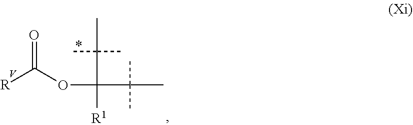

wherein
the dashed line marked with an asterisk indicates the attachment to -L²- and the unmarked dashed line indicates the attachment to the π-electron-pair-donating heteroaromatic N of -D;
—R^{v} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R¹ is used as defined in formula (X).

In certain embodiments, —R^{v} of formula (Xi) is selected from the group consisting of methyl, ethyl and propyl. In certain embodiments, —R^{v} of formula (Xi) is methyl. In certain embodiments, —R^{v} of formula (Xi) is ethyl. In certain embodiments, —R^{v} of formula (Xi) is propyl.

In certain embodiments, -L¹- is of formula (Xii)

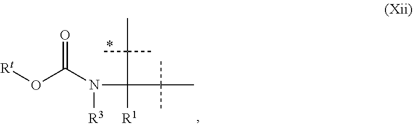

wherein
the dashed line marked with an asterisk indicates the attachment to -L²- and the unmarked dashed line indicates the attachment to the π-electron-pair-donating heteroaromatic N of -D;
—R^{t} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R¹ and —R³ are used as defined in formula (X).

In certain embodiments, —R³ of formula (Xii) is selected from the group consisting of —H, methyl and ethyl. In certain embodiments, —R³ of formula (Xii) is —H. In certain embodiments, —R³ of formula (Xii) is methyl. In certain embodiments, —R³ of formula (Xii) is ethyl.

In certain embodiments, —R^{t} of formula (Xii) is selected from the group consisting of methyl, ethyl and propyl. In certain embodiments, —R^{t} of formula (Xii) is methyl. In certain embodiments, —R^{t} of formula (Xii) is ethyl. In certain embodiments, —R^{t} of formula (Xii) is propyl.

In certain embodiments, -L¹- is of formula (Xiii)

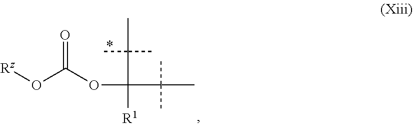

wherein
the dashed line marked with an asterisk indicates the attachment to -L²- and the unmarked dashed line indicates the attachment to the π-electron-pair-donating heteroaromatic N of -D;
—R^{z} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R¹ is used as defined in formula (X).

In certain embodiments, —R^{z} of formula (Xiii) is selected from the group consisting of methyl, ethyl and propyl. In certain embodiments, —R^{z} of formula (Xiii) is methyl. In certain embodiments, —R^{z} of formula (Xiii) is ethyl. In certain embodiments, —R^{z} of formula (Xiii) is propyl.

A moiety -L¹- suitable for drugs D that when bound to -L¹- comprise an electron-donating heteroaromatic N⁺ moiety or a quaternary ammonium cation and becomes a moiety -D⁺ upon linkage with -L¹- is of formula (XI)

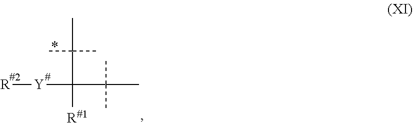

wherein
the dashed line marked with an asterisk indicates the attachment to -L²-, the unmarked dashed line indicates the attachment to the N⁺ of -D⁺;
—Y^{#}— is selected from the group consisting of —N(R^{#3})—, —O— and —S—;
—R^{#1}, —R^{#2} and —R^{#3} are independently selected from the group consisting of —H, -T^{#}, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴⁴ which are the same or different; and wherein C₁₋₆ alkyl, C₂₋₆ alkenyl and C₂₋₆ alkynyl are optionally interrupted by one or more groups selected from the group consisting of -T^{#}-, —C(O)O—, —O—, —C(O)—, —C(O)N(R^{#5})—, —S(O)₂N(R^{#5})—, —S(O)N(R^{#5})—, —S(O)₂—, —S(O)—, —N(R^{#5})S(O)₂N(R^{#5a})—, —S—, —N(R^{#5}), —OC(OR^{#5})(R^{#5a})—, —N(R^{#5})C(O)N(R^{#5a})— and —OC(O)N(R^{#5})—;
   each T^{#} is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T^{#} is independently optionally substituted with one or more —R⁴⁴, which are the same or different; and
   wherein —R^{#4}, —R^{#5} and —R^{#5a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It is understood that in certain embodiments -D⁺ may comprise both an electron-donating heteroaromatic N⁺ and a quaternary ammonium cation and analogously the corresponding D may comprise both an electron-donating heteroaromatic N and a tertiary amine. It is also understood that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

Such drug moiety -D⁺ comprises at least one, such as one, two, three, four, five, six, seven, eight, nine or ten electron-donating heteroaromatic N⁺ or quaternary ammonium cations and analogously the corresponding released drug D comprises at least one, such as one, two, three, four, five, six, seven, eight, nine or ten electron-donating heteroaromatic N or tertiary amines. Examples of chemical structures including heteroaromatic nitrogens i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic π-system is marked with “§”:

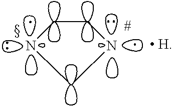

Such electron-donating heteroaromatic nitrogen atoms do not comprise heteroaromatic nitrogen atoms which donate one electron pair (i.e. not one electron) to the aromatic π-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

In certain embodiments —Y^{#}— of formula (XI) is —N(R^{#3})—. In certain embodiments —Y^{#}— of formula (XI) is —O—. In certain embodiments —Y^{#}— of formula (XI) is —S—.

In certain embodiments —R^{#1}, —R^{#2} and —R^{#3} of formula (XI) are independently selected from the group consisting of —H, -T^{#}, C₁₋₆ alkyl, C₂₋₆ alkenyl and C₂₋₆ alkynyl.

In certain embodiments —R^{#1} of formula (XI) is independently selected from the group consisting of —H, -T^{#}, C₁₋₆ alkyl, C₂₋₆ alkenyl and C₂₋₆ alkynyl. In certain embodiments —R^{#1} of formula (XI) is —H. In certain embodiments —R^{#1} of formula (XI) is -T^{#}. In certain embodiments —R^{#1} of formula (XI) is C₁₋₆ alkyl. In certain embodiments —R^{#1} of formula (XI) is C₂₋₆ alkenyl. In certain embodiments —R^{#1} of formula (XI) is C₂₋₆ alkynyl.

In certain embodiments —R^{#2} of formula (XI) is independently selected from the group consisting of —H, -T^{#}, C₁₋₆ alkyl, C₂₋₆ alkenyl and C₂₋₆ alkynyl. In certain embodiments —R^{#2} of formula (XI) is —H. In certain embodiments —R² of formula (XI) is -T^{#}. In certain embodiments —R^{#2} of formula (XI) is C₁₋₆ alkyl. In certain embodiments —R^{#2} of formula (XI) is C₂₋₆ alkenyl. In certain embodiments —R^{#2} of formula (XI) is C₂₋₆ alkynyl.

In certain embodiments, —R^{#3} of formula (XI) is independently selected from the group consisting of —H, -T^{#}, C₁₋₆ alkyl, C₂₋₆ alkenyl and C₂₋₆ alkynyl. In certain embodiments —R^{#3} of formula (XI) is —H. In certain embodiments —R^{#3} of formula (XI) is -T^{#}. In certain embodiments, —R^{#3} is C₁₋₆ alkyl. In certain embodiments —R^{#3} of formula (XI) is C₂₋₆ alkenyl. In certain embodiments —R^{#3} of formula (XI) is C₂₋₆ alkynyl.

In certain embodiments T^{#} of formula (XI) is selected from the group consisting of phenyl, naphthyl, indenyl, indanyl, tetralinyl, C₃₋₁₀ cycloalkyl, 3- to 10-membered heterocyclyl and 8- to 11- heterobicyclyl. In certain embodiments T^{#} of formula (XI) is phenyl. In certain embodiments T^{#} of formula (XI) is naphthyl. In certain embodiments T^{#} of formula (XI) is indenyl. In certain embodiments T^{#} of formula (XI) is indanyl. In certain embodiments T^{#} of formula (XI) is tetralinyl. In certain embodiments T^{#} of formula (XI) is C₃₋₁₀ cycloalkyl. In certain embodiments T^{#} of formula (XI) is 3- to 10-membered heterocyclyl. In certain embodiments T^{#} of formula (XI) is 8- to 11-heterobicyclyl. In certain embodiments T^{#} of formula (XI) is substituted with one or more —R⁴ of formula (XI).

In certain embodiments T^{#} of formula (XI) is substituted with one —R⁴ of formula (XI).

In certain embodiments T^{#} of formula (XI) is not substituted with —R⁴ of formula (XI). In certain embodiments —R^{#4}, —R^{#5} and —R^{#5a} of formula (XI) are independently selected from the group consisting of —H and C₁₋₆ alkyl.

In certain embodiments —R^{#4} of formula (XI) is selected from the group consisting of —H and C₁₋₆ alkyl. In certain embodiments —R^{#4} of formula (XI) is —H. In certain embodiments —R⁴⁴ of formula (XI) is C₁₋₆ alkyl.

In certain embodiments —R^{#5} of formula (XI) is selected from the group consisting of —H and C₁₋₆ alkyl. In certain embodiments —R⁵ of formula (XI) is —H. In certain embodiments —R^{#5} of formula (XI) is C₁₋₆ alkyl.

In certain embodiments —R^{#5a} of formula (XI) is selected from the group consisting of —H and C₁₋₆ alkyl. In certain embodiments —R^{#5a} of formula (XI) is —H. In certain embodiments —R^{#5a} of formula (XI) is C₁₋₆ alkyl.

In certain embodiments, —Y^{#}— of formula (XI) is —O— and —R^{#2} of formula (XI) is C₁₋₆ alkyl. In certain embodiments, —Y^{#}— of formula (XI) is —O— and —R^{#2} of formula (XI) is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments, —Y^{#}- of formula (XI) is —O— and —R⁴² of formula (XI) is methyl. In certain embodiments, —Y^{#}— of formula (XI) is —O— and —R^{#2} of formula (XI) is ethyl.

In certain embodiments, —Y^{#}— of formula (XI) is —O— and —R^{#2} of formula (XI) is C₁₋₆ alkyl, wherein C₁₋₆ alkyl is interrupted by —C(O)—.

In certain embodiments, —Y^{#}— of formula (XI) is —N(R³)— and —R^{#2} of formula (XI) is C₁₋₆ alkyl, wherein C₁₋₆ alkyl is interrupted by —C(O)O— and —R^{#3} is as defined in formula (XI).

In certain embodiments, —Y^{#}— of formula (XI) is —N(R³)— and —R^{#2} of formula (XI) is C₁₋₆ alkyl, wherein C₁₋₆ alkyl is interrupted by —C(O)O— and —R^{#3} of formula (XI) is selected from the group consisting of —H, methyl, ethyl and propyl.

In certain embodiments, -L¹- is of formula (XIi)

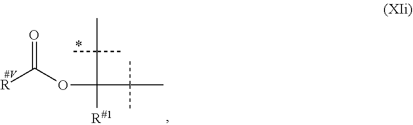

wherein
the dashed line marked with an asterisk indicates the attachment to -L²- and the unmarked dashed line indicates the attachment to the π-electron-pair-donating heteroaromatic N of -D;
—R^{#v} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} is used as defined in formula (XI).

In certain embodiments, —R⁴ of formula (XIi) is selected from the group consisting of methyl, ethyl and propyl. In certain embodiments, —R^{#v} of formula (XIi) is methyl. In certain embodiments, —R^{#v} of formula (XIi) is ethyl. In certain embodiments, —R^{#v} of formula (XIi) is propyl.

In certain embodiments, -L¹- of formula (XIii)

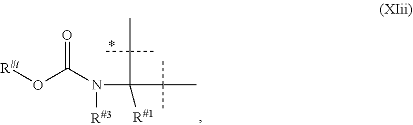

wherein
the dashed line marked with an asterisk indicates the attachment to -L²- and the unmarked dashed line indicates the attachment to the π-electron-pair-donating heteroaromatic N of -D;
—R^{#t} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} and —R^{#3} are used as defined in formula (XI).

In certain embodiments, —R^{#3} of formula (XIii) is selected from the group consisting of —H, methyl and ethyl. In certain embodiments, —R^{#3} of formula (XIii) is —H. In certain embodiments, —R^{#3} of formula (XIii) is methyl. In certain embodiments, —R^{#3} of formula (XIii) is ethyl.

In certain embodiments, —R^{#t} of formula (XIii) is selected from the group consisting of methyl, ethyl and propyl. In certain embodiments, —R^{#t} of formula (XIii) is methyl. In certain embodiments, —R^{#t} of formula (XIii) is ethyl. In certain embodiments, —R^{#t} of formula (XIii) is propyl.

In certain embodiments, -L¹- is of formula (XIiii)

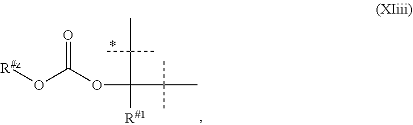

wherein
the dashed line marked with an asterisk indicates the attachment to -L²- and the unmarked dashed line indicates the attachment to the π-electron-pair-donating heteroaromatic N of -D;
—R^{#z} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} is used as defined in formula (XI).

In certain embodiments, —R^{#z} of formula (XIiii) is selected from the group consisting of methyl, ethyl and propyl. In certain embodiments, —R^{#z} of formula (XIiii) is methyl. In certain embodiments, —R^{#z} of formula (XIiii) is ethyl. In certain embodiments, —R^{#z} of formula (XIiii) is propyl.

A moiety -L¹- suitable for drugs D that when bound to -L¹- comprise an electron-donating heteroaromatic N⁺ moiety or a quaternary ammonium cation and becomes a moiety -D⁺ upon linkage with -L¹- is of formula (XII)

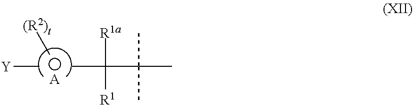

wherein
the dashed line indicates the attachment to the N⁺ of -D⁺;
t is selected from the group consisting of 0, 1, 2, 3, 4, 5 and 6;
-A- is a ring selected from the group consisting of monocyclic or bicyclic aryl and heteroaryl, provided that -A- is connected to —Y and —C(R¹)(R^{1a})— via carbon atoms; wherein said monocyclic or bicyclic aryl and heteroaryl are optionally substituted with one or more —R², which are the same or different;
—R¹, —R^{1a} and each —R² are independently selected from the group consisting of —H, —C(O)OH, -halogen, —NO₂, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
   each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R³, which are the same or different;
   wherein —R³ is selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
   wherein —R⁴ and —R^{4a} are independently selected from the group consisting of —H and C₁₋₆ alkyl; wherein C₁₋₆ alkyl is optionally substituted with one or more halogen, which are the same or different;
—Y is selected from the group consisting of:

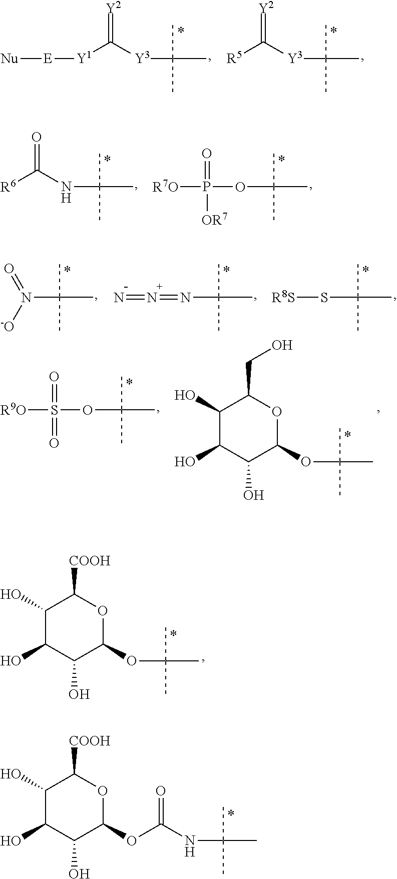

and a peptidyl moiety;
   wherein
   the dashed line marked with an asterisk indicates the attachment to -A-;
   —Nu is a nucleophile;
   —Y¹— is selected from the group consisting of —O—, —C(R¹⁰)(R^{10a})—, —N(R¹¹)— and —S—;
   ═Y² is selected from the group consisting of ═O, ═S and ═N(R¹²);
   —Y³— is selected from the group consisting of —O—, —S— and —N(R¹³)—;
   -E- is selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R¹⁴, which are the same or different;
   —R⁵, —R⁶, each —R⁷, —R⁸, —R⁹, —R¹⁰, —R^{10a}, —R¹¹, —R¹² and —R¹³ are independently selected from the group consisting of C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹⁴, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹⁵)—, —S(O)₂N(R¹⁵)—, —S(O)N(R¹⁵)—, —S(O)₂—, —S(O)—, —N(R¹⁵)S(O)₂N(R^{15a})—, —S—, —N(R¹⁵)—, —OC(OR¹⁵)R^{15a}—, —N(R¹⁵)C(O)N(R^{15a})— and —OC(O)N(R¹⁵)—;
      each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹⁴, which are the same or different;
         wherein —R¹⁴, —R¹⁵ and —R^{15a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It is understood that in certain embodiments -D⁺ may comprise both an electron-donating heteroaromatic N⁺ and a quaternary ammonium cation and analogously the corresponding D may comprise both an electron-donating heteroaromatic N and a tertiary amine. It is also understood that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

The optional further substituents of -L¹- of formula (XII) are as described elsewhere herein.

In certain embodiments -L¹- of formula (XII) is not further substituted.

Such drug moiety -D⁺ comprises at least one, such as one, two, three, four, five, six, seven, eight, nine or ten electron-donating heteroaromatic N⁺ or quaternary ammonium cations and analogously the corresponding released drug D comprises at least one, such as one, two, three, four, five, six, seven, eight, nine or ten electron-donating heteroaromatic N or tertiary amines. Examples of chemical structures including heteroaromatic nitrogens i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic π-system is marked with

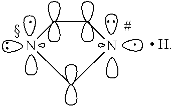

Such electron-donating heteroaromatic nitrogen atoms do not comprise heteroaromatic nitrogen atoms which donate one electron pair (i.e. not one electron) to the aromatic π-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

As used herein, the term “monocyclic or bicyclic aryl” means an aromatic hydrocarbon ring system which may be monocyclic or bicyclic, wherein the monocyclic aryl ring consists of at least 5 ring carbon atoms and may comprise up to 10 ring carbon atoms and wherein the bicyclic aryl ring consists of at least 8 ring carbon atoms and may comprise up to 12 ring carbon atoms. Each hydrogen atom of a monocyclic or bicyclic aryl may be replaced by a substituent as defined below.

As used herein, the term “monocyclic or bicyclic heteroaryl” means a monocyclic aromatic ring system that may comprise 2 to 6 ring carbon atoms and 1 to 3 ring heteroatoms or a bicyclic aromatic ring system that may comprise 3 to 9 ring carbon atoms and 1 to 5 ring heteroatoms, such as nitrogen, oxygen and sulfur. Examples for monocyclic or bicyclic heteroaryl groups include, but are not limited to, benzofuranyl, benzothiophenyl, furanyl, imidazolyl, indolyl, azaindolyl, azabenzimidazolyl, benzoxazolyl, benzthiazolyl, benzthiadiazolyl, benzotriazolyl, tetrazinyl, tetrazolyl, isothiazolyl, oxazolyl, isoxazolyl, pyrazinyl, pyrazolyl, pyridazinyl, pyridinyl, pyrimidinyl, pyrrolyl, quinolinyl, quinazolinyl, quinoxalinyl, triazolyl, thiazolyl and thiophenyl. Each hydrogen atom of a monocyclic or bicyclic heteroaryl may be replaced by a substituent as defined below.

As used herein, the term “nucleophile” refers to a reagent or functional group that forms a bond to its reaction partner, i.e. the electrophile by donating both bonding electrons.

In certain embodiments t of formula (XII) is 0. In certain embodiments t of formula (XII) is 1. In certain embodiments t of formula (XII) is 2. In certain embodiments t of formula (XII) is3. In certain embodiments t of formula (XII) is 4. In certain embodiments t of formula (XII) is 5. In certain embodiments t of formula (XII) is 6.

In certain embodiments -A- of formula (XII) is a ring selected from the group consisting of monocyclic or bicyclic aryl and heteroaryl, provided that -A- is connected to —Y and —C(R¹)(R^{1a})— via carbon atoms. In certain embodiments -A- of formula (XII) is substituted with one or more —R² of formula (XII) which are the same or different. In certain embodiments -A- of formula (XII) is not substituted with —R² of formula (XII). In certain embodiments -A- of formula (XII) is selected from the group consisting of.

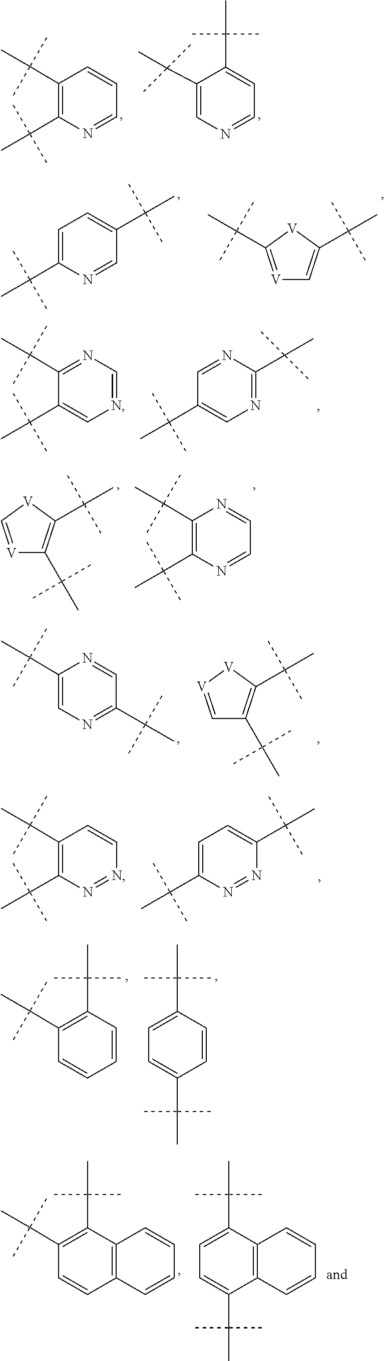

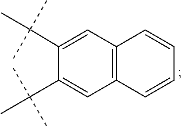

wherein each V is independently selected from the group consisting of O, S and N.

In certain embodiments —R¹, —R^{1a} and each —R² of formula (XII) are independently selected from the group consisting of —H, —C(O)OH, -halogen, —CN, —NO₂, —OH, C₁₋₆ alkyl, C₂₋₆ alkenyl and C₂₋₆ alkynyl. In certain embodiments —R¹, —R^{1a} and each —R² of formula (XII) are independently selected from the group consisting of —H, —C(O)OH, —CN, C₁₋₆ alkyl, C₂₋₆ alkenyl and C₂₋₆ alkynyl. In certain embodiments —R¹ of formula (XII) is —H. In certain embodiments —R¹ of formula (XII) is —C(O)OH. In certain embodiments —R¹ of formula (XII) is -halogen. In certain embodiments —R¹ of formula (XII) is —F. In certain embodiments —R¹ of formula (XII) is —CN. In certain embodiments —R¹ of formula (XII) is —NO₂. In certain embodiments —R¹ of formula (XII) is —OH. In certain embodiments —R¹ of formula (XII) is C₁₋₆ alkyl. In certain embodiments —R¹ of formula (XII) is C₂₋₆ alkenyl. In certain embodiments —R¹ of formula (XII) is C₂₋₆ alkynyl. In certain embodiments —R^{1a} of formula (XII) is —H. In certain embodiments —R^{1a} of formula (XII) is —C(O)OH. In certain embodiments —R^{1a} of formula (XII) is -halogen. In certain embodiments —R^{1a} of formula (XII) is —F. In certain embodiments —Ria of formula (XII) is —CN. In certain embodiments —R^{1a} of formula (XII) is —NO₂. In certain embodiments —R^{1a} of formula (XII) is —OH. In certain embodiments —R^{1a} of formula (XII) is C₁₋₆ alkyl. In certain embodiments —R^{1a} of formula (XII) is C₂₋₆ alkenyl. In certain embodiments —R^{1a} of formula (XII) is C₂₋₆ alkynyl.

In certain embodiments each of —R² of formula (XII) is independently selected from the group consisting of —H, —C(O)OH, -halogen, —CN, —NO₂, —OH, C₁₋₆ alkyl, C₂₋₆ alkenyl and C₂₋₆ alkynyl. In certain embodiments each of —R² of formula (XII) is —H. In certain embodiments each of —R² of formula (XII) is —C(O)OH. In certain embodiments each of —R² of formula (XII) is -halogen. In certain embodiments each of —R² of formula (XII) is —F. In certain embodiments each of —R² of formula (XII) is —CN. In certain embodiments each of —R² of formula (XII) is —NO₂. In certain embodiments each of —R² of formula (XII) is —OH. In certain embodiments each of —R² of formula (XII) is C₁₋₆ alkyl. In certain embodiments each of —R² of formula (XII) is C₂₋₆ alkenyl. In certain embodiments each of —R² of formula (XII) is C₂₋₆ alkynyl.

In certain embodiments T of formula (XII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments T of formula (XII) is phenyl. In certain embodiments T of formula (XII) is naphthyl. In certain embodiments T of formula (XII) is indenyl. In certain embodiments T of formula (XII) is indanyl. In certain embodiments T of formula (XII) is tetralinyl. In certain embodiments T of formula (XII) is C₃₋₁₀ cycloalkyl. In certain embodiments T of formula (XII) is 3- to 10-membered heterocyclyl. In certain embodiments T of formula (XII) is 8- to 11-membered heterobicyclyl.

In certain embodiments T of formula (XII) is substituted with one or more —R³ of formula (XII), which are the same or different. In certain embodiments T of formula (XII) is substituted with one —R³ of formula (XII). In certain embodiments T of formula (XII) is not substituted with —R³ of formula (XII).

In certain embodiments —R³ of formula (XII) is selected from the group consisting of —H, —NO₂, —OCH₃, —CN, —N(R⁴)(R^{4a}), —OH, —C(O)OH and C₁₋₆ alkyl. In certain embodiments —R³ of formula (XII) is —H. In certain embodiments —R³ of formula (XII) is —NO₂. In certain embodiments —R³ of formula (XII) is —OCH₃. In certain embodiments —R³ of formula (XII) is —CN. In certain embodiments —R³ of formula (XII) is —N(R⁴)(R^{4a}). In certain embodiments —R³ of formula (XII) is —OH. In certain embodiments —R³ of formula (XII) is —C(O)OH. In certain embodiments —R³ of formula (XII) is C₁₋₆ alkyl. In certain embodiments —R⁴ and —R^{4a} of formula (XII) are independently selected from the group consisting of —H and C₁₋₆ alkyl. In certain embodiments —R⁴ of formula (XII) is —H. In certain embodiments —R⁴ is C₁₋₆ alkyl. In certain embodiments —R^{4a} of formula (XII) is —H. In certain embodiments —R^{4a} of formula (XII) is C₁₋₆ alkyl.

In certain embodiments, —Y of formula (XII) is selected from the group consisting of

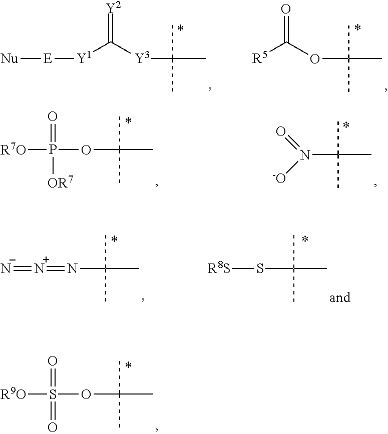

wherein —Nu, -E-, —Y¹—, ═Y², —Y³—, —R⁵, —R⁷, —R⁸ and —R⁹ are defined as above.

In certain embodiments —Y of formula (XII) is

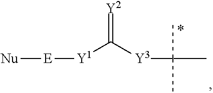

wherein —Nu, -E, —Y¹—, ═Y² and —Y³— are as defined elsewhere herein and the dashed line marked with an asterisk indicates the attachment to -A- of formula (XII). It is understood that in this instance the release of the drug D is not triggered by an enzyme, and that the drug is released in its unmodified, pharmacologically fully active form in the absence of an enzyme.

In certain embodiments —Nu of formula (XII) is a nucleophile selected from the group consisting of primary, secondary, or tertiary amine and amide. In certain embodiments —Nu of formula (XII) is a primary amine. In certain embodiments —Nu of formula (XII) is a secondary amine. In certain embodiments —Nu of formula (XII) is a tertiary amine. In certain embodiments —Nu of formula (XII) is an amide.

In certain embodiments —Y¹— of formula (XII) is selected from the group consisting of —O—, —C(R¹⁰)(R^{10a})—, —N(R¹¹)— and —S—. In certain embodiments —Y¹— of formula (XII) is —O—. In certain embodiments —Y¹— of formula (XII) is —C(R¹⁰)(R^{10a})—. In certain embodiments —Y¹— of formula (XII) is —N(R¹¹)—. In certain embodiments —Y¹— of formula (XII) is —S—.

In certain embodiments ═Y² of formula (XII) is selected from the group consisting of ═O, ═S and ═N(R¹²). In certain embodiments ═Y² of formula (XII) is ═O. In certain embodiments ═Y² of formula (XII) is ═S. In certain embodiments ═Y² of formula (XII) is ═N(R¹²).

In certain embodiments —Y³— of formula (XII) is selected from the group consisting of —O—, —S— and —N(R¹³). In certain embodiments —Y³— of formula (XII) is —O—. In certain embodiments —Y³— of formula (XII) is —S—. In certain embodiments —Y³— of formula (XII) is —N(R¹³)—.

In certain embodiments —Y¹— of formula (XII) is —N(R¹¹)—, ═Y² of formula (XII) is ═O and —Y³— is —O—.

In certain embodiments —Y¹— of formula (XII) is —N(R¹¹)—, ═Y² of formula (XII) is ═O, —Y³— of formula (XII) is —O— and —Nu of formula (XII) is —N(CH₃)₂.

In certain embodiments -E- of formula (XII) is selected from the group consisting of C₁₋₆ alkyl, C₂₋₆ alkenyl, C₂₋₆ alkynyl and -Q-. In certain embodiments -E- of formula (XII) is C₁₋₆ alkyl. In certain embodiments -E- of formula (XII) is C₂₋₆ alkenyl. In certain embodiments -E- of formula (XII) is C₂₋₆ alkynyl. In certain embodiments -E- of formula (XII) is -Q-.

In certain embodiments Q of formula (XII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q of formula (XII) is phenyl. In certain embodiments Q of formula (XII) is naphthyl. In certain embodiments Q of formula (XII) is indenyl. In certain embodiments Q of formula (XII) is indanyl. In certain embodiments Q of formula (XII) is tetralinyl. In certain embodiments Q of formula (XII) is C₃₋₁₀ cycloalkyl. In certain embodiments Q of formula (XII) is 3- to 10-membered heterocyclyl. In certain embodiments Q of formula (XII) is 8- to 11-membered heterobicyclyl.

In certain embodiments Q of formula (XII) is substituted with one or more —R¹⁴. In certain embodiments Q of formula (XII) is not substituted with —R¹⁴.

In certain embodiments —R⁵, —R⁶, each —R⁷, —R⁸, —R⁹, —R¹⁰, —R^{10a}, —R¹¹, —R¹² and —R¹³ of formula (XII) are independently selected from the group consisting of C₁₋₂₀ alkyl, C₂₋₂₀ alkenyl, C₂₋₂₀ alkynyl and -Q.

In certain embodiments —R⁵ of formula (XII) is C₁₋₂₀ alkyl. In certain embodiments —R⁵ of formula (XII) is C₂₋₂₀ alkenyl. In certain embodiments —R⁵ of formula (XII) is C₂₋₂₀ alkynyl. In certain embodiments —R⁵ of formula (XII) is -Q.

In certain embodiments —R⁶ of formula (XII) is C₁₋₂₀ alkyl. In certain embodiments —R⁶ of formula (XII) is C₂₋₂₀ alkenyl. In certain embodiments —R⁶ of formula (XII) is C₂₋₂₀ alkynyl. In certain embodiments —R⁶ is -Q.

In certain embodiments each of —R⁷ of formula (XII) is independently selected from the group consisting of C₁₋₂₀ alkyl, C₂₋₂₀ alkenyl, C₂₋₂₀ alkynyl and -Q. In certain embodiments each of —R⁷ of formula (XII) is C₁₋₂₀ alkyl. In certain embodiments each of —R⁷ of formula (XII) is C₂₋₂₀ alkenyl. In certain embodiments each of —R⁷ of formula (XII) is C₂₋₂₀ alkynyl. In certain embodiments each of —R⁷ of formula (XII) is -Q.

In certain embodiments —R⁸ of formula (XII) is C₁₋₂₀ alkyl. In certain embodiments —R⁸ of formula (XII) is C₂₋₂₀ alkenyl. In certain embodiments —R⁸ of formula (XII) is C₂₋₂₀ alkynyl. In certain embodiments —R⁸ of formula (XII) is -Q.

In certain embodiments —R⁹ of formula (XII) is C₁₋₂₀ alkyl. In certain embodiments —R⁹ of formula (XII) is C₂₋₂₀ alkenyl. In certain embodiments —R⁹ of formula (XII) is C₂₋₂₀ alkynyl. In certain embodiments —R⁹ of formula (XII) is -Q.

In certain embodiments —R¹⁰ of formula (XII) is C₁₋₂₀ alkyl. In certain embodiments —R¹⁰ of formula (XII) is C₂₋₂₀ alkenyl. In certain embodiments —R¹⁰ of formula (XII) is C₂₋₂₀ alkynyl.

In certain embodiments —R¹⁰ of formula (XII) is -Q.

In certain embodiments —R^{10a} of formula (XII) is C₁₋₂₀ alkyl. In certain embodiments —R^{10a} of formula (XII) is C₂₋₂₀ alkenyl. In certain embodiments —R^{10a} of formula (XII) is C₂₋₂₀ alkynyl.

In certain embodiments —R^{10a} of formula (XII) is -Q.

In certain embodiments —R¹¹ of formula (XII) is C₁₋₂₀ alkyl. In certain embodiments —R¹¹ of formula (XII) is C₂₋₂₀ alkenyl. In certain embodiments —R¹¹ of formula (XII) is C₂₋₂₀ alkynyl.

In certain embodiments —R¹¹ of formula (XII) is -Q.

In certain embodiments —R¹² of formula (XII) is C₁₋₂₀ alkyl. In certain embodiments —R¹² of formula (XII) is C₂₋₂₀ alkenyl. In certain embodiments —R¹² of formula (XII) is C₂₋₂₀ alkynyl. In certain embodiments —R¹² of formula (XII) is -Q.

In certain embodiments —R¹³ of formula (XII) is C₁₋₂₀ alkyl. In certain embodiments —R¹³ of formula (XII) is C₂₋₂₀ alkenyl. In certain embodiments —R¹³ of formula (XII) is C₂₋₂₀ alkynyl. In certain embodiments —R¹³ of formula (XII) is -Q.

In certain embodiments —R¹⁴, —R¹⁵ and —R^{15a} of formula (XII) are selected from the group consisting of —H and C₁₋₆ alkyl.

In certain embodiments —R¹⁴ of formula (XII) is —H. In certain embodiments —R¹⁴ of formula (XII) is C₁₋₆ alkyl.

In certain embodiments —R¹⁵ of formula (XII) is —H. In certain embodiments —R¹⁵ of formula (XII) is C₁₋₆ alkyl.

In certain embodiments —R^{15a} of formula (XII) is —H. In certain embodiments —R^{15a} of formula (XII) is C₁₋₆ alkyl.

In certain embodiments —Y of formula (XII) is

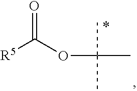

wherein —R⁵ is as defined above and the dashed line marked with an asterisk indicates the attachment to -A-.

In certain embodiments —Y of formula (XII) is

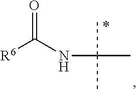

wherein —R⁶ is as defined above and the dashed line marked with an asterisk indicates the attachment to -A-.

In certain embodiments —R⁶ of formula (XII) is of formula (XIIa):

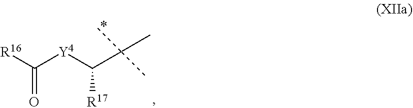

wherein —Y⁴— is selected from the group consisting of C₃₋₁₀ cycloalkyl, 3- to 10-membered heterocyclyl and 8- to 11-membered heterobicyclyl, which are optionally substituted with one or more —R¹⁸ which are the same or different;
—R¹⁶ and —R¹⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R¹⁸ which are the same or different; and
wherein C₁₋₁₀ alkyl, C₂₋₁₀ alkenyl and C₂₋₁₀ alkynyl are optionally interrupted by one or more groups selected from the group consisting of -A′-, —C(O)O—, —O—, —C(O)—, —C(O)N(R¹⁹)—, —S(O)₂N(R¹⁹), —S(O)N(R¹⁹)—, —S(O)₂—, —S(O)—, —N(R¹⁹)S(O)₂N(R^{19a})—, —S—, —N(R¹⁹)—, —OC(OR¹⁹)R^{19a}—, —N(R¹⁹)C(O)N(R^{19a})—, —OC(O)N(R¹⁹)— and —N(R¹⁹)C(NH₂)N(R^{19a})—;
   each A′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each A′ is independently optionally substituted with one or more —R¹⁸ which are the same or different;
   wherein —R¹⁸, —R¹⁹ and —R^{19a} are independently selected from the group consisting of —H and C₁₋₆ alkyl; wherein C₁₋₆ alkyl is optionally substituted with one or more halogen, which are the same or different; and
wherein the dashed line marked with an asterisk indicates the attachment to the rest of —Y.

In certain embodiments —Y⁴— of formula (XIIa) is selected from the group consisting of C₃₋₁₀ cycloalkyl, 3- to 10-membered heterocyclyl and 8- to 11-membered heterobicyclyl. In certain embodiments —Y⁴— of formula (XIIa) is C₃₋₁₀ cycloalkyl. In certain embodiments —Y⁴— of formula (XIIa) is 3- to 10-membered heterocyclyl. In certain embodiments —Y⁴— of formula (XIIa) is 8- to 11-membered heterobicyclyl. In certain embodiments —Y⁴— of formula (XIIa) is substituted with one or more —R¹⁸ which are the same or different. In certain embodiments —Y⁴— of formula (XIIa) is not substituted with —R¹⁸.

In certain embodiments —R¹⁶ and —R¹⁷ of formula (XIIa) are selected from the group consisting of C₁₋₁₀ alkyl, C₂₋₁₀ alkenyl and C₂₋₁₀ alkynyl. In certain embodiments —R¹⁶ of formula (XIIa) is C₁₋₁₀ alkyl. In certain embodiments —R¹⁶ of formula (XIIa) is C₂₋₁₀ alkenyl. In certain embodiments —R¹⁶ of formula (XIIa) is C₂₋₁₀ alkynyl. In certain embodiments —R¹⁷ of formula (XIIa) is C₁₋₁₀ alkyl. In certain embodiments —R¹⁷ of formula (XIIa) is C₂₋₁₀ alkenyl. In certain embodiments —R¹⁷ of formula (XIIa) is C₂₋₁₀ alkynyl.

In certain embodiments A′ of formula (XIIa) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments A′ of formula (XIIa) is phenyl. In certain embodiments A′ of formula (XIIa) is naphthyl. In certain embodiments A′ of formula (XIIa) is indenyl. In certain embodiments A′ of formula (XIIa) is indanyl. In certain embodiments A′ of formula (XIIa) is tetralinyl. In certain embodiments A′ of formula (XIIa) is C₃₋₁₀ cycloalkyl. In certain embodiments A′ of formula (XIIa) is 3- to 10-membered heterocyclyl. In certain embodiments A′ of formula (XIIa) is 8- to 11-membered heterobicyclyl.

In certain embodiments A′ of formula (XIIa) is substituted with one or more —R¹⁸, which are the same or different. In certain embodiments A′ of formula (XIIa) is not substituted with —R¹⁸.

In certain embodiments —R¹⁸, —R¹⁹ and —R^{19a} of formula (XIIa) are selected from the group consisting of —H and C₁₋₆ alkyl.

In certain embodiments —R¹⁸ of formula (XIIa) is —H. In certain embodiments —R¹⁸ of formula (XIIa) is C₁₋₆ alkyl. In certain embodiments —R¹⁹ of formula (XIIa) is —H. In certain embodiments —R¹⁹ of formula (XIIa) is C₁₋₆ alkyl. In certain embodiments —R^{19a} of formula (XIIa) is —H. In certain embodiments —R^{19a} of formula (XIIa) is C₁₋₆ alkyl.

In certain embodiments —R⁶ of formula (XII) is of formula (XIIb):

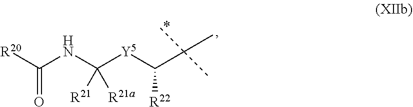

wherein —Y⁵— is selected from the group consisting of -Q′-, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³, which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—;
—R²⁰, —R²¹, —R^{21a} and —R²² are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³ which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴), —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH)N(R^{24a})—;
   each Q′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q′ is independently optionally substituted with one or more —R²³, which are the same or different;
   wherein —R²³, —R²⁴ and —R^{24a} are independently selected from the group consisting of —H and C₁₋₆ alkyl; wherein C₁₋₆ alkyl is optionally substituted with one or more halogen, which are the same or different;
optionally, the pair —R²¹/—R^{21a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of —Y.

In certain embodiments —Y⁵— of formula (XIIb) is selected from the group consisting of -Q′-, C₁₋₁₀ alkyl, C₂₋₁₀ alkenyl and C₂₋₁₀ alkynyl. In certain embodiments —Y⁵— of formula (XIIb) is -Q′-. In certain embodiments —Y⁵— of formula (XIIb) is C₁₋₁₀ alkyl. In certain embodiments —Y⁵— of formula (XIIb) is C₂₋₁₀ alkenyl. In certain embodiments —Y⁵— of formula (XIIb) is C₂₋₁₀ alkynyl.

In certain embodiments Q′ of formula (XIIb) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q′ of formula (XIIb) is phenyl. In certain embodiments Q′ of formula (XIIb) is naphthyl. In certain embodiments Q′ of formula (XIIb) is indenyl. In certain embodiments Q′ of formula (XIIb) is indanyl. In certain embodiments Q′ of formula (XIIb) is C₃₋₁₀ cycloalkyl. In certain embodiments Q′ of formula (XIIb) is 3- to 10-membered heterocyclyl. In certain embodiments Q′ of formula (XIIb) is 8- to 11-membered heterobicyclyl. In certain embodiments Q′ of formula (XIIb) is substituted with one or more —R²³ which are the same or different. In certain embodiments Q′ of formula (XIIb) is not substituted with —R²³.

In certain embodiments —R²⁰, —R²¹, —R^{21a} and —R²² of formula (XIIb) are selected from the group consisting of —H, C₁₋₁₀ alkyl, C₂₋₁₀ alkenyl and C₂₋₁₀ alkynyl. In certain embodiments —R²⁰ of formula (XIIb) is —H. In certain embodiments —R²⁰ of formula (XIIb) is C₁₋₁₀ alkyl. In certain embodiments —R²⁰ of formula (XIIb) is C₂₋₁₀ alkenyl. In certain embodiments —R²⁰ of formula (XIIb) is C₂₋₁₀ alkynyl. In certain embodiments —R²¹ of formula (XIIb) is —H. In certain embodiments —R²¹ of formula (XIIb) is C₁₋₁₀ alkyl. In certain embodiments —R²¹ of formula (XIIb) is C₂₋₁₀ alkenyl. In certain embodiments —R²¹ of formula (XIIb) is C₂₋₁₀ alkynyl. In certain embodiments —R^{21a} of formula (XIIb) is —H. In certain embodiments —R^{21a} of formula (XIIb) is C₁₋₁₀ alkyl. In certain embodiments —R^{21a} of formula (XIIb) is C₂₋₁₀ alkenyl.

In certain embodiments —R^{21a} of formula (XIIb) is C₂₋₁₀ alkynyl. In certain embodiments —R²² of formula (XIIb) is —H. In certain embodiments —R²² of formula (XIIb) is C₁₋₁₀ alkyl. In certain embodiments —R²² of formula (XIIb) is C₂₋₁₀ alkenyl. In certain embodiments —R²² of formula (XIIb) is C₂₋₁₀ alkynyl.

In certain embodiments —R²³, —R²⁴ and —R^{24a} of formula (XIIb) are selected from the group consisting of —H and C₁₋₆ alkyl. In certain embodiments —R²³ of formula (XIIb) is —H. In certain embodiments —R²³ of formula (XIIb) is C₁₋₆ alkyl. In certain embodiments —R²⁴ of formula (XIIb) is —H. In certain embodiments —R²⁴ of formula (XIIb) is C₁₋₆ alkyl. In certain embodiments —R^{24a} of formula (XIIb) is —H. In certain embodiments —R^{24a} of formula (XIIb) is C₁₋₆ alkyl.

In certain embodiments the pair —R²¹/—R^{21a} of formula (XIIb) is joined together with the atoms to which is attached to form a C₃₋₁₀ cycloalkyl.

In certain embodiments —R⁶ of formula (XIIb) is of formula (XIIc):

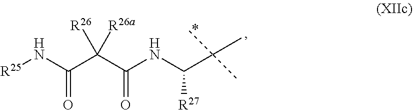

wherein
—R²⁵, —R²⁶, —R^{26a} and —R^{2′} are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²⁸ which are the same or different; and wherein C₁₋₁₀ alkyl, C₂₋₁₀ alkenyl and C₂₋₁₀ alkynyl are optionally interrupted by one or more groups selected from the group consisting of -Q*-, —C(O)O—, —O—, —C(O)—, —C(O)N(R²⁹)—, —S(O)₂N(R²⁹)—, —S(O)N(R²⁹)—, —S(O)₂—, —S(O)—, —N(R²⁹)S(O)₂N(R^{29a})—, —S—, —N(R²⁹)—, —OC(OR²⁹)R^{29a}—, —N(R²⁹)C(O)N(R^{29a})—, —OC(O)N(R²⁹)— and —N(R²⁹)C(NH₂)N(R^{29a})—;
   each Q* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q* is independently optionally substituted with one or more —R²⁸, which are the same or different;
   wherein —R²⁸, —R²⁹ and —R^{29a} are independently selected from the group consisting of —H and C₁₋₆ alkyl; wherein C₁₋₆ alkyl is optionally substituted with one or more halogen, which are the same or different;
optionally, the pair —R²⁶/—R^{26a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of —Y.

In certain embodiments —R²⁵, —R²⁶, —R^{26a} and —R²⁷ of formula (XIIc) are selected from the group consisting of —H, C₁₋₁₀ alkyl, C₂₋₁₀ alkenyl and C₂₋₁₀ alkynyl. In certain embodiments —R²⁵ of formula (XIIc) is —H. In certain embodiments —R²⁵ of formula (XIIc) is C₁₋₁₀ alkyl. In certain embodiments —R²⁵ of formula (XIIc) is C₂₋₁₀ alkenyl. In certain embodiments —R²⁵ of formula (XIIc) is C₂₋₁₀ alkynyl. In certain embodiments —R²⁶ of formula (XIIc) is —H. In certain embodiments —R²⁶ of formula (XIIc) is C₁₋₁₀ alkyl. In certain embodiments —R²⁶ of formula (XIIc) is C₂₋₁₀ alkenyl. In certain embodiments —R²⁶ of formula (XIIc) is C₂₋₁₀ alkynyl. In certain embodiments —R^{26a} of formula (XIIc) is —H. In certain embodiments —R^{26a} of formula (XIIc) is C₁₋₁₀ alkyl. In certain embodiments —R^{26a} of formula (XIIc) is C₂₋₁₀ alkenyl. In certain embodiments —R^{26a} of formula (XIIc) is C₂₋₁₀ alkynyl. In certain embodiments —R²⁷ of formula (XIIc) is —H. In certain embodiments —R²⁷ of formula (XIIc) is C₁₋₁₀ alkyl. In certain embodiments —R²⁷ of formula (XIIc) is C₂₋₁₀ alkenyl. In certain embodiments —R²⁷ of formula (XIIc) is C₂₋₁₀ alkynyl.

In certain embodiments Q* of formula (XIIc) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q* of formula (XIIc) is phenyl. In certain embodiments Q* of formula (XIIc) is naphthyl. In certain embodiments Q* of formula (XIIc) is indenyl. In certain embodiments Q* of formula (XIIc) is indanyl. In certain embodiments Q* of formula (XIIc) is tetralinyl. In certain embodiments Q* of formula (XIIc) is C₃₋₁₀ cycloalkyl. In certain embodiments Q* of formula (XIIc) is 3- to 10-membered heterocyclyl. In certain embodiments Q* of formula (XIIc) is 8- to 11-membered heterobicyclyl. In certain embodiments Q* of formula (XIIc) is substituted with one or more —R²⁸, which are the same or different. In certain embodiments Q* of formula (XIIc) is not substituted with —R²⁸.

In certain embodiments —R²⁸, —R²⁹ and —R^{29a} of formula (XIIc) are selected from the group consisting of —H and C₁₋₆ alkyl. In certain embodiments —R²⁸ of formula (XIIc) is —H. In certain embodiments —R²⁸ of formula (XIIc) is C₁₋₆ alkyl. In certain embodiments —R²⁹ of formula (XIIc) is —H. In certain embodiments —R²⁹ of formula (XIIc) is C₁₋₆ alkyl. In certain embodiments —R^{29a} of formula (XIIc) is —H. In certain embodiments —R^{29a} of formula (XIIc) is C₁₋₆ alkyl.

In certain embodiments the pair —R²⁶/—R^{26a} of formula (XIIc) is joined together with the atoms to which is attached to form a C₃₋₁₀ cycloalkyl. In certain embodiments the pair —R²⁶/—R^{26a} of formula (XIIc) is joined together with the atoms to which is attached to form a cyclobutyl.

In certain embodiments —Y of formula (XII) is

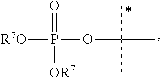

wherein each —R⁷ is as defined above and the dashed line marked with an asterisk indicates the attachment to -A-. It is understood that in this instance the release of the drug D may be triggered by an enzyme, such as phosphatase.

In certain embodiments —Y of formula (XII) is

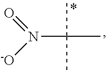

wherein the dashed line marked with an asterisk indicates the attachment to -A-.

In certain embodiments —Y of formula (XII) is

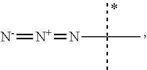

wherein the dashed line marked with an asterisk indicates the attachment to -A-.

In certain embodiments —Y of formula (XII) is

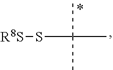

wherein —R⁸ is as defined above and the dashed line marked with an asterisk indicates the attachment to -A-.

In certain embodiments —Y of formula (XII) is

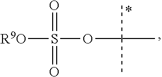

wherein —R⁹ is as defined above and the dashed line marked with an asterisk indicates the attachment to -A-. It is understood that in this instance the release of the drug D may be triggered by an enzyme, such as sulfatase.

In certain embodiments —Y of formula (XII) is

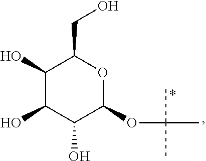

wherein the dashed line marked with an asterisk indicates the attachment to -A-. It is understood that in this instance the release of the drug D may be triggered by an enzyme, such as α-galactosidase.

In certain embodiments —Y of formula (XII) is

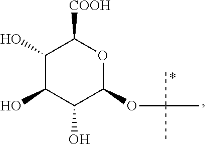

wherein the dashed line marked with an asterisk indicates the attachment to -A-. It is understood that in this instance the release of the drug D may be triggered by an enzyme, such as β-glucuronidase.

In certain embodiments —Y of formula (XII) is

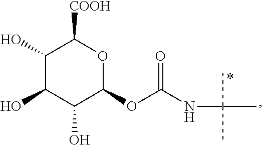

wherein the dashed line marked with an asterisk indicates the attachment to -A-. It is understood that in this instance the release of the drug D may be triggered by an enzyme, such as β-glucuronidase.

In certain embodiments —Y of formula (XII) is a peptidyl moiety.

It is understood that if —Y of formula (XII) is a peptidyl moiety, then the release of the drug D may be triggered by an enzyme, such as protease. In certain embodiments the protease is selected from the group consisting of cathepsin B and cathepsin K. In certain embodiments the protease is cathepsin B. In certain embodiments the protease is cathepsin K.

In certain embodiments —Y of formula (XII) is a peptidyl moiety, such as a dipeptidyl, tripeptidyl, tetrapeptidyl, pentapeptidyl or hexapeptidyl moiety. In certain embodiments —Y of formula (XII) is a dipeptidyl moiety. In certain embodiments —Y of formula (XII) is a tripeptidyl moiety. In certain embodiments —Y of formula (XII) is a tetrapeptidyl moiety. In certain embodiments —Y of formula (XII) is a pentapeptidyl moiety. In certain embodiments —Y of formula (XII) is a hexapeptidyl moiety.

In certain embodiments —Y of formula (XII) is a peptidyl moiety selected from the group consisting of

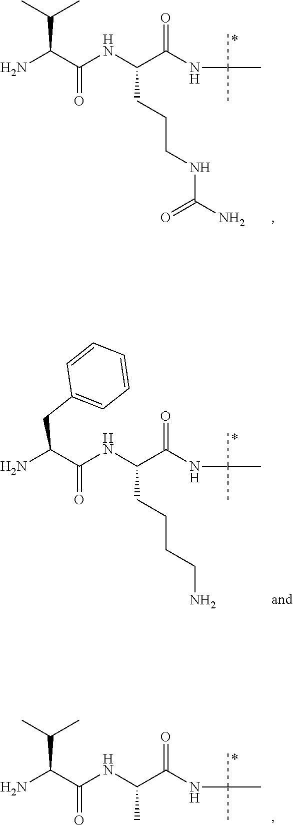

wherein the dashed line marked with an asterisk indicates the attachment to -A-.

In certain embodiments —Y of formula (XII) is

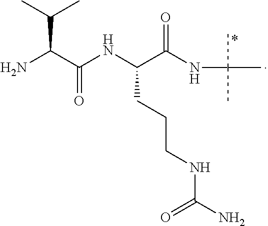

In certain embodiments —Y of formula (XII) is

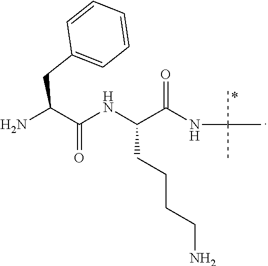

In certain embodiments —Y of formula (XII) is

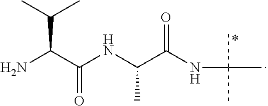

In certain embodiments one hydrogen given by —R^{1a} of formula (XII) is replaced by -L²- and -L¹- is of formula (XII′):

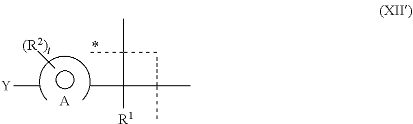

wherein
the unmarked dashed line indicates the attachment to the N⁺ of -D⁺, the dashed line marked with an asterisk indicates the attachment to -L²-; and
—R¹, -A-, —Y, R² and t are defined as in formula (XII).

In certain embodiments one hydrogen given by —R² of formula (XII) is replaced by -L²- and -L¹- is of formula (XII″):

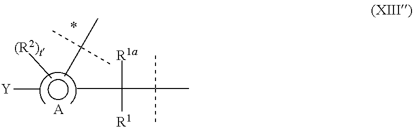

wherein
the unmarked dashed line indicates the attachment to the N⁺ of -D⁺, the dashed line marked with an asterisk indicates the attachment to -L²-;
—R¹, —R^{1a}—, -A-, —Y and R² are defined as in formula (XII); and
t′ is selected from the group consisting of 0, 1, 2, 3, 4 and 5.

In certain embodiments t′ of formula (XII″) is 0. In certain embodiments t′ of formula (XII″) is 1. In certain embodiments t′ of formula (XII″) is 2. In certain embodiments t′ of formula (XII″) is 3. In certain embodiments t′ of formula (XII″) is 4. In certain embodiments t′ of formula (XII″) is 5.

In certain embodiments -L¹- is of formula (XIII):

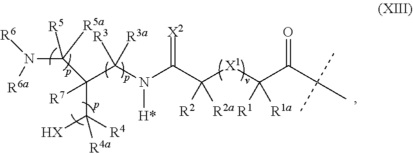

wherein
the dashed line indicates the attachment to the nitrogen of the primary or secondary amine of -D;
v is selected from the group consisting of 0 or 1;
—X¹— is selected from the group consisting of —C(R⁵)(R^{8a})—, —N(R⁹)— and —O—;
═X² is selected from the group consisting of ═O and ═N(R¹⁰);
—X³ is selected from the group consisting of —O, —S and —Se;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R¹¹, —R², —R^{2a}, —R³, —R^{3a}, —R⁴, —R^{4a}, —R⁵, —R^{5a}, —R⁷, —R⁸, —R^{8a}, —R¹¹, —R^{11a} and —R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   —R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³ which are the same or different and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
      wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
      wherein each T is independently optionally substituted with one or more —R¹³, which are the same or different;
—R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O) N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
   wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁴/—R^{4a}, —R⁵/—R^{5a} or —R⁸/—R^{5a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R¹, —R¹/—R⁹, —R²/—R⁹ or —R²/—R¹⁰ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
   wherein -A′- is selected from the group consisting of 3- to 10-membered heterocyclyl and 8- to 11-membered heterobicyclyl; and
-L¹- is substituted with at least one -L²- and optionally further substituted provided that the hydrogen marked with the asterisk in formula (XIII) is not replaced by a substituent.

In certain embodiments the dashed line in formula (XIII) indicates attachment to a nitrogen of a primary amine of -D. In certain embodiments the dashed line in formula (XIII) indicates attachment to a nitrogen of a secondary amine of -D.

In certain embodiments, —X³ of formula (XIII) is —O. In certain embodiments, —X³ of formula (XIII) is —S. In certain embodiments, —X³ of formula (XIII) is —Se.

In certain embodiments, —R⁶ of formula (XIII) is —H. In certain embodiments, —R⁶ of formula (XIII) is —C(R¹¹)(R^{11a})(R^{11b}). In certain embodiments, —R⁶ of formula (XIII) is -T.

In certain embodiments, —R^{6a} of formula (XIII) is —H. In certain embodiments, —R^{6a} of formula (XIII) is —C(R¹¹)(R^{11a})(R^{11b}). In certain embodiments, —R^{6a} of formula (XIII) is -T.

In certain embodiments, both —R⁶ and —R^{6a} of formula (XIII) are —H.

In certain embodiments, v of formula (XIII) is 0. In certain embodiments, v of formula (XIII) is 1.

In certain embodiments, —X¹— of formula (XIII) is —C(R⁵)(R^{8a})—. In certain embodiments, —X¹— of formula (XIII) is —N(R⁹)—. In certain embodiments, —X¹— of formula (XIII) is —O—.

In certain embodiments, ═X² of formula (XIII) is ═O. In certain embodiments, ═X² of formula (XIII) is ═N(R¹⁰).

In certain embodiments, —R⁹ of formula (XIII) is —C(R¹¹)(R^{11a})(R^{11b}). In certain embodiments, —R⁹ of formula (XIII) is -T.

In certain embodiments, —R¹⁰ of formula (XIII) is —H. In certain embodiments, —R¹⁰ of formula (XIII) is —C(R¹¹)(R^{11a})(R^{11b}). In certain embodiments, —R¹⁰ of formula (XIII) is -T.

In certain embodiments, —R¹ of formula (XIII) is selected from the group consisting of —H, halogen, —CN, —C(O)OR¹², —OR¹², —C(O)R¹², —C(O)N(R¹)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R¹ of formula (XIII) is —H. In certain embodiments, —R¹ of formula (XIII) is halogen. In certain embodiments, —R¹ of formula (XIII) is -T. In certain embodiments, —R¹ of formula (XIII) is C₁₋₆ alkyl. In certain embodiments, —R¹ of formula (XIII) is C₂₋₆ alkenyl. In certain embodiments, —R¹ of formula (XIII) is C₂₋₆ alkynyl. In certain embodiments, —R¹ of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{1a} of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{1a} of formula (XIII) is —H. In certain embodiments, —R^{1a} of formula (XIII) is halogen. In certain embodiments, —R^{1a} of formula (XIII) is -T. In certain embodiments, —R^{1a} of formula (XIII) is C₁₋₆ alkyl. In certain embodiments, —R^{1a} of formula (XIII) is C₂₋₆ alkenyl. In certain embodiments, —R^{1a} of formula (XIII) is C₂₋₆ alkynyl. In certain embodiments, —R^{1a} of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R² of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R² of formula (XIII) is —H. In certain embodiments, —R² of formula (XIII) is halogen. In certain embodiments, —R² of formula (XIII) is -T. In certain embodiments, —R² of formula (XIII) is C₁₋₆ alkyl. In certain embodiments, —R² of formula (XIII) is C₂₋₆ alkenyl. In certain embodiments, —R² of formula (XIII) is C₂₋₆ alkynyl. In certain embodiments, —R² of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{2a} of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{2a} of formula (XIII) is —H. In certain embodiments, —R^{2a} of formula (XIII) is halogen. In certain embodiments, —R^{2a} of formula (XIII) is -T. In certain embodiments, —R^{2a} of formula (XIII) is C₁₋₆ alkyl. In certain embodiments, —R^{2a} of formula (XIII) is C₂₋₆ alkenyl. In certain embodiments, —R^{2a} of formula (XIII) is C₂₋₆ alkynyl. In certain embodiments, —R^{2a} of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R³ of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R³ of formula (XIII) is —H. In certain embodiments, —R³ of formula (XIII) is halogen. In certain embodiments, —R³ of formula (XIII) is -T. In certain embodiments, —R³ of formula (XIII) is C₁₋₆ alkyl. In certain embodiments, —R³ of formula (XIII) is C₂₋₆ alkenyl. In certain embodiments, —R³ of formula (XIII) is C₂₋₆ alkynyl. In certain embodiments, —R³ of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{3a} of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{3a} of formula (XIII) is —H. In certain embodiments, —R^{3a} of formula (XIII) is halogen. In certain embodiments, —R^{3a} of formula (XIII) is -T. In certain embodiments, —R^{3a} of formula (XIII) is C₁₋₆ alkyl. In certain embodiments, —R^{3a} of formula (XIII) is C₂₋₆ alkenyl. In certain embodiments, —R^{3a} of formula (XIII) is C₂₋₆ alkynyl. In certain embodiments, —R^{3a} of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁴ of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁴ of formula (XIII) is —H. In certain embodiments, —R⁴ of formula (XIII) is halogen. In certain embodiments, —R⁴ of formula (XIII) is -T. In certain embodiments, —R⁴ of formula (XIII) is C₁₋₆ alkyl. In certain embodiments, —R⁴ of formula (XIII) is C₂₋₆ alkenyl. In certain embodiments, —R⁴ of formula (XIII) is C₂₋₆ alkynyl. In certain embodiments, —R⁴ of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{4a} of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{4a} of formula (XIII) is —H. In certain embodiments, —R^{4a} of formula (XIII) is halogen. In certain embodiments, —R^{4a} of formula (XIII) is -T. In certain embodiments, —R^{4a} of formula (XIII) is C₁₋₆ alkyl. In certain embodiments, —R^{4a} of formula (XIII) is C₂₋₆ alkenyl. In certain embodiments, —R^{4a} of formula (XIII) is C₂₋₆ alkynyl. In certain embodiments, —R^{4a} of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁵ of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁵ of formula (XIII) is —H. In certain embodiments, —R⁵ of formula (XIII) is halogen. In certain embodiments, —R⁵ of formula (XIII) is -T. In certain embodiments, —R⁵ of formula (XIII) is C₁₋₆ alkyl. In certain embodiments, —R⁵ of formula (XIII) is C₂₋₆ alkenyl. In certain embodiments, —R⁵ of formula (XIII) is C₂₋₆ alkynyl. In certain embodiments, —R⁵ of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{5a} of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{5a} of formula (XIII) is —H. In certain embodiments, —R^{5a} of formula (XIII) is halogen. In certain embodiments, —R^{5a} of formula (XIII) is -T. In certain embodiments, —R^{5a} of formula (XIII) is C₁₋₆ alkyl. In certain embodiments, —R^{5a} of formula (XIII) is C₂₋₆ alkenyl. In certain embodiments, —R^{5a} of formula (XIII) is C₂₋₆ alkynyl. In certain embodiments, —R^{5a} of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁷ of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁷ of formula (XIII) is —H. In certain embodiments, —R⁷ of formula (XIII) is halogen. In certain embodiments, —R⁷ of formula (XIII) is -T. In certain embodiments, —R⁷ of formula (XIII) is C₁₋₆ alkyl. In certain embodiments, —R⁷ of formula (XIII) is C₂₋₆ alkenyl. In certain embodiments, —R⁷ of formula (XIII) is C₂₋₆ alkynyl. In certain embodiments, —R⁷ of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R⁸ of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R⁸ of formula (XIII) is —H. In certain embodiments, —R⁸ of formula (XIII) is halogen. In certain embodiments, —R⁸ of formula (XIII) is -T. In certain embodiments, —R⁸ of formula (XIII) is C₁₋₆ alkyl. In certain embodiments, —R⁸ of formula (XIII) is C₂₋₆ alkenyl. In certain embodiments, —R⁸ of formula (XIII) is C₂₋₆ alkynyl. In certain embodiments, —R⁸ of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{8a} of formula (XIII) is selected from the group consisting of —H, halogen, —CN, —C(O)OR¹², —OR¹², —C(O)R¹², —C(O)N(R¹)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{8a} of formula (XIII) is —H. In certain embodiments, —R^{8a} of formula (XIII) is halogen. In certain embodiments, —R^{8a} of formula (XIII) is -T. In certain embodiments, —R^{8a} of formula (XIII) is C₁₋₆ alkyl. In certain embodiments, —R^{8a} of formula (XIII) is C₂₋₆ alkenyl. In certain embodiments, —R^{8a} of formula (XIII) is C₂₋₆ alkynyl. In certain embodiments, —R^{8a} of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹¹ of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R¹¹ of formula (XIII) is —H. In certain embodiments, —R¹¹ of formula (XIII) is halogen. In certain embodiments, —R¹¹ of formula (XIII) is -T. In certain embodiments, —R¹¹ of formula (XIII) is C₁₋₆ alkyl. In certain embodiments, —R¹¹ of formula (XIII) is C₂₋₆ alkenyl. In certain embodiments, —R¹¹ of formula (XIII) is C₂₋₆ alkynyl. In certain embodiments, —R¹¹ of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R^{11a} of formula (XIII) is selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl. In certain embodiments, —R^{11a} of formula (XIII) is —H. In certain embodiments, —R^{11a} of formula (XIII) is halogen. In certain embodiments, —R^{11a} of formula (XIII) is -T. In certain embodiments, —R^{11a} of formula (XIII) is C₁₋₆ alkyl. In certain embodiments, —R^{11a} of formula (XIII) is C₂₋₆ alkenyl. In certain embodiments,—of formula (XIII) is C₂₋₆ alkynyl. In certain embodiments, —R^{11a} of formula (XIII) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments, —R¹² of formula (XIII) is selected from the group consisting of —H, -T, C₁₋₆ alkyl, C₂₋₆ alkenyl and C₂₋₆ alkynyl. In certain embodiments, —R¹² of formula (XIII) is —H. In certain embodiments, —R¹² of formula (XIII) is -T. In certain embodiments, —R¹² of formula (XIII) is C₁₋₆ alkyl. In certain embodiments, —R¹² of formula (XIII) is C₂₋₆ alkenyl. In certain embodiments, —R¹² of formula (XIII) is C₂₋₆ alkynyl.

In certain embodiments, —R^{12a} of formula (XIII) is selected from the group consisting of —H, -T, C₁₋₆ alkyl, C₂₋₆ alkenyl and C₂₋₆ alkynyl. In certain embodiments, —R^{12a} of formula (XIII) is —H. In certain embodiments, —R^{12a} of formula (XIII) is -T. In certain embodiments, —R^{12a} of formula (XIII) is C₁₋₆ alkyl. In certain embodiments, —R^{12a} of formula (XIII) is C₂₋₆ alkenyl. In certain embodiments, —R^{12a} of formula (XIII) is C₂₋₆ alkynyl.

In certain embodiments, —R^{12b} of formula (XIII) is selected from the group consisting of —H, -T, C₁₋₆ alkyl, C₂₋₆ alkenyl and C₂₋₆ alkynyl. In certain embodiments, —R^{12b} of formula (XIII) is —H. In certain embodiments, —R^{12b} of formula (XIII) is -T. In certain embodiments, —R^{12b} of formula (XIII) is C₁₋₆ alkyl. In certain embodiments, —R^{12b} of formula (XIII) is C₂₋₆ alkenyl. In certain embodiments, —R^{12b} of formula (XIII) is C₂₋₆ alkynyl.

In certain embodiments, T of formula (XIII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, T of formula (XIII) is phenyl. In certain embodiments, T of formula (XIII) is naphthyl. In certain embodiments, T of formula (XIII) is indenyl. In certain embodiments, T of formula (XIII) is indanyl. In certain embodiments, T of formula (XIII) is tetralinyl. In certain embodiments, T of formula (XIII) is tetralinyl. In certain embodiments, T of formula (XIII) is C₃₋₁₀ cycloalkyl. In certain embodiments, T of formula (XIII) is 3- to 10-membered heterocyclyl. In certain embodiments, T of formula (XIII) is 8- to 11-membered heterobicyclyl.

In certain embodiments, T of formula (XIII) is substituted with one or more —R¹³ of formula (XIII), which are the same of different.

In certain embodiments, T of formula (XIII) is substituted with one —R¹³ of formula (XIII).

In certain embodiments, T of formula (XIII) is not substituted with —R¹³.

In certain embodiments, —R¹³ of formula (XIII) is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹⁵, —S(O)R¹⁵, —N(R¹⁵)S(O)₂N(R^{15a})(R^{15b}), —SR¹⁵, —N(R¹⁵)(R^{15a}), —NO₂, —OC(O)R¹⁵, —N(R¹⁵)C(O)R^{15a}, —N(R¹)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl. In certain embodiments, —R¹³ of formula (XIII) is halogen. In certain embodiments, —R¹³ of formula (XIII) is —CN. In certain embodiments, —R¹³ of formula (XIII) is oxo. In certain embodiments, —R¹³ of formula (XIII) is —C(O)OR¹⁵. In certain embodiments, —R¹³ of formula (XIII) is —OR¹⁵. In certain embodiments, —R¹³ of formula (XIII) is —C(O)R¹⁵. In certain embodiments, —R¹³ of formula (XIII) is —C(O)N(R¹⁵)(R^{15a}). In certain embodiments, —R¹³ of formula (XIII) is —S(O)₂N(R¹⁵)(R^{15a}). In certain embodiments, —R¹³ of formula (XIII) is —S(O)N(R¹⁵)(R^{15a}). In certain embodiments, —R¹³ of formula (XIII) is —S(O)₂R¹⁵. In certain embodiments, —R¹³ of formula (XIII) is —S(O)R¹⁵. In certain embodiments, —R¹³ of formula (XIII) is —N(R¹⁵)S(O)₂N(R^{15a})(R^{15b}). In certain embodiments, —R¹³ of formula (XIII) is —SR¹⁵.

In certain embodiments, —R¹³ of formula (XIII) is —N(R¹⁵)(R^{15a}). In certain embodiments, —R¹³ of formula (XIII) is —NO₂. In certain embodiments, —R¹³ of formula (XIII) is —OC(O)R¹⁵. In certain embodiments, —R¹³ of formula (XIII) is —N(R¹⁵)C(O)R^{15a}. In certain embodiments, —R¹³ of formula (XIII) is —N(R¹⁵)S(O)₂R^{15a}. In certain embodiments, —R¹³ of formula (XIII) is —N(R¹⁵)S(O)R^{15a}. In certain embodiments, —R¹³ of formula (XIII) is —N(R¹⁵)C(O)OR^{15a}. In certain embodiments, —R¹³ of formula (XIII) is —N(R¹⁵)C(O)N(R^{15a})(R^{15b}). In certain embodiments, —R¹³ of formula (XIII) is —OC(O)N(R¹⁵)(R^{15a}). In certain embodiments, —R¹³ of formula (XIII) is C₁₋₆ alkyl.

In certain embodiments, —R¹⁴ of formula (XIII) is selected from the group consisting of —H and C₁₋₆ alkyl. In certain embodiments, —R¹⁴ of formula (XIII) is —H. In certain embodiments, —R¹⁴ of formula (XIII) is C₁₋₆ alkyl.

In certain embodiments, —R^{14a} of formula (XIII) is selected from the group consisting of —H and C₁₋₆ alkyl. In certain embodiments, —R^{14a} of formula (XIII) is —H. In certain embodiments, —R^{14a} of formula (XIII) is C₁₋₆ alkyl.

In certain embodiments, —R¹⁵ of formula (XIII) is selected from the group consisting of —H and C₁₋₆ alkyl. In certain embodiments, —R¹⁵ of formula (XIII) is —H. In certain embodiments, —R¹⁵ of formula (XIII) is C₁₋₆ alkyl.

In certain embodiments, —R^{15a} of formula (XIII) is selected from the group consisting of —H and C₁₋₆ alkyl. In certain embodiments, —R^{15a} of formula (XIII) is —H. In certain embodiments, —R^{15a} of formula (XIII) is C₁₋₆ alkyl.

In certain embodiments, —R^{15b} of formula (XIII) is selected from the group consisting of —H and C₁₋₆ alkyl. In certain embodiments, —R^{15b} of formula (XIII) is —H. In certain embodiments, —R^{15b} of formula (XIII) is C₁₋₆ alkyl.

In certain embodiments, —R¹ and —R^{1a} of formula (XIII) are joined together with the atom to which they are attached to form C₃₋₁₀ cycloalkyl. In certain embodiments, —R¹ and —R^{1a} of formula (XIII) are joined together with the atom to which they are attached to form 3- to 10-membered heterocyclyl. In certain embodiments, —R¹ and —R^{1a} of formula (XIII) are joined together with the atom to which they are attached to form an 8- to 11-membered heterobicyclyl.

In certain embodiments, —R² and —R^{2a} of formula (XIII) are joined together with the atom to which they are attached to form C₃₋₁₀ cycloalkyl. In certain embodiments, —R² and —R^{2a} of formula (XIII) are joined together with the atom to which they are attached to form 3- to 10-membered heterocyclyl. In certain embodiments, —R² and —R^{2a} of formula (XIII) are joined together with the atom to which they are attached to form an 8- to 11-membered heterobicyclyl.

In certain embodiments, —R³ and —R^{3a} of formula (XIII) are joined together with the atom to which they are attached to form C₃₋₁₀ cycloalkyl. In certain embodiments, —R³ and —R^{3a} of formula (XIII) are joined together with the atom to which they are attached to form 3- to 10-membered heterocyclyl. In certain embodiments, —R³ and —R^{3a} of formula (XIII) are joined together with the atom to which they are attached to form an 8- to 11-membered heterobicyclyl.

In certain embodiments, —R⁴ and —R^{4a} of formula (XIII) are joined together with the atom to which they are attached to form C₃₋₁₀ cycloalkyl. In certain embodiments, —R⁴ and —R^{4a} of formula (XIII) are joined together with the atom to which they are attached to form 3- to 10-membered heterocyclyl. In certain embodiments, —R⁴ and —R^{4a} of formula (XIII) are joined together with the atom to which they are attached to form an 8- to 11-membered heterobicyclyl.

In certain embodiments, —R⁵ and —R^{5a} of formula (XIII) are joined together with the atom to which they are attached to form C₃₋₁₀ cycloalkyl. In certain embodiments, —R⁵ and —R^{5a} of formula (XIII) are joined together with the atom to which they are attached to form 3- to 10-membered heterocyclyl. In certain embodiments, —R⁵ and —R^{5a} of formula (XIII) are joined together with the atom to which they are attached to form an 8- to 11-membered heterobicyclyl.

In certain embodiments, —R⁸ and —R^{8a} of formula (XIII) are joined together with the atom to which they are attached to form C₃₋₁₀ cycloalkyl. In certain embodiments, —R⁸ and —R^{8a} of formula (XIII) are joined together with the atom to which they are attached to form 3- to 10-membered heterocyclyl. In certain embodiments, —R⁸ and —R^{8a} of formula (XIII) are joined together with the atom to which they are attached to form an 8- to 11-membered heterobicyclyl.

In certain embodiments, —R¹ and —R² of formula (XIII) are joined together with the atoms to which they are attached to form a ring -A- of formula (XIII).

In certain embodiments, —R¹ and —R⁸ of formula (XIII) are joined together with the atoms to which they are attached to form a ring -A- of formula (XIII).

In certain embodiments, —R¹ and —R⁹ of formula (XIII) are joined together with the atoms to which they are attached to form a ring -A- of formula (XIII).

In certain embodiments, —R² and —R⁹ of formula (XIII) are joined together with the atoms to which they are attached to form a ring -A- of formula (XIII).

In certain embodiments, —R² and —R¹⁰ of formula (XIII) are joined together with the atoms to which they are attached to form a ring -A- of formula (XIII).

In certain embodiments, -A- of formula (XIII) is phenyl. In certain embodiments, -A- of formula (XIII) is naphthyl. In certain embodiments, -A- of formula (XIII) is indenyl. In certain embodiments, -A- of formula (XIII) is indanyl. In certain embodiments, -A- of formula (XIII) is tetralinyl. In certain embodiments, -A- of formula (XIII) is C₃₋₁₀ cycloalkyl.

In certain embodiments, -A- of formula (XIII) is 3- to 10-membered heterocyclyl. In certain embodiments, -A- of formula (XIII) is 8- to 11-membered heterobicyclyl.

In certain embodiments, —R³ and —R⁶ of formula (XIII) are joined together with the atoms to which they are attached to form a ring -A′- of formula (XIII).

In certain embodiments, —R⁴ and —R⁶ of formula (XIII) are joined together with the atoms to which they are attached to form a ring -A′- of formula (XIII).

In certain embodiments, —R⁵ and —R⁶ of formula (XIII) are joined together with the atoms to which they are attached to form a ring -A′- of formula (XIII).

In certain embodiments, —R⁶ and —R^{6a} of formula (XIII) are joined together with the atoms to which they are attached to form a ring -A′- of formula (XIII).

In certain embodiments, —R⁶ and —R⁷ of formula (XIII) are joined together with the atoms to which they are attached to form a ring -A′- of formula (XIII).

In certain embodiments, -A′- of formula (XIII) or (II) is 3- to 10-membered heterocyclyl. In certain embodiments, -A′- of formula (XIII) is 8- to 11-membered heterobicyclyl.

In certain embodiments -L¹- is of formula (XIIIa)

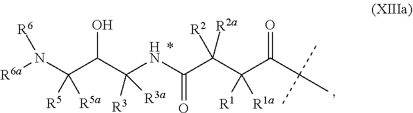

wherein
the dashed line indicates attachment to the nitrogen of the primary or secondary amine of -D;
—R¹, —R^{1a}, —R², —R^{2a}, —R³, —R^{3a}, —R⁵, —R^{5a}, —R⁶ and —R^{6a} are used as defined in formula (XIII); and
-L¹- is substituted with at least one moiety -L²- and is optionally further substituted, provided that the hydrogen marked with the asterisk in formula (XIIIa) is not replaced by a substituent.

In certain embodiments the dashed line in formula (XIIIa) indicates attachment to a nitrogen of a primary amine of -D. In certain embodiments the dashed line in formula (XIIIa) indicates attachment to a nitrogen of a secondary amine of -D.

In certain embodiments —R¹ is —H. In certain embodiments —R^{1a} is —H. In certain embodiments —R² is —H. In certain embodiments —R^{2a} is —H. In certain embodiments —R³ is —H. In certain embodiments —R^{3a} is —H. In certain embodiments —R⁵ is —H. In certain embodiments —R^{5a} is —H. In certain embodiments —R⁶ is —H. In certain embodiments —R^{6a} is —H.

In certain embodiments -L¹- of formula (XIIIa) is not further substituted.

In certain embodiments —R¹ is —H, which —H is substituted with -L². In certain embodiments —R^{1a} is —H, which —H is substituted with -L²-. In certain embodiments —R² is —H, which —H is substituted with -L²-. In certain embodiments —R^{2a} is —H, which —H is substituted with -L²-. In certain embodiments —R³ is —H, which —H is substituted with -L²-. In certain embodiments —R^{3a} is —H, which —H is substituted with -L²-. In certain embodiments —R⁵ is —H, which —H is substituted with -L²-. In certain embodiments —R^{5a} is —H, which —H is substituted with -L²-. In certain embodiments —R⁶ is —H, which —H is substituted with -L²-. In certain embodiments —R^{6a} is —H, which —H is substituted with -L²-.

In certain embodiments -L¹- is of formula (XIIIb)

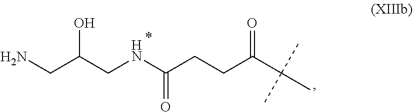

wherein
the dashed line indicates attachment to the nitrogen of the primary or secondary amine of -D; and
-L¹- is substituted with at least one moiety -L²- and is optionally further substituted, provided that the hydrogen marked with the asterisk in formula (XIIIb) is not replaced by a substituent.

In certain embodiments the dashed line in formula (XIIIb) indicates attachment to a nitrogen of a primary amine of -D. In certain embodiments the dashed line in formula (XIIIb) indicates attachment to a nitrogen of a secondary amine of -D.

In certain embodiments -L¹- of formula (XIIIb) is not further substituted.

In certain embodiments -L¹- is of formula (XIIIc)

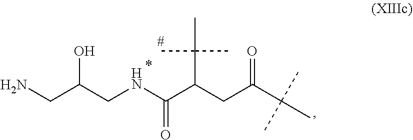

wherein
the unmarked dashed line indicates attachment to the nitrogen of the primary or secondary amine of -D, and
the dashed line marked with # indicates attachment to -L²-.

In certain embodiments the unmarked dashed line in formula (XIIIc) indicates attachment to a nitrogen of a primary amine of -D. In certain embodiments the unmarked dashed line in formula (XIIIc) indicates attachment to a nitrogen of a secondary amine of -D.

It is understood that a moiety -L²-L¹-D is connected to Z through covalent attachment of -L²- to —Z. In certain embodiments -L²- is connected to Z through a stable covalent linkage. In certain embodiments -L¹- is connected to -L²- through a stable covalent linkage.

In certain embodiments all moieties -L²- of the conjugate of the present invention are identical. In certain embodiments a conjugate of the present invention comprises more than one type of -L²-, such as two, three or four different types of -L²-.

In the conjugate of the present invention -L²- is a chemical bond or a spacer moiety. In certain embodiments -L²- does not comprise a reversible linkage, i.e. all linkages in -L²- are stable linkages. In certain embodiments -L²- is connected to Z via a stable linkage.

In certain embodiments -L²- is chemical bond.

In certain embodiments -L²- is a spacer moiety.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R_{y1a})—, —N(R^{y1})C(O)N(R_{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -L²- is a spacer moiety selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1}a are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen, and C₁₋₆ alkyl; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight ranging from 14 g/mol to 750 g/mol.

In certain embodiments -L²- comprises a moiety

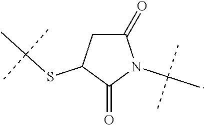

In certain embodiments -L²- has a chain length of 1 to 20 atoms.

As used herein the term “chain length” with regard to the moiety -L²- refers to the number of atoms of -L²- present in the shortest connection between -L¹- and —Z.

In certain embodiments -L²- comprises a moiety of formula (XIVa)

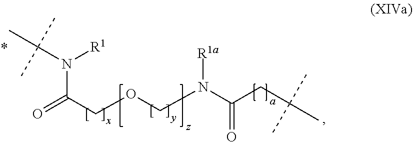

wherein
the dashed line marked with the asterisk indicates attachment to -L¹- and unmarked dashed line indicates attachment to the remainder of -L²- or to Z;
—R¹ and —R^{1a} are independently selected from the group consisting of —H and C₁₋₆ alkyl; a, y and x are independently selected from the group consisting of 1, 2, 3, 4, 5, 6, 7, 8, 9 or 10; and
z is selected from 1, 2, 3, 4, 5, 6, 7, 8, 9, 10, 11, 12, 13, 14, 15, 16, 17, 18, 19 and 20.

In certain embodiments -L²- comprises a moiety of formula (XIVa-i)

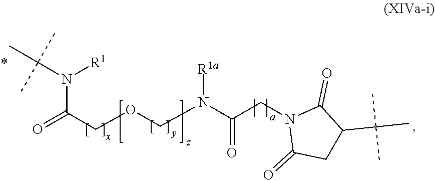

wherein
the dashed line marked with the asterisk indicates attachment to -L¹- and unmarked dashed line indicates attachment to the remainder of -L²- or to Z;
—R¹, —R^{1a}, a, x, y and z are used as defined in formula (XIVa).

In certain embodiments —R¹ of formula (XIVa) or (XIVa-i) is —H. In certain embodiments —R¹ of formula (XIVa) or (XIVa-i) is methyl. In certain embodiments —R¹ of formula (XIVa) or (XIVa-i) is ethyl. In certain embodiments —R¹ of formula (XIVa) or (XIVa-i) is n-propyl. In certain embodiments —R¹ of formula (XIVa) or (XIVa-i) is isopropyl.

In certain embodiments a of formula (XIVa) or (XIVa-i) is 1. In certain embodiments a of formula (XIVa) or (XIVa-i) is 2. In certain embodiments a of formula (XIVa) or (XIVa-i) is 3. In certain embodiments a of formula (XIVa) or (XIVa-i) is 4. In certain embodiments a of formula (XIVa) or (XIVa-i) is 5. In certain embodiments a of formula (XIVa) or (XIVa-i) is 6.

In certain embodiments x of formula (XIVa) or (XIVa-i) is 1. In certain embodiments x of formula (XIVa) or (XIVa-i) is 2. In certain embodiments x of formula (XIVa) or (XIVa-i) is 3. In certain embodiments x of formula (XIVa) or (XIVa-i) is 4. In certain embodiments x of formula (XIVa) or (XIVa-i) is 5. In certain embodiments x of formula (XIVa) or (XIVa-i) is 6.

In certain embodiments y of formula (XIVa) or (XIVa-i) is 1. In certain embodiments y of formula (XIVa) or (XIVa-i) is 2. In certain embodiments y of formula (XIVa) or (XIVa-i) is 3. In certain embodiments y of formula (XIVa) or (XIVa-i) is 4. In certain embodiments y of formula (XIVa) or (XIVa-i) is 5. In certain embodiments y of formula (XIVa) or (XIVa-i) is 6.

In certain embodiments z of formula (XIVa) or (XIVa-i) is 1. In certain embodiments z of formula (XIVa) or (XIVa-i) is 2. In certain embodiments z of formula (XIVa) or (XIVa-i) is 3. In certain embodiments z of formula (XIVa) or (XIVa-i) is 4. In certain embodiments z of formula (XIVa) or (XIVa-i) is 5. In certain embodiments z of formula (XIVa) or (XIVa-i) is 6.

In certain embodiments -L²- comprises a moiety of formula (XIV)

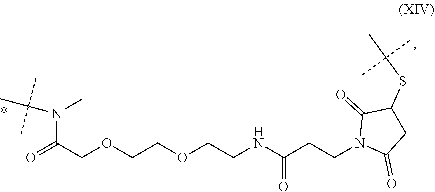

wherein
the dashed line marked with the asterisk indicates attachment to -L¹- and the unmarked dashed line indicates attachment to the remainder of -L²- or to Z.

In certain embodiments the anti-CTLA4 conjugate comprises a moiety of formula (XV)

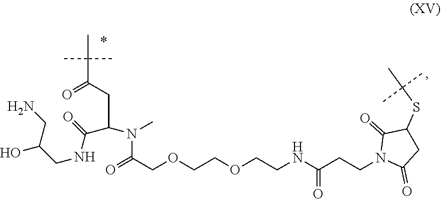

wherein
the dashed line marked with the asterisk indicates attachment to -D and the unmarked dashed line indicates attachment to the remainder of -L²- or to Z.

It is understood that in formula (XV) the dashed line marked with the asterisk indicates attachment to the nitrogen of the primary or secondary amine of -D. In certain embodiments the dashed line marked with the asterisk in formula (XV) indicates attachment to a nitrogen of a primary amine of -D. In certain embodiments the dashed line marked with the asterisk in formula (XV) indicates attachment to a nitrogen of a secondary amine of -D.

In certain embodiments Z comprises a polymer.

In certain embodiments Z is not degradable. In certain embodiments Z is degradable. A degradable moiety Z has the effect that the carrier moiety degrades over time which may be advantageous in certain applications.

In certain embodiments Z is a hydrogel. Such hydrogel may be degradable or non-degradable, i.e. stable. In certain embodiments such hydrogel is degradable. In certain embodiments such hydrogel is non-degradable.

In certain embodiments such hydrogel Z comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(alkylene glycols), such as poly(ethylene glycols) and poly(propylene glycol), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments Z is a poly(alkylene glycol)-based hydrogel, such as a poly(propylene glycol)-based hydrogel or a poly(ethylene glycol)-based (PEG-based) hydrogel, or a hyaluronic acid-based hydrogel. In certain embodiments such hydrogel is degradable. In certain embodiments such hydrogel is non-degradable, i.e. stable.

In certain embodiments Z is a PEG-based hydrogel. Suitable hydrogels are known in the art. Examples are WO2006/003014, WO2011/012715 and WO2014/056926, which are herewith incorporated by reference.

In certain embodiments such PEG-based hydrogel comprises a plurality of backbone moieties that are crosslinked via crosslinker moieties -CL^{p}-. Optionally, there is a spacer moiety —SP¹— between a backbone moiety and a crosslinker moiety. In certain embodiments such spacer —SP¹— is defined as described above for -L²-.

In certain embodiments a backbone moiety has a molecular weight ranging from 1 kDa to 20 kDa.

In certain embodiments a backbone moiety is of formula (A)

B*-(A-Hyp)ₓ (A),
wherein
B* is a branching core,
A is a PEG-based polymer,
Hyp is a branched moiety,
x is an integer of from 3 to 16;
and wherein each backbone moiety is connected to one or more crosslinker moieties and to one or more moieties -L²-, which crosslinker moieties and moieties -L²- are connected to Hyp, either directly or through a spacer moiety —SP¹—.

In certain embodiments B* of formula (A) is selected from the group consisting of polyalcohol moieties and polyamine moieties. In certain embodiments B* of formula (A) is a polyalcohol moiety. In certain embodiments B* of formula (A) is a polyamine moiety.

In certain embodiments the polyalcohol moieties for B* of formula (A) are selected from the group consisting of a pentaerythritol moiety, tripentaerythritol moiety, hexaglycerine moiety, sucrose moiety, sorbitol moiety, fructose moiety, mannitol moiety and glucose moiety. In certain embodiments B* of formula (A) is a pentaerythritol moiety, i.e. a moiety of formula

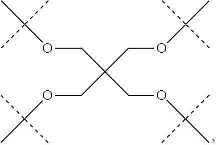

wherein dashed lines indicate attachment to -A-.

In certain embodiments the polyamine moieties for B* of formula (A) is selected from the group consisting of an ornithine moiety, diaminobutyric acid moiety, trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety and pentadecalysine moiety. In certain embodiments B* of formula (A) is selected from the group consisting of an ornithine moiety, diaminobutyric acid moiety and a trilysine moiety.

A backbone moiety of formula (A) may consist of the same or different PEG-based moieties -A- and each moiety -A- may be chosen independently. In certain embodiments all moieties -A- present in a backbone moiety of formula (A) have the same structure. It is understood that the phrase “have the same structure” with regard to polymeric moieties, such as with regard to the PEG-based polymer -A-, means that the number of monomers of the polymer, such as the number of ethylene glycol monomers, may vary due to the polydisperse nature of polymers. In certain embodiments the number of monomer units does not vary by more than a factor of 2 between all moieties -A- of a hydrogel.

In certain embodiments each -A- of formula (A) has a molecular weight ranging from 0.3 kDa to 40 kDa; e.g. from 0.4 to 30 kDa, from 0.4 to 25 kDa, from 0.4 to 20 kDa, from 0.4 to 15 kDa, from 0.4 to 10 kDa or from 0.4 to 5 kDa. In certain embodiments each -A- has a molecular weight from 0.4 to 5 kDa. In certain embodiments -A- has a molecular weight of about 0.5 kDa. In certain embodiments -A- has a molecular weight of about 1 kDa. In certain embodiments -A- has a molecular weight of about 2 kDa. In certain embodiments -A- has a molecular weight of about 3 kDa. In certain embodiments -A- has a molecular weight of about 5 kDa.

In certain embodiments -A- of formula (A) is of formula (A-i)

—(CH₂)ₙ₁(OCH₂CH₂)ₙX— (A-i),
wherein
n1 is 1 or 2;
n is an integer ranging from 3 to 250, such as from 5 to 200, such as from 8 to 150 or from 10 to 100; and
X is a chemical bond or a linkage covalently linking A and Hyp.

In certain embodiments -A- of formula (A) is of formula (A-ii)

—(CH₂)ₙ₁(OCH₂CH₂)ₙ—(CH₂)ₙ₂X— (A-ii),
wherein
n1 is 1 or 2;
n is an integer ranging from 3 to 250, such as from 5 to 200, such as from 8 to 150 or
from 10 to 100;
n2 is 0 or 1; and
X is a chemical bond or a linkage covalently linking A and Hyp.

In certain embodiments -A- of formula (A) is of formula (A-iii)

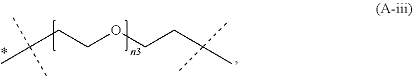

wherein
the dashed line marked with the asterisk indicates attachment to B*,
the unmarked dashed line indicates attachment to -Hyp; and
n3 is an integer ranging from 10 to 50.

In certain embodiments n3 of formula (A-iii) is 25. In certain embodiments n3 of formula (A-iii) is 26. In certain embodiments n3 of formula (A-iii) is 27. In certain embodiments n3 of formula (A-iii) is 28. In certain embodiments n3 of formula (A-iii) is 29. In certain embodiments n3 of formula (A-iii) is 30.

In certain embodiments a moiety B*-(A)₄ is of formula (A-iv)

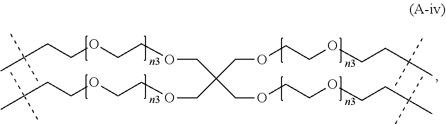

wherein
dashed lines indicate attachment to Hyp; and
each n3 is independently an integer selected from 10 to 50.

In certain embodiments n3 of formula (A-iv) is 25. In certain embodiments n3 of formula (A-iv) is 26. In certain embodiments n3 of formula (A-iv) is 27. In certain embodiments n3 of formula (B-a) is 28. In certain embodiments n3 of formula (A-iv) is 29. In certain embodiments n3 of formula (A-iv) is 30.

A backbone moiety of formula (A) may consist of the same or different dendritic moieties -Hyp and that each -Hyp can be chosen independently. In certain embodiments all moieties -Hyp present in a backbone moiety of formula (A) have the same structure.

In certain embodiments each -Hyp of formula (A) has a molecular weight ranging from 0.3 kDa to 5 kDa.

In certain embodiments -Hyp is selected from the group consisting of a moiety of formula (A-va)

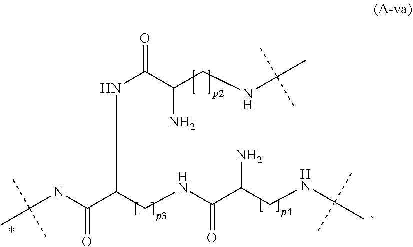

wherein
the dashed line marked with the asterisk indicates attachment to -A-,
the unmarked dashed lines indicate attachment to a spacer moiety —SP¹—, a crosslinker moiety -CL^{p}- or to -L²-; and
p2, p3 and p4 are identical or different and each is independently of the others an integer from 1 to 5;
   a moiety of formula (A-vb)

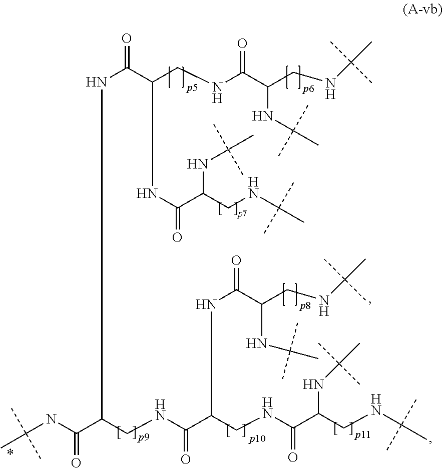

wherein
the dashed line marked with the asterisk indicates attachment to -A-,
the unmarked dashed lines indicate attachment to a spacer moiety —SP¹—, a crosslinker moiety -CL^{p}- or to -L²-; and
p5 to p11 are identical or different and each is independently of the others an integer from 1 to 5;
   a moiety of formula (A-vc)
wherein
the dashed line marked with the asterisk indicates attachment to -A-,
the unmarked dashed lines indicate attachment to a spacer moiety —SP¹—, a crosslinker moiety -CL^{p}- or to -L²-; and
p12 to p26 are identical or different and each is independently of the others an integer from 1 to 5; and
   a moiety of formula (A-vd)

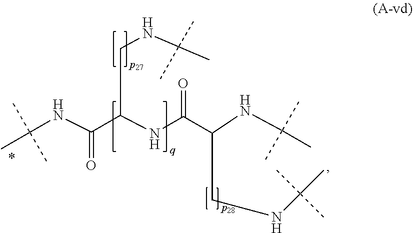

wherein
the dashed line marked with the asterisk indicates attachment to -A-,
the unmarked dashed lines indicate attachment to a spacer moiety —SP¹—, a crosslinker moiety -CL^{p}- or to -L²-;
p27 and p28 are identical or different and each is independently of the other an integer from 1 to 5; and
q is an integer from 1 to 8;
wherein the moieties (A-va) to (A-vd) may at each chiral center be in either R- or S-configuration.

In certain embodiments all chiral centers of a moiety (A-va), (A-vb), (A-vc) or (A-vd) are in the same configuration. In certain embodiments all chiral centers of a moiety (A-va), (A-vb), (A-vc) or (A-vd) are in R-configuration. In certain embodiments all chiral centers of a moiety (A-va), (A-vb), (A-vc) or (A-vd) are in S-configuration.

In certain embodiments p2, p3 and p4 of formula (A-va) are 4.

In certain embodiments p5 to p11 of formula (A-vb) are 4.

In certain embodiments p12 to p26 of formula (A-vc) are 4.

In certain embodiments q of formula (A-vd) is 2 or 6. In certain embodiments q of formula (A-vd) q is 6.

In certain embodiments p27 and p28 of formula (A-vd) are 4.

In certain embodiments -Hyp of formula (A) comprises a branched polypeptide moiety.

In certain embodiments -Hyp of formula (A) comprises a lysine moiety. In certain embodiments each -Hyp of formula (A) is independently selected from the group consisting of a trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety, pentadecalysine moiety, hexadecalysine moiety, heptadecalysine moiety, octadecalysine moiety and nonadecalysine moiety.

In certain embodiments -Hyp comprises 3 lysine moieties. In certain embodiments -Hyp comprises 7 lysine moieties. In certain embodiments -Hyp comprises 15 lysine moieties. In certain embodiments -Hyp comprises heptalysinyl.

In certain embodiments x of formula (A) is 3. In certain embodiments x of formula (A) is 4.

In certain embodiments x of formula (A) is 6. In certain embodiments x of formula (A) is 8.

In certain embodiments the backbone moiety is of formula (A-vi)

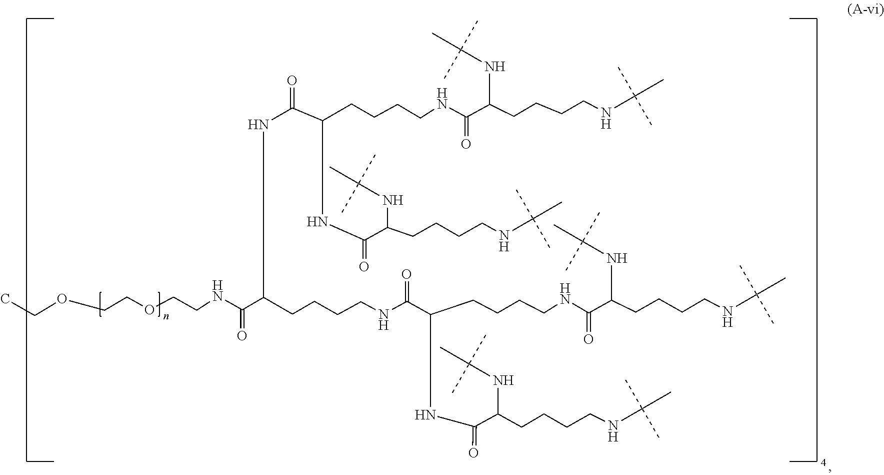

wherein
dashed lines indicate attachment to a spacer moiety —SP¹—, a crosslinker moiety -CL^{p}- or to -L²-; and
n ranges from 10 to 40.

In certain embodiments n of formula (A-vi) is about 28.

In certain embodiments the backbone moiety is of formula (A-vii)

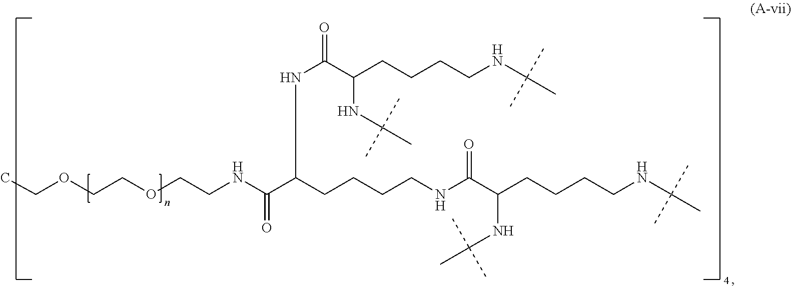

wherein
dashed lines indicate attachment to a spacer moiety —SP¹—, a crosslinker moiety -CL^{p}- or to -L²-; and
n ranges from 10 to 40.

In certain embodiments there is no spacer moiety —SP¹— between a backbone moiety and a crosslinker moiety -CL^{p}-, i.e. -CL^{p}- is directly linked to -Hyp.

The crosslinker -CL^{p}- of the PEG-based hydrogel is in certain embodiments poly(alkylene glycol) (PAG)-based. In certain embodiments the crosslinker is poly(propylene glycol)-based.

In certain embodiments the crosslinker -CL^{p}- is PEG-based.

In certain embodiments such PAG-based crosslinker moiety -CL^{p}- is of formula (A-viii)

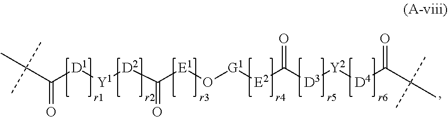

wherein
dashed lines indicate attachment to a backbone moiety or to a spacer moiety —SP¹—; —Y¹— is of formula

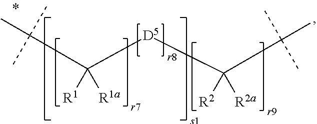

wherein the dashed line marked with the asterisk indicates attachment to -D¹- and the unmarked dashed line indicates attachment to -D²-;
—Y²— is of formula

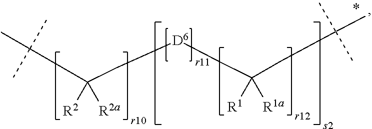

wherein the dashed line marked with the asterisk indicates attachment to -D⁴- and the unmarked dashed line indicates attachment to -D³-;
-E¹- is of formula

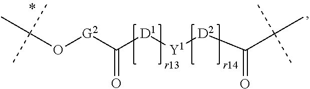

wherein the dashed line marked with the asterisk indicates attachment to —(C═O)— and the unmarked dashed line indicates attachment to —O—;
-E²- is of formula

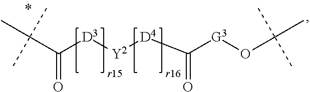

wherein the dashed line marked with the asterisk indicates attachment to -G¹- and the unmarked dashed line indicates attachment to —(C═O)—;
-G¹- is of formula

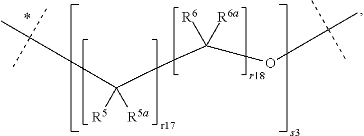

wherein the dashed line marked with the asterisk indicates attachment to —O— and the unmarked dashed line indicates attachment to -E²-;
-G²- is of formula

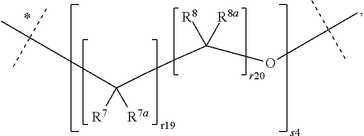

wherein the dashed line marked with the asterisk indicates attachment to —O— and the unmarked dashed line indicates attachment to —(C═O)—;
-G³- is of formula

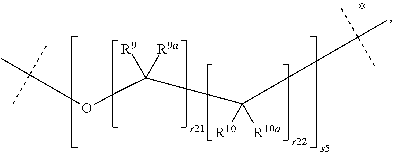

wherein the dashed line marked with the asterisk indicates attachment to —O— and the unmarked dashed line indicates attachment to —(C═O)—;
-D¹-, -D²-, -D³-, -D⁴-, -D⁵- and -D⁶- are identical or different and each is independently of the others selected from the group comprising —O—, —NR¹¹—, —N⁺R¹²R^{12a}—, —S—, —(S═O)—, —(S(O)₂)—, —C(O)—, —P(O)R¹³—, —P(O)(OR¹³) and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹, —R^{9a}, —R¹⁰, —R^{10a}, —R¹¹, —R¹, —R^{12a}, —R¹³, R¹⁴ and —R^{14a} are identical or different and each is independently of the others selected from the group consisting of —H and C₁₋₆ alkyl;
optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
r1, r2, r5, r6, r13, r14, r15 and r16 are independently 0 or 1;
r3, r4, r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10;
s1, s2, s4, s5 are independently 1, 2, 3, 4, 5 or 6; and
s3 ranges from 1 to 900.

In certain embodiments s3 ranges from 1 to 500. In certain embodiments s3 ranges from 1 to 200.

In certain embodiments r1 of formula (A-viii) is 0. In certain embodiments r1 of formula (A-viii) is 1. In certain embodiments r2 of formula (A-viii) is 0. In certain embodiments r2 of formula (A-viii) is 1. In certain embodiments r5 of formula (A-viii) is 0. In certain embodiments r5 of formula (A-viii) is 1.

In certain embodiments r1, r2, r5 and r6 of formula (A-viii) are 0.

In certain embodiments r6 of formula (A-viii) is 0. In certain embodiments r6 of formula (A-viii) is 1. In certain embodiments r13 of formula (A-viii) is 0. In certain embodiments r13 of formula (A-viii) is 1. In certain embodiments r14 of formula (A-viii) is 0. In certain embodiments r14 of formula (A-viii) is 1. In certain embodiments r15 of formula (A-viii) is 0. In certain embodiments r15 of formula (A-viii) is 1. In certain embodiments r16 of formula (A-viii) is 0. In certain embodiments r16 of formula (A-viii) is 1.

In certain embodiments r3 of formula (A-viii) is 1. In certain embodiments r3 of formula (A-viii) is 2. In certain embodiments r4 of formula (A-viii) is 1. In certain embodiments r4 of formula (A-viii) is 2. In certain embodiments r3 and r4 of formula (A-viii) are both 1. In certain embodiments r3 and r4 of formula (A-viii) are both 2. In certain embodiments r3 and r4 of formula (A-viii) are both 3.

In certain embodiments r7 of formula (A-viii) is 0. In certain embodiments r7 of formula (A-viii) is 1. In certain embodiments r7 of formula (A-viii) is 2. In certain embodiments r8 of formula (A-viii) is 0. In certain embodiments r8 of formula (A-viii) is 1. In certain embodiments r8 of formula (A-viii) is 2. In certain embodiments r9 of formula (A-viii) is 0. In certain embodiments r9 of formula (A-viii) is 1. In certain embodiments r9 of formula (A-viii) is 2. In certain embodiments r10 of formula (A-viii) is 0. In certain embodiments r10 of formula (A-viii) is 1. In certain embodiments r10 of formula (A-viii) is 2. In certain embodiments r11 of formula (A-viii) is 0. In certain embodiments r11 of formula (A-viii) is 1. In certain embodiments r11 of formula (A-viii) is 2. In certain embodiments r12 of formula (A-viii) is 0. In certain embodiments r12 of formula (A-viii) is 1. In certain embodiments r12 of formula (A-viii) is 2.

In certain embodiments r17 of formula (A-viii) is 1. In certain embodiments r18 of formula (A-viii) is 1. In certain embodiments r19 of formula (A-viii) is 1. In certain embodiments r20 of formula (A-viii) is 1. In certain embodiments r21 of formula (A-viii) is 1.

In certain embodiments s1 of formula (A-viii) is 1. In certain embodiments s1 of formula (A-viii) is 2. In certain embodiments s2 of formula (A-viii) is 1. In certain embodiments s2 of formula (A-viii) is 2. In certain embodiments s4 of formula (A-viii) is 1. In certain embodiments s4 of formula (A-viii) is 2.

In certain embodiments s3 of formula (A-viii) ranges from 5 to 500. In certain embodiments s3 of formula (A-viii) ranges from 10 to 250. In certain embodiments s3 of formula (A-viii) ranges from 12 to 150. In certain embodiments s3 of formula (A-viii) ranges from 15 to 100.

In certain embodiments s3 of formula (A-viii) ranges from 18 to 75. In certain embodiments s3 of formula (A-viii) ranges from 20 to 50.

In certain embodiments —R¹ of formula (A-viii) is —H. In certain embodiments —R¹ of formula (A-viii) is methyl. In certain embodiments —R¹ of formula (A-viii) is ethyl. In certain embodiments —R^{1a} of formula (A-viii) is —H. In certain embodiments —R^{1a} of formula (A-viii) is methyl. In certain embodiments —R^{1a} of formula (A-viii) is ethyl. In certain embodiments —R² of formula (A-viii) is —H. In certain embodiments —R² of formula (A-viii) is methyl. In certain embodiments —R² of formula (A-viii) is ethyl. In certain embodiments —R^{2a} of formula (A-viii) is —H. In certain embodiments —R^{2a} of formula (A-viii) is methyl. In certain embodiments —R^{2a} of formula (A-viii) is ethyl. In certain embodiments —R³ of formula (A-viii) is —H. In certain embodiments —R³ of formula (A-viii) is methyl. In certain embodiments —R³ of formula (A-viii) is ethyl. In certain embodiments —R^{3a} of formula (A-viii) is —H. In certain embodiments —R^{3a} of formula (A-viii) is methyl. In certain embodiments —R^{3a} of formula (A-viii) is ethyl. In certain embodiments —R⁴ of formula (A-viii) is —H. In certain embodiments —R⁴ of formula (A-viii) is methyl. In certain embodiments —R⁴ of formula (A-viii) is methyl. In certain embodiments —R^{4a} of formula (A-viii) is —H. In certain embodiments —R^{4a} of formula (A-viii) is methyl. In certain embodiments —R^{4a} of formula (A-viii) is ethyl. In certain embodiments —R⁵ of formula (A-viii) is —H. In certain embodiments —R⁵ of formula (A-viii) is methyl. In certain embodiments —R⁵ of formula (A-viii) is ethyl. In certain embodiments —R^{5a} of formula (A-viii) is —H. In certain embodiments —R^{5a} of formula (A-viii) is methyl. In certain embodiments —R^{5a} of formula (A-viii) is ethyl. In certain embodiments —R⁶ of formula (A-viii) is —H. In certain embodiments —R⁶ of formula (A-viii) is methyl. In certain embodiments —R⁶ of formula (A-viii) is ethyl. In certain embodiments —R^{6a} of formula (A-viii) is —H. In certain embodiments —R^{6a} of formula (A-viii) is methyl. In certain embodiments —R^{6a} of formula (A-viii) is ethyl. In certain embodiments —R⁷ of formula (A-viii) is —H. In certain embodiments —R⁷ of formula (A-viii) is methyl. In certain embodiments —R⁷ of formula (A-viii) is ethyl. In certain embodiments —R⁸ of formula (A-viii) is —H. In certain embodiments —R⁸ of formula (A-viii) is methyl. In certain embodiments —R⁸ of formula (A-viii) is ethyl. In certain embodiments —R^{8a} of formula (A-viii) is —H. In certain embodiments —R^{8a} of formula (A-viii) is methyl. In certain embodiments —R^{8a} of formula (A-viii) is ethyl. In certain embodiments —R⁹ of formula (A-viii) is —H. In certain embodiments —R⁹ of formula (A-viii) is methyl. In certain embodiments —R⁹ of formula (A-viii) is ethyl. In certain embodiments —R^{9a} of formula (A-viii) is —H. In certain embodiments —R^{9a} of formula (A-viii) is methyl. In certain embodiments —R^{9a} of formula (A-viii) is ethyl. In certain embodiments —R^{9a} of formula (A-viii) is —H. In certain embodiments —R^{9a} of formula (A-viii) is methyl. In certain embodiments —R^{9a} of formula (A-viii) is ethyl. In certain embodiments —R¹⁰ of formula (A-viii) is —H. In certain embodiments —R¹⁰ of formula (A-viii) is methyl. In certain embodiments —R¹⁰ of formula (A-viii) is ethyl. In certain embodiments —R^{10a} of formula (A-viii) is —H. In certain embodiments —R^{10a} of formula (A-viii) is methyl. In certain embodiments —R^{10a} of formula (A-viii) is ethyl. In certain embodiments —R¹¹ of formula (A-viii) is —H. In certain embodiments —R¹¹ of formula (A-viii) is methyl. In certain embodiments —R¹¹ of formula (A-viii) is ethyl. In certain embodiments —R¹² of formula (A-viii) is —H. In certain embodiments —R¹² of formula (A-viii) is methyl. In certain embodiments —R¹² of formula (A-viii) is ethyl. In certain embodiments —R^{12a} of formula (A-viii) is —H. In certain embodiments —R^{12a} of formula (A-viii) is methyl. In certain embodiments —R^{12a} of formula (A-viii) is ethyl. In certain embodiments —R¹³ of formula (A-viii) is —H. In certain embodiments —R¹³ of formula (A-viii) is methyl. In certain embodiments —R¹³ of formula (A-viii) is ethyl. In certain embodiments —R¹⁴ of formula (A-viii) is —H. In certain embodiments —R¹⁴ of formula (A-viii) is methyl. In certain embodiments —R¹⁴ of formula (A-viii) is ethyl. In certain embodiments —R^{14a} of formula (A-viii) is —H. In certain embodiments —R^{14a} of formula (A-viii) is methyl. In certain embodiments —R^{14a} of formula (A-viii) is ethyl.

In certain embodiments -D¹- of formula (A-viii) is —O—. In certain embodiments -D¹- of formula (A-viii) is —NR¹¹—. In certain embodiments -D¹- of formula (A-viii) is —N⁺R¹²R^{12a}—. In certain embodiments -D¹- of formula (A-viii) is —S—. In certain embodiments -D¹- of formula (A-viii) is —(S═O). In certain embodiments -D¹- of formula (A-viii) is —(S(O)₂)—. In certain embodiments -D¹- of formula (A-viii) is —C(O)—. In certain embodiments -D¹- of formula (A-viii) is —P(O)R¹³—. In certain embodiments -D¹- of formula (A-viii) is —P(O)(OR¹³)—. In certain embodiments -D¹- of formula (A-viii) is —CR¹⁴R^{14a}—.

In certain embodiments -D²- of formula (A-viii) is —O—. In certain embodiments -D²- of formula (A-viii) is —NR¹¹—. In certain embodiments -D²- of formula (A-viii) is —N⁺R¹²R^{12a}—. In certain embodiments -D²- of formula (A-viii) is —S—. In certain embodiments -D²- of formula (A-viii) is —(S═O). In certain embodiments -D²- of formula (A-viii) is —(S(O)₂)—. In certain embodiments -D²- of formula (A-viii) is —C(O)—. In certain embodiments -D²- of formula (A-viii) is —P(O)R¹³—. In certain embodiments -D²- of formula (A-viii) is —P(O)(OR¹³)—. In certain embodiments -D²- of formula (A-viii) is —CR¹⁴R^{14a}—.

In certain embodiments -D³- of formula (A-viii) is —O—. In certain embodiments -D³- of formula (A-viii) is —NR¹¹—. In certain embodiments -D³- of formula (A-viii) is —N⁺R¹²R^{12a}—. In certain embodiments -D³- of formula (A-viii) is —S—. In certain embodiments -D³- of formula (A-viii) is —(S═O). In certain embodiments -D³- of formula (A-viii) is —(S(O)₂)—. In certain embodiments -D³- of formula (A-viii) is —C(O)—. In certain embodiments -D³- of formula (A-viii) is —P(O)R¹³—. In certain embodiments -D³- of formula (A-viii) is —P(O)(OR¹³)—. In certain embodiments -D³- of formula (A-viii) is —CR¹⁴R^{14a}—.

In certain embodiments -D⁴- of formula (A-viii) is —O—. In certain embodiments -D⁴- of formula (A-viii) is —NR¹¹—. In certain embodiments -D⁴- of formula (A-viii) is —N⁺R¹²R^{12a}—. In certain embodiments -D⁴- of formula (A-viii) is —S—. In certain embodiments -D⁴- of formula (A-viii) is —(S═O). In certain embodiments -D⁴- of formula (A-viii) is —(S(O)₂)—. In certain embodiments -D⁴- of formula (A-viii) is —C(O)—. In certain embodiments -D⁴- of formula (A-viii) is —P(O)R¹³—. In certain embodiments -D⁴- of formula (A-viii) is —P(O)(OR¹³)—. In certain embodiments -D⁴- of formula (A-viii) is —CR¹⁴R^{14a}—.

In certain embodiments -D⁵- of formula (A-viii) is —O—. In certain embodiments -D⁵- of formula (A-viii) is —NR¹¹—. In certain embodiments -D⁵- of formula (A-viii) is —N⁺R¹²R^{12a}—. In certain embodiments -D⁵- of formula (A-viii) is —S—. In certain embodiments -D⁵- of formula (A-viii) is —(S═O)—. In certain embodiments -D⁵- of formula (A-viii) is —(S(O)₂)—. In certain embodiments -D⁵- of formula (A-viii) is —C(O)—. In certain embodiments -D⁵- of formula (A-viii) is —P(O)R¹³—. In certain embodiments -D⁵- of formula (A-viii) is —P(O)(OR¹³)—. In certain embodiments -D⁵- of formula (A-viii) is —CR¹⁴R^{14a}—.

In certain embodiments -D⁶- of formula (A-viii) is —O—. In certain embodiments -D⁶- of formula (A-viii) is —NR¹¹—. In certain embodiments -D⁶- of formula (A-viii) is —N⁺R¹²R^{12a}—. In certain embodiments -D⁶- of formula (A-viii) is —S—. In certain embodiments -D⁶- of formula (A-viii) is —(S═O). In certain embodiments -D⁶- of formula (A-viii) is —(S(O)₂)—. In certain embodiments -D⁶- of formula (A-viii) is —C(O)—. In certain embodiments -D⁶- of formula (A-viii) is —P(O)R¹³—. In certain embodiments -D⁶- of formula (A-viii) is —P(O)(OR¹³)—. In certain embodiments -D⁶- of formula (A-viii) is —CR¹⁴R^{14a}—.

In one embodiment -CL^{p}- is of formula (A-ix)

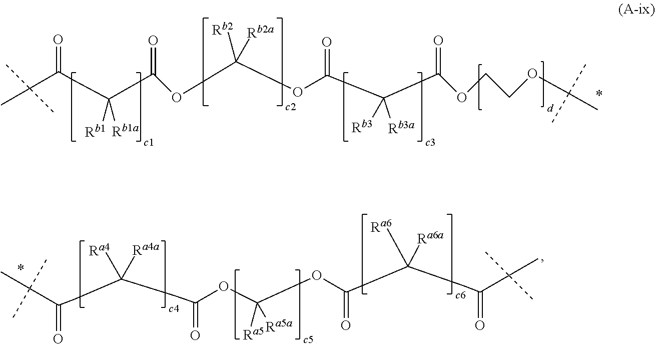

wherein
dashed lines marked with an asterisk indicate the connection point between the upper and the lower substructure,
unmarked dashed lines indicate attachment to a backbone moiety or to a spacer moiety —SP¹—;
—R^{b1}, —R^{b1a}, —R^{b2}, —R^{b2a}, —R^{b3}, —R^{b3a}, —R^{b4}, —R^{b4a}, —R^{b5}, —R^{b5a}, —R^{b6} and —R^{b6} are independently selected from the group consisting of —H and C₁₋₆ alkyl;
c1, c2, c3, c4, c5 and c6 are independently selected from the group consisting of 1, 2, 3, 4, 5 and 6;
d is an integer ranging from 2 to 250.

In certain embodiments d of formula (A-ix) ranges from 3 to 200. In certain embodiments d of formula (A-ix) ranges from 4 to 150. In certain embodiments d of formula (A-ix) ranges from 5 to 100. In certain embodiments d of formula (A-ix) ranges from 10 to 50. In certain embodiments d of formula (A-ix) ranges from 15 to 30. In certain embodiments d of formula (A-ix) is about 23.

In certain embodiments —R^{b1} and —R^{b1a} of formula (A-ix) are —H. In certain embodiments —R^{b1} and —R^{b1a} of formula (A-ix) are —H. In certain embodiments —R^{b2} and —R^{b2a} of formula (A-ix) are —H. In certain embodiments —R^{b3} and —R^{b3a} of formula (A-ix) are —H. In certain embodiments —R^{b4} and —R^{b4a} of formula (A-ix) are —H. In certain embodiments —R^{b5} and —R^{b5a} of formula (A-ix) are —H. In certain embodiments —R^{b6} and —R^{b6a} of formula (A-ix) are —H.

In certain embodiments —R^{b1}, —R^{b1a}, —R^{b2}, —R^{b2a}, —R^{b3}, —R^{b3a}, —R^{b4}, —R^{b4a}, —R^{b5}, —R^{b5a}, —R^{b6} and —R^{b6} of formula (A-ix) are all —H.

In certain embodiments c1 of formula (A-ix) is 1. In certain embodiments c1 of formula (A-ix) is 2. In certain embodiments c1 of formula (A-ix) is 3. In certain embodiments c1 of formula (A-ix) is 4. In certain embodiments c1 of formula (A-ix) is 5. In certain embodiments c1 of formula (A-ix) is 6.

In certain embodiments c2 of formula (A-ix) is 1. In certain embodiments c2 of formula (A-ix) is 2. In certain embodiments c2 of formula (A-ix) is 3. In certain embodiments c2 of formula (A-ix) is 4. In certain embodiments c2 of formula (A-ix) is 5. In certain embodiments c2 of formula (A-ix) is 6.

In certain embodiments c3 of formula (A-ix) is 1. In certain embodiments c3 of formula (A-ix) is 2. In certain embodiments c3 of formula (A-ix) is 3. In certain embodiments c3 of formula (A-ix) is 4. In certain embodiments c3 of formula (A-ix) is 5. In certain embodiments c3 of formula (A-ix) is 6.

In certain embodiments c4 of formula (A-ix) is 1. In certain embodiments c4 of formula (A-ix) is 2. In certain embodiments c4 of formula (A-ix) is 3. In certain embodiments c4 of formula (A-ix) is 4. In certain embodiments c4 of formula (A-ix) is 5. In certain embodiments c4 of formula (A-ix) is 6.

In certain embodiments c5 of formula (A-ix) is 1. In certain embodiments c5 of formula (A-ix) is 2. In certain embodiments c5 of formula (A-ix) is 3. In certain embodiments c5 of formula (A-ix) is 4. In certain embodiments c5 of formula (A-ix) is 5. In certain embodiments c5 of formula (A-ix) is 6.

In certain embodiments c6 of formula (A-ix) is 1. In certain embodiments c6 of formula (A-ix) is 2. In certain embodiments c6 of formula (A-ix) is 3. In certain embodiments c6 of formula (A-ix) is 4. In certain embodiments c6 of formula (A-ix) is 5. In certain embodiments c6 of formula (A-ix) is 6.

In certain embodiments a crosslinker moiety -CL^{p}- is of formula (A-x)

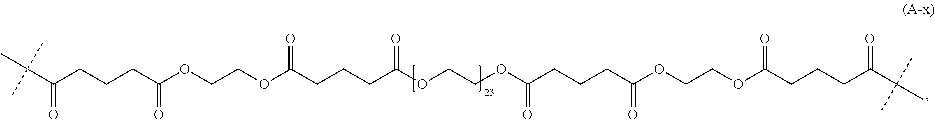

wherein

dashed lines indicate attachment to a backbone moiety or to a spacer moiety —SP¹—.

In certain embodiments —Z is a hyaluronic acid-based hydrogel. Such hyaluronic acid-based hydrogels are known in the art, such as for example from WO2018/175788, which is incorporated herewith by reference.

If —Z is a hyaluronic acid-based hydrogel, a conjugate of the present invention is in certain embodiments a conjugate comprising crosslinked hyaluronic acid strands to which a plurality of drug moieties is covalently and reversibly conjugated, wherein the conjugate comprises a plurality of connected units selected from the group consisting of

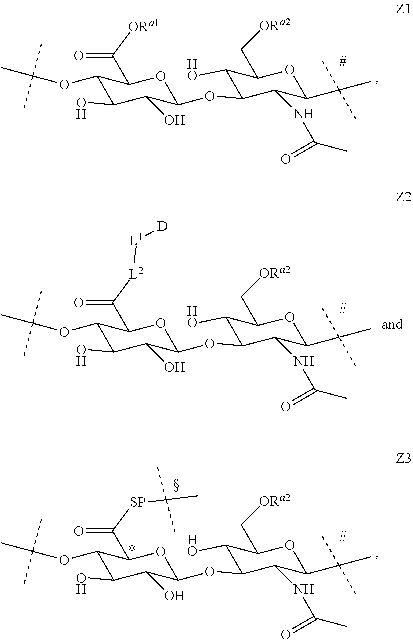

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
each -D, -L¹-, and -L² are used as defined above;
each -CL- is independently a moiety connecting at least two units Z³ and wherein there is at least one degradable bond in the direct connection between any two carbon atoms marked with the * connected by a moiety -CL-;
each —SP— is independently absent or a spacer moiety;
each —R^{a1} is independently selected from the group consisting of —H, C₁₋₄ alkyl, an ammonium ion, a tetrabutylammonium ion, a cetyl methylammonium ion, an alkali metal ion and an alkaline earth metal ion;
each —R^{a2} is independently selected from the group consisting of —H and C₁₋₁₀ alkyl;
wherein
all units Z¹ present in the conjugate may be the same or different;
all units Z² present in the conjugate may be the same or different;
all units Z³ present in the conjugate may be the same or different;
at least one unit Z³ is present per hyaluronic acid strand which is connected to at least one unit Z³ on a different hyaluronic acid strand; and
the conjugate comprises at least one moiety -L²-L¹-D.

The presence of at least one degradable bond between the carbon atom marked with the * of a first moiety Z³ and the direct connection to the carbon atom marked with the * of a second moiety Z³ ensures that after cleavage of all such degradable bonds the hyaluronic acid strands present in said conjugate are no longer crosslinked, which allows clearance of the hyaluronic acid network

It is understood that in case a degradable bond is located in a ring structure present in the direct connection of the carbon atom marked with the * of a first moiety Z³ and the carbon atom marked with the * of a second moiety Z³ such degradable bond is not sufficient to allow complete cleavage and accordingly one or more additional degradable bonds are present in the direct connection of the carbon atom marked with the * of a first moiety Z³ and the carbon atom marked with the * of a second moiety Z³.

It is understood that the phrase “a dashed line marked with § indicates a point of connection between at least two units Z³ via a moiety -CL-” refers to the following structure

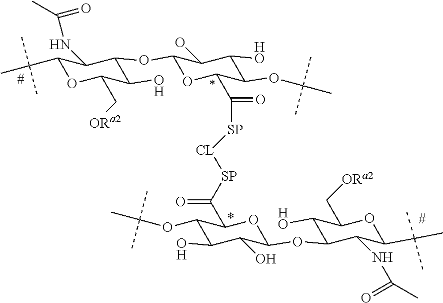

if -CL- is for example connected to two units Z³, which two moieties Z³ are connected at the position indicated with § via a moiety -CL-.

It is understood that no three-dimensionally crosslinked hydrogel can be formed if all hyaluronic acid strands of the present conjugate comprise only one unit Z³, which is connected to only one unit Z³ on a different hyaluronic acid strand. However, if a first unit Z³ is connected to more than one unit Z³ on a different strand, i.e. if -CL- is branched, such first unit Z³ may be crosslinked to two or more other units Z³ on two or more different hyaluronic acid strands. Accordingly, the number of units Z³ per hyaluronic acid strand required for a crosslinked hyaluronic acid hydrogel depends on the degree of branching of -CL-. In certain embodiments at least 30% of all hyaluronic acid strands present in the conjugate are connected to at least two other hyaluronic acid strands. It is understood that it is sufficient if the remaining hyaluronic acid strands are connected to only one other hyaluronic acid strand.

It is understood that such hydrogel also comprises partly reacted or unreacted units and that the presence of such moieties cannot be avoided. In certain embodiments the sum of such partly reacted or unreacted units is no more than 25% of the total number of units present in the conjugate, such as no more than 10%, such as no more than 15% or such as no more than 10%.

Furthermore, it is understood that in addition to units Z¹, Z² and Z³, partly reacted and unreacted units a conjugate may also comprise units that are the result of cleavage of the reversible bond between -D and -L¹- or of one or more of the degradable bonds present in the direct connection between any two carbon atoms marked with the * connected by a moiety -CL-, i.e. units resulting from degradation of the conjugate.

In certain embodiments each strand present in the conjugates of the present invention comprises at least 20 units, such as from 20 to 2500 units, from 25 to 2200 units, from 50 to 2000 units, from 75 to 100 units, from 75 to 100 units, from 80 to 560 units, from 100 to 250 units, from 200 to 800 units, from 20 to 1000, from 60 to 1000, from 60 to 400 or from 200 to 600 units.

In certain embodiments the moieties -CL- present in Z have different structures. In certain embodiments the moieties -CL- present in Z have the same structure.

In general, any moiety that connects at least two other moieties is suitable for use as a moiety -CL-, which may also be referred to as a “crosslinker moiety”.

The at least two units Z³ that are connected via a moiety -CL- may either be located on the same hyaluronic acid strand or on different hyaluronic acid strands.

The moiety -CL- may be linear or branched. In certain embodiments -CL- is linear. In certain embodiments -CL- is branched.

In certain embodiments -CL- connects two units Z³. In certain embodiments -CL- connects three units Z³. In certain embodiments -CL- connects four units Z³. In certain embodiments -CL- connects five units Z³. In certain embodiments -CL- connects six units Z³. In certain embodiments -CL- connects seven units Z³. In certain embodiments -CL- connects eight units Z³. In certain embodiments -CL- connects nine units Z³.

If -CL- connects two units Z³-CL- may be linear or branched. If -CL- connects more than two units Z³-CL- is branched.

A branched moiety -CL- comprises at least one branching point from which at least three branches extend, which branches may also be referred to as “arms”. Such branching point may be selected from the group consisting of

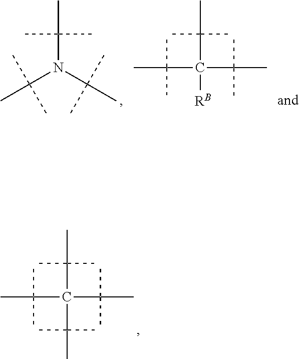

wherein
dashed lines indicate attachment to an arm; and
—R^{B} is selected from the group consisting of —H, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{B1}, which are the same or different, and wherein C₁₋₆ alkyl, C₂₋₆ alkenyl and C₂₋₆ alkynyl are optionally interrupted with —C(O)O—, —O—, —C(O)—, —C(O)N(R^{B2})—, —S(O)₂N(R^{B2})—, —S(O)N(R^{B2})—, —S(O)₂—, —S(O)—, —N(R^{B2})S(O)₂N(R^{B2a})—, —S—, —N(R^{B2})—, —OC(OR^{B2})(R^{B2a})—, —N(R^{B2})C(O)N(R^{B2a})—, and —OC(O)N(R^{B2})—; wherein —R^{B1}, —R^{B2} and —R^{B2a} are selected from —H, C₁₋₆ alkyl, C₂₋₆ alkenyl and C₂₋₆ alkynyl.

In certain embodiments —R^{B} is selected from the group consisting of —H, methyl and ethyl.

A branched moiety -CL- may comprise a plurality of branching points, such as 1, 2, 3, 4, 5, 6, 7 or more branching points, which may be the same or different.

If a moiety -CL- connects three units Z³, such moiety -CL- comprises at least one branching point from which at least three arms extend.

If a moiety -CL- connects four units Z³, such moiety -CL- may comprise one branching point from which four arms extend. However, alternative geometries are possible, such as at least two branching points from which at least three arms each extend. The larger the number of connected units Z³, the larger the number of possible geometries is.

In a first embodiment at least 70%, such as at least 75%, such as at least 80%, such as at least 85%, such as at least 90% or such as at least 95% of the number of hyaluronic acid strands of the conjugate of the present invention comprise at least one moiety Z² and at least one moiety Z³. In such embodiment units Z² and Z³ can be found in essentially all hyaluronic acid strands present in the conjugates of the present invention.

Accordingly, a conjugate of this first embodiment comprises crosslinked hyaluronic acid strands to which a plurality of drug moieties are covalently and reversibly conjugated, wherein the conjugate of the present invention comprises a plurality of connected units selected from the group consisting of

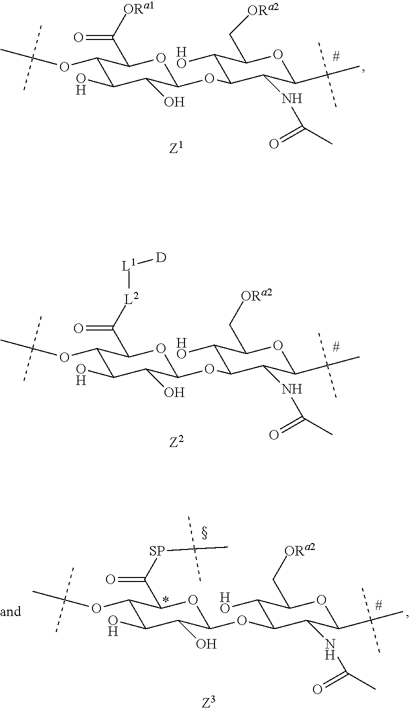

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
-D, -L¹-, -L²-, are used as defined above;
wherein
all units Z¹ present in the conjugate may be the same or different;
all units Z² present in the conjugate may be the same or different;
all units Z³ present in the conjugate may be the same or different;
the number of Z¹ units ranges from 1% to 98% of the total number of units present in the conjugate of the present invention;
the number of Z² units ranges from 1% to 98% of the total number of units present in the conjugate, provided at least one unit Z² is present in the conjugate of the present invention;
the number of Z³ units ranges from 1% to 97% of the total number of units present in the conjugate of the present invention, provided that at least one unit Z³ is present per strand; and
wherein at least 70% of all hyaluronic acid strands comprise at least one moiety Z² and at least one moiety Z³.

In a conjugate of the present invention according to this first embodiment the number of units Z² ranges from 1 to 70% of all units present in the conjugate of the present invention, such as from 2 to 15%, from 2 to 10%, from 16 to 39, from 40 to 65%, or from 50 to 60% of all units present in the conjugate of the present invention.

In a conjugate of the present invention according to this first embodiment the number of units Z³ ranges from 1 to 30% of all units present in conjugate of the present invention, such as from 2 to 5%, from 5 to 20%, from 10 to 18%, or from 14 to 18% of all units present in the conjugate of the present invention.

In a conjugate of the present invention according to this first embodiment the number of units Z¹ ranges from 10 to 97% of all units present in the conjugate of the present invention, such as from 20 to 40%, such as from 25 to 35%, such as from 41 to 95%, such as from 45 to 90%, such as from 50 to 70% of all units present in the conjugate of the present invention.

Each degradable bond present in the direct connection between any two carbon atoms marked with the * connected by a moiety -CL- may be different or all such degradable bonds present in the conjugate of the present invention may be the same.

Each direct connection between two carbon atoms marked with the * connected by a moiety -CL- may have the same or a different number of degradable bonds.

In certain embodiments the number of degradable bonds present in the conjugate of the present invention between all combinations of two carbon atoms marked with the * connected by a moiety -CL- is the same and all such degradable bonds have the same structure.

In the first embodiment the at least one degradable bond present in the direct connection between any two carbon atoms marked with the * connected by a moiety -CL- may be selected from the group consisting of ester, carbonate, sulfate, phosphate bonds, carbamate and amide bonds. It is understood that carbamates and amides are not reversible per se, and that in this context neighboring groups render these bonds reversible. In certain embodiments there is one degradable bond selected from the group consisting of ester, carbonate, sulfate, phosphate bonds, carbamate and amide bonds in the direct connection between any two carbon atoms marked with the * connected by a moiety -CL-. In certain embodiments there are two degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are three degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are four degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are five degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. In certain embodiments there are six degradable bonds selected from the group consisting of ester, carbonate, sulfate, phosphate bonds, carbamate and amide bonds in the direct connection between any two carbon atoms marked with the * connected by a moiety -CL-, which degradable bonds may be the same or different. It is understood that if more than two units Z³ are connected by -CL- there are more than two carbons marked with * that are connected and thus there is more than one shortest connection with at least one degradable bond present. Each shortest connection may have the same or different number of degradable bonds.

In certain embodiments the at least one degradable bond, such as one, two, three, four, five, six degradable bonds, are located within -CL-.

In certain embodiments the at least one degradable bond present in the direct connection between any two carbon atoms marked with * connected by a moiety -CL- is one ester bond.

In other embodiments the at least one degradable bond are two ester bonds. In other embodiments the at least one degradable bond are three ester bonds. In other embodiments the at least one degradable bond are four ester bonds. In other embodiments the at least one degradable bond are five ester bonds. In other embodiments the at least one degradable bond are six ester bonds.

In certain embodiments the at least one degradable bond present in the direct connection between any two carbon atoms marked with * connected by a moiety -CL- is one carbonate bond. In other embodiments the at least one degradable bond are two carbonate bonds. In other embodiments the at least one degradable bond are three carbonate bonds. In other embodiments the at least one degradable bond are four carbonate bonds. In other embodiments the at least one degradable bond are five carbonate bonds. In other embodiments the at least one degradable bond are six carbonate bonds.

In certain embodiments the at least one degradable bond present in the direct connection between any two carbon atoms marked with * connected by a moiety -CL- is one phosphate bond. In other embodiments the at least one degradable bond are two phosphate bonds. In other embodiments the at least one degradable bond are three phosphate bonds. In other embodiments the at least one degradable bond are four phosphate bonds. In other embodiments the at least one degradable bond are five phosphate bonds. In other embodiments the at least one degradable bond are six phosphate bonds.

In certain embodiments the at least one degradable bond present in the direct connection between any two carbon atoms marked with * connected by a moiety -CL- is one sulfate bond. In other embodiments the at least one degradable bond are two sulfate bonds. In other embodiments the at least one degradable bond are three sulfate bonds. In other embodiments the at least one degradable bond are four sulfate bonds. In other embodiments the at least one degradable bond are five sulfate bonds. In other embodiments the at least one degradable bond are six sulfate bonds.

In certain embodiments the at least one degradable bond present in the direct connection between any two carbon atoms marked with * connected by a moiety -CL- is one carbamate bond. In other embodiments the at least one degradable bond are two carbamate bonds. In other embodiments the at least one degradable bond are three carbamate bonds. In other embodiments the at least one degradable bond are four carbamate bonds. In other embodiments the at least one degradable bond are five carbamate bonds. In other embodiments the at least one degradable bond are six carbamate bonds.

In certain embodiments the at least one degradable bond present in the direct connection between any two carbon atoms marked with * connected by a moiety -CL- is one amide bond. In other embodiments the at least one degradable bond are two amide bonds. In other embodiments the at least one degradable bond are three amide bonds. In other embodiments the at least one degradable bond are four amide bonds. In other embodiments the at least one degradable bond are five amide bonds. In other embodiments the at least one degradable bond are six amide bonds.

In some embodiments -CL- is C₁₋₅₀ alkyl, which is optionally interrupted by one or more atoms or groups selected from the group consisting of -T-, —C(O)O—, —O—, —C(O)—, —C(O)N(R^{c1})—, —S(O)₂—, —S(O)—, —S—, —N(R^{c1})—, —OC(OR^{c1})(R^{c1a})— and —OC(O)N(R^{c1})—;
wherein -T- is selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; and
—R^{c1} and —R^{c1a} are selected from the group consisting of —H and C₁₋₆ alkyl.

In certain embodiments -CL- is a moiety of formula (B)

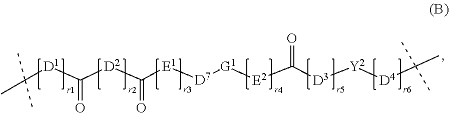

wherein
—Y¹— is of formula

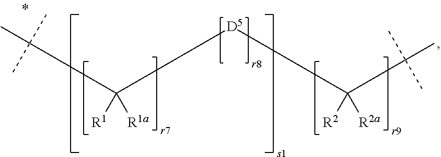

wherein the dashed line marked with the asterisk indicates attachment to -D¹- and the unmarked dashed line indicates attachment to -D²-;
—Y²— is of formula

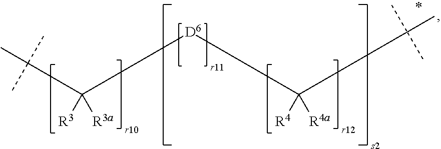

wherein the dashed line marked with the asterisk indicates attachment to -D⁴- and the unmarked dashed line indicates attachment to -D³-;
-E¹- is of formula

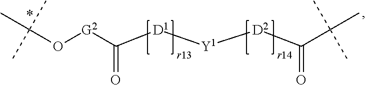

wherein the dashed line marked with the asterisk indicates attachment to —(C═O)— and the unmarked dashed line indicates attachment to —O—;
-E²- is of formula

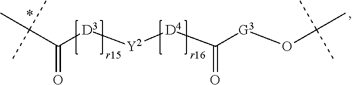

wherein the dashed line marked with the asterisk indicates attachment to -G¹- and the unmarked dashed line indicates attachment to —(C═O)—;
-G¹- is of formula

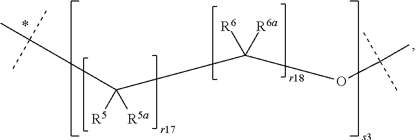

wherein the dashed line marked with the asterisk indicates attachment to —O— and the unmarked dashed line indicates attachment to -E²-;
-G²- is of formula

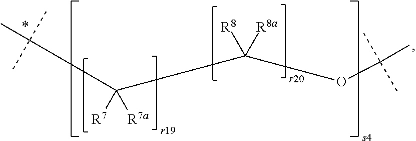

wherein the dashed line marked with the asterisk indicates attachment to —O— and the unmarked dashed line indicates attachment to —(C═O)—;
-G³- is of formula (C-vii),

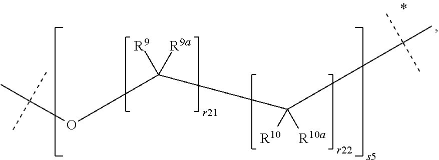

wherein the dashed line marked with the asterisk indicates attachment to —O— and the unmarked dashed line indicates attachment to —(C═O)—;
-D¹-, -D²-, -D³-, -D⁴-, -D⁵-, -D⁶- and -D⁷- are identical or different and each is independently of the others selected from the group comprising —O—, —NR¹¹—, —NR¹²R^{12a}—, —S—, —(S═O)—, —(S(O)₂), —C(O)—, —P(O)R¹³ and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹, —R^{9a}, —R¹⁰, —R^{10a}, —R¹¹, —R¹, —R^{12a}, —R¹³, —R¹⁴ and —R^{14a} are identical or different and each is independently of the others selected from the group comprising —H and C₁₋₆ alkyl;
optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
r1, r2, r5, r6, r13, r14, r15 and r16 are independently 0 or 1;
r3, r4, r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10; and
s1, s2, s4, s5 are independently 1, 2, 3, 4, 5 or 6.
s3 ranges from 1 to 200, preferably from 1 to 100 and more preferably from 1 to 50.

In certain embodiments r1 of formula (B) is 0. In certain embodiments r1 of formula (B) is 1. In certain embodiments r2 of formula (B) is 0. In certain embodiments r2 of formula (B) is 1. In certain embodiments r5 of formula (B) is 0. In certain embodiments r5 of formula (B) is 1. In certain embodiments r6 of formula (B) is 0. In certain embodiments r6 of formula (B) is 1. In certain embodiments r13 of formula (B) is 0. In certain embodiments r13 of formula (B) is 1. In certain embodiments r14 of formula (B) is 0. In certain embodiments r14 of formula (B) is 1. In certain embodiments r15 of formula (B) is 0. In certain embodiments r15 of formula (B) is 1. In certain embodiments r16 of formula (B) is 0. In certain embodiments r16 of formula (B) is 1.

In certain embodiments r3 of formula (B) is 0. In certain embodiments r3 of formula (B) is 1. In certain embodiments r4 of formula (B) is 0. In certain embodiments r4 of formula (B) is 1. In certain embodiments r3 of formula (B) and r4 of formula (B) are both 0.

In certain embodiments r7 of formula (B) is 0. In certain embodiments r7 of formula (B) is 1. In certain embodiments r7 of formula (B) is 2. In certain embodiments r8 of formula (B) is 0. In certain embodiments r8 of formula (B) is 1. In certain embodiments r8 of formula (B) of formula (B) is 2. In certain embodiments r9 of formula (B) is 0. In certain embodiments r9 of formula (B) is 1. In certain embodiments r9 of formula (B) is 2. In certain embodiments r10 of formula (B) is 0. In certain embodiments r10 of formula (B) is 1. In certain embodiments r10 of formula (B) is 2. In certain embodiments r11 of formula (B) is 0. In certain embodiments r11 of formula (B) is 1. In certain embodiments r11 of formula (B) is 2. In certain embodiments r12 of formula (B) is 0. In certain embodiments r12 of formula (B) is 1. In certain embodiments r12 of formula (B) is 2.

In certain embodiments r17 of formula (B) is 1. In certain embodiments r18 of formula (B) is 1. In certain embodiments r19 of formula (B) is 1. In certain embodiments r20 of formula (B) is 1. In certain embodiments r21 of formula (B) is 1.

In certain embodiments s1 of formula (B) is 1. In certain embodiments s1 of formula (B) is 2. In certain embodiments s2 of formula (B) is 1. In certain embodiments s2 of formula (B) is 2. In certain embodiments s4 of formula (B) is 1. In certain embodiments s4 of formula (B) is 2.

In certain embodiments s3 of formula (B) ranges from 1 to 100. In certain embodiments s3 of formula (B) ranges from 1 to 75. In certain embodiments s3 of formula (B) ranges from 2 to 50. In certain embodiments s3 of formula (B) ranges from 2 to 40. In certain embodiments s3 of formula (B) ranges from 3 to 30. In certain embodiments s3 of formula (B) is about 3.

In certain embodiments —R¹ of formula (B) is —H. In certain embodiments —R¹ of formula (B) is methyl. In certain embodiments —R¹ of formula (B) is ethyl. In certain embodiments —R^{1a} of formula (B) is —H. In certain embodiments —R^{1a} of formula (B) is methyl. In certain embodiments —R^{1a} of formula (B) is ethyl. In certain embodiments —R² of formula (B) is —H. In certain embodiments —R² of formula (B) is methyl. In certain embodiments —R² of formula (B) is ethyl. In certain embodiments —R^{2a} of formula (B) is —H. In certain embodiments —R^{2a} of formula (B) is methyl. In certain embodiments —R^{2a} of formula (B) is ethyl. In certain embodiments —R³ of formula (B) is —H. In certain embodiments —R³ of formula (B) is methyl. In certain embodiments —R³ of formula (B) is ethyl. In certain embodiments —R^{3a} of formula (B) is —H. In certain embodiments —R^{3a} of formula (B) is methyl. In certain embodiments —R^{3a} of formula (B) is ethyl. In certain embodiments —R⁴ of formula (B) is —H. In certain embodiments —R⁴ of formula (B) is methyl. In certain embodiments —R⁴ of formula (B) is methyl. In certain embodiments —R^{4a} of formula (B) is —H. In certain embodiments —R^{4a} of formula (B) is methyl. In certain embodiments —R^{4a} of formula (B) is ethyl. In certain embodiments —R⁵ of formula (B) is —H. In certain embodiments —R⁵ of formula (B) is methyl. In certain embodiments —R⁵ of formula (B) is ethyl. In certain embodiments —R^{5a} of formula (B) is —H. In certain embodiments —R^{5a} of formula (B) is methyl. In certain embodiments —R^{5a} of formula (B) is ethyl. In certain embodiments —R⁶ of formula (B) is —H. In certain embodiments —R⁶ of formula (B) is methyl. In certain embodiments —R⁶ of formula (B) is ethyl. In certain embodiments —R^{6a} of formula (B) is —H. In certain embodiments —R^{6a} of formula (B) is methyl. In certain embodiments —R^{6a} of formula (B) is ethyl. In certain embodiments —R⁷ of formula (B) is —H. In certain embodiments —R⁷ of formula (B) is methyl. In certain embodiments —R⁷ of formula (B) is ethyl. In certain embodiments —R⁸ of formula (B) is —H. In certain embodiments —R⁸ of formula (B) is methyl. In certain embodiments —R⁸ of formula (B) is ethyl. In certain embodiments —R^{8a} of formula (B) is —H. In certain embodiments —R^{8a} of formula (B) is methyl. In certain embodiments —R^{8a} of formula (B) is ethyl. In certain embodiments —R⁹ of formula (B) is —H. In certain embodiments —R⁹ of formula (B) is methyl. In certain embodiments —R⁹ of formula (B) is ethyl. In certain embodiments —R^{9a} of formula (B) is —H. In certain embodiments —R^{9a} of formula (B) is methyl. In certain embodiments —R^{9a} of formula (B) is ethyl. In certain embodiments —R^{9a} of formula (B) is —H. In certain embodiments —R^{9a} of formula (B) is methyl. In certain embodiments —R^{9a} of formula (B) is ethyl. In certain embodiments —R¹⁰ of formula (B) is —H. In certain embodiments —R¹⁰ of formula (B) is methyl. In certain embodiments —R¹⁰ of formula (B) is ethyl. In certain embodiments —R^{10a} of formula (B) is —H. In certain embodiments —R^{10a} of formula (B) is methyl. In certain embodiments —R^{10a} of formula (B) is ethyl. In certain embodiments —R¹¹ of formula (B) is —H. In certain embodiments —R¹¹ of formula (B) is methyl. In certain embodiments —R¹¹ of formula (B) is ethyl. In certain embodiments —R¹² of formula (B) is —H. In certain embodiments —R¹² of formula (B) is methyl. In certain embodiments —R¹² of formula (B) is ethyl. In certain embodiments —R^{12a} of formula (B) is —H. In certain embodiments —R^{12a} of formula (B) is methyl. In certain embodiments —R^{12a} of formula (B) is ethyl. In certain embodiments —R¹³ of formula (B) is —H. In certain embodiments —R¹³ of formula (B) is methyl. In certain embodiments —R¹³ of formula (B) is ethyl In certain embodiments —R¹⁴ of formula (B) is —H. In certain embodiments —R¹⁴ of formula (B) is methyl. In certain embodiments —R¹⁴ of formula (B) is ethyl. In certain embodiments —R^{14a} of formula (B) is —H. In certain embodiments —R^{14a} of formula (B) is methyl. In certain embodiments —R^{14a} of formula (B) is ethyl.

In certain embodiments -D¹- of formula (B) is —O—. In certain embodiments -D¹- of formula (B) is —NR¹¹—. In certain embodiments -D¹- of formula (B) is —N⁺R¹²R^{12a}—. In certain embodiments -D¹- of formula (B) is —S—. In certain embodiments -D¹- of formula (B) is —(S═O). In certain embodiments -D¹- of formula (B) is —(S(O)₂)—. In certain embodiments -D¹- of formula (B) is —C(O)—. In certain embodiments -D¹- of formula (B) is —P(O)R¹³—. In certain embodiments -D¹- of formula (B) is —P(O)(OR¹³)—. In certain embodiments -D¹- of formula (B) is —CR¹⁴R^{14a}—.

In certain embodiments -D²- of formula (B) is —O—. In certain embodiments -D²- of formula (B) is —NR¹¹—. In certain embodiments -D²- of formula (B) is —N⁺R¹²R^{12a}—. In certain embodiments -D²- of formula (B) is —S—. In certain embodiments -D²- of formula (B) is —(S═O). In certain embodiments -D²- of formula (B) is —(S(O)₂)—. In certain embodiments -D²- of formula (B) is —C(O)—. In certain embodiments -D²- of formula (B) is —P(O)R¹³—. In certain embodiments -D²- of formula (B) is —P(O)(OR¹³)—. In certain embodiments -D²- of formula (B) is —CR¹⁴R^{14a}—.

In certain embodiments -D³- of formula (B) is —O—. In certain embodiments -D³- of formula (B) is —NR¹¹—. In certain embodiments -D³- of formula (B) is —N⁺R¹²R^{12a}—. In certain embodiments -D³- of formula (B) is —S—. In certain embodiments -D³- of formula (B) is —(S═O). In certain embodiments -D³- of formula (B) is —(S(O)₂)—. In certain embodiments -D³- of formula (B) is —C(O)—. In certain embodiments -D³- of formula (B) is —P(O)R¹³—. In certain embodiments -D³- of formula (B) is —P(O)(OR¹³)—. In certain embodiments -D³- of formula (B) is —CR¹⁴R^{14a}—.

In certain embodiments -D⁴- of formula (B) is —O—. In certain embodiments -D⁴- of formula (B) is —NR¹¹—. In certain embodiments -D⁴- of formula (B) is —N⁺R¹²R^{12a}—. In certain embodiments -D⁴- of formula (B) is —S—. In certain embodiments -D⁴- of formula (B) is —(S═O). In certain embodiments -D⁴- of formula (B) is —(S(O)₂)—. In certain embodiments -D⁴- of formula (B) is —C(O)—. In certain embodiments -D⁴- of formula (B) is —P(O)R¹³—. In certain embodiments -D⁴- of formula (B) is —P(O)(OR¹³)—. In certain embodiments -D⁴- of formula (B) is —CR¹⁴R^{14a}—.

In certain embodiments -D⁵- of formula (B) is —O—. In certain embodiments -D⁵- of formula (B) is —NR¹¹—. In certain embodiments -D⁵- of formula (B) is —N⁺R¹²R^{12a}—. In certain embodiments -D⁵- of formula (B) is —S—. In certain embodiments -D⁵- of formula (B) is —(S═O)—. In certain embodiments -D⁵- of formula (B) is —(S(O)₂)—. In certain embodiments -D⁵- of formula (B) is —C(O)—. In certain embodiments -D⁵- of formula (B) is —P(O)R¹³—. In certain embodiments -D⁵- of formula (B) is —P(O)(OR¹³)—. In certain embodiments -D⁵- of formula (B) is —CR¹⁴R^{14a}—.

In certain embodiments -D⁶- of formula (B) is —O—. In certain embodiments -D⁶- of formula (B) is —NR¹—. In certain embodiments -D⁶- of formula (B) is —N⁺R¹²R^{12a}—. In certain embodiments -D⁶- of formula (B) is —S—. In certain embodiments -D⁶- of formula (B) is —(S═O). In certain embodiments -D⁶- of formula (B) is —(S(O)₂)—. In certain embodiments -D⁶- of formula (B) is —C(O)—. In certain embodiments -D⁶- of formula (B) is —P(O)R¹³—. In certain embodiments -D⁶- of formula (B) is —P(O)(OR¹³)—. In certain embodiments -D⁶- of formula (B) is —CR¹⁴R^{14a}—.

In certain embodiments -D⁷- of formula (B) is —O—. In certain embodiments -D⁷- of formula (B) is —NR¹¹—. In certain embodiments -D⁷- of formula (B) is —N⁺R¹²R^{12a}—. In certain embodiments -D⁷- of formula (B) is —S—. In certain embodiments -D⁷- of formula (B) is —(S═O). In certain embodiments -D⁷- of formula (B) is —(S(O)₂)—. In certain embodiments -D⁷- of formula (B) is —C(O)—. In certain embodiments -D⁷- of formula (B) is —P(O)R¹³—. In certain embodiments -D⁷- of formula (B) is —P(O)(OR¹³)—. In certain embodiments -D⁷- of formula (B) is —CR¹⁴R^{14a}—.

In certain embodiments -CL- is of formula (B-i)

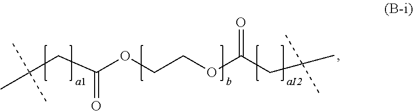

wherein
a1 and a2 are independently selected from the group consisting of a1 and a2 are independently selected from the group consisting of 1, 2, 3, 4, 5, 6, 7, 8, 9, 10, 11, 12, 13 and 14; and
b is an integer ranging from 1 to 50.

In certain embodiments a1 and a2 of formula (B-i) are different. In certain embodiments a1 and a2 of formula (B-i) are the same.

In certain embodiments a1 of formula (B-i) is 1. In certain embodiments a1 of formula (B-i) is 2. In certain embodiments a1 of formula (B-i) is 3. In certain embodiments a1 of formula (B-i) is 4. In certain embodiments a1 of formula (B-i) is 5. In certain embodiments a1 of formula (B-i) is 6. In certain embodiments a1 of formula (B-i) is 7. In certain embodiments a1 of formula (B-i) is 8. In certain embodiments a1 of formula (B-i) is 9. In certain embodiments a1 of formula (B-i) is 10.

In certain embodiments a2 of formula (B-i) is 1. In certain embodiments a2 of formula (B-i) is 2. In certain embodiments a2 of formula (B-i) is 3. In certain embodiments a2 of formula (B-i) is 4. In certain embodiments a2 of formula (B-i) is 5. In certain embodiments a2 of formula (B-i) is 6. In certain embodiments a2 of formula (B-i) is 7. In certain embodiments a2 of formula (B-i) is 8. In certain embodiments a2 of formula (B-i) is 9. In certain embodiments a2 of formula (B-i) is 10.

In certain embodiments b of formula (B-i) ranges from 1 to 500. In certain embodiments b of formula (B-i) ranges from 2 to 250. In certain embodiments b of formula (B-i) ranges from 3 to 100. In certain embodiments b of formula (B-i) ranges from 3 to 50. In certain embodiments b of formula (B-i) ranges from 3 to 25. In certain embodiments b of formula (B-i) is 3. In certain embodiments b of formula (B-i) is 25.

In certain embodiments -CL- is of formula (B-i)

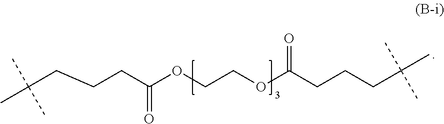

In certain embodiments -CL- is of formula (B-ii)

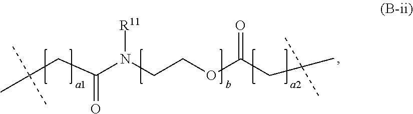

wherein
a1 and a2 are independently selected from the group consisting of 1, 2, 3, 4, 5, 6, 7, 8, 9, 10, 11, 12, 13 and 14;
b is an integer ranging from 1 to 50; and
—R¹¹ is selected from the group comprising —H and C₁₋₆ alkyl.

In certain embodiments a1 and a2 of formula (B-ii) are different. In certain embodiments a1 and a2 of formula (B) are the same.

In certain embodiments a1 of formula (B-ii) is 1. In certain embodiments a1 of formula (B-ii) is 2. In certain embodiments a1 of formula (B-ii) is 3. In certain embodiments a1 of formula (B-ii) is 4. In certain embodiments a1 of formula (B-ii) is 5. In certain embodiments a1 of formula (B-ii) is 6. In certain embodiments a1 of formula (B-ii) is 7. In certain embodiments a1 of formula (B-ii) is 8. In certain embodiments a1 of formula (B-ii) is 9. In certain embodiments a1 of formula (B-ii) is 10.

In certain embodiments a2 of formula (B-ii) is 1. In certain embodiments a2 of formula (B-ii) is 2. In certain embodiments a2 of formula (B-ii) is 3. In certain embodiments a2 of formula (B-ii) is 4. In certain embodiments a2 of formula (B-ii) is 5. In certain embodiments a2 of formula (B-ii) is 6. In certain embodiments a2 of formula (B-ii) is 7. In certain embodiments a2 of formula (B-ii) is 8. In certain embodiments a2 of formula (B-ii) is 9. In certain embodiments a2 of formula (B-ii) is 10.

In certain embodiments b of formula (B-ii) ranges from 1 to 500. In certain embodiments b of formula (B-ii) ranges from 2 to 250. In certain embodiments b of formula (B-ii) ranges from 3 to 100. In certain embodiments b of formula (B-ii) ranges from 3 to 50. In certain embodiments b of formula (B-ii) ranges from 3 to 25. In certain embodiments b of formula (B-ii) is 3. In certain embodiments b of formula (B-ii) is 25.

In certain embodiments —R¹¹ of formula (B-ii) is —H. In certain embodiments —R¹¹ of formula (B-ii) is methyl. In certain embodiments —R¹¹ of formula (B-ii) is ethyl. In certain embodiments —R¹¹ of formula (B-ii) is n-propyl. In certain embodiments —R¹¹ of formula (B-ii) is isopropyl. In certain embodiments —R¹¹ of formula (B-ii) is n-butyl. In certain embodiments —R¹¹ of formula (B-ii) is isobutyl. In certain embodiments —R¹¹ of formula (B-ii) is sec-butyl. In certain embodiments —R¹¹ of formula (B-ii) is tert-butyl. In certain embodiments —R¹¹ of formula (B-ii) is n-pentyl. In certain embodiments —R¹¹ of formula (B-ii) is 2-methylbutyl. In certain embodiments —R¹¹ of formula (B-ii) is 2,2-dimethylpropyl. In certain embodiments —R¹¹ of formula (B-ii) is n-hexyl. In certain embodiments —R¹¹ of formula (B-ii) is 2-methylpentyl. In certain embodiments —R¹¹ of formula (B-ii) is 3-methylpentyl. In certain embodiments —R¹¹ of formula (B-ii) is 2,2-dimethylbutyl. In certain embodiments —R¹¹ of formula (B-ii) is 2,3-dimethylbutyl. In certain embodiments —R¹¹ of formula (B-ii) is 3,3-dimethylpropyl.

In certain embodiments -CL- is of formula (B-iii)

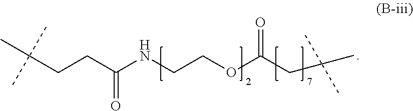

In a second embodiment the moiety -CL- is selected from the group consisting of

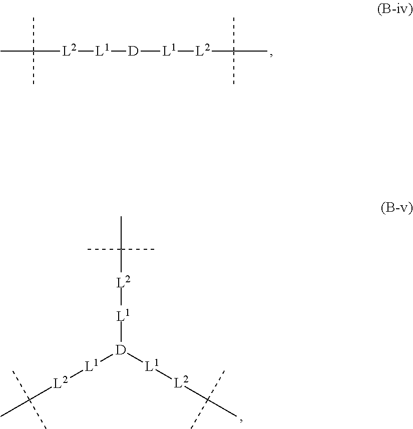

wherein
each dashed line indicates attachment to a unit Z³; and
-L¹-, -L²- and -D are used as defined for Z².

It is understood that in formula (B-iv) two functional groups of the drug are conjugated to one moiety -L¹- each and that in formula (B-v) three functional groups of the drug are conjugated to one moiety -L¹- each. The moiety -CL- of formula (B-iv) connects two moieties Z³ and the moiety -CL- of formula (B-v) connects three moieties Z³, which may be on the same or different hyaluronic acid strand. In this embodiment -CL- comprises at least two degradable bonds, if -CL- is of formula (B-iv) or at least three degradable bonds, if -CL- is of formula (B-v), namely the degradable bonds that connect D with a moiety -L¹-. A conjugate of the present invention may only comprise moieties -CL- of formula (B-iv), may only comprise moieties -CL- of formula (B-v) or may comprise moieties -CL- of formula (B-iv) and formula (B-v).

Accordingly, a conjugate of the present invention of this second embodiment comprises crosslinked hyaluronic acid strands to which a plurality of drug moieties are covalently and reversibly conjugated, wherein the conjugate of the present invention comprises a plurality of connected units selected from the group consisting of

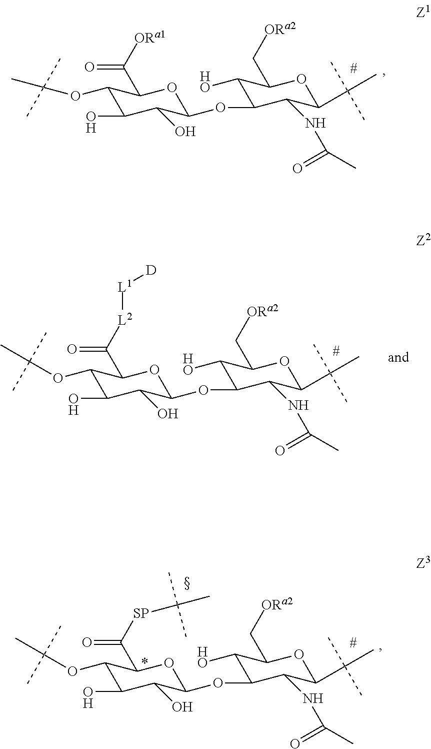

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between at least two units Z³ via a moiety -CL-;
each -CL- comprises at least one degradable bond between the two carbon atoms marked with the * connected by a moiety -CL- and each -CL- is independently selected from the group consisting of formula (B-iv) and (B-v)

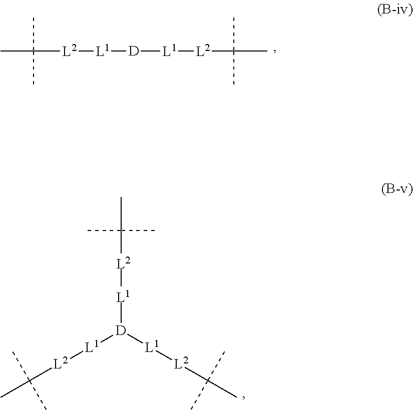

wherein
dashed lines indicate attachment to a unit Z³; -D, -L¹-, -L²-, —SP—, —R^{a1} and —R^{a2} are used as defined for Z¹, Z² and Z³;
wherein
all units Z¹ present in the conjugate may be the same or different;
all units Z² present in the conjugate may be the same or different;
all units Z³ present in the conjugate may be the same or different;
the number of Z¹ units ranges from 1% to 98% of the total number of units present in the conjugate of the present invention;
the number of Z² units ranges from 0% to 98% of the total number of units present in the conjugate of the present invention;
the number of Z³ units ranges from 1% to 97% of the total number of units present in the conjugate of the present invention, provided that at least one unit Z³ is present per strand which is connected to at least one unit Z³ on a different hyaluronic acid strand.

It is understood that such hydrogel according to the second embodiment also comprises partly reacted or unreacted units and that the presence of such moieties cannot be avoided. In certain embodiments the sum of such partly reacted or unreacted units is no more than 25% of the total number of units present in the conjugate, such as no more than 10%, such as no more than 15% or such as no more than 10%.

In a conjugate of the present invention according to this second embodiment the number of units Z² ranges from 0 to 70% of all units present in the conjugate of the present invention, such as from 2 to 15%, from 2 to 10%, from 16 to 39, from 40 to 65%, or from 50 to 60% of all units present in the conjugate of the present invention.

In a conjugate of the present invention according to this second embodiment the number of units Z³ ranges from 1 to 30% of all units present in the conjugate of the present invention, such as from 2 to 5%, from 5 to 20%, from 10 to 18%, or from 14 to 18% of all units present in the conjugate of the present invention.

In a conjugate of the present invention according to this second embodiment the number of units Z¹ ranges from 10 to 97% of all units present in the conjugate of the present invention, such as from 20 to 40%, such as from 25 to 35%, such as from 41 to 95%, such as from 45 to 90%, such as from 50 to 70% of all units present in the conjugate of the present invention.

More specific embodiments for -D, -L¹-, -L²-, —SP—, —R^{a1} and —R^{a2} of the second embodiment are as described elsewhere herein.

In a third embodiment the moiety -CL- is a moiety

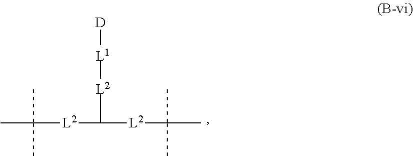

wherein
each dashed line indicates attachment to a unit Z³.

It is understood that a moiety -CL- of formula (B-vi) comprises at least one branching point, which branching point may be selected from the group consisting of

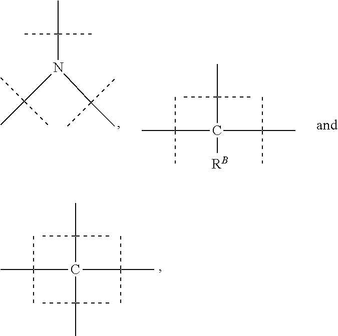

wherein
dashed lines indicate attachment to an arm; and
—R^{B} is selected from the group consisting of —H, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{B1}, which are the same or different, and wherein C₁₋₆ alkyl, C₂₋₆ alkenyl and C₂₋₆ alkynyl are optionally interrupted with —C(O)O—, —O—, —C(O)—, —C(O)N(R^{B2})—, —S(O)₂N(R^{B2})—, —S(O)N(R^{B2})—, —S(O)₂—, —S(O)—, —N(R^{B2})S(O)₂N(R^{B2a})—, —S—, —N(R^{B2})—, —OC(OR^{B2})(R^{B2a})—, —N(R^{B2})C(O)N(R^{B2a})—, and —OC(O)N(R^{B2})—; wherein —R^{B1}, —R^{B2} and —R^{B2a} are selected from —H, C₁₋₆ alkyl, C₂₋₆ alkenyl and C₂₋₆ alkynyl.

In certain embodiments —R^{B} is selected from the group consisting of —H, methyl and ethyl.

Accordingly, a conjugate of the present invention of the third embodiment comprises crosslinked hyaluronic acid strands to which a plurality of drug moieties are covalently and reversibly conjugated, wherein the conjugate of the present invention comprises a plurality of connected units selected from the group consisting of

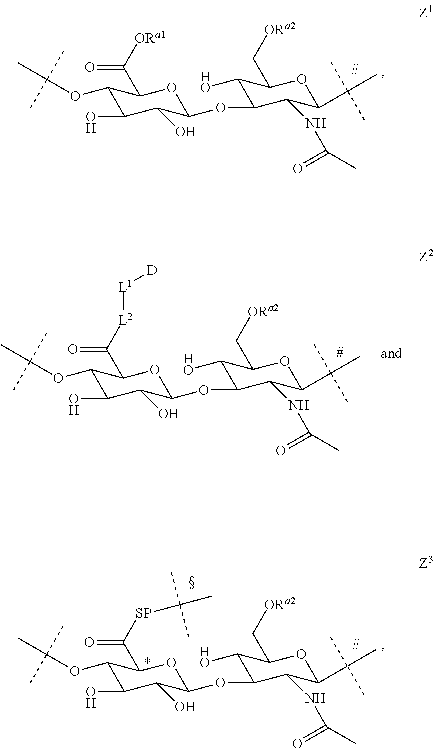

wherein
an unmarked dashed line indicates a point of attachment to an adjacent unit at a dashed line marked with # or to a hydrogen;
a dashed line marked with # indicates a point of attachment to an adjacent unit at an unmarked dashed line or to a hydroxyl;
a dashed line marked with § indicates a point of connection between two units Z³ via a moiety -CL-;
each -CL- comprises at least one degradable bond between the two carbon atoms marked with the * connected by a moiety -CL- and each -CL- is independently of formula (B-vi)

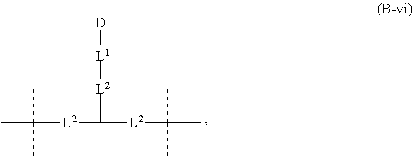

wherein
dashed lines indicate attachment to a unit Z³;
-D, -L¹-, -L²-, —SP—, —R^{a1} and —R^{a2} are used as defined for Z¹, Z² and Z³;
wherein
all units Z¹ present in the conjugate may be the same or different;
all units Z² present in the conjugate may be the same or different;
all units Z³ present in the conjugate may be the same or different;
the number of units Z¹ ranges from 1% to 99% of the total number of units present in the conjugate of the present invention;
the number of units Z² ranges from 0% to 98% of the total number of units present in the conjugate of the present invention; and
the number of units Z³ ranges from 1% to 97% of the total number of units present in the conjugate of the present invention, provided that at least one unit Z³ is present per strand.

It is understood that such hydrogel according to the third embodiment also comprises partly reacted or unreacted units and that the presence of such moieties cannot be avoided. In certain embodiments the sum of such partly reacted or unreacted units is no more than 25% of the total number of units present in the conjugate of the present invention, such as no more than 10%, such as no more than 15% or such as no more than 10%.

In a conjugate of the present invention according to this third embodiment the number of units Z² ranges from 0 to 70% of all units present in the conjugate of the present invention, such as from 2 to 15%, from 2 to 10%, from 16 to 39, from 40 to 65%, or from 50 to 60% of all units present in the conjugate of the present invention.

In a conjugate of the present invention according to this third embodiment the number of units Z³ ranges from 1 to 30% of all units present in the conjugate of the present invention, such as from 2 to 5%, from 5 to 20%, from 10 to 18%, or from 14 to 18% of all units present in the conjugate of the present invention.

In a conjugate of the present invention according to this third embodiment the number of units Z¹ ranges from 10 to 97% of all units present in the conjugate of the present invention, such as from 20 to 40%, such as from 25 to 35%, such as from 41 to 95%, such as from 45 to 90%, such as from 50 to 70% of all units present in the conjugate of the present invention.

In this third embodiment -CL- comprises a moiety -L²- L¹-D, so the presence of units Z² is optional in this embodiment. In certain embodiment no units Z² are present in the third embodiment. In certain embodiments the conjugate of the present invention according to the third embodiment also comprises units Z². The presence of units Z² may have the effect that in case of a high drug loading is desired, which in this embodiment also means a high degree of crosslinking, an undesired high degree of crosslinking can be avoided by the presence of units Z².

More specific embodiments for -D, -L¹-, -L²-, —SP—, —R^{a1} and —R^{a2} of the second embodiment are as described elsewhere herein.

—SP— is absent or a spacer moiety. In certain embodiments —SP— does not comprise a reversible linkage, i.e. all linkages in —SP— are stable linkages.

In certain embodiments —SP— is absent.

In certain embodiments —SP— is a spacer moiety.

In certain embodiments —SP— does not comprise a degradable bond, i.e. all bonds of —SP— are stable bonds. In certain embodiments at least one of the at least one degradable bond in the direct connection between two carbon atoms marked with the * connected by a moiety -CL- is provided by —SP—.

In certain embodiments —SP— is a spacer moiety selected from the group consisting of -T-, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments —SP— is a spacer moiety selected from the group consisting of -T-, C₁. 50 alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y51} is independently of each other selected from the group consisting of —H, and C₁₋₆ alkyl; wherein C₁₋₆ alkyl is optionally substituted with one or more halogen, which are the same or different.

In certain embodiments —SP— is a spacer moiety selected from the group consisting of -T-, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen and C₁₋₆ alkyl; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments —SP— is a C₁₋₂₀ alkyl chain, which is optionally interrupted by one or more groups independently selected from —O—, -T-, —N(R^{y3})— and —C(O)N(R^{y1})—; and which C₁₋₂₀ alkyl chain is optionally substituted with one or more groups independently selected from —OH, -T, —N(R^{y3})— and —C(O)N(R^{y6}R^{y6a}); wherein —R^{y1}, —R^{y6}, —R^{y6a} are independently selected from the group consisting of H and C₁₋₄ alkyl, wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments —SP— has a molecular weight ranging from 14 g/mol to 750 g/mol.

In certain embodiments —SP— has a chain length ranging from 1 to 20 atoms.

In certain embodiments all moieties —SP— of a conjugate are identical.

In certain embodiments —SP— is a C₁₋₁₀ alkyl. In certain embodiments —SP— is a C₁ alkyl. In certain embodiments —SP— is a C₂ alkyl. In certain embodiments —SP— is a C₃ alkyl. In certain embodiments —SP— is a C₄ alkyl. In certain embodiments —SP— is a C₅ alkyl. In certain embodiments —SP— is a C₆ alkyl. In certain embodiments —SP— is a C₇ alkyl. In certain embodiments —SP— is a C₈ alkyl. In certain embodiments —SP— is a C₉ alkyl. In certain embodiments —SP— is a C₁₀ alkyl.

In certain embodiments the anti-CTLA4 conjugate is selected from the group consisting of crystals, nanoparticles, microparticles, nanospheres, microspheres, particles with a diameter larger than about 1 mm and continuous gels. In certain embodiments the anti-CTLA4 conjugate is a crystal. In certain embodiments the anti-CTLA4 conjugate is a nanoparticle, such as a nanoparticle with an average diameter ranging from 5 to 800 nm, a nanoparticle with an average diameter ranging from 10 to 600 nm or a nanoparticle with an average diameter ranging from 20 to 500 nm. In certain embodiments the anti-CTLA4 conjugate is a microparticle, such as microparticle with an average diameter ranging from 10 to 950 μm, such as a microparticle with an average diameter ranging from 20 to 500 μm, such as a microparticle with an average diameter ranging from 25 to 250 μm, such as a microparticle with an average diameter ranging from 30 to 250 μm or a microparticle with an average diameter ranging from 35 to 150 μm. In certain embodiments the anti-CTLA4 conjugate is a nanosphere, such as a nanosphere with an average diameter ranging from 5 to 800 nm, a nanosphere with an average diameter ranging from 10 to 600 nm or a nanosphere with an average diameter ranging from 20 to 500 nm. In certain embodiments the anti-CTLA4 conjugate is a microsphere, such as microsphere with an average diameter ranging from 10 to 700 μm, such as a microsphere with an average diameter ranging from 20 to 500 μm, such as a microsphere with an average diameter ranging from 25 to 250 μm, such as a microsphere with an average diameter ranging from 30 to 250 μm or a microsphere with a n average diameter ranging from 35 to 150 μm. In certain embodiments the anti-CTLA4 conjugate is a particle with an average diameter larger than about 1 mm, such as with an average diameter of at least 2 mm, with an average diameter of at least 4 mm or with an average diameter of at least 5 mm. In certain embodiments the anti-CTLA4 conjugate is a continuous gel.

In certain embodiments intra-tissue administration of the anti-CTLA4 conjugate induces local anti-CTLA4-induced T cell activation.

Said local anti-CTLA4-induced T cell activation is in certain embodiments an at least 10% increase, such as an at least 20% increase, an at least 30% increase or an at least 40% increase, compared to baseline or compared to no CTLA4 controls in at least two, such as 2, 3, 4 or 5, of the markers selected from the group consisting of the percentage of ICOS⁺ cells, the percentage of CD25⁺ cells, the percentage of CD69⁺ cells, the percentage of Ki67⁺ cells, or the percentage of CD44⁺⁺CD62L^{1ow} effector cells within CD4 T cells locally or in draining lymph nodes associated with the injection site.

In certain embodiments the local anti-CTLA4-induced T cell activation is measured 7 days after intra-tissue administration. In certain embodiments essentially no systemic anti-CTLA4-induced response is elicited after said intra-tissue administration.

In certain embodiments said essentially no systemic CTLA4-induced response is a less than 10%, such as a no more than 8%, no more than 6% or no more than 5%, increase compared to baseline or compared no CTLA4 controls of at least four of the features selected from the group consisting of the percentage of CD4 T cells of total T cells, the percentage of FOXP3⁺ T cells within CD4 T cells, the percentage of ICOS⁺ cells within CD4 T cells, the percentage of CD25⁺ cells within CD4 T cells, the percentage of CD69⁺ cells within CD4 T cells, the percentage of Ki67⁺ cells within CD4 T cells, the percentage of CD44⁺⁺CD62L^{1ow} effector cells within CD4 T cells, the percentage of ICOS⁺ cells within CD4⁺ CD25⁺ FOXP3⁺ regulatory T cells (“T_{regs}”), the percentage of CD69⁺ cells within T_{regs}, and the percentage of Ki67⁺ cells within T_{regs} in the blood, or the spleen or in lymph nodes which are contralateral to or do not drain from the injection site.

In certain embodiments said less than 10% increase is measured 7 days after said intra-tissue administration.

When the anti-CTLA4 compound of the present invention is administered in a dose that results in the same +/−20% tumor growth inhibition as systemic anti-CTLA4 administration, a less than 10% or even a less than 5% increase in at least four of the features selected from the group consisting of the percentage of ICOS⁺ cells within blood CD4 T cells, the percentage of Ki67⁺ cells within blood CD4 T cells, the percentage of CD44⁺⁺CD62L^{1ow} effector cells within blood CD4 T cells, the percentage of Ki67+ cells within spleen CD4 T cells, and the percentage of Ki67+ cells within spleen T_{reg} cells is observed compared to baseline or compared to no CTLA4 controls. The lack of induction of these systemic activation markers with water-insoluble controlled-release anti-CLTA4 compound of the present invention is noteworthy as these markers are typically induced by systemic anti-CTLA4 therapy which is known to be associated with systemic adverse events.

In certain embodiments a single intra-tissue injection results in anti-tumor activity 7 days after intra-tissue administration.

In certain embodiments the anti-CTLA4 conjugate is administered by intra-tissue administration in a dose of 10 mg anti-CTLA4 equivalents/kg body weight and the systemic response measured in blood or spleen or lymph nodes which are contralateral to or do not drain from the injection site 7 days after said intra-tissue administration is at least 10-fold, such as at least 12-fold, at least 15-fold or at least 20-fold, lower compared to the systemic response in blood or spleen 7 days after systemic administration of 10 mg/kg body weight of the corresponding free anti-CTLA4 drug.

In certain embodiments the at least 10-fold lower systemic response is an at least 10-fold lower increase, such as an at least 12-fold lower increase, an at least 15-fold lower increase, an at least 20-fold lower increase or an at least 25-fold lower increase, in at least four events selected from the group consisting of an increase in CD4 T cells as a percentage of total T cells; an increase in the % of FOXP3+ cells within CD4 T cells; an increase in Median Fluorescence Intensity or % Positivity for ICOS within CD4 T cells; an increase in Median Fluorescence Intensity or % Positivity for CD25 within CD4 T cells, an increase in Median Fluorescence Intensity or % Positivity for CD69 within CD4 T cells; an increase in Median Fluorescence Intensity or % Positivity for Ki67 within CD4 T cells; an increase in Median Fluorescence Intensity or % Positivity for ICOS within T_{regs}; an increase in Median Fluorescence Intensity or % Positivity for CD69 within T_{regs}; and an increase in Median Fluorescence Intensity or % Positivity for Ki67 within T_{regs}.

In certain embodiments a single intra-tissue injection results in anti-tumor activity 7 days after intra-tissue administration. In certain embodiments a single intra-tissue injection results in anti-tumor activity on or before 20 days after intra-tissue administration.

In certain embodiments the anti-CTLA4 conjugate is administered by intra-tissue administration in a dose comprising at most 50%, such as no more than 45%, no more than 40%, no more than 35% or no more than 30%, of the equimolar dose of anti-CTLA4 moieties or drug molecules of a therapeutically effective dose of the systemically administered corresponding free anti-CTLA4 drug and wherein anti-tumor activity is observed 7 days after said intra-tissue administration.

In certain embodiments intra-tissue administration of a therapeutic dose of the anti-CTLA4 conjugate of the present invention results in a maximum systemic concentration of anti-CTLA4 drug within 48 hours after said intra-tissue administration that is at least 2-fold lower compared to the maximum systemic concentration of the corresponding anti-CTLA4 drug within 48 hours after systemic administration of a dose of said anti-CTLA4 drug that provides essentially the same anti-tumor activity.

The maximum systemic concentration of anti-CTLA4 drug within 48 hours after administration is determined in plasma or serum measured in weight per volume, such as in ng anti-CTLA4 drug per ml plasma or serum. In certain embodiments the maximum systemic concentration is measured in plasma. In certain embodiments the maximum systemic concentration is measured in serum. Maximum systemic concentration may be determined by taking multiple plasma or serum samples within a time period ranging from 0 to 48 hours, determining the anti-CTLA4 drug content in each of them, plotting the anti-CTLA4 drug concentrations as a function of time and determining the maximum concentration using suitable mathematical models. Exemplary time points for taking of the samples may be 1 hour, 4 hours, 8 hours, 16 hours, 24 hours and 48 hours after intra-tissue administration.

The maximum systemic concentration of anti-CTLA4 drug within 48h after is at least 2-fold lower compared to the maximum systemic concentration of anti-CTLA4 drug within 48 hours after systemic administration of a dose of anti-CTLA4 drug that provides essentially the same anti-tumor activity, such as at least 3-fold lower, at least 4-fold lower, at least 5-fold lower or at least 10-fold lower.

In certain embodiments the anti-CTLA4 conjugate is administered by intra-tissue administration and at 72 hours after a single such intra-tissue administration in a dose of 1 mg anti-CTLA4 equivalents/kg body weight systemic concentrations of released anti-CTLA4 drug are less than 1 μg/ml.

It is understood that no general dosage information can be provided for the anti-CTLA4 conjugate of the present invention based on the compound as such due to their varying amount of non-anti-CTLA4 moieties, i.e. due to the presence of for example varying amounts of polymeric moieties. Therefore, any dosage given is as mg anti-CTLA4 equivalents per kg body weight which ignores any non-anti-CTLA4 moieties present in the anti-CTLA4 conjugate.

In certain embodiments the systemic concentrations of released anti-CTLA4 at 72 hours are less than 0.9 μg/ml. In certain embodiments the systemic concentrations of released anti-CTLA4 at 72 hours are less than 0.8 μg/ml. In certain embodiments the systemic concentrations of released anti-CTLA4 at 72 hours are less than 0.7 μg/ml. In certain embodiments the systemic concentrations of released anti-CTLA4 at 72 hours are less than 0.6 μg/ml. In certain embodiments the systemic concentrations of released anti-CTLA4 at 72 hours are less than 0.5 μg/ml. In certain embodiments the systemic concentrations of released anti-CTLA4 at 72 hours are less than 0.4 μg/ml.

In certain embodiments the anti-CTLA4 conjugate is administered by intra-tissue administration and the systemic concentration of released anti-CTLA4 drug at 72 hours after such intra-tissue administration is at least 80%, such as at least 85%, at least 90% or at least 95%, of the systemic concentration at 1 hour after such intra-tissue administration. It is understood that the systemic concentration of released anti-CTLA4 may also be higher at 72 hours after intra-tissue administration compared to the systemic concentration at 1 hour after the same intra-tissue administration.

In certain embodiments the water-insoluble controlled-release anti-CTLA4 compound is administered by intra-tissue administration and at 24 hours after such intra-tissue administration systemic concentration levels of anti-CTLA4 drug are at least 50%, such as at least 55%, at least 60%, at least 65%, at least 70% at least 75%, at least 80%, at least 85% or at least 90% lower than the systemic concentration levels of anti-CTLA4 compound at 24 hours after intra-tissue injection, such as for example subcutaneous injection or intravenous injection, of an equimolar dose of the corresponding free anti-CTLA4 drug. This is significant and noteworthy as higher exposure of CTLA4 mAb in patients is significantly associated with higher rates of adverse events clinically (Feng et al. Exposure-Response Relationships on the Efficacy and Safety of Ipilimumab in Patients with Advanced Melanoma.” Clinical Cancer Research. 2013. 19 (14); 3997-86).

In certain embodiments the anti-CTLA4 conjugate is administered by intra-tissue administration and the total amount of anti-CTLA4 moieties and anti-CTLA4 drug molecules remaining locally in such tissue 3 days after said intra-tissue administration is at least 25%, such as at least 30%, at least 35%, at least 40%, at least 45% or at least 50%, of the amount of anti-CTLA4 moieties or anti-CTLA4 drug molecules administered by said intra-tissue administration.

In another aspect the present invention relates to a pharmaceutical composition comprising at least one anti-CTLA4 conjugate or a pharmaceutically acceptable salt thereof of the present invention and at least one excipient. In certain embodiments such pharmaceutical composition has a pH ranging from and including pH 3 to pH 8. In certain embodiments such pharmaceutical composition is a suspension formulation. In certain embodiments such pharmaceutical composition is a dry formulation.

Such suspension or dry pharmaceutical composition comprises at least one excipient. Excipients used in parenteral formulations may be categorized as, for example, buffering agents, isotonicity modifiers, preservatives, stabilizers, anti-adsorption agents, oxidation protection agents, viscosifiers/viscosity enhancing agents, or other auxiliary agents. However, in some cases, one excipient may have dual or triple functions. In certain embodiments the at least one excipient comprised in the pharmaceutical composition of the present invention is selected from the group consisting of
(i) Buffering agents: physiologically tolerated buffers to maintain pH in a desired range, such as sodium phosphate, bicarbonate, succinate, histidine, citrate and acetate, sulphate, nitrate, chloride, pyruvate; antacids such as Mg(OH)₂ or ZnCO₃ may be also used;
(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;
(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride;
(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;
(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;
(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs; and
(ix) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In another aspect the present invention relates to the anti-CTLA4 conjugate for use as a medicament, such as a medicament for the treatment of a cell-proliferation disorder.

In another aspect the present invention relates to the anti-CTLA4 conjugate for use in the manufacture of a medicament, such as for the manufacture of a medicament for the treatment of a cell-proliferation disorder.

In another aspect the present invention relates to the anti-CTLA4 conjugate of the present invention for use in the treatment a cell-proliferation disorder.

In another aspect the present invention relates to a method of treating in a mammalian patient in need of the treatment of one or more diseases which can be treated with an anti-CTLA4 drug, comprising the step of administering to said patient in need thereof a therapeutically effective amount of the anti-CTLA4 conjugate or a pharmaceutically acceptable salt thereof or a pharmaceutical composition comprising the anti-CTLA4 conjugate of the present invention.

In certain embodiments the treatment of the cell-proliferation disorder is in a patient undergoing treatment with at least one additional drug or therapy selected from the group consisting of anti-PD1 and anti-PDL1 compounds, other immune checkpoint antagonist therapies, pattern recognition receptor agonist compounds, immune agonist therapy, oncolytic viral therapy, anti-cancer vaccination, immunostimulatory cytokines, kinase inhibitors, transcription factor inhibitors, DNA repair inhibitors, cellular therapy, chemotherapy, radiotherapy and surgery.

Such at least one additional drug may be administered to the patient prior to, simultaneously with or after administration of the anti-CTLA4 conjugate. In certain embodiments at least one additional drug may be administered to the patient prior to administration of the anti-CTLA4 conjugate. In certain embodiments at least one additional drug may be administered to the patient simultaneously with administration of the anti-CTLA4 conjugate. In certain embodiments at least one additional drug may be administered to the patient after administration of the anti-CTLA4 conjugate.

In certain embodiments the treatment of a cell-proliferation disorder is administered to a mammalian patient together with one or more further drug molecules or treatments. It is understood that the one or more further drug molecules may be administered in the form of a pharmaceutically acceptable salt or as a pharmaceutical composition comprising such one or more further drug molecules or their pharmaceutically acceptable salts.

In certain embodiments the patient, such as a mammalian patient, is selected from mouse, rat, non-human primate and human. In certain embodiments the mammalian patient is a human patient.

In certain embodiments the treatment with the anti-CTLA4 conjugate, its pharmacologically acceptable salt or the pharmaceutical composition of the present invention may be initiated prior to, concomitant with, or following surgical removal of a tumor or radiation therapy. In addition, such treatment may optionally be combined with at least one other cancer therapeutic, such as systemic immunotherapy. Examples for the at least one cancer therapeutic, such as systemic immunotherapy, are as provided elsewhere herein for the one or more further drug molecules. In certain embodiments the anti-CTLA4 conjugate, its pharmacologically acceptable salt or the pharmaceutical composition of the present invention is administered intratumorally prior to, concomitant with, or following combination with at least one systemic immunotherapy, prior to radiation therapy or surgical removal of the injected tumor. In certain embodiments the anti-CTLA4 conjugate, its pharmacologically acceptable salt or the pharmaceutical composition of the present invention is administered intratumorally prior to, concomitant with, or following combination with at least one systemic immunotherapy, following radiation therapy or surgical removal of a tumor. In certain embodiments the anti-CTLA4 conjugate, its pharmacologically acceptable salt or the pharmaceutical composition of the present invention is administered into tumor draining lymph nodes prior to, concomitant with, or following surgical removal of a tumor or radiation therapy. In certain embodiments the anti-CTLA4 conjugate, its pharmacologically acceptable salt or the pharmaceutical composition of the present invention is administered into tumor draining lymph nodes prior to, concomitant with, or following combination with at least one systemic immunotherapy, and prior to, concomitant with, or following surgical removal of a tumor or radiation therapy. In certain embodiments the anti-CTLA4 conjugate, its pharmacologically acceptable salt or the pharmaceutical composition of the present invention is administered intratumorally into metastatic tumors that may arise prior to or following surgical removal or radiation therapy of primary tumor. In certain embodiments the anti-CTLA4 conjugate, its pharmacologically acceptable salt or the pharmaceutical composition of the present invention is administered intratumorally into metastatic tumors that may arise prior to, concomitant with, or following combination with at least one systemic immunotherapy, and prior to, concomitant with, or following surgical removal or radiation therapy of primary tumor. In certain embodiments at least one systemic therapy is administered prior to surgical removal of a tumor or radiation therapy, followed by intratumoral administration of the anti-CTLA4 conjugate, its pharmacologically acceptable salt or the pharmaceutical composition of the present invention. In certain embodiments intratumoral administration of the anti-CTLA4 conjugate, its pharmacologically acceptable salt or the pharmaceutical composition of the present invention is administered first, followed by subsequent treatment in combination with at least one systemic therapy. In certain embodiments at least one systemic therapy is administered prior to surgical removal of a tumor, followed by administration of the anti-CTLA4 conjugate, its pharmacologically acceptable salt or the pharmaceutical composition of the present invention to the tumor bed following surgery or by intratumoral administration in tumor not removed by surgery.

Said one or more further drug molecules may be administered to said patient prior to, together with or after administration of the conjugate of the present invention or the pharmaceutically acceptable salt thereof or the pharmaceutical composition comprising the conjugate of the present invention. If the one or more further drug molecules are administered together with the conjugate of the present invention or a pharmaceutically acceptable salt thereof or the pharmaceutical composition comprising the conjugate said one or more further drug molecules may be either present in the same preparation, such as the same pharmaceutical composition, may be present in the conjugate of the present invention or may be present in a different preparation.

In certain embodiments such one or more further drug molecules are selected from the group cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones.

In certain embodiments the one or more further drug is a cytotoxic/chemotherapeutic agent. In certain embodiments the one or more further drug is an immune checkpoint inhibitor or antagonist. In certain embodiments the one or more further drug is a multi-specific drug. In certain embodiments the one or more further drug is an antibody-drug conjugate (ADC). In certain embodiments the one or more further drug is a radionuclide or a targeted radionuclide therapeutic. In certain embodiments the one or more further drug is DNA damage repair inhibitor. In certain embodiments the one or more further drug is a tumor metabolism inhibitor. In certain embodiments the one or more further drug is a pattern recognition receptor agonist. In certain embodiments the one or more further drug is a chemokine and chemoattractant receptor agonist. In certain embodiments the one or more further drug is a chemokine or chemokine receptor antagonist. In certain embodiments the one or more further drug is a cytokine receptor agonist. In certain embodiments the one or more further drug is a death receptor agonist. In certain embodiments the one or more further drug is a CD47 antagonist. In certain embodiments the one or more further drug is a SIRPα antagonist. In certain embodiments the one or more further drug is an oncolytic drug. In certain embodiments the one or more further drug is a signal converter protein. In certain embodiments the one or more further drug is an epigenetic modifier. In certain embodiments the one or more further drug is a tumor peptide or tumor vaccine. In certain embodiments the one or more further drug is a heat shock protein (HSP) inhibitor. In certain embodiments the one or more further drug is a proteolytic enzyme. In certain embodiments the one or more further drug is a ubiquitin and proteasome inhibitor. In certain embodiments the one or more further drug is an adhesion molecule antagonist. In certain embodiments the one or more further drug is a hormone including hormone peptides and synthetic hormones.

In certain embodiments said one or more further drug is an inhibitor of PD-1. In certain embodiments said one or more further drug is an inhibitor of PD-L1.

Examples for cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones are as described elsewhere herein.

In certain embodiments the cell-proliferation disorder is cancer. Such cancer may be selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastatic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.

Examples for lung cancer are non-small cell lung cancer and small cell lung cancer. In certain embodiments the cancer is a non-small cell lung cancer. In certain embodiment the cancer is a small cell lung cancer.

Example for lymphomas are AIDS-related lymphoma, primary central nervous system lymphoma, T-cell lymphoma, cutaneous T-cell lymphoma, Hodgkin's lymphoma, Hodgkin's lymphoma during pregnancy, non-Hodgkin's lymphoma, follicular lymphoma, marginal zone lymphoma, diffuse large B-cell lymphoma, non-Hodgkin's lymphoma during pregnancy and angioimmunoblastic lymphoma. In certain embodiments the cancer is a cutaneous T-cell lymphoma.

Examples for skin cancer are melanoma and Merkel cell carcinoma. In certain embodiments the cancer is a skin cancer. In certain embodiments the cancer is a Merkel cell carcinoma.

An ovarian cancer may for example be an epithelial cancer, a germ cell tumor or a low malignant potential tumor. In certain embodiments the cancer is an epithelial cancer. In certain embodiments the cancer is a germ cell tumor. In certain embodiments the cancer is a low malignant potential tumor.

A pancreatic cancer may for example be an exocrine tumor/adenocarcinoma, pancreatic endocrine tumor (PET) or neuroendocrine tumor (NET). In certain embodiments the cancer is an exocrine tumor/adenocarcinoma. In certain embodiments the tumor is a pancreatic endocrine tumor. In certain embodiments the cancer is a neuroendocrine tumor.

A brain and nervous system cancer may be for example be a medulloblastoma, such as a childhood medulloblastoma, astrocytoma, ependymoma, neuroectodermal tumors, schwannoma, meningioma, pituitary adenoma and glioma. In certain embodiment the cancer is a medullablastoma. In certain embodiments the cancer is a childhood medullablastoma. In certain embodiments the cancer is an astrocytoma. In certain embodiments the cancer is an ependymoma. In certain embodiments the cancer is a neuroectodermal tumor. In certain embodiments the tumor is a schwannoma. In certain embodiments the cancer is a meningioma. In certain embodiments the cancer is a pituitary adenoma. In certain embodiments the cancer is a glioma.

An astrocytoma may be selected from the group consisting of giant cell glioblastoma, glioblastoma, secondary glioblastoma, primary adult glioblastoma, primary pediatric glioblastoma, oligodendroglial tumor, oligodendroglioma, anaplastic oligodendroglioma, oligoastrocytic tumor, oligoastrocytoma, anaplastic oligodendroglioma, oligoastrocytic tumor, oligoastrocytoma, anaplastic oligoastrocytoma, anaplastic astrocytoma, pilocytic astrocytoma, subependymal giant-cell astrocytoma, diffuse astrocytoma, pleomorphic xanthoastrocytoma and cerebellar astrocytoma.

Examples for a neuroectodermal tumor are a pineal primitive neuroectodermal tumor and a supratentorial primitive neuroectodermal tumor.

An ependymoma may be selected from the group consisting of subependymoma, ependymoma, myxopapillary ependymoma and anaplastic ependymoma.

A meningioma may be an atypical meningioma or an anaplastic meningioma.

A glioma may be selected from the group consisting of glioblastoma multiforme, paraganglioma, suprantentorial primordial neuroectodermal tumor (sPNET), brain stem glioma, childhood brain stem glioma, hypothalamic and visual pathway glioma, childhood hypothalamic and visual pathway glioma and malignant glioma.

Examples for breast cancer are breast cancer during pregnancy, triple negative breast cancer, ductal carcinoma in situ (DCIS), invasive ductal carcinoma (IDC), tubular carcinoma of the breast, medullary carcinoma of the breast, mucinous carcinoma of the breast, papillary carcinoma of the breast, cribriform carcinoma of the breast, invasive lobular carcinoma (ILC), inflammatory breast cancer, lobular carcinoma in situ (LCIS), male breast cancer, Paget's disease of the nipple, phyllodes tumors of the breast and metastatic breast cancer. In certain embodiments the cancer is a breast cancer during pregnancy. In certain embodiments the cancer is a triple negative breast cancer. In certain embodiments the cancer is a ductal carcinoma in situ. In certain embodiments the cancer is an invasive ductal carcinoma. In certain embodiments the cancer is a tubular carcinoma of the breast. In certain embodiments the cancer is a medullary carcinoma of the breast. In certain embodiments the cancer is a mucinous carcinoma of the breast. In certain embodiments the cancer is a papillary carcinoma of the breast. In certain embodiments the cancer is a cribriform carcinoma of the breast. In certain embodiments the cancer is an invasive lobular carcinoma. In certain embodiments the cancer is an inflammatory breast cancer. In certain embodiments the cancer is a lobular carcinoma in situ. In certain embodiments the cancer is a male breast cancer. In certain embodiments the cancer is a Paget's disease of the nipple. In certain embodiments the cancer is a phyllodes tumor of the breast. In certain embodiments the cancer is a metastatic breast cancer.

Examples for a carcinoma are neuroendocrine carcinoma, adrenocortical carcinoma and Islet cell carcinoma. In certain embodiments the cancer is a neuroendocrine carcinoma. In certain embodiments the cancer is an adrenocortical carcinoma. In certain embodiments the cancer is an Islet cell carcinoma.

Examples for a colorectal cancer are colon cancer and rectal cancer. In certain embodiments the cancer is a colon cancer. In certain embodiments the cancer is a rectal cancer.

A sarcoma may be selected from the group consisting of Kaposi's sarcoma, osteosarcoma/malignant fibrous histiocytoma of bone, soft tissue sarcoma, Ewing's family of tumors/sarcomas, rhabdomyosarcoma, clear cell sarcoma of tendon sheaths, central chondrosarcoma, central and periosteal chondroma, fibrosarcoma and uterine sarcoma. In certain embodiments the cancer may be a Kaposi's sarcoma. In certain embodiments the cancer may be an osteosarcoma/malignant fibrous histiocytoma of bone. In certain embodiments the cancer may be a soft tissue sarcoma. In certain embodiments the cancer may be an Ewing's family of tumors/sarcomas. In certain embodiments the cancer may be a rhabdomyosarcoma. In certain embodiments the cancer may be a clear cell sarcoma of tendon sheaths. In certain embodiments the cancer may be a central chondrosarcoma. In certain embodiments the cancer may be a central and periosteal chondroma. In certain embodiments the cancer may be a fibrosarcoma. In certain embodiments the cancer may be a uterine sarcoma.

Examples for a genitourinary cancer are testicular cancer, urethral cancer, vaginal cancer, cervical cancer, penile cancer and vulvar cancer. In certain embodiments the cancer may be a testicular cancer. In certain embodiments the cancer may be a urethral cancer. In certain embodiments the cancer may be a vaginal cancer. In certain embodiments the cancer may be a cervical cancer. In certain embodiments the cancer may be a penile cancer. In certain embodiments the cancer may be a vaginal cancer.

In certain embodiments the cell-proliferation disorder is a glioblastoma. Especially with brain tumors intra-tumoral administration has the advantage of bypassing the blood-brain-barrier and the anti-CTLA4 conjugate allows treatment of these hard-to-inject tumors that otherwise cannot be injected frequently enough with the corresponding free drug molecules.

In certain embodiments the cell-proliferation disorder is an inoperable or surgically challenging cancer of the lung, liver or pancreas.

In certain embodiments the anti-CTLA4 conjugate is administered to a patient via intra-tissue administration, which in certain embodiments is intra-tumoral administration or an administration into one or more tumor-associated draining lymph nodes. In certain embodiments the intra-tissue administration is an intra-tumoral administration. In certain embodiments the intra-tissue administration is an administration into one or more tumor-associated draining lymph nodes.

In certain embodiments an intra-tumoral administration is an administration into a solid tumor.

In certain embodiments the tumor for intra-tumoral administration or the tumor of the tumor-associated draining lymph nodes is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastatic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma. Examples for these tumors and cancers are as described elsewhere herein.

In certain embodiments the tumor for intra-tumoral administration or the tumor of the tumor-associated draining lymph nodes is an inoperable or surgically challenging cancer of the lung, liver or pancreas.

Materials and Methods

Materials

All materials were commercially available except where stated otherwise.

Monoclonal antibody CTLA-4 mAB (AMO-M6104, CAS No. 477202-00-9) was obtained from AbMole Bioscience Inc., Houston, Tex., US.

Sunbright ME-200SH (PEG20-SH) was obtained from NOF America Corporation, White Plains, N.Y., US

HHC^{MET} (EVQLVESGGGLVQAGGSLRLSCAASGGTFSFYGMGWFRQAPGKEQEFVA DIRTSAGRTYYADSVKGRFTISRDNAKNTVYLQMNSLKPEDTAVYYCAAEMSGISG WDYWGQGTQVTVSSGGGGSGGGSEVQLVESGGGLVQAGGSLRLSCAASGGTFSFY GMGWFRQAPGKEQEFVADIRTSAGRTYYADSVKGRFTISRDNAKNTVYLQMNSLKP EDTAVYYCAAEMSGISGWDYWGQGTQVTVSS; SEQ ID NO:2) was custom made and sourced from an external supplier where expression of the protein was performed from E. coli followed by standard purification strategies known to the one skilled in the art.

Reactions

Reactions were performed with anhydrous solvents (CH₂Cl₂, DMSO, THF, acetonitrile) purchased from Sigma-Aldrich Chemie GmbH, Munich, Germany. Generally, reactions were stirred at room temperature and monitored by LC-MS

Methods

Preparative RP-HPLC purifications were performed with a Waters 600 controller with a 2487 Dual Absorbance Detector or an Agilent Infinity 1260 preparative system using a Waters XBridge BEH300 Prep C18 10 μm, 150×30 mm column as stationary phase. Products were detected at 215 nm. Linear gradients of solvent system A (water containing 0.1% TFA v/v) and solvent system B (acetonitrile containing 0.1% TFA v/v) were used. HPLC fractions containing product were pooled and lyophilized if not stated otherwise.

Analytical ultra-performance LC (UPLC)-MS was performed on a Waters Acquity system or an Agilent 1290 Infinity II equipped with a Waters BEH300 C18 column (2.1×50 mm, 1.7 μm particle size; solvent A: water containing 0.05% TFA (v/v), solvent B: acetonitrile containing 0.04% TFA (v/v)) coupled to an LTQ Orbitrap Discovery mass spectrometer from Thermo Scientific or coupled to a Waters Micromass ZQ system.

Flash chromatography purifications were performed on an Isolera One system from Biotage AB, Sweden, using Biotage KP-Sil silica cartridges. Products were detected at 254 nm and 280 nm.

Low pressure RP chromatography purifications were performed on an Isolera One system from Biotage AB, Sweden, using Biotage SNAP C18 cartridges. Products were detected at 215 nm, 254 nm or 280 nm. Linear gradients of solvent system A (water containing 0.1% TFA v/v) and solvent system B (acetonitrile containing 0.1% TFA v/v). Fractions containing product were pooled and lyophilized if not stated otherwise.

Amino group content of the PEG-hydrogel was determined by conjugation of an Fmoc-amino acid to the free amino groups on the hydrogel and subsequent Fmoc-determination as described by Gude, M., J. Ryf, et al. (2002) Letters in Peptide Science 9(4): 203-206.

Amine content of the amine-HA was determined by reacting the free amino groups with o-phthalaldehyde (OPA) and N-acetylcysteine under alkaline conditions and photometric quantification of the formed chromophores, as methodically described by Molnir-Perl (Ed.) (2015), Journal of Chromatography Library 70: 405-444.

The content of a hydrogel suspension was determined by successive washing of representative aliquots of the suspension in syringe reactors with PE frits with water and absolute ethanol and subsequent drying of the solid hydrogel portions in vacuum. The hydrogel content was calculated from the mass of the hydrogel residue per syringe and the respective aliquot volume of the hydrogel suspension.

The MTS load of an adequate hydrogel was determined by quantification of free thiols on the hydrogel by an Ellman assay after removal of the MTS groups by means of TCEP reduction. The determination was performed with aliquots of the appropriate MTS-hydrogel suspensions in syringe reactors with PE frits. By using the hydrogel content of the suspensions, the MTS load of the dry hydrogel was calculated.

Concentration determinations of protein solutions were performed on a Tecan Infinite M200 using UV-cuvette micro (neoLAB) and the following conditions: path length 1 cm; absorbance wavelength 280 nm; absorbance wavelength bandwidth 5 nm; reference wavelength 338 nm; reference wavelength bandwidth 25 nm; number of flashes 25. Extinction coefficient of HHC^{MET}: ε=2.052 mL/(mg*cm). Concentrations of conjugate mixtures containing HHC^{MET} were determined by using the extinction coefficient of HHC^{MET} Extinction coefficient of CTLA-4 mAB: ε=1.53 mL/(mg*cm). Concentrations of conjugate mixtures containing CTLA-4 mAB were determined by using the extinction coefficient of CTLA-4 mAB.

Concentration of HHC^{MET} solutions was performed with Centricons VivaSpin Turbo 15, MWCO 5 kDa (Sartorius).

Buffer exchange was performed on a HiPrep column (GE Healthcare) connected to an Aekta Purifier 100 system with a flow rate of 8 mL/min.

SE-HPLC analysis of HHC^{MET} containing samples was performed on an Agilent 1200 system equipped with a Superdex 200 Increase 10/300 GL column (GE Healthcare) with a flow rate of 0.75 mL/min and PBS-T as mobile phase.

SE-HPLC analysis of CTLA-4 mAB containing samples was performed on an Agilent 1200 system equipped with a Tosoh TSKgel UP-SW3000 column (300×4.6 mm, 2 μm particle size) with a flow rate of 0.35 mL/min and 100 mM KH₂PO₄, 100 mM Na₂SO₄, pH 6.7 as mobile phase.

HPLC-Electrospray ionization mass spectrometry (HPLC-ESI-MS) measurements of HHC^{MET} and its conjugation mixtures were performed on a Waters Acquity UPLC with an Acquity PDA detector coupled to a Thermo LTQ Orbitrap Discovery high resolution/high accuracy mass spectrometer equipped with TOSOH TSKgel SuperAW3000 column for HHC^{MET} (flow 0.4 ml/min, solvent A: UP-H₂O+0.05% TFA, solvent B: UP-Acetonitrile+0.04% TFA, isocratic elution with 50% solvent A at 60° C.). For analysis of CTLA-4 mAB, the mass spectrometer was equipped with a Waters Bioresolve RP mAb, Polyphenyl column (150×2.1 mm, 450 Å, 2.7 μm particle size) (flow 0.5 ml/min, solvent A: UP-H₂O+0.05% TFA, solvent B: UP-Acetonitrile+0.04% TFA, gradient elution with 0-80% solvent B at 60° C.).

Analytical ultra-performance LC (UPLC)-MS of small molecules was performed on a Waters Acquity system or an Agilent 1290 Infinity II equipped with a Waters BEH300 C18 column (2.1×50 mm, 1.7 μm particle size or 2.1×100 mm, 1.7 μm particle size); solvent A: water containing 0.05% TFA (v/v), solvent B: acetonitrile containing 0.04% TFA (v/v) coupled to a Waters Micromass ZQ or coupled to an Agilent Single Quad MS system.

Ultra-/Diafiltration of CTLA-4 mAB solutions were performed with a Sartocon Slice 50 Eco, Hydrosart 30 kDa, 50 cm² membrane (Sartorius) connected to an Aekta flux S system (GE Healthcare).

Cation exchange chromatography (CIEC) of CTLA-4 mAB containing samples was performed on an Aekta Pure system equipped with an Eshmuno CPX column using 20 mM succinate, pH 5.5 and 20 mM succinate, 1 M NaCl, pH 5.5 as mobile phase A and B, respectively.

### EXAMPLE 1: SYNTHESIS OF CARBAMATE 2

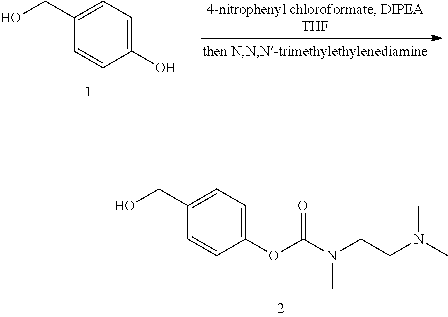 [SMILES: OCc1ccc(O)cc1 ; CN(C)CCN(C)C(=O)Oc1ccc(CO)cc1]

4-Hydroxybenzyl alcohol (1.70 g; 13.69 mmol; 1.00 eq.) was dissolved in THF (20.5 mL) and DIPEA (4.8 mL; 27.39 mmol; 2.00 eq.) was added with stirring. 4-Nitrophenyl chloroformate (2.90 g; 14.38 mmol; 1.05 eq.) in THF (5 mL) and was added dropwise over 25 min. The reaction was stirred for additional 20 minutes at room temperature. N,N,N′-trimethylethylenediamine (2.21 mL; 17.12 mmol; 1.25 eq.) was slowly added to the solution and the reaction mixture was stirred for additional 30 min. The reaction was cooled in an ice-bath, quenched with TFA (3.17 mL; 41.08 mmol; 3.00 eq.) and diluted with water. The aqueous phase was washed with ethyl acetate (3×100 mL). The aqueous phase was lyophilized to yield an oily residue. The residue was co-evaporated with ethyl acetate (3×), dissolved in DCM and dried (Na₂SO₄). After filtration the solvent was evaporated and the oily residue was dried under high vacuum (2 h). A QC by LC-MS revealed a purity of 2 of 94% at 215 nm. The crude material was used in the next step without purification. 11.76 g crude TFA salt of carbamate 2 (max. 13.69 mmol, max. purity of 43 wt %) were obtained.

### EXAMPLE 2: SYNTHESIS OF PFP-CARBONATE 3

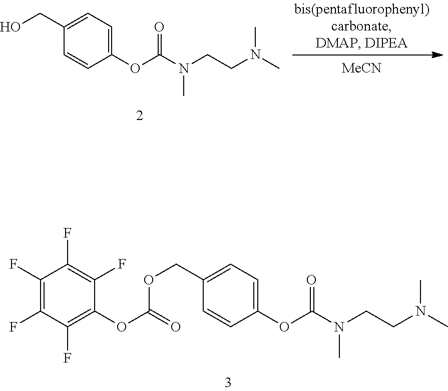 [SMILES: "MeCN" ; CN(C)CCN(C)C(=O)Oc1ccc(CO)cc1 ; CN(C)CCN(C)C(=O)Oc1ccc(COC(=O)Oc2c(F)c(F)c(F)c(F)c2F)cc1]

Carbamate 2 (11.76 g; 13.69 mmol; 1.00 eq.) was dissolved in acetonitrile (24 mL) and the solution was cooled in an ice-bath. Bis(pentafluorophenyl) carbonate (10.15 g; 25.75 mmol; 1.88 eq.), DMAP (315 mg; 2.58 mmol; 0.19 eq.) and DIPEA (9.0 mL; 51.53 mmol; 3.76 eq.) were added with stirring. The reaction mixture was stirred for 15 minutes. Formation of product 3 was confirmed by LC-MS. The reaction mixture was cooled to −15° C. and was quenched with a mixture of water with 0.1% TFA (12.4 mL) and neat TFA (3.9 mL; 51.48 mmol; 3.76 eq.). The yellow solution was purified by RP-LPLC. The pure fractions were combined, frozen and lyophilized to yield 4.73 g TFA salt of PFP-carbonate 3 as yellow oil (8.21 mmol, 60% over 3 steps).

### EXAMPLE 3: PREPARATION OF FMOC PROTECTED AMINE 6

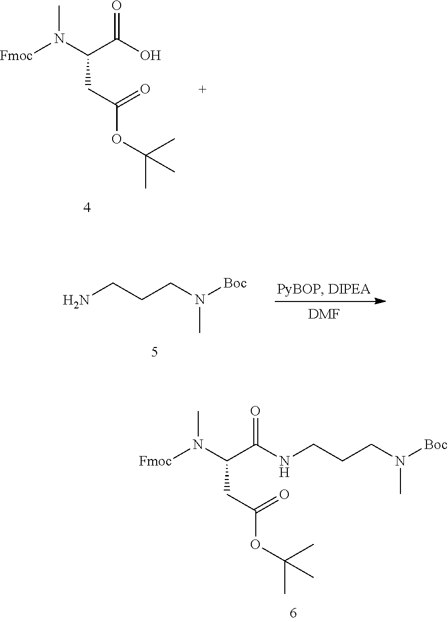

Fmoc-N-Me-Asp(tBu)-OH (4, 6.96 g; 16.36 mmol; 1.00 eq.) was dissolved in DMF (139 mL). PyBOP (12.77 g; 24.54 mmol; 1.50 eq.) and DIPEA (14.3 mL; 81.79 mmol; 5.00 eq.) were added. Finally, N-Boc-N-methyl-1,3-diaminopropane hydrochlorid (5, 4.04 g; 17.99 mmol; 1.10 eq.) was added and the reaction mixture was stirred at room temperature for 1 hour. Complete conversion to the product was observed by LCMS. The reaction mixture was diluted with 385 mL of dichloromethane and was washed three times with 385 mL of 0.1 N HCl. The organic layer was washed two times with 385 mL of saturated NaHCO₃ solution and once with 200 mL of brine. The organic layer was dried over MgSO₄, filtered and concentrated. The residue was dried under high vacuum overnight to yield the crude product as orange oil (16.25 g). The product was purified by normal phase flash chromatography. The product containing fractions were pooled and the solvent was evaporated. The final material was dried under high vacuum overnight to yield amide 6 (8.59 g, 14.42 mmol, 88%) as white foam.

### EXAMPLE 4: PREPARATION OF AMINE 7

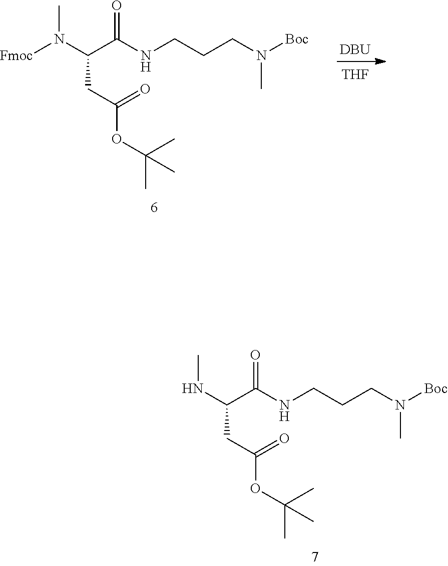

Fmoc protected amine 6 (8.59 g; 14.42 mmol; 1.00 eq.) was dissolved in THF (125 mL). DBU (2.50 mL; 16.73 mmol; 1.16 eq.) was added and the mixture was stirred at room temperature for 12 minutes. An LC-MS chromatogram showed complete conversion of the starting material. The solvent was evaporated. The residue was dissolved in 15 mL of ethyl acetate and purified by flash chromatography. The product containing fractions were pooled and the solvent was evaporated. The final material was dried under high vacuum for 1 hour to yield amine 7 (5.07 g, 13.57 mmol, 94%) as colorless oil.

### EXAMPLE 5: COUPLING OF FMOC-ADO-OH TO AMINE 7

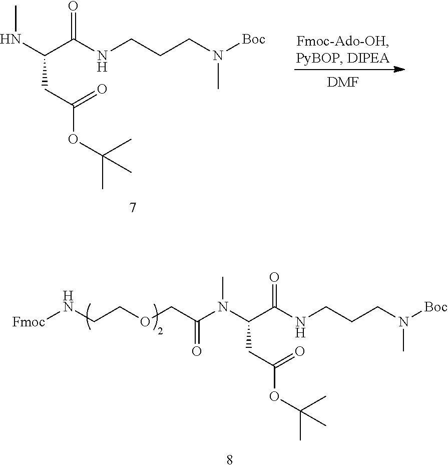

Fmoc-8-amino-3,6-dioxaoctanoic acid (Fmoc-Ado-OH) (5.76 g; 14.93 mmol; 1.10 eq.) and PyBOP (7.77 g; 14.93 mmol; 1.10 eq.) were dissolved in 38 mL of dichloromethane. Then DIPEA (7.09 mL; 40.72 mmol; 3.00 eq.) was added and the carboxylic acid was activated for 1 minute. A solution of amine 7 (5.07 g; 13.57 mmol; 1.00 eq.) in 38 mL of dichloromethane was added to the activated carboxylic acid and the reaction mixture was stirred at room temperature for 2 h. LC-MS analysis showed complete conversion of the starting material. The reaction mixture was diluted with 785 mL of ethyl acetate and was washed three times with 630 mL of 0.1 N HCl. The organic layer was washed once with 471 mL of brine. The organic layer was dried over MgSO₄, filtered and concentrated. The residue was dried under high vacuum for three days (13.59 g crude material). The residue was dissolved in 20 mL of ethyl acetate and purified by flash chromatography. The product containing fractions were pooled and the solvent was evaporated. The final material was dried under high vacuum overnight to yield amide 9 (8.59 g, 11.59 mmol, 85%) as white foam.

### EXAMPLE 6: SYNTHESIS OF LINKER CORE UNIT 9

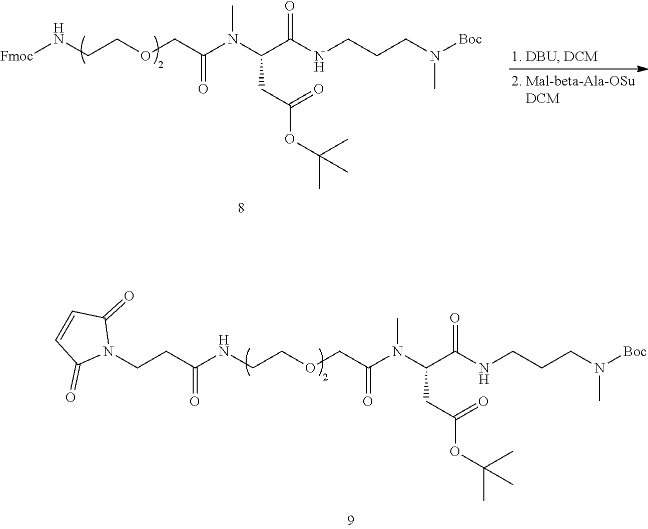

Reagent 8 (2.19 g; 2.96 mmol; 1.00 eq.) was dissolved in dichloromethane (26 mL). DBU (512 μL; 3.43 mmol; 1.16 eq.) was added to the solution and stirred for 10 min at room temperature. A solution of 3-Maleimidopropionic acid N-hydroxysuccinimide ester (1.18 g; 4.43 mmol; 1.50 eq.) in dichloromethane (46 mL) was added to the reaction mixture. The solution was stirred for 1 min. The reaction mixture was diluted with 500 mL of ethyl acetate. The organic phase was washed twice with a mixture of 400 mL of 0.5% citric acid solution and 100 mL of brine. The organic layer was dried over MgSO₄, filtered and the solvent was evaporated (3.16 g crude material). The crude material was dissolved in 10 mL of ethyl acetate and purified by flash chromatography. Product containing fractions were pooled and the solvent was evaporated to yield linker core 9 (1.68 g, 2.51 mmol, 85%) as oil.

### EXAMPLE 7: DEPROTECTION OF LINKER CORE UNIT 9

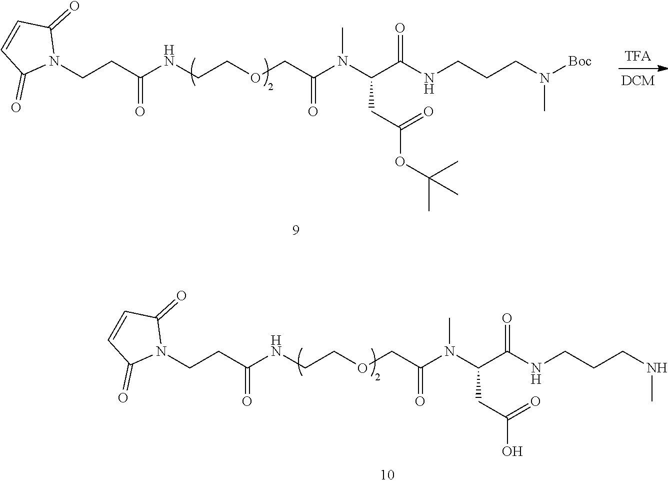

Linker 9 (2.84 g; 4.24 mmol; 1.00 eq.) was dissolved in dichloromethane (28.4 mL) and TFA (28.4 mL) was added. The reaction mixture was stirred for 70 minutes at room temperature. Volatiles were removed in a stream of argon and the resulting residue was dried under controlled conditions (rotary evaporator at 40° C. and 12 mbar for 20 min, then high vacuum at room temperature for 45 min). Crude intermediate 10 was immediately used in the next step without further purification. Crude yield was determined as 5.43 g (maximal 4.24 mmol, thus maximal 49% purity of the TFA salt of 10)

### EXAMPLE 8: COUPLING OF PFP-CARBONATE 3 TO LINKER 10

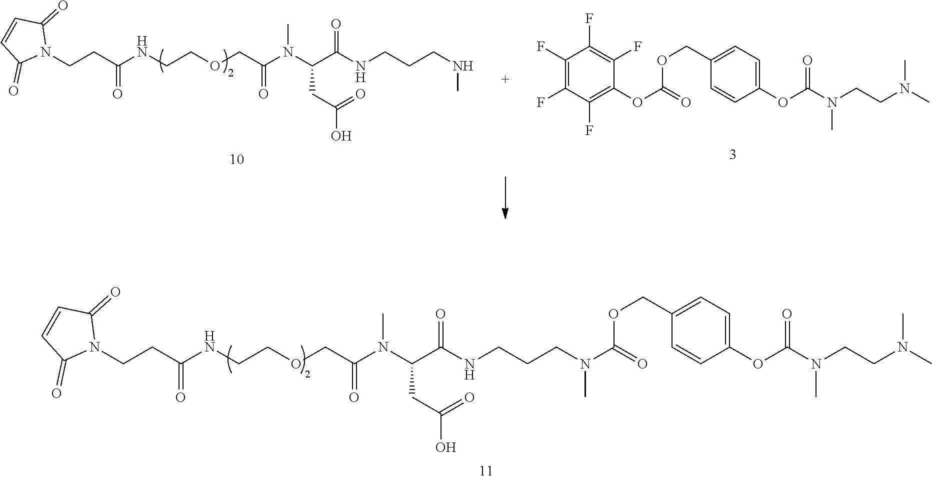

PFP-carbonate 3 (3.18 g; 5.51 mmol; 1.30 eq.) was dissolved in acetonitrile (28.4 mL), the solution was cooled in an ice bath and DIPEA (7.4 mL; 42.40 mmol; 10.00 eq.) was added. A solution of crude intermediate 10 (5.43 g crude, 4.24 mmol; 1.00 eq) in acetonitrile (28.4 mL) was added dropwise over 10 min to the stirred reaction mixture. After 5 min stirring in the ice bath, TFA (1.6 mL; 21.20 mmol; 5.00 eq.) was added to quench the reaction. The reaction mixture was concentrated and the residue dissolved in 1:1 MeCN/water+0.1% TFA (6 mL) and water+0.1% TFA (6 mL). The crude mixture was purified by RP-LPLC. Product containing fractions were combined, frozen and lyophilized to yield 3.16 g TFA salt of linker 11 (3.49 mmol, 82%).

### EXAMPLE 9: NHS ACTIVATION OF LINKER 11

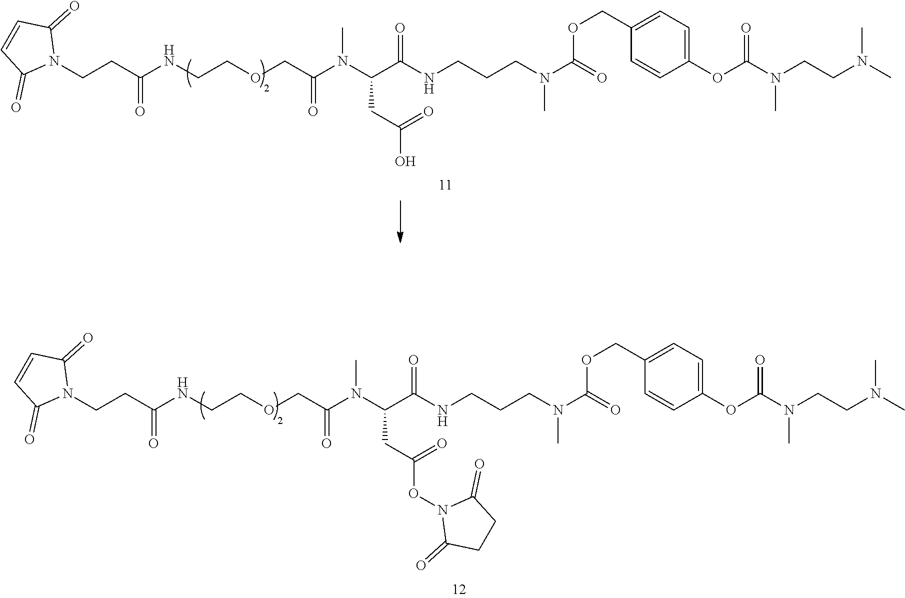

Linker 11 (3.08 g; 3.40 mmol; 1.00 eq.) was dissolved in dichloromethane (31 mL). N-Hydroxysuccinimide (1.18 g; 10.24 mmol; 3.01 eq.), EDC*HCl (1.96 g; 10.2 mmol; 3.00 eq.) and DMAP (41 mg; 0.34 mmol; 0.10 eq.) were added and the reaction mixture was stirred for 1 h at room temperature. The reaction mixture was diluted with 90 mL of dichloromethane and was washed with 90 mL of acidic brine (250 mL brine were acidified with 2.5 mL 1 M HCl, this solution was saturated with additional NaCl). The aqueous phase was extracted with 60 mL of dichloromethane (pH-value aqueous phase 3-3.5). The combined organic phases were dried over Na₂SO₄ and filtered. TFA (0.26 mL; 3.40 mmol; 1.00 eq.) was added. The solvent was removed (rotary evaporator, 40° C., approx. 20 min) to yield 3.53 g of raw product as white foam. The crude product was dissolved in 8 mL of anhydrous acetonitrile (total volume approx. 10 mL, yellowish solution) and purified by LPLC. Product containing fractions were immediately cooled (ice bath) and pooled. The product containing fractions were frozen and lyophilized as soon as possible. The lyophilized, dry material was combined with anhydrous dichloromethane (circa 81 mL in total). The solvent was carefully removed (rotary evaporator, 40° C., foam formation) and dried under high vacuum for 30 min to yield linker 12 as colorless foam with a yield of 2.86 g (2.85 mmol, 84%), 78% purity at 215 nm.

The product was stored under argon at −80° C. for 16 h. The material was brought to room temperature and dissolved in 28.5 mL of anhydrous DMSO to yield a “100 mM” solution. (No volume correction for the dissolved material was applied). The DMSO solution was sterile filtered (PTFE syringe filters, Millipore Millex-LG, 25 mm, 0.2 μm) to yield about 30 mL of a clear, colorless solution. The material was stored in aliquots under argon at −80° C.

### EXAMPLE 10: SYNTHESIS OF BACKBONE REAGENT 13

Backbone reagent 13 was synthesized as HCl salt using L-lysine building blocks, analogously to an earlier described procedure (WO2013/053856, example 1, compound 1 g therein):

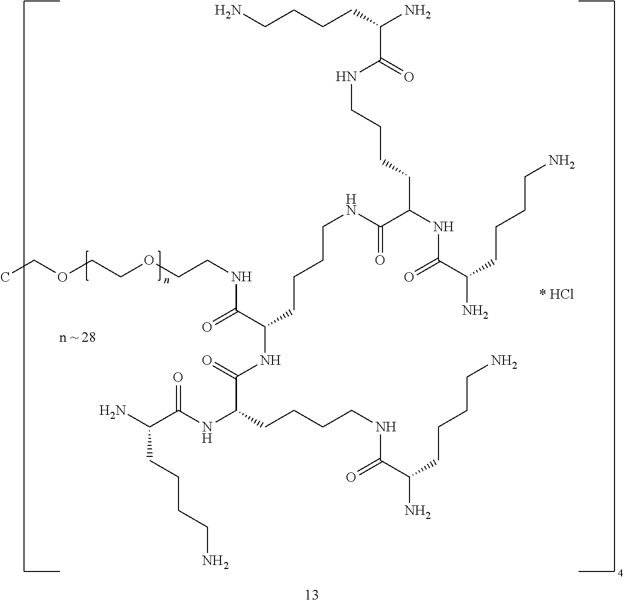

### EXAMPLE 11: SYNTHESIS OF CROSS-LINKER REAGENT 14C

Cross-linker reagent 14c was synthesized as shown below. Theoretical calculations of the Mw of the polydisperse PEG conjugates were exemplarily performed for a PEG 3300 with an assumed average Mw of 3300 g/mol. For LC-MS analyses, exact masses of the most abundant PEG molecule species with n=77 or 78 ethylene glycol units, were used.

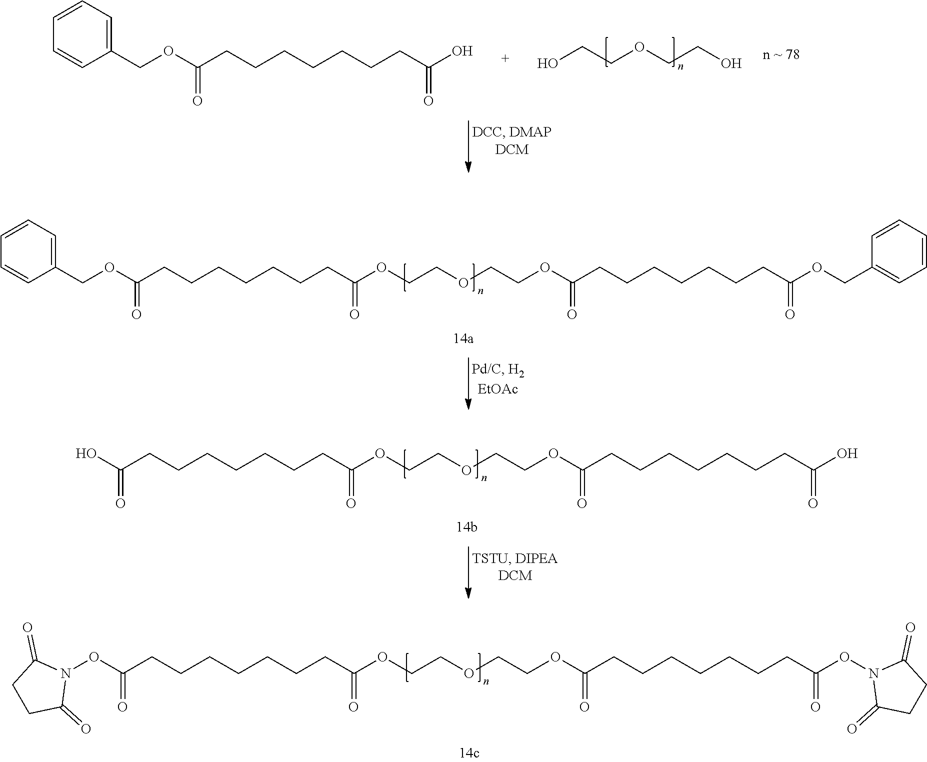

Azelaic acid monobenzyl ester (11.8 g, 42.4 mmol, 3.5 eq.) and PEG3300 (40.0 g, 12.1 mmol, 1.0 eq.) were dissolved in DCM (64 mL) and cooled to 0° C. Under stirring, a solution of DCC (8.75 g, 42.4 mmol, 3.5 eq.) and DMAP (74 mg, 0.61 mmol, 0.05 eq.) in DCM (32 mL) was added and the reaction mixture was stirred at room temperature for 17 hours. The mixture was cooled to 0° C. and the precipitated DCU was removed by filtration. The solvent was evaporated in vacuo completely and the residue was dissolved in DCM (50 mL). MTBE (450 mL) was added and the mixture was cooled to −30° C. The precipitate was collected by filtration, washed with pre-cooled MTBE (−20° C., 500 mL) and dried in high vacuum to yield intermediate 14a (41.8 g, 10.9 mmol, 90%).

MS: m/z 795.88=[M+5H]⁵⁺, (calculated monoisotopic mass: [M]=3972.34, n=78)

Palladium on charcoal (10% Pd, 199 mg) was added to a solution of intermediate 14a (41.6 g, 10.9 mmol) in EtOAc (280 mL). Under stirring, hydrogen was passed through the mixture for 3 minutes. The mixture was then stirred under hydrogen atmosphere for 16 hours. After removal of the catalyst by filtration through a pad of Celite® 503, all volatiles were removed from the filtrate in vacuo to give intermediate 14b (36.8 g, 10.1 mmol, 93%).

MS: m/z 751.05=[M+5H]⁵⁺, (calculated monoisotopic mass: [M]=3748.22, n=77)

Within one minute and under stirring, DIPEA (5.2 g, 40.4 mmol, 4.0 eq.) was added dropwise to a slightly turbid solution of intermediate 14b (36.8 g, 10.1 mmol, 1.0 eq.) and TSTU (12.2 g, 40.4 mmol, 4.0 eq.) in DCM (110 mL). After one hour, the reaction mixture was filtered through a PE frit in a syringe and the filtrate diluted with DCM (110 mL). The organic phase was washed with a solution prepared from NaOH (3 g) and NaCl (197 g) in water (750 g). Afterwards, the organic phase was dried over MgSO₄, filtered and freed from all volatiles in vacuo. The crude product was dissolved in toluene (260 mL), whereupon an orange-colored solid precipitated, which was removed by filtration. MTBE (500 mL) was added to the filtrate and the mixture was cooled to −20° C. overnight. The precipitate was collected by filtration and dried in high vacuum for three days to yield crosslinker 14c (34.9 g, 9.1 mmol, 90%).

MS: m/z 798.66=[M+5H]⁵⁺, (calculated monoisotopic mass: [M]=3986.28, n=78)

### EXAMPLE 12: SYNTHESIS OF PEG-HYDROGEL MICROPARTICLES 15A, 15B AND 15C CONTAINING FREE AMINO GROUPS

A cylindrical 250 mL reactor with bottom outlet, diameter 60 mm, equipped with baffles, was charged with an emulsion of Cithrol™ DPHS (266 mg) in heptane (80 mL). The reactor content was stirred with a pitch-blade stirrer, diameter 45 mm, at 420 rpm, at room temperature. A solution of cross-linker 14c (2373 mg) and backbone reagent 13 (550 mg) in DMSO (26.39 g) was added to the reactor and stirred for 10 min to form an emulsion. TMEDA (2.5 mL) was added to effect polymerization and the mixture was stirred at room temperature for 40 h. Acetic acid (3.8 mL) was added while stirring. After 10 min, a sodium chloride solution (15 wt %, 100 mL) was added under stirring. After 10 min, the stirrer was stopped and phases were allowed to separate. After 30 min, the aqueous phase containing the PEG-hydrogel microparticles was drained.

For microparticle classification, the water-hydrogel suspension was diluted with ethanol (40 mL) and wet-sieved on 100, 75, 63, 50 and 40 μm (mesh opening) stainless steel sieves, diameter 200 mm using a sieving machine for 15 min. Sieving amplitude was 1.5 mm, liquid flow was 250 mL/min. Water (4000 mL) was used as the liquid for wet-sieving. The bead fractions on the different sieves were transferred into 50 mL Falcon tubes (max. 14 mL bead suspension per tube) and successively washed with AcOH (0.1% v/v, 3× approx. 40 mL) and ethanol (8× approx. 40 mL) by addition, shaking, centrifugation and decantation. The bead fractions from the sieves with 50, 63 and 75 μm mesh openings were transferred into 20 mL syringes with PE frits and dried in high vacuum for three days to yield amine hydrogels 15a, 15b and 15c. The amine content of the hydrogels was determined for bead fraction 15a, representatively for all batches, by conjugation of an Fmoc-amino acid to the free amino groups on the hydrogel and subsequent Fmoc determination. The following yields were obtained: 15a (50 μm sieve fraction): 183 mg; 15b (63 μm sieve fraction): 398 mg; 15c (75 μm sieve fraction): 337 mg. Amine content was determined as 0.210 mmol/g.

### EXAMPLE 13: SYNTHESIS OF MTS-PEG12-NHS ESTER 16C

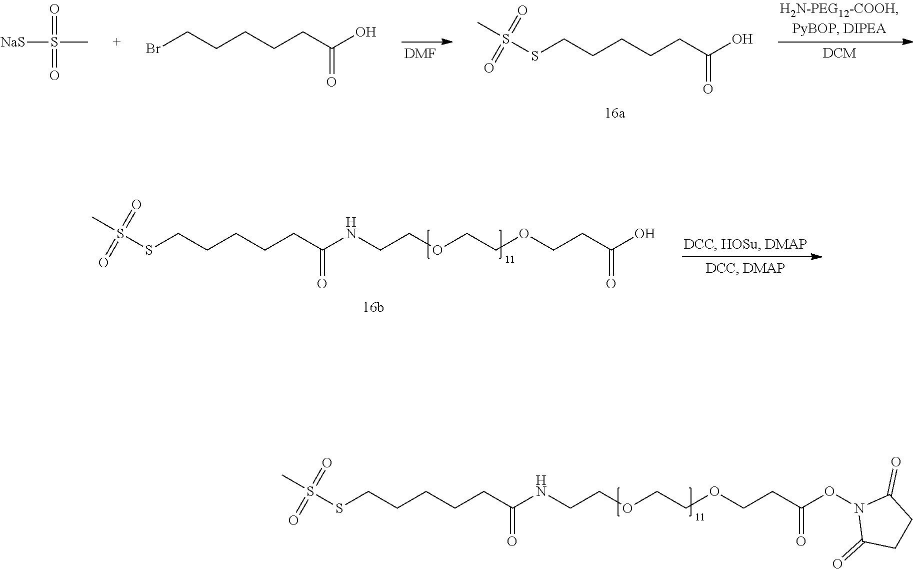

6-Bromohexanoic acid (5.89 g, 30.2 mmol, 1.0 eq.) and sodium methanethio-sulfonate (4.05 g, 30.2 mmol, 1.0 eq.) were dissolved in anhydrous DMF (47.1 mL) under argon atmosphere and stirred at 80° C. for three hours. After cooling to r.t., the mixture was diluted with water (116 mL) and extracted with diethyl ether (3×233 mL). The combined organic layers were washed with brine (350 mL), dried over MgSO₄, filtered and concentrated under reduced pressure to a volume of 40 mL. The solution was split and added to two portions of cold n-heptane (2×1150 mL) and the mixtures were cooled to −18° C. overnight. The supernatant solutions were decanted and the precipitates were dissolved in diethylether (80 mL combined). This solution was split and added to two portions of cold n-heptane (2×1000 mL) and the mixtures were cooled to −18° C. for two hours. The precipitate was collected by filtration and dried in high vacuum overnight to yield intermediate 16a (5.62 g, 24.8 mmol, 82%).

MS: m/z 249.02=[M+Na]⁺, (calculated monoisotopic mass: [M]=226.03)

DIPEA (2.76 mL, 15.9 mmol, 3.28 eq.) was added to a stirring solution of 16a (1.15 g, 5.08 mmol, 1.05 eq.) and PyBOP (2.64 g, 5.08 mmol, 1.05 eq.) in anhydrous DCM (54.8 mL). After stirring for 30 minutes, 1-amino-3,6,9,12,15,18,21,24,27,30,33,36-dodecaoxanonatriacontan-39-oic acid (2.99 g, 4.84 mmol, 1.00 eq.) was added and the mixture was stirred at room temperature for additional 30 minutes. Cold MTBE (55 mL) was added to the slightly yellow reaction mixture and it was cooled to −20° C. overnight. No precipitate was formed. All volatiles were removed in vacuo and the residue was dissolved in DCM. After addition of TFA (1.2 mL), the solution was concentrated to 10 mL. Cold MTBE (55 mL) was added to the slightly yellow solution and it was cooled to −20° C. overnight. The supernatant was decanted and the yellow precipitate was washed with cold MTBE (55 mL). The now white residue was dried on the rotavapor. After further purification by preparative RP-HPLC, intermediate 16b (2.81 g, 3.40 mmol, 70%) was obtained as white solid.

MS: m/z 826.35=[M+H]⁺, (calculated monoisotopic mass: [M]=825.39)

16b (2.81 g, 3.40 mmol, 1.0 eq.), HOSu (470 mg, 4.08 mmol, 1.2 eq.), DMAP (41.6 mg, 0.34 mmol; 0.1 eq.) and DCC (842 mg, 4.08 mmol, 1.2 eq.) were dissolved in anhydrous DCM (32.6 mL) and the mixture was stirred at room temperature for 30 minutes. The precipitated DCU was removed by filtration and the solvent was evaporated from the filtrate. The residue was purified by preparative RP-HPLC to yield pure handle reagent 16c (1.74 g; 1.88 mmol, 55%).

MS: m/z 923.45=[M+H]⁺, (calculated monoisotopic mass: [M]=922.40)

### EXAMPLE 14: SYNTHESIS OF MTS-FUNCTIONALIZED HYDROGEL 17

A PEG-hydrogel, comparable to 15c (500 mg, amine content: 0.212 mmol/g, 0.106 mmol, 1.0 eq.), present as a suspension in a mixture of NMP/n-propylamine (99:1 v/v) was partitioned between five 20 mL syringe reactors with PE frits in equal aliquots. Each hydrogel portion was successively washed with anhydrous NMP (5×8 mL), NMP/DIPEA (99:1 v/v, 5×8 mL) and all solvents were expelled completely after complete washing. To each hydrogel portion, an aliquot of 2.46 mL of a freshly prepared solution of 16c (295 mg, 0.32 mmol, 3.0 eq.) in anhydrous NMP (12 mL) and NMP/DIPEA (99:1 v/v, 500 μL) were drawn. The syringe reactors were agitated at 500 rpm for 180 minutes. The reaction mixtures were expelled from all syringes and each hydrogel portion was successively washed with anhydrous NMP (5×8 mL), water containing 0.1% AcOH and 0.01% Tween 20 (5×8 mL) and 20 mM succinate 0.01% Tween 20 pH 4.0 buffer (5×8 mL). The hydrogel aliquots were combined in a 50 mL Falcon tube with additional 20 mM succinate 0.01% Tween 20 pH 4.0 buffer. After brief centrifugation, the volume of the suspension was adjusted to 25 mL by removing an adequate volume of the clear supernatant to yield a suspension of MTS-hydrogel 17 in 20 mM succinate 0.01% Tween 20 pH 4.0 buffer with 25 mL volume and a hydrogel content of 23.0 mg/mL. The MTS load for dry hydrogel was determined as 0.161 mmol/g.

### EXAMPLE 15: PREPARATION OF HHC^{MET}-LINKER CONJUGATE MIXTURE 18

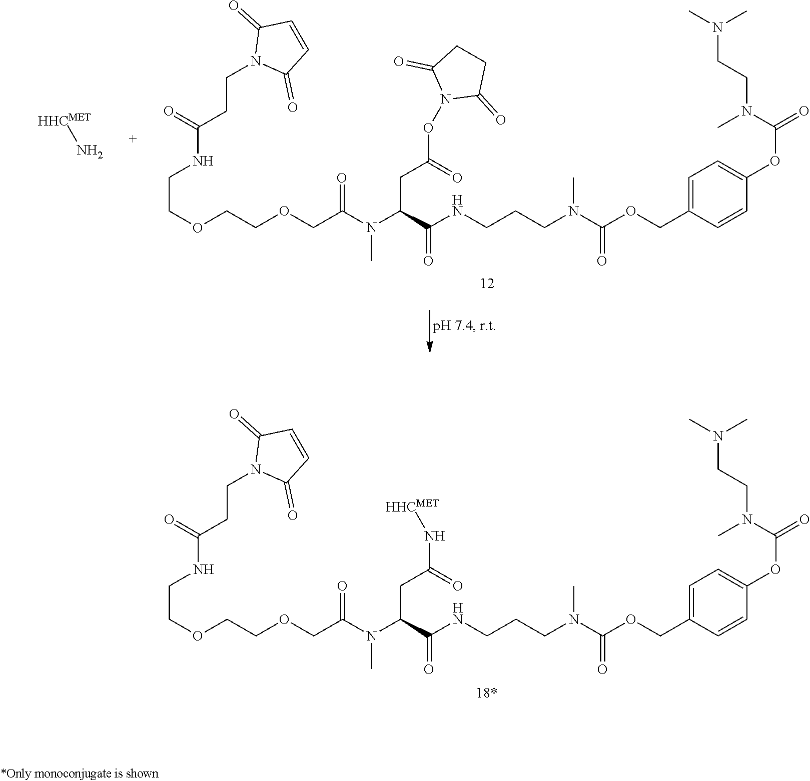

35 mL of HHC^{MET} (depicted above as HHC^{MET}-NH₂) at 4.5 mg/mL in PBS buffer was used in this example. HHC^{MET} was concentrated, and protein concentration was determined. 14.47 mL HHC^{MET} in PBS, pH 7.4 at a concentration of 9.7 mg/mL were prepared. 38 mol eq. (2.64 mL) of linker reagent 12 (example 9) (corrected with respect to NHS content, nominal 100 mM stock solution in DMSO) relative to the amount of HHC^{MET} were added in 30 seconds intervals (4×0.66 mL) to 14.38 mL of the HHC^{MET} solution. The reaction mixture was mixed carefully after each addition of linker reagent 12 and incubated in total for 8 min at ambient temperature counting from the first addition. The reaction yielded a mixture of unmodified HHC^{MET} and protected HHC^{MET}-linker conjugates (e.g. monoconjugates, bisconjugates) 18 (only monoconjugate is exemplary shown above).

The linker-conjugation reaction was immediately followed by a pH shift towards about pH 4 and a buffer exchange was performed to remove excess linker species from the HHC^{MET}/HHC^{MET}-linker conjugate mixture 18. The buffer shift was achieved by addition of 0.047 vol. eq. (0.676 mL) of 0.4 M succinic acid pH 3.0 with respect to the volume of the HHC^{MET} solution (14.38 mL), and the solution was mixed carefully end-over-end. The buffer exchange to 20 mM succinic acid, pH 4.0 was performed using an Äkta purifier 100 system equipped with a GE HiPrep column at a flow rate of 8.0 mL/min. Four runs with approx. 4.5 mL injection volume per run were performed. After buffer exchange, the HHC^{MET}/protected HHC^{MET}-linker conjugate mixture 18 was concentrated using VivaSpin Turbo 15, MWCO 5 kDa centrifugal filters yielding a solution of 11.9 g with a concentration of 10.96 mg/mL.

To estimate the content of protected HHC^{MET}-linker conjugates within the HHC^{MET}/protected HHC^{MET}-linker conjugate mixture 18, an HPLC-ESI-MS analysis was performed. 0.27 μL of the HHC^{MET}/protected HHC^{MET}-linker conjugate mixture 18 (c=10.96 mg/mL) were injected into Waters Acquity UPLC coupled to a Thermo LTQ Orbitrap Discovery high resolution/high accuracy mass spectrometer equipped with TOSOH TSKgel SuperAW3000 column (flow rate 0.4 mL/min, solvent A: UP-H₂O+0.05% TFA, solvent B: UP-Acetonitrile +0.04% TFA, isocratic elution with 50% solvent A at 60° C.).

The comparison of the relative intensities of the MS peaks corresponding to the unmodified HHC^{MET} (calculated m/z 1648.76 for [M+16H]¹⁶⁺ ion), mono- (calculated m/z 1697.09 for [M+16H]¹⁶⁺ ion) and bis-conjugate (calculated m/z 1745.43 for [M+16H]¹⁶⁺ ion) provided an estimate of the presence of about 45% of HHC^{MET}-linker monoconjugates within the HHC^{MET}/HHC^{MET}-linker conjugate mixture 18.

After analysis, 11.9 mL of the HHC^{MET}/protected HHC^{MET}-linker conjugate mixture 18 (c=10.96 mg/mL) were pH adjusted to pH 5.5 for hydrogel loading by addition of 0.154 vol. eq. 0.5 M succinic acid, pH 6.2 (1.83 mL). To reach the desired content of EDTA (target concentration 5 mM) and Tween20 (target concentration 0.01%) the obtained solution was supplemented with 1/19 vol. eq. 20 mM succinic acid, 100 mM EDTA, 0.2% Tween20, pH 5.5 (0.722 mL) and the solution was mixed end-over-end. The final volume after pH and buffer adjustment was 14.51 mL with a theoretical concentration of 9.05 mg/mL.

### EXAMPLE 16: SYNTHESIS OF TRANSIENT HHC^{MET}-LINKER-HYDROGEL PRODRUG 20

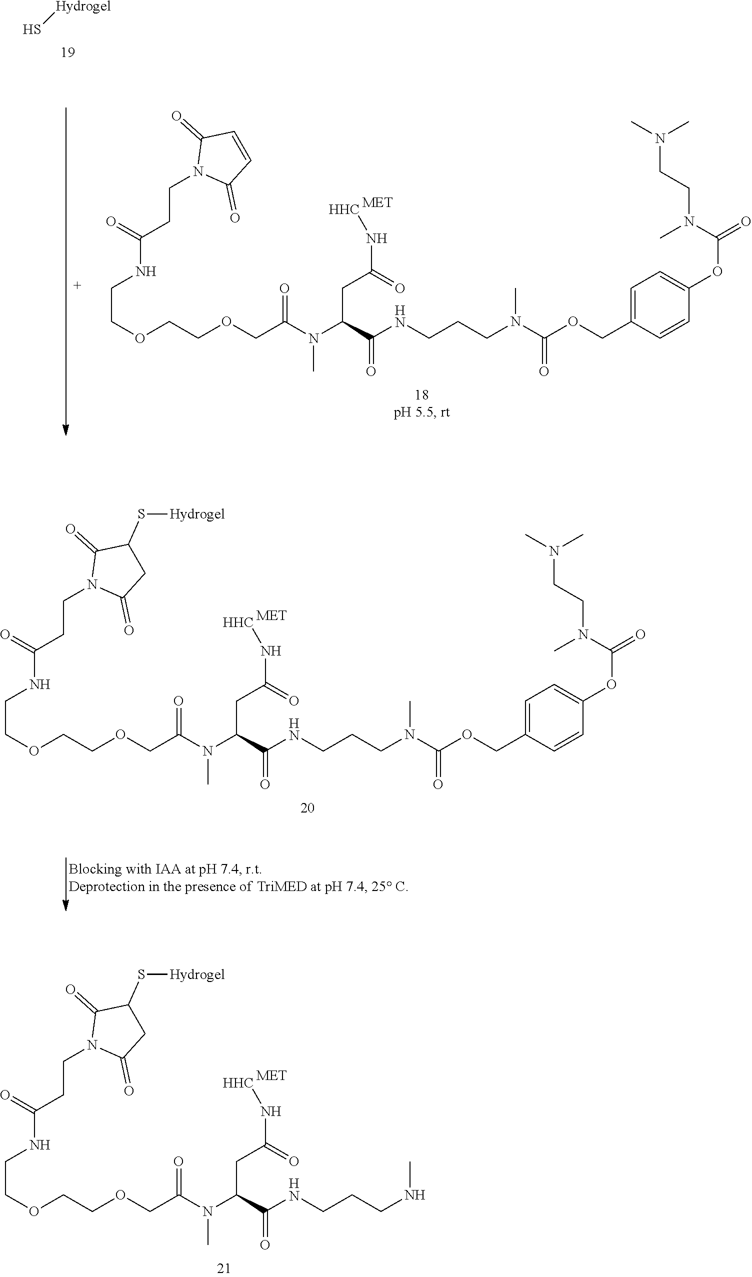

Conjugation of HHC^{MET}/protected HHC^{MET}-linker conjugate mixture 18 to the reduced thiol functionalized PEG hydrogel 19 was performed by addition of HHC^{MET}/protected HHC^{MET}-linker conjugate mixture to aim for 65% (w/w) protein content within HHC^{MET}-linker-hydrogel conjugate.

1.33 mL of MTS functionalized PEG hydrogel 17 (23.8 mg/mL nominal gel content with a thiol content of 0.161 mmol/g) in 20 mM succinic acid, 0.01% Tween20, pH 4.0 were transferred into a 20 mL syringe with a frit. The thiol functionalized PEG hydrogel beads were reduced by replacement of the storage solution by 10 mL of 50 mM TCEP solution in PBS-T and incubation for 15 minutes at ambient temperature. Afterwards, the 50 mM TCEP solution was removed from the syringe, and thiol functionalized hydrogel beads were washed in the syringe 10 times with 5 mL 20 mM succinic acid, 5 mM EDTA, 0.01% Tween20, pH 5.5 to yield 19. Afterwards, 14.51 mL of the HHC^{MET}/protected HHC^{MET}-linker conjugate mixture 18 (c_{theoretical}=9.05 mg/mL, 131.3 mg) at pH 5.5 were drawn up into the syringe. The resulting suspension was mixed well and incubated at ambient temperature under gentle rotation overnight yielding protected transient HHC^{MET}-linker hydrogel prodrug 20.

After overnight incubation, the protected transient HHC^{MET}-linker hydrogel prodrug 20 was washed in the syringe once with 5 mL 20 mM succinic acid, 5 mM EDTA, 0.01% Tween20, pH 5.5 and two times with 5 mL 10 mM iodoacetamide in 30 mM sodium phosphate, 50 mM TriMED, 0.01% Tween20, pH 7.4. The remaining free thiol groups in the prodrug 20 were blocked by 60 minutes incubation with gentle rotation in 30 mM sodium phosphate, 10 mM iodoacetamide, 50 mM TriMED, 0.01% Tween20, pH 7.4 buffer in the syringe at ambient temperature. Removal of iodoacetamide blocking solution was accomplished via ten washing steps in the syringe with 5 mL 30 mM sodium phosphate, 50 mM TriMED, 0.01% Tween20, pH 7.4.

For deprotection of the protected transient HHC^{MET}-linker hydrogel prodrug, 5 mL 30 mM sodium phosphate, 50 mM TriMED, 0.01% Tween20, pH 7.4 buffer were drawn up into the syringe and the resulting suspension was incubated at 25° C. overnight in the syringe yielding transient HHC^{MET}-linker hydrogel prodrug 21.

Final formulation of transient HHC^{MET}-linker hydrogel prodrug 21 was achieved by washing the transient HHC^{MET}-linker hydrogel prodrug 21 ten times in the syringe with 5 mL 20 mM succinic acid, 0.01% Tween20, pH 4.0. The suspension containing transient HHC^{MET}-linker hydrogel prodrug 21 was transferred to a 5 mL Eppendorf tube and a dense suspension of the transient HHC^{MET}-linker hydrogel prodrug 21 in 20 mM succinic acid, 0.01% Tween20, pH 4.0 was prepared by removal of the supernatant.

### EXAMPLE 17: SYNTHESIS OF PROTECTED DIAMINO ALCOHOL 22B

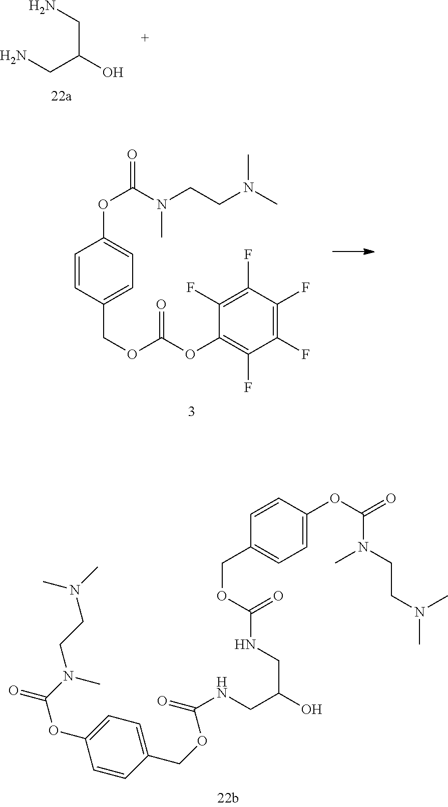 [SMILES: NCC(O)CN ; CN(C)CCN(C)C(=O)Oc1ccc(COC(=O)Oc2c(F)c(F)c(F)c(F)c2F)cc1 ; CN(C)CCN(C)C(=O)Oc1ccc(COC(=O)NCC(O)CNC(=O)OCc2ccc(OC(=O)N(C)CCN(C)C)cc2)cc1]

3 (352 mg, 0.61 mmol) was dissolved in acetonitrile (2.50 mL) and the solution cooled in an ice-bath. DIPEA (242 μL, 1.39 mmol) was added and the reaction was mixed. 22a 1,3-diamino-2-propanol (25 mg, 0.28 mmol) was dissolved in acetonitrile (1.00 mL) and added to the reaction. The reaction was mixed and incubated in the ice-bath. A reaction control after 5 min indicated complete reaction.

After ca. 15 min TFA (106 μL, 1.39 mmol) was added to the ice cooled reaction. The reaction was diluted with 4 mL water containing 0.1% TFA. The product 22b was purified by RP-HPLC.

Yield: 204 mg (84%, 2× TFA salt)

MS: m/z 647.34=[M+H]⁺, (calculated=647.34).

### EXAMPLE 18: SYNTHESIS OF LINKER REAGENT 23G

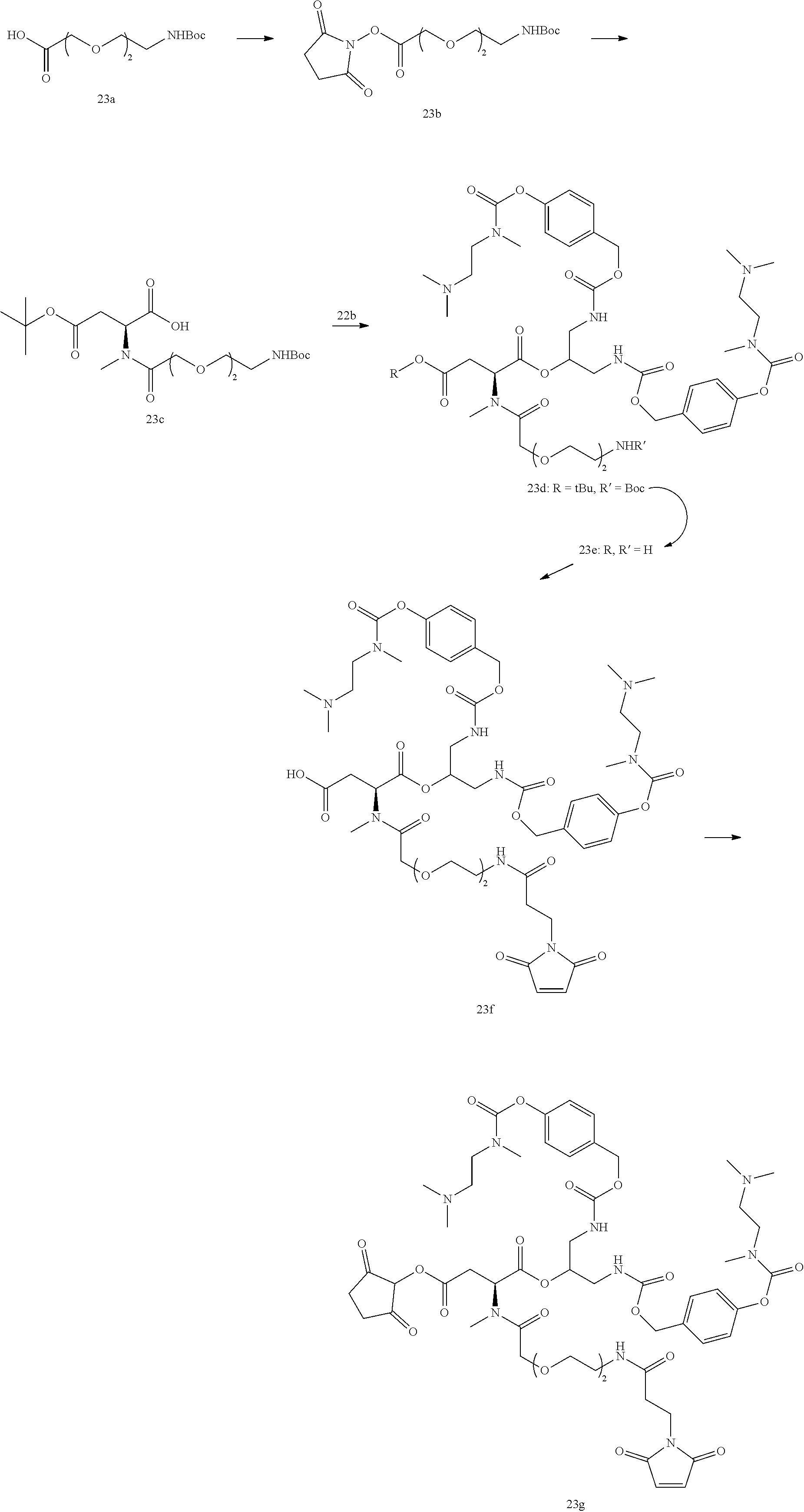

23a (1.5 g, 5.7 mmol) was dissolved in THF (37.5 mL). TSTU (2.6 g 8.6 mmol) and DIPEA (3.97 mL, 22.8 mmol) were added. Upon stirring a turbid suspension was formed. The mixture was stirred for 22 h. TSTU (1.7 g 5.5 mmol), DIPEA (2 mL, 11.5 mmol) and DMF (13 mL) were added and the color of the reaction turned dark brown. After a total of 26 h the reaction mixture was diluted with 350 mL of ethyl acetate and washed with 2×200 mL 0.1N HCl and 1× with 100 mL of brine. The organic phase was dried over Na₂SO₄ and evaporated.

The residue was dried under high vacuum overnight. The product was purified using flash chromatography yielding 23b as colorless oil.

Yield: 1.65 g (81%)

MS: m/z 361.17=[M+H]⁺, (calculated=361.16).

23b (1.65 g, 4.58 mmol) was dissolved in DCM (11.6 mL) and N-Me-L-Asp(tBu)-OH (932 mg, 4.59 mmol) and DIPEA (1.6 mL, 9.2 mmol) were added. The white suspension was stirred at rt. The mixture slowly became a light yellow solution over time.

Acetic acid (786 μL, 13.7 mmol) was added after 1h. The solvent was evaporated, and the product purified by RP-LPLC yielding 23c.

Yield: 1.77 g (86%)

MS: m/z 449.15=[M+H]⁺, (calculated=449.25).

23c (1.23 g, 2.74 mmol) and 22b (1.99 g, 2.28 mmol) were dissolved in acetonitrile (53 mL). DMAP (557 mg, 4.56 mmol) was added under stirring and to the resulting solution DIC (1.41 mL, 9.12 mmol) was given. After 1 h 0.7 mL TFA were added and the solvent removed in vacuo. The product was purified by RP-LPLC yielding 23d.

Yield: 2.33 g (78%, 2× TFA salt)

MS: m/z 1077.65=[M+H]⁺, (calculated=1077.57).

23d (2.33 g, 1.78 mmol) was dissolved in DCM (10 mL). TFA (10 mL, 131 mmol) was added under stirring. After 45 min the solvent was evaporated and the residue was co-evaporated with 50 mL of DCM. The residue was dried under high vacuum overnight yielding 2.90 g of 23e, which was used without further purification. 23e was dissolved in acetonitrile (68 mL) and 3-maleimidopropionic acid N-hydroxysuccinimide ester (1.19 g, 4.45 mmol) was added under stirring. DIPEA (3.1 mL, 17.8 mmol) was added. After 80 min the reaction was quenched by addition of TFA (1.36 mL, 17.8 mmol). The reaction was concentrated in vacuo to a volume of 40 mL and the product purified by RP-LPLC yielding 23f.

Yield: 1.73 g (75% over 2 steps, 2× TFA salt)

MS: m/z 1072.60=[M+H]⁺, (calculated=1072.49).

23f (1.73 g, 1.33 mmol) was dissolved in acetonitrile (17 mL) and EDC (767 mg, 4 mmol), HOSu (462 mg, 4 mmol) and DMAP (19 mg, 0.15 mmol) were added under stirring. After 1.5 h the reaction was quenched by addition of TFA (100 μL, 1.3 mmol) and the reaction was concentrated in vacuo to a volume of 8.5 mL and the product purified by RP-LPLC yielding 23 g.

Yield: 1.36 g (73%, 2× TFA salt)

MS: m/z 1169.71=[M+H]⁺, (calculated=1169.50).

### EXAMPLE 19: PREPARATION OF AMINE-HAS 24A AND 24B

Hyaluronic acid sodium salt (90-130 kDa, 504 mg, 1.26 mmol COOH, 1.00 eq.) was dissolved in 100 mM MES 400 mM 1,3-diaminopropane buffer pH 5.5 (62.5 mL) under vigorous stirring. HOBt (573 mg; 3.74 mmol, 3.00 eq.) and EDC.HCl (223 mg; 1.17 mmol, 0.93 eq.) were added. The suspension was stirred at ambient temperature overnight. Sodium acetate trihydrate (8.48 g) was added, whereupon the suspension turned into a solution. The crude amine-modified HA was precipitated by addition of absolute ethanol, washed with 80% (v/v) ethanol and absolute ethanol and was dried under high vacuum for 1 hour. The pellets were dissolved in water (40 mL) to form a clear solution. 4 M NaOH (13.3 mL) was added and the solution was stirred at ambient temperature for two hours before of acetic acid (3.05 mL) was added. The product was precipitated by addition of absolute ethanol, washed with 80% (v/v) ethanol and absolute ethanol and was dried under high vacuum to give amine-functionalized HA 24a as acetate salt. The amine content of the material was determined by an OPA assay.

Yield: 432 mg (acetate salt, amine-content: 0.253 mmol/g, 10.4% DS)

Another amine-HA 24b was prepared analogously to the procedure described above, only using a different amount of EDC.HCl (95.8 mg; 0.50 mmol, 0.404 eq.).

Yield: 449 mg (acetate salt, amine-content: 0.114 mmol/g, 4.6% DS)

### EXAMPLE 20: PREPARATION OF THIOL-HA 25 FROM AMINE-HA 24A

Amine-functionalized HA 24a (400 mg, 0.101 mmol amines, 1.0 eq.) was dissolved in 100 mM HEPES buffer pH 8.4 (33.25 mL). A freshly prepared solution of SPDP (318 mg, 1.02 mmol, 10.1 eq.) in acetonitrile (18 mL) was added to the mixture while stirring. The mixture was stirred at ambient temperature for 120 minutes before a freshly prepared solution of TCEP (582 mg, 2.03 mmol, 20.1 eq.) in water (5.13 mL) was added to the reaction mixture. The solution was stirred for one hour at ambient temperature before 1 M sodium acetate buffer pH 5.5 (56.4 mL) was added. The product was collected by addition of absolute ethanol and centrifugation. After washing with 80% (v/v) ethanol, absolute ethanol and drying in high vacuum for five hours, crude thiol-HA was obtained as white solid. The crude material was dissolved in 1% acetic acid (40 mL) by vigorous stirring under an argon atmosphere. 1 M sodium acetate buffer pH 5.5 (40 mL) was added to the solution and the resulting mixture was filtered through a 0.22 μm PES bottle-top filter. The product was precipitated from the filtrate by addition of absolute ethanol and centrifugation. After washing with 80% (v/v) ethanol and absolute ethanol, the material was dried in high vacuum for six hours to give thiol-HA 25 as off-white pellets. Thiol content was determined via Ellman assay.

Yield: 366 mg (thiol-content: 0.209 mmol/g)

### EXAMPLE 21: PREPARATION OF MALEIMIDE-HA 26 FROM AMINE-HA 24B

Amine-functionalized HA 24b (443 mg, 0.05 mmol amines, 1.0 eq.) was dissolved in 100 mM HEPES buffer pH 7.4 (44.25 mL). A freshly prepared solution of 3-maleimidopropionic acid NHS ester (134 mg, 0.49 mmol, 10.0 eq.) in acetonitrile (9.7 mL) was added to the mixture while stirring. The mixture was stirred at ambient temperature for 60 minutes before 1 M sodium acetate buffer pH 5.5 (54 mL) was added. The product was collected by addition of absolute ethanol and centrifugation. After washing with 80% (v/v) ethanol, followed by washing with absolute ethanol, the material was stored at −20° C. overnight and was dried in high vacuum for two hours the next day to yield crude maleimide-HA as white solid. The crude material was dissolved in 1% acetic acid (44.25 mL) by vigorous stirring. 1 M sodium acetate buffer pH 5.5 (54 mL) was added to the solution and the resulting mixture was filtered through a 0.22 μm PES bottle-top filter. The product was precipitated from the filtrate by addition of absolute ethanol and centrifugation. After washing with 80% (v/v) ethanol and absolute ethanol, the material was dried in high vacuum for six hours to give maleimide-HA 26 as white pellets. Maleimide content was determined via reverse-Ellman assay.

Yield: 376 mg (maleimide-content: 0.109 mmol/g)

### EXAMPLE 22: PREPARATION OF CROSSLINKED HA MICROPARTICLES WITH FREE THIOLS 27

Thiol-HA 25 (90.5 mg) was dissolved in 200 mM MES, 3 mM EDTA buffer pH 5.5 (3015 μL) by vigorous shaking under an argon atmosphere to produce a 30 mg/mL solution of the compound in buffer (solution A). Maleimide-HA 26 (70.7 mg) was dissolved in 200 mM MES, 3 mM EDTA buffer pH 5.5 (2355 μL) by vigorous shaking to produce a 30 mg/mL solution of the compound in buffer (solution B). In a 2 mL Eppendorf tube, equipped with a magnetic stirring bar, 200 mM MES, 3 mM EDTA buffer pH 5.5 (94.2 μL) was mixed with solution A (717.7 μL) and solution B (688.1 μL) under vigorous shaking. For gelling, the mixture was left standing at r.t. under an argon atmosphere overnight. The gel was transferred into a 5 mL Luer-Lock syringe to which a line of a male/female Luer Lock adapter, a 2×1 mm PTFE o-ring, a 144 μm stainless steel mesh (4 mm diameter), a 2×1 mm PTFE o-ring, a male/female Luer Lock adapter, a 2×1 mm PTFE o-ring, a 144 μm stainless steel mesh (4 mm diameter), a 2×1 mm PTFE o-ring and a male/female Luer Lock adapter was connected. The gel portion in the syringe was passed through the two 144 μm stainless steel meshes into 200 mM MES, 3 mM EDTA buffer pH 5.50 in a 15 mL Falcon tube. The hydrogel was successively washed with 3 mM EDTA buffer pH 5.5 followed by 200 mM succinate, 3 mM EDTA buffer pH 4.0, and 200 mM succinate, 3 mM EDTA, 0.5% Tween 20 buffer pH 4.0 by shaking, centrifugation and supernatant removal. After the last washing step, the volume of the gel suspension was adjusted to 10 mL with 3 mM EDTA, 0.5% Tween 20 buffer pH 4.0 in a 15 mL Falcon tube to yield the cross-linked HA with free thiol groups as colorless and almost completely transparent suspension. The thiol content of the hydrogel suspension was determined by Ellman assay.

Yield: 10 mL

Hydrogel content: 4.2 mg/mL (nominal, not experimentally determined)

Thiol content (suspension, fresh): 192 μmol/L

After 4 weeks storage at 5° C. under an argon atmosphere, the thiol content of the hydrogel suspension 27 and the particle-free supernatant of the latter after thorough centrifugation was determined by Ellman assay.

Thiol content (suspension, 4 weeks): 184 μmol/L

Thiol content (supernatant, 4 weeks): 1 μmol/L

### EXAMPLE 23: PREPARATION OF CTLA-4 MAB-LINKER CONJUGATE MIXTURE 28

204.13 mL of CTLA-4 mAB at 5.341 mg/mL in 26 mM Tris-HCl, 100 mM NaCl, 55 mM mannitol, 0.1 mM pentetic acid (DTPA), 0.01% Tween80, pH 7.0 was used in this example. CTLA-4 mAB was buffer exchanged to 30 mM sodium phosphate, pH 7.4, concentrated, and the protein concentration was adjusted to nominal 10 mg/mL. 103.14 mL CTLA-4 mAB in 30 mM sodium phosphate, pH 7.4 at a concentration of 9.74 mg/mL were prepared.

3 mol eq. (218.6 μL) of linker reagent 23 g (corrected with respect to NHS content, 100 mM stock solution in DMSO) relative to the amount of CTLA-4 mAB were added to the protein solution. The reaction mixture was mixed carefully and incubated for 5 min at ambient temperature yielding a mixture of unmodified CTLA-4 mAB and the protected CTLA-4 mAB-linker conjugates (e.g. monoconjugate, bisconjugate) 28a.

The conjugation reaction was immediately followed by a pH shift towards about pH 4 and a cation exchange chromatography (CIEC) step was performed to remove excess linker species from the CTLA-4 mAB/protected CTLA-4 mAB-linker conjugate mixture 28. The pH shift was achieved by addition of 0.12 vol. eq. (12.4 mL) of 0.5 M succinic acid, pH 3.0 with respect to the volume of the CTLA-4 mAB solution (103.1 mL), and the solution was mixed carefully. The CIEC step was performed using an Aekta pure system equipped with an Eshmuno CPX column (8 mm ID×200 mm length, CV=10 mL) with 20 mM succinic acid, pH 5.5 as mobile phase and a linear salt gradient elution with sodium chloride (0-60% 20 mM succinic acid, 1 M NaCl, pH 5.5 in 15 CV) at a flow rate of 4.0 mL/min. Three runs with ˜39 mL injection volume (˜337 mg) per run were performed and 119.27 mL of CTLA-4 mAB/protected CTLA-4 mAB-linker conjugate mixture was collected at a concentration of 7.32 mg/mL. The collected CIEC fractions were analyzed by HPLC-MS to confirm the removal of unreacted, free linker reagent 23 g.

To determine the content of protected CTLA-4 mAB-linker mono-, bis-, and tris-conjugate within CTLA-4 mAB/protected CTLA-4 mAB-linker conjugate mixture 28, a PEGylation with 20 kDa PEG thiol and subsequent SE-HPLC analysis was performed.

20 μL unmodified CTLA-4 mAB/protected CTLA-4 mAB-linker conjugate mixture 28 (c=7.32 mg/mL) were adjusted to 5 mg/mL by the addition of 9.28 μL of 20 mM succinic acid, pH 5.5 immediately followed by the addition of 1/19 vol. eq. 20 mM succinic acid, 100 mM EDTA, 0.2% Tween20, pH 5.5 (1.5 μL) with respect to the volume of 29.3 μL. 4.6 mg of 20 kDa PEG thiol were dissolved in water (115 μL) and 10 mol. eq. relative to the amount of CTLA-4 mAB were added (5 μL). After 45 minutes incubation at ambient temperature, SE-HPLC was performed using an Agilent 1200 system connected to a Tosoh TSKgel UP-SW3000 column with 100 mM KH₂PO₄, 100 mM Na₂SO₄, pH 6.7 as mobile phase. A total maleimide content of 41% was determined for the CTLA-4 mAB/protected CTLA-4 mAB-linker conjugate mixture 28.

After analysis and overnight storage at 4° C., 118.38 mL of the CTLA-4 mAB/protected CTLA-4 mAB-linker conjugate mixture 28 (c=7.32 mg/mL) were adjusted to a final concentration of 5 mM EDTA and 0.01% Tween20 with 1/19 vol. eq. of 20 mM succinic acid, 100 mM EDTA, 0.2% Tween20, pH 5.5 (6.2 mL) with respect to the volume of 118.38 mL and the solution was shaken carefully. The sample was filtered using one qpore Plastic vacuum filter (PVDF membrane) with a pore size of 0.22 μm. 122.67 mL of the adjusted CTLA-4 mAB/protected CTLA-4 mAB-linker conjugate mixture 28 at a concentration of 7.82 mg/mL was obtained.

### EXAMPLE 24: SYNTHESIS OF TRANSIENT CTLA-4 MAB-LINKER-HYDROGEL PRODRUG 29B AND 29C

Conjugation of CTLA-4 mAB/protected CTLA-4 mAB-linker conjugate mixture 28 to thiol functionalized, crosslinked HA hydrogel 27 was performed by addition of CTLA-4 mAB/protected CTLA-4 mAB-linker conjugate mixture 28 to 1.5 mol. hydrogel 27 with respect to determined total maleimide content of 41% (4 μM) as described in example 23 in the CTLA-4 mAB/protected CTLA-4 mAB-linker conjugate mixture 28.

7.5 mL of hydrogel suspension prepared according to example 22 (4.22 mg/mL nominal gel content with a thiol content of 200.8 μM) in 20 mM succinic acid, 150 mM NaCl, 3 mM EDTA, 0.1% Tween20, pH 4.0 were transferred into a 15 mL Falcon tube. Four 15 mL Falcon tubes were prepared in total. The hydrogel particles were sedimented by centrifugation at 4000 rcf for 1 minute and the supernatant was removed by pipetting. Washing of the particles was accomplished via five cycles of washing steps, which included addition of 10 mL 20 mM succinic acid, 5 mM EDTA, 0.01% Tween20, pH 5.5 buffer, centrifugation at 1000 rcf for 1 minute and careful removal of the supernatant by pipetting. After the last washing step, each of the four falcon tubes was filled up to a nominal total volume of suspension of 4 mL with above mentioned buffer. 2.6 mL (nominal) of the hydrogel suspension were transferred into a separate 50 mL Falcon tube resulting in four Falcon tubes each containing nominal 2.6 mL of washed hydrogel suspension.

122.62 mL of the adjusted CTLA-4 mAB/protected CTLA-4 mAB-linker conjugate mixture 28 (c=7.82 mg/mL, 958.3 mg) at pH 5.5 were divided in four parts and approx. 33 mL were added to each of the four 50 mL Falcon tubes containing the hydrogel suspension described above. The resulting suspensions were mixed end-over-end and incubated at ambient temperature under gentle agitation overnight yielding protected transient CTLA-4 mAB-linker hydrogel prodrug 29a.

The protected transient CTLA-4 mAB-linker hydrogel prodrug 29a was sedimented by centrifugation at 1000 ref for 1 minute and resting for 3 minutes. The supernatants after the hydrogel loading were transferred in a 250 mL Corning bottle by pipetting. The protected transient CTLA-4 mAB-linker hydrogel prodrug 29a were combined in one 50 mL Falcon tube.

For blocking of the remaining unreacted thiols, the protected transient CTLA-4 mAB-linker hydrogel prodrug 29a was first washed seven times with 30 mL 10 mM iodoacetamide (IAA) in 30 mM sodium phosphate, 50 mM N,N,N′-Trimethylethylendiamine (TriMED), 0.01% Tween20, pH 7.4. Afterwards, 30 mL 10 mM IAA in 30 mM sodium phosphate, 50 mM TriMED, 0.01% Tween20, pH 7.4 were added to the sedimented protected transient CTLA-4 mAB-linker hydrogel prodrug 29a and incubated at ambient temperature under gentle agitation for 1 h. Removal of IAA blocking solution was accomplished via ten cycles of washing, which included addition of 30 mL 30 mM sodium phosphate, 50 mM TriMED, 0.01% Tween20, pH 7.4 buffer, centrifugation at 1000 rcf for 1 minute and careful removal of the supernatant by pipetting after 3 minutes resting.

Afterwards, for deprotection of the protected transient CTLA-4 mAB-linker hydrogel prodrug 29a, 30 mL 30 mM sodium phosphate, 50 mM TriMED, 0.01% Tween20, pH 7.4 buffer were added to the sedimented hydrogel and the resulting suspension was incubated at 25° C. overnight yielding transient CTLA-4 mAB-linker hydrogel prodrug 29b. Final formulation of transient CTLA-4 mAB-linker hydrogel prodrug 29b was performed by washing the transient CTLA-4 mAB-linker hydrogel prodrug 29b ten times with 20 mM succinic acid, 10 w % a-a-D-trehalose, 0.01% Tween20, pH 5.5. 2.5 mL of 29b were diluted with 10 mL of 20 mM succinic acid, 10 w % α-α-D-trehalose, 0.01% Tween20, pH 5.5 to give 29c.

### EXAMPLE 25: IN VITRO RELEASE KINETICS FOR 29B

25 mg of dense CTLA-4 mAB-linker hydrogel prodrug 29b (corresponding to approximately 0.45 mg protein) were transferred in a sterile, 1.5 mL Eppendorf tube. Eight tubes were prepared in total. 1 mL 60 mM sodium phosphate, 3 mM EDTA, 0.01% Tween20, pH 7.4 was added to each tube, which was subsequently mixed end-over-end and incubated without agitation for 5 minutes. The supernatant was removed to a final volume of 0.5 mL suspension per vial. The suspensions were incubated at 37° C. in a water bath. After different time intervals, one vial was removed from 37° C., centrifuged and the supernatant was analyzed by A280 measurement and SE-HPLC at 215 nm. The relative amount of released CTLA-4 mAB based on concentration determination of the supernatant with respect to the total amount of CTLA-4 mAB in each vial was plotted against the incubation time in days.

Release Kinetics from 29b:

| | CTLA-4 mAB | CTLA-4 mAB |
|---|---|---|
| t [d] | release [μg] | release [%] |
| 1.92 | 25.9 | 5.5 |
| 8.92 | 83.2 | 18.5 |
| 18.92 | 149.9 | 33.0 |
| 27.92 | 189.4 | 42.9 |
| 39.92 | 226.2 | 49.2 |

### EXAMPLE 26: SYNTHESIS OF PLACEBO HYDROGEL 30

16.7 mL of hydrogel suspension 27 were distributed over four 15 mL Falcon tubes (4.16 mL each). The tubes were briefly centrifuged, and the volume of the suspension was reduced to 3 mL by partial removal of the supernatant. 10 mL 10 mM iodoacetamide (IAA) in 30 mM sodium phosphate, 50 mM N,N,N′-Trimethylethylendiamine, 0.01% Tween20, pH 7.4 (blocking solution) were added into each of the four Falcon tubes. The suspension was gently mixed and briefly centrifuged. 10 mL of the supernatant were discarded. This procedure was performed overall 7 times. 10 mL of the blocking solution were added into each of the 4 Falcon tubes. The suspension was gently mixed, and the resulting suspension was incubated at ambient temperature for 60 min. The suspension was gently mixed and briefly centrifuged. 10 mL of the supernatant were discarded. The hydrogel suspension was washed 10 times with 20 mM succinic acid, 10 wt % α-α-D-trehalose, 0.01% Tween20, pH 5.5. For this purpose, 10 mL buffer were added, and the suspension was gently mixed and briefly centrifuged. 10 mL of the supernatant were discarded.

After the tenth washing cycle, 5 mL of fresh buffer were added to two of the four tubes and the suspension was mixed well. The resulting suspensions were each transferred into another Falcon tube in which hydrogel suspension without fresh buffer was present. The suspensions were mixed well and briefly centrifuged. The supernatant was reduced to an overall volume of 6 mL and the suspensions were combined in one single Falcon tube. The resulting suspension was briefly centrifuged, and the volume of the suspension was reduced to 11 mL to give the placebo hydrogel 30 with an approximate hydrogel content of 7.1 mg/mL.

### EXAMPLE 27: PLASMA PHARMACOKINETICS OF CTLA-4 MAB IN WISTAR RATS AFTER SUBCUTANEOUS (SC) INJECTIONS OF A TRANSIENT CTLA-4 MAB-LINKER-HYDROGEL PRODRUG (COMPOUND 29B) AND AFTER INTRAVENOUS (IV) AND SUBCUTANEOUS (SC) INJECTIONS OF FREE CTLA-4 MAB

This study was performed in order to investigate the plasma pharmacokinetics of CTLA-4 mAB in Wistar rats following subcutaneous administration of transient CTLA-4 mAB-linker-hydrogel prodrug 29b or following subcutaneous or intravenous administration of free CTLA-4 mAB. Animals (n=3 per group) received either a single SC injection of a 29b (1 mg/kg CTLA-4 mAB equivalents) in the neck region or a single SC injection in the neck region or IV injection in the tail vein of an CTLA-4 mAB formulation (1 mg/kg CTLA-4 mAB). At selected time points, 200 μL blood were collected in L¹-Heparin tubes and processed to plasma by centrifugation at 3,000 g for 10 minutes at 4° C.

CTLA-4 mAB concentrations in rat plasma were determined with a commercial ELISA setup obtained from BioVision Inc. (CA, USA, order number E4384-100). The manufacturer's instructions were followed with minor changes to the protocol. Specifically, the plasma samples were used undiluted (except for samples above the upper limit of quantification which were diluted with blank plasma prior to the measurement) and the sample incubation time was prolonged to 60 minutes.

Calibration standards of CTLA-4 mAB in blank plasma were prepared as follows: thawed L¹-Heparin Wistar rat plasma was homogenized. The free CTLA-4 mAB formulation was spiked into blank plasma at concentrations between 5,000 ng/mL and 50 ng/mL with additional higher and lower anchor points. These solutions were used for the generation of a calibration curve. Calibration curves were analyzed via a 4-parameter logarithmic fit and 1/Y² weighted. The determined CTLA-4 mAB plasma concentrations are depicted in Table 1.

TABLE 1
Determined mean CTLA-4 mAB plasma concentrations
in ng/mL per time point and group (n = 3).

| | Time (h) | | | | | | | |
|---|---|---|---|---|---|---|---|---|
| Group | 1 | 4 | 24 | 32 | 48 | 72 | 96 | 168 |
| 1 | <LLOQ | <LLOQ | 170 | 240 | 370 | 560 | 650 | 780 |
| 2 | 170 | 600 | 3700 | 4800 | 6200 | 6900 | 6100 | 5700 |
| 3 | 18000 | 14000 | 9600 | 7500 | 6000 | 4900 | 4000 | 3100 |

Group 1: transient CTLA-4 mAB-linker-hydrogel prodrug (compound 29b; 1 mg/kg CTLA-4 mAB equivalents - subcutaneous administration);
Group 2: CTLA-4 mAB (1 mg/kg - subcutaneous administration);
Group 3: CTLA-4 mAB (1 mg/kg - intravenous administration); LLOQ at 50 ng/mL

Specifically, CTLA-4 mAB concentration after intra-tissue (subcutaneous) injection of a transient CTLA-4 mAB-linker-hydrogel prodrug (compound 29b; 1 mg/kg) were below 1 μg/mL 72 h after administration.

Also, CTLA-4 mAB concentration 72 h after intra-tissue (subcutaneous) injection of a transient CTLA-4 mAB-linker-hydrogel prodrug (compound 29b; 1 mg/kg) were at least 80% of CTLA-4 mAB concentration 1 h after intra-tissue (subcutaneous) injection of a transient CTLA-4 mAB-linker HA-hydrogel conjugate (compound 29b; 1 mg/kg).

In addition, CTLA-4 mAB concentration 24 h after intra-tissue (subcutaneous) injection of transient CTLA-4 mAB-linker-hydrogel prodrug (compound 29b; 1 mg/kg) were at least 50% lower than CTLA-4 mAB concentration 24 h after intra-tissue (subcutaneous) of an CTLA-4 mAB formulation (1 mg/kg CTLA-4 mAB).). In fact, levels of CTLA-4 mAB concentration 24 h after intra-tissue (subcutaneous) injection of transient CTLA-4 mAB-linker-hydrogel prodrug (compound 29b; 1 mg/kg) were more than 90% lower than CTLA-4 mAB concentration 24 h after intra-tissue (subcutaneous) injection of an CTLA-4 mAB formulation (1 mg/kg CTLA-4 mAB). This is significant and noteworthy as higher exposure of CTLA4 mAb in patients is significantly associated with higher rates of adverse events clinically (Feng et al. Exposure-Response Relationships on the Efficacy and Safety of Ipilimumab in Patients with Advanced Melanoma.” Clinical Cancer Research. 2013. 19 (14); 3997-86).

In addition, CTLA-4 mAB plasma concentrations after intra-tissue (subcutaneous) injection of transient CTLA-4 mAB-linker-hydrogel prodrug (compound 29b; 1 mg/kg) were substantially lower than CTLA-4 mAB concentrations from intravenous injection of an CTLA-4 mAB formulation (1 mg/kg CTLA-4 mAB) at every timepoint measured such as 56.5 fold lower at 24h, 31.3 fold lower at 32h, 16.2 fold lower at 48h, 8.8 fold lower at 72h, 6.2 fold lower at 96h and 4.0 fold lower even after 168h. This is significant and noteworthy as higher exposure of CTLA4 mAb in patients is significantly associated with higher rates of adverse events clinically (Feng et al. Exposure-Response Relationships on the Efficacy and Safety of Ipilimumab in Patients with Advanced Melanoma.” Clinical Cancer Research. 2013. 19 (14); 3997-86).

### EXAMPLE 28: IN VIVO ANTI-TUMOR EFFICACY

The study was conducted in female C57BL/6 mice with the human CTLA-4 gene knocked into the endogenous CTLA-4 locus with an age of 6-11 weeks at the day of tumor inoculation. This model is also known as a Human Genetically Engineered Mouse Model or HuGEMM. Mice were subcutaneously implanted with 1×10⁶ MC38 tumor cells in the right flank. When tumors to be injected were grown to a mean tumor volume of ˜65 mm³, mice were randomized into treatment cohorts (day 0) and treated with either: 1) a single 50 μL intratumoral injection of control hydrogel 30 or 2) a single 50 μL intratumoral injection of 840 μg of CTLA-4 mAB-linker-hydrogel prodrug 29b or 3) four total 200 μL intraperitoneal doses of 210 μg given on Days 0, 4, 8, and 12 of CTLA-4 mAB. Hydrogels were administered as suspensions in 20 mM succinate, 135 mM NaCl, 0.01% Tween-20, pH 4.0 buffer. Following treatment initiation, anti-tumor efficacy was assessed by determination of tumor volumes at various time points from tumor size measurements with a caliper. Tumor volumes were calculated according to the formula:

Tumor volume=(L×W²)×0.5 where L is the length of the tumor and W the width (both in mm). Mice were removed from the study once tumors were greater than 3000 mm³.

Results: Significant tumor growth inhibition was observed with either systemically delivered free CTLA-4 mAB (CTLA-4 mAB IP) or intratumorally delivered CTLA4 mAb Hydrogel 29b (CTLA4 mAb Hydrogel IT 29b) as compared to treatment with intratumorally delivered control hydrogel (control hydrogel 30) with respective average tumor volumes at day 20 post initiation of dosing of 60.5, 203.9, and 2142.5 mm³ respectively (Table 2). One-way ANOVA demonstrated that CTLA-4 mAB IP or CTLA-4 mAB hydrogel 29b IT treatment was statistically significant vs. control hydrogel 30 IT treatment (p values of <0.0001 for both treatments) with no significant difference between CTLA-4 mAB IP systemic or CTLA-4 mAB hydrogel 29b IT treatment (p=0.9068, Table 2).

TABLE 2
Summary of CTLA4 treatment efficacy
results in MC38 tumor bearing mice

| | Mean | SEM | | P-value* | P-value* |
|---|---|---|---|---|---|
| | Tumor | Tumor | | vs | vs |
| | Volume | Volume | | Control | CTLA4 |
| | (mm3) | (mm3) | N | Hydrogel | Ab IP |
| Group | Day 20 | Day 20 | Day 20 | Overall | Overall |
| Control | 2142.5 | 406.7 | 8 | NA | <0.0001 |
| hydrogel 30 | | | | | |
| CTLA-4 mAB IP | 60.5 | 10.2 | 8 | <0.0001 | NA |
| CTLA-4 mAB | 203.9 | 56.1 | 8 | <0.0001 | 0.9046 |
| Hydrogel 29b IT | | | | | |

SEM = standard error of the mean, N = sample size;
*Significance was determined by One-way ANOVA followed by multiple comparisons using Tukey's Honest Significant Differences (HSD) post-hoc test.

### EXAMPLE 29: PREPARATION OF HHC^{MET}-LINKER CONJUGATE MIXTURE 31

154 mL of HHC^{MET} at 5.94 mg/mL in PBS, pH 7.4 was used in this example. HHC^{MET} was concentrated using centrifugal filters, and the protein concentration was determined. 28.18 mL HHC^{MET} in PBS, pH 7.4 at a concentration of 30.3 mg/mL were prepared.

1.5 mol eq. (508 μL) of linker reagent 23 g (corrected with respect to NHS content, 100 mM stock solution in DMSO) relative to the amount of HHC^{MET} were added to the protein solution. The reaction mixture was mixed carefully and incubated for 5 min at ambient temperature yielding a mixture of unmodified HHC^{MET} and the protected HHC^{MET} conjugates (e.g. monoconjugate, bisconjugate) 31.

The linker-conjugation reaction was immediately followed by a pH shift towards about pH 4 and a buffer exchange was performed to remove excess linker species from the HHC^{MET}/HHC^{MET}-linker conjugate mixture 31. The buffer shift was achieved by addition of 0.047 vol. eq. (1.324 mL) of 0.4 M succinic acid, pH 3.0 with respect to the volume of the HHC^{MET}solution (28.18 mL), and the solution was mixed carefully end-over-end. The buffer exchange to 20 mM succinic acid, pH 4.0 was performed using an Äkta purifier 100 system equipped with a GE HiPrep column at a flow rate of 8.0 mL/min. Six runs with approx. 5 mL injection volume per run were performed.

To determine the content of protected HHC^{MET}-linker mono-, bis-, and tris-conjugate within HHC^{MET}/protected HHC^{MET}-linker conjugate mixture 31, a PEGylation with 20 kDa PEG thiol and subsequent SE-HPLC analysis was performed. 24.4 μL unmodified HHC^{MET}/protected HHC^{MET}-linker conjugate mixture 31 (c=10.89 mg/mL) were pH-adjusted to pH 5.5 by addition of 0.154 vol. eq. (3.8 μL) of 0.5 M succinic acid, pH 6.2 with respect to the volume of the HHC^{MET} solution (24.4 μL). The obtained solution was then supplemented with 1/19 vol. eq. 20 mM succinic acid, 100 mM EDTA, 0.2% Tween20, pH 5.5 (1.5 μL) with respect to the volume of 28.2 μL. The protein concentration of the unmodified HHC^{MET}/protected HHC^{MET}-linker conjugate mixture 31 at pH 5.5 was adjusted to 4 mg/mL by mixing 15.8 μL of the solution with 20.2 μL of 20 mM succinic acid, 5 mM EDTA, 0.01% Tween20, pH 5.5. The PEGylation reaction was started by the addition of 4 μL of 15 mM PEG20-SH solution in water. After 15 minutes incubation at ambient temperature, SE-HPLC was performed using an Agilent 1200 system connected to a Superdex 200 Increase 10/300 GL column with PBS-T, pH 7.4 as mobile phase. Maleimide content was calculated with the use of the peak area of the conjugates and multiplied with the number of attached PEG reagents. A total maleimide content of 47.7% was determined for the HHC^{MET}/protected HHC^{MET}-linker conjugate mixture 31.

After analysis and overnight storage at 4° C., 71.98 mL of the HHC^{MET}/protected HHC^{MET}-linker conjugate mixture 31 (c=10.89 mg/mL) were pH-adjusted to pH 5.5 by addition of 0.154 vol. eq. (11.08 mL) of 0.5 M succinic acid pH 6.2. The obtained solution was supplemented with 1/19 vol. eq. 20 mM succinic acid, 100 mM EDTA, 0.2% Tween20, pH 5.5 (4.37 mL) and the solution was mixed end-over-end. The sample was filtered using one qpore Plastic vacuum filter (PVDF membrane) with a pore size of 0.22 μm.

### EXAMPLE 30: SYNTHESIS OF TRANSIENT HHC^{MET}-LINKER-HYDROGEL PRODRUG 32

Conjugation of HHC^{MET}/HHC^{MET}-linker conjugate mixture 31 to the reduced thiol functionalized hydrogel 19 was performed by addition of HHC^{MET}/HHC^{MET}-linker conjugate mixture 31 to 1.75 mol. eq. of thiol groups in hydrogel 19 with respect to determined total maleimide content of 47.7% (19.13 μmol) as described in example 29 in the HHC^{MET}/HHC^{MET}-linker conjugate mixture 31.

8.5 mL of MTS functionalized hydrogel 18 (23.7 mg/mL nominal gel content with a thiol content of 0.183 mmol/g) in 20 mM succinic acid, 0.01% Tween20, pH 4.0 were transferred into a 20 mL syringe with a frit. The thiol functionalized hydrogel was reduced by replacement of the storage solution by 20 mL of 50 mM TCEP solution in PBS-T and incubation for 15 minutes at ambient temperature. Afterwards, the 50 mM TCEP solution was removed from the syringe, and the hydrogel was washed in the syringe 10 times with 20 mL 20 mM succinic acid, 5 mM EDTA, 0.01% Tween20, pH 5.5 and resuspended in ˜ 6.7 mL of 20 mM succinic acid, 5 mM EDTA, 0.01% Tween20, pH 5.5 to yield 19.

3.06 mL of hydrogel 19 were transferred into two 50 mL falcon tubes. Afterwards, 43.2 mL of the HHC^{MET}/protected HHC^{MET}-linker conjugate mixture 31 (c=9.26 mg/mL) at pH 5.5 were added into each falcon tube containing hydrogel 19. The resulting suspensions were mixed well and incubated at ambient temperature under gentle rotation overnight yielding protected transient HHC^{MET}-linker hydrogel prodrug.

After overnight incubation, the protected transient HHC^{MET}-linker hydrogel prodrug was transferred into a 20 mL syringe equipped with a frit, and washed in the syringe once with 20 mL 20 mM succinic acid, 5 mM EDTA, 0.01% Tween20, pH 5.5 and two times with 20 mL 10 mM iodoacetamide in 30 mM sodium phosphate, 50 mM TriMED, 0.01% Tween20, pH 7.4. The protected transient HHC^{MET}-linker hydrogel prodrug was incubated for 60 minutes with gentle rotation in 30 mM sodium phosphate, 10 mM iodoacetamide, 50 mM TriMED, 0.010% Tween20, pH 7.4 buffer in the syringe at ambient temperature. After, the hydrogel was washed ten times in the syringe with 20 mL 30 mM sodium phosphate, 200 mM TriMED, 0.01% Tween20, pH 7.4. The solvent was each time discarded.

20 mL 30 mM sodium phosphate, 200 mM TriMED, 0.01% Tween20, pH 7.4 buffer were drawn up into the syringe and the resulting suspension was incubated at 25° C. for 26 hours under gentle rotation yielding transient HHC^{MET}-linker hydrogel prodrug 32. Formulation of transient HHC^{MET}-linker hydrogel prodrug 32 was achieved by washing the hydrogel ten times in the syringe with 20 mL 20 mM succinic acid, 8.5% α-α-D-trehalose, 1% carboxymethylcellulose, 0.01% Tween20, pH 5.0.

### EXAMPLE 31: SYNTHESIS OF TRANSIENT CTLA-4 MAB-LINKER-HYDROGEL PRODRUG 33

Preparation of the transient CTLA-4 mAB-linker hydrogel prodrug was performed as described in example 24 yielding transient CTLA-4 mAB-linker hydrogel prodrug 29b. However, following the overnight incubation in 30 mM sodium phosphate, 50 mM TriMED, 0.01% Tween20, pH 7.4 buffer, the transient CTLA-4 mAB-linker hydrogel prodrug was washed ten times with 20 mM succinic acid, 135 mM NaCl, 1 w % carboxymethylcellulose (CMC), 0.01% Tween20, pH 4.0 instead of 20 mM succinic acid, 10 w % α-α-D-trehalose, 0.01% Tween20, pH 5.5 for final formulation to give 33.

### EXAMPLE 32: PLASMA PHARMACOKINETICS OF HHC^{MET} IN WISTAR RATS AFTER SUBCUTANEOUS (SC) AND INTRAMUSCULAR (IM) INJECTIONS OF A TRANSIENT HHC^{MET}-LINKER HYDROGEL PRODRUG 32 AND AFTER INTRAVENOUS (IV) AND SUBCUTANEOUS (SC) INJECTIONS OF FREE HHC^{MET}

This study was performed in order to investigate the plasma pharmacokinetics of HHC^{MET} in Wistar rats following subcutaneous and intramuscular administration of transient HHC^{MET}-linker hydrogel prodrug 32 or following subcutaneous or intravenous administration of free HHC^{MET}. Animals (n=3 per group) received either a single SC injection in the neck region or a single IM injection in the thigh musculature of a formulation of 32 (10 mg/kg HHC^{MET} equivalents) or a single SC injection in the neck region or IV injection in the tail vein of an HHC^{MET} formulation (10 mg/kg HHCMet). At selected time points, 200 μL blood were collected in L¹-Heparin tubes and processed to plasma by centrifugation at 3,000 g for 10 minutes at 4° C.

HHC^{MET} concentrations in rat plasma were determined with an in-house developed sandwich ELISA setup. For capturing HHC^{MET}, a human CTLA-4 (AA Ala37-Ser160)—Fc Tag fusion protein (Supplier AcroBiosystem, Newark, Del.; USA, catalog no. CT4-H5255) was coated to the ELISA plate wells and read-out was performed via a rabbit anti-camelid VHH antibody conjugated with horseradish peroxidase (supplier Genscript, Piscataway, N.J., USA, catalog no. A01861-200).

Calibration standards of HHC^{MET} in blank plasma were prepared as follows: thawed L¹-Heparin Wistar rat plasma was homogenized. The free HHC^{MET} formulation was spiked into blank plasma at concentrations between 96.0 ng/mL and 3.00 ng/mL with additional higher and lower anchor points. These solutions were used for the generation of a calibration curve. Calibration curves were analyzed via a 4-parameter logarithmic fit and 1/Y weighted. Calibration curves were confirmed via separately prepared quality control standards at 10, 40 and 80 ng/mL.

The determined HHC^{MET} plasma concentrations are depicted in Table 3.

TABLE 3
Determined mean HHC^{MET} concentrations in
ng/mL per time point and group (n = 3).

| | Time (h) | | | | | | | | |
|---|---|---|---|---|---|---|---|---|---|
| Group | 0.25 | 1 | 4 | 24 | 32 | 48 | 72 | 96 | 168 |
| 1 | — | 5.40 | 30.2 | 52.9 | 39.7 | 41.2 | 33.7 | 34.8 | 38.7 |
| 2 | 2540 | 5090 | 2280 | 2.60 | <LLOQ | <LLOQ | 2.07 | <LLOQ | <LLOQ |
| 3 | 17400 | 3530 | 1720 | 8.27 | <LLOQ | <LLOQ | 1.15 | <LLOQ | <LLOQ |
| 4 | — | 5.09 | 27.5 | 55.3 | 54.4 | 46.2 | 37.1 | 38.9 | 56.4 |

Group 1: Transient HHC^{MET}-linker hydrogel prodrug 32 (10 mg/kg HHC^{MET} equivalents - subcutaneous administration);
Group 2: HHC^{MET} (10 mg/kg - subcutaneous administration);
Group 3: HHC^{MET} (10 mg/kg - intravenous administration);
Group 4: Transient HHC^{MET}-linker hydrogel prodrug 32 (10 mg/kg HHC^{MET} equivalents - intramuscular administration); method LLOQ at 3.00 ng/mL; ,,
—″ denotes sample not taken

Specifically, HHC^{MET} concentration 72 h after intra-tissue (subcutaneous or intramuscular) injection of transient HHC^{MET}-linker hydrogel prodrug 32 (10 mg/kg HHC^{MET} equivalents) is at least 80% of HHC^{MET} concentration 1 h after intra-tissue (subcutaneous or intramuscular) injection of transient HHC^{MET}-linker hydrogel prodrug 32 (10 mg/kg HHC^{MET} equivalents).

### EXAMPLE 33: IN VIVO ANTI-TUMOR EFFICACY

The study was conducted in female C57BL/6 mice with the human CTLA4 gene knocked into the endogenous CTLA4 locus with an age of 6-11 weeks at the day of tumor inoculation. This model is also known as a Human Genetically Engineered Mouse Model or HuGEMM. Mice were subcutaneously implanted with 1×10⁶ MC38 tumor cells in the right flank. When tumors to be injected were grown to a mean tumor volume of ˜65 mm³, mice were randomized into treatment cohorts (day 0) and treated with either: 1) a single 50 μL intratumoral injection of control hydrogel 30 or 2) four total 200 μL intraperitoneal doses of 18 μg given on days 0, 4, 8, and 12 of free CTLA-4 mAB, or 3) a single 50 μL intratumoral injection of 840 μg of CTLA-4 mAB-linker-hydrogel prodrug 29b or 3) a single 50 μL intratumoral injection of 72 μg of CTLA-4 mAB-linker-hydrogel prodrug 29c. Hydrogels were administered as suspensions in 20 mM succinate, 135 mM NaCl, 0.01% Tween20, pH 4.0 buffer. Following treatment initiation, anti-tumor efficacy was assessed by determination of tumor volumes at various time points from tumor size measurements with a caliper. Tumor volumes were calculated according to the formula: Tumor volume=(L×W²)×0.5 where L is the length of the tumor and W the width (both in mm). Mice were removed from the study once tumors were greater than 3000 mm³. Percent tumor growth inhibition (TGI) was calculated according to the formula: [1−(Mean Tumor Volume in treated animals)/(Mean Tumor Volume in control animals)]*100.

Results: Intratumorally delivered control Hydrogel 30 resulted in an average tumor volume of 2142.5 mm³ at day 20 post initiation of dosing. As compared to 30, significantly lower tumor sizes were observed with either systemically delivered free CTLA-4 mAB (IP) or intratumorally delivered 29b resulting in average tumor volumes of 57.1 and 203.9 mm³, respectively and percent TGI values of 97.3%, 90.5%, respectively at day 20 post initiation of dosing (Table 4). One-way ANOVA demonstrated that free CTLA-4 mAB and 29b treatment were statistically significant vs 30 treatment (p-values of 0.0014 and 0.0008, respectively) with no significance comparing free CTLA-4 mAB and 29b treatment (p=0.9906, Table 4). A non-significant decrease in tumor growth was seen in the 29c treated animals (average tumor volume: 1580.2 mm³, 26.2% TGI)

TABLE 4
Summary of CTLA4 treatment efficacy results
in MC38 tumor bearing mice at Day 20

| | Mean | SEM | | | P-value* | Percent |
|---|---|---|---|---|---|---|
| | Tumor | Tumor | | P-value* | vs G3: | Tumor |
| | Volume | Volume | | vs G1: | Free CTLA4 | Growth |
| Group | (mm3) | (mm3) | N | Control | Ab IP | Inhibition |
| Control hydrogel 30 | 2142.5 | 406.7 | 8 | NA | 0.0014 | NA |
| CTLA-4 mAB | 57.1 | 3.7 | 5 | 0.0014 | NA | 97.3% |
| 29b | 203.9 | 56.1 | 8 | 0.0008 | 0.9906 | 90.5% |
| 29c | 1580.2 | 405.4 | 8 | 0.5700 | 0.0233 | 26.2% |

SEM = standard error of the mean, N = sample size;
*Significance between Mean Tumor Volumes was determined by One-way ANOVA followed by multiple comparisons using Tukey's Honest Significant Differences (HSD) post-hoc test.

### EXAMPLE 34: FLOW CYTOMETRIC PROFILING OF BLOOD, SPLEEN AND TUMOR IMMUNE CELLS

The study was conducted in female C57BL/6 mice with the human CTLA4 gene knocked into the endogenous CTLA4 locus with an age of 6-11 weeks at the day of tumor inoculation. This model is also known as a Human Genetically Engineered Mouse Model or HuGEMM. Mice were subcutaneously implanted with 1×10⁶ MC38 tumor cells in the right flank. When tumors to be injected were grown to a mean tumor volume of ˜65 mm³, mice were randomized into treatment cohorts (day 0) and treated with either: 1) a single 50 μL intratumoral injection of control hydrogel 30 or 2) four total 200 μL intraperitoneal doses of 18 μg given on days 0, 4, 8, and 12 of free CTLA-4 mAB, or 3) a single 50 μL intratumoral injection of 840 μg of CTLA-4 mAB-linker-hydrogel prodrug 29b or 3) a single 50 μL intratumoral injection of 72 μg of CTLA-4 mAB-linker-hydrogel prodrug 29c. Hydrogels were administered as suspensions in 20 mM succinate, 135 mM NaCl, 0.01% Tween20, pH 4.0 buffer.

Mice were sacrificed 7 days after randomization (D0). Following sacrifice, blood, spleen and tumor were isolated. Spleen samples were dissociated mechanically to generate single suspensions. Tumor samples were enzymatically and mechanically dissociated to generate single cell suspensions. Cell suspensions from spleen and tumor were centrifuged at 300 g for 5 minutes and 2×10⁶ cells were used for staining. For whole blood, approximately 100 μL was used for staining. Cells were resuspended in FACS buffer with 1 μg/ml Fc-Block and incubated at 4° C. for 10 minutes in the dark. Surface marker antibody mixtures in FACS buffer were added to each sample and samples were incubated in the dark at 4° C. for 30 minutes. Red blood cell lysis buffer (Bio-gems) was added if needed and cells were further incubated at 4° C. for 10 minutes. Cells were washed twice with FACS buffer then fixed and permeabilized for 30 minutes at room temperature with Fix/Perm buffer (eBioscience). Cells were washed twice in Permeabilization Buffer and stained with intracellular antibodies in Permeabilization buffer for 30 minutes at room temperature. Cells were washed twice in FACS buffer and acquired in the presence of 123count Ebeads (eBioscience). After collection, FACS data was analyzed using FlowJo Version 10.6.1. Compensation was digitally adjusted using single antibody-stained beads, single antibody-stained cells, and fluorescence minus one (FMO) controls. CD4⁺ T cells were gated as live, intact CD45+ CD3+ CD4+ events. Tregs were gated as the CD25+ FOXP3+ subset of CD4 T cells. CD8⁺ T cells were gated as live, intact CD45+ CD3+ CD8+ events. Subgates were then defined using the controls mentioned above. The change in the mean percentage of positive cells for indicated populations relative to control treatment was calculated by the formula: mean percentage of positive cells in treated animals—mean percentage of positive cells in control treated animals.

Summary of Antibodies Used for FACS Profiling:

| Markers | Fluorochrome | Clone | Isotype |
|---|---|---|---|
| CD45 | FITC | 30-F11 | Rat IgG2b, κ |
| CD3 | BUV395 | 17A2 | Rat IgG2b, κ |
| CD4 | BUV737 | GK1.5 | Rat IgG2b, κ |
| CD8 | PE-eFluor610 | 53-6.7 | Rat IgG2a, k |
| CD25 | BV510 | PC61 | Rat IgG1, λ |
| FOXP3 | PE | FJK-16S | Rat IgG2a, k |
| Ki67 | PerCP/Cy 5.5 | 16A8 | Rat IgG2a, k |
| ICOS | BV421 | C398.4A | Armenian Hamster |
| | | | IgG |
| CD69 | BV605 | H1.2F3 | Armenian Hamster |
| | | | IgG |
| CD44 | BV711 | IM7 | Rat IgG2b, κ |
| CD62L | APC | MEL-14 | Rat IgG2a, k |
| CD335 | PE/CY7 | 29A1.4 | Rat IgG2a, k |
| Live/Dead | efluo780 | NA | NA |

Results:

TABLE 5
Increase in Percentage Positive from control hydrogel 30

Tissue + Readout

| | | | |
|---|---|---|---|
| Tumor | Tumor | Tumor | Tumor |

Treatment

| | CD44++ | | | CD44++ |
|---|---|---|---|---|
| | CD62L− | CD69+ | ICOS+ | CD62L− |
| | of CD4s | of CD4s | of CD4s | of CD8s |
| CTLA-4 mAB | 10.73% | 2.37% | 13.77% | 6.51% |
| 29b | 34.33% | 6.85% | 18.12% | 48.45% |
| 29c | 40.55% | 13.54% | 11.42% | 44.73% |

TABLE 6
Increase in Percentage Positive from control hydrogel 30

| | Tissue + Readout | | | | | |
|---|---|---|---|---|---|---|
| | Blood | Blood | Blood | Blood | Blood | Blood |
| | Treatment | | | | | |
| | CD4 | Treg | CD25+ | CD69+ | CD69+ | ICOS+ |
| | of T | of CD4 | of CD4 | of CD4 | of Treg | of CD8s |
| CTLA-4 mAB | 0.53% | 2.22% | 2.88% | 3.35% | 3.69% | 9.26% |
| 29b | −11.03% | 2.58% | 6.58% | 3.83% | 3.21% | 5.86% |
| 29c | −0.43% | 1.20% | 2.09% | 1.47% | 2.77% | 1.86% |

TABLE 7
Increase in Percentage Positive from control hydrogel 30

| | Tissue + Readout | | | | | |
|---|---|---|---|---|---|---|
| | Spleen | Spleen | Spleen | Spleen | Spleen | Spleen |
| | Treatment | | | | | |
| | CD4 | Treg | CD25+ | CD69+ | CD69+ | ICOS+ |
| | of T | of CD4 | of CD4 | of CD4 | of Treg | of CD8s |
| CTLA-4 mAB | 4.90% | 6.64% | 4.38% | 8.33% | 9.17% | 3.18% |
| 29b | −2.10% | 5.84% | 4.25% | 7.33% | 9.87% | 0.65% |
| 29c | −0.83% | 1.57% | 1.49% | 2.87% | 4.73% | 0.17% |

TABLE 8
Increase in Percentage Positive from control hydrogel 30

| | Tissue + Readout | | | | |
|---|---|---|---|---|---|
| | Blood | Blood | Blood | Spleen | Spleen |
| | Treatment | | | | |
| | CD44++ | | | | |
| | CD62L− | ICOS+ | Ki67+ | Ki67+ | Ki67+ |
| | of CD4s | of CD4s | of CD4s | of CD4s | of Tregs |
| CTLA-4 mAB | 15.28% | 18.80% | 14.64% | 11.75% | 16.97% |
| 29b | 8.51% | 9.04% | 8.28% | 3.82% | 5.40% |
| 29c | 3.60% | 2.27% | 2.32% | 0.36% | −0.15% |

In an analysis of intratumoral lymphocytes for measures of local T cell activation, treatment with 29b or 29c resulted in increased local T cell activation in tumors as compared to 30 (Table 5). For example, as compared to 30, treatment with 29b or 29c resulted in increases in the percentage of CD44⁺⁺CD62L^{1ow} effector cells within CD4 T cells (34.33% and 40.55%, respectively); increases in the percentage of CD69⁺ cells within CD4 T cells (6.85% and 13.54%, respectively); increases in the percentage of ICOS⁺ cells within CD4 T cells (18.12% and 11.42%, respectively); and increases in the percentage of CD44⁺⁺CD62L^{1ow} effector cells within CD8 T cells (48.45% and 44.73%, respectively).

In an analysis of peripheral blood or splenic lymphocytes as measures of systemic anti-CTLA4 induced responses, treatment with 29b or 29c resulted in limited systemic CTLA4 induced responses as compared to 30 (Tables 6, 7, 8). For example, compared to 30, treatment with 29b or 29c resulted, in peripheral blood (Table 6), in no increases in the percentages of CD4 T cells within total T cells and less than 10% increases in: the percentages of T_{regs} within CD4 T cells (increases of 2.58% and 1.20%, respectively); the percentage of CD25⁺ T cells within CD4 T cells (increases of 6.58% and 2.09%, respectively); the percentage of CD69⁺ T cells within CD4 T cells (increases of 3.83% and 1.47%, respectively); the percentage of CD69⁺ cells within T_{regs}. (increases of 3.21% and 2.77%, respectively); or the percentage of ICOS⁺ cells within CD8 T cells (increases of 5.86% and 1.86%, respectively). Similarly, compared to 30, treatment with 29b or 29c resulted, in spleen (Table 7), in no increases in the percentages of CD4 T cells within total T cells and less than 10% increases in: the percentages of T_{regs} within CD4 T cells (increases of 5.84% and 1.57%, respectively); the percentage of CD25⁺ T cells within CD4 T cells (increases of 4.25% and 1.49%, respectively); the percentage of CD69⁺ T cells within CD4 T cells (increases of 7.33% and 2.87%, respectively); the percentage of CD69⁺ cells within T_{regs}. (increases of 9.87% and 4.73%, respectively); or the percentage of ICOS⁺ cells within CD8 T cells (increases of 0.65% and 0.17%, respectively).

In an analysis of peripheral blood or splenic lymphocytes as measures of systemic anti-CTLA4 induced responses, as compared to 30, treatment with 29b or 29c resulted in limited systemic CTLA4 induced responses as compared to treatment with CTLA-4 mAB (Table 8). This is noteworthy as both CTLA-4 mAB and 29b resulted in similar efficacy of anti-tumor growth inhibition (Table 4). Uniquely, as compared to treatment with 30, treatment with 29b or 29c but not CTLA-4 mAB resulted in less than 10% increases in: the percentage of CD44⁺⁺CD62L^{1ow} effector cells within CD4 T cells in blood (increases of only 8.51% and 3.60%, respectively); the percentage of ICOS⁺ cells within CD4 T cells in blood (increases of only 9.04% and 2.27%, respectively), the percentage of Ki67⁺ cells within CD4 T cells in blood (increases of only 8.28% and 2.32%, respectively), the percentage of Ki67⁺ cells within CD4 T cells in the spleen (increases of only 3.82% and 0.36%, respectively), the percentage of Ki67⁺ cells within T_{regs} in spleen (an increase of only 5.40% and a decrease of 0.15%, respectively). The lack of induction of these systemic activation markers with water-insoluble controlled-release anti-CLTA4 compound of the present invention at a dose demonstrating TGI as demonstrated here is significant and noteworthy as these markers are typically induced by systemic anti-CTLA4 therapy which is known to be associated with systemic adverse events.

### Abbreviation

AcOH Acetic acid
Asp aspartate
Boc tert-butyloxycarbonyl
DCC N,N′-Dicyclohexylcarbodiimide
DCM Dichloromethane
DBU 1,8-Diazabicyclo[5.4.0]undec-7-ene
DCU N,N′-Dicyclohexylurea
DIC N,N′-diisopropylcarbodiimide
DIPEA N,N-Diisopropylethylamine
DMAP 4-(Dimethylamino)-pyridine
DMF N,N-Dimethylformamide
DMSO Dimethyl sulfoxide
DS Degree of substitution
EDC N-(3-Dimethylaminopropyl)-N′-ethylcarbodiimide HCl salt
EDTA Ethylenediaminetetraacetic acid
EtOAc Ethyl acetate
Fmoc Fluorenylmethyloxycarbonyl
HA Hyaluronic acid
HEPES 4-(2-Hydroxyethyl)piperazine-1-ethanesulfonic acid
HOBt 1-Hydroxybenzotriazole
HOSu N-Hydroxysuccinimide
HPLC High-performance liquid chromatography
IAA iodoacetamide
LC Liquid chromatography
LC-MS Mass spectrometer coupled liquid chromatography
LPLC low pressure liquid chromatography
MES 4-Morpholineethanesulfonic acid
MTBE Methyl tert-butyl ether
MTS Methanethiosulfonyl
Mw Molecular weight
NHS N-Hydroxysuccinimide
NMP N-Methyl-2-pyrrolidone
OPA o-Phthalaldehyde
PE Polyethylene
PEG Polyethylene glycol
PES Polyethersulfone
PTFE Polytetrafluoroethylene
PyBOP Benzotriazol-1-yl-oxytripyrrolidinophosphonium hexafluorophosphat
r.t./rt Room temperature
RP reversed phase
RP-HPLC Reversed-phase high-performance liquid chromatography
SPDP N-Succinimidyl 3-(2-pyridyldithio)propionate
tBu and t-Bu tert.-butyl
TCEP Tris(2-carboxyethyl)phosphine hydrochloride
TFA Trifluoroacetic acid
THF tetrahydrofurane
TMEDA Tetramethylethylenediamine
TSTU N,N,N′,N′-Tetramethyl-O—(N-succinimidyl)uroniumtetrafluorborate
UPLC Ultra performance liquid chromatography

1. An anti-CTLA4 conjugate or a pharmaceutically acceptable salt thereof, wherein said conjugate comprises a plurality of anti-CTLA4 moieties -D covalently conjugated via at least one moiety -L¹-L²- to a polymeric moiety Z, wherein -L¹- is covalently and reversibly conjugated to -D and -L²- is covalently conjugated to Z and wherein -L¹- is a linker moiety and -L²- is a chemical bond or a spacer moiety.

2. The anti-CTLA4 conjugate or a pharmaceutically acceptable salt thereof of claim 1, wherein Z comprises a polymer.

3. The anti-CTLA4 conjugate or a pharmaceutically acceptable salt thereof of claim 1, wherein Z is a hydrogel.

4. The anti-CTLA4 conjugate or a pharmaceutically acceptable salt thereof of claim 1, wherein Z is a PEG-based or hyaluronic acid-based hydrogel.

5. The anti-CTLA4 conjugate or a pharmaceutically acceptable salt thereof of claim 3, wherein Z is a hyaluronic acid-based hydrogel.

6. The anti-CTLA4 conjugate or a pharmaceutically acceptable salt thereof of claim 3, wherein the hydrogel is non-degradable.

7. The anti-CTLA4 conjugate or a pharmaceutically acceptable salt thereof of claim 1, wherein -D is selected from the group consisting of wild-type F, anti-CTLA4 antibodies, Fc enhanced for effector function/FcγR binding anti-CTLA4 antibodies, anti-CTLA4 antibodies conditionally active in tumor microenvironment, anti-CTLA4 small molecules, CTLA4 antagonist fusion proteins, anti-CTLA4 anticalins, anti-CTLA4 nanobodies and anti-CTLA4 multispecific biologics based on antibodies, scFVs or other formats.

8. The anti-CTLA4 conjugate or a pharmaceutically acceptable salt thereof of claim 1, wherein -D is ipilimumab.

9. The anti-CTLA4 conjugate or a pharmaceutically acceptable salt thereof of claim 1, wherein -D is tremelimumab.

10. The anti-CTLA4 conjugate or a pharmaceutically acceptable salt thereof of claim 1, wherein the anti-CTLA4 conjugate further comprises non-anti-CTLA4 moieties -D.

11. The anti-CTLA4 conjugate or a pharmaceutically acceptable salt thereof of claim 10, wherein the non-anti-CTLA4 moieties -D are selected from the group consisting of cytotoxic/chemotherapeutic agents, immune checkpoint inhibitors or antagonists, immune agonists, multi-specific drugs, antibody-drug conjugates (ADC), radionuclides or targeted radionuclide therapeutics, DNA damage repair inhibitors, tumor metabolism inhibitors, pattern recognition receptor agonists, protein kinase inhibitors, chemokine and chemoattractant receptor agonists, chemokine or chemokine receptor antagonists, cytokine receptor agonists, death receptor agonists, CD47 or SIRPα antagonists, oncolytic drugs, signal converter proteins, epigenetic modifiers, tumor peptides or tumor vaccines, heat shock protein (HSP) inhibitors, proteolytic enzymes, ubiquitin and proteasome inhibitors, adhesion molecule antagonists, and hormones including hormone peptides and synthetic hormones.

12. The anti-CTLA4 conjugate of claim 1, wherein -L¹- is of formula (XIII): 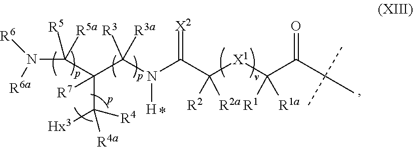
wherein
the dashed line indicates the attachment to the nitrogen of the primary or secondary amine of -D;
v is selected from the group consisting of 0 or 1;
—X¹— is selected from the group consisting of —C(R⁸)(R^{8a})—, —N(R⁹)— and —O—;
═X² is selected from the group consisting of ═O and ═N(R¹⁰);
—X³ is selected from the group consisting of —O, —S and —Se;
each p is independently selected from the group consisting of 0 or 1, provided that at most one p is 0;
—R⁶, —R^{6a}, —R¹⁰ are independently selected from the group consisting of —H, —C(R¹¹)(R^{11a})(R^{11b}) and -T;
—R⁹ is selected from the group consisting of —C(R¹¹)(R^{11a})(R^{11b}) and -T;
—R¹, —R^{1a}, —R², —R^{2a}, —R³, —R^{3a}, —R⁴, —R^{4a}, —R⁵, —R^{5a}, —R⁷, —R⁸, —R^{5a}, —R¹¹, —R^{11a} and —R^{11b} are independently selected from the group consisting of —H, halogen, —CN, —C(O)OR¹², —OR¹², —C(O)R¹², —C(O)N(R¹²)(R^{12a}), —S(O)₂N(R¹²)(R^{12a}), —S(O)N(R¹²)(R^{12a}), —S(O)₂R¹², —S(O)R¹², —N(R¹²)S(O)₂N(R^{12a})(R^{12b}), —SR¹², —NO₂, —N(R¹²)C(O)OR^{12a}, —N(R¹²)C(O)N(R^{12a})(R^{12b}), —OC(O)N(R¹²)(R^{12a}), -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
—R¹², —R^{12a}, —R^{12b} are independently selected from the group consisting of —H, -T, C₁₋₆ alkyl, C₂₋₆ alkenyl and C₂₋₆ alkynyl; wherein -T, C₁₋₆ alkyl, C₂₋₆ alkenyl and C₂₋₆ alkynyl are optionally substituted with one or more —R¹³, which are the same or different and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
wherein each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
—R¹³ is selected from the group consisting of halogen, —CN, oxo, —C(O)OR¹⁵, —OR¹⁵, —C(O)R¹⁵, —C(O)N(R¹⁵)(R^{15a}), —S(O)₂N(R¹⁵)(R^{15a}), —S(O)N(R¹⁵)(R^{15a}), —S(O)₂R¹, —S(O)R¹⁵, —N(R¹⁵)S(O)₂N(R^{15a})(R^{15b}), —SR¹⁵, —N(R⁵)(R^{15a}), —NO₂, —OC(O)R¹⁵, —N(R¹⁵)C(O)R^{15a}, —N(R⁵)S(O)₂R^{15a}, —N(R¹⁵)S(O)R^{15a}, —N(R¹⁵)C(O)OR^{15a}, —N(R¹⁵)C(O)N(R^{15a})(R^{15b}), —OC(O)N(R¹⁵)(R^{15a}) and C₁₋₆ alkyl; wherein C₁₋₆ alkyl is optionally substituted with one or more halogen, which are the same or different;
wherein —R¹⁴, —R^{14a}, —R¹⁵, —R^{15a} and —R^{15b} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, -R³/—R^{3a}, —R⁴/—R^{4a}, —R⁵/—R^{5a} or —R⁸/—R^{8a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁸, —R¹/—R⁹, —R²/—R⁹ or —R²/—R¹⁰ are joined together with the atoms to which they are attached to form a ring -A-;
wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, one or more of the pairs —R³/—R⁶, —R⁴/—R⁶, —R⁵/—R⁶, —R⁶/—R^{6a} or —R⁶/—R⁷ form together with the atoms to which they are attached a ring -A′-;
wherein -A′- is selected from the group consisting of 3- to 10-membered heterocyclyl and 8- to 11-membered heterobicyclyl; and
each -L¹- is substituted with at least one -L²- and optionally further substituted provided that the hydrogen marked with the asterisk in formula (XIII) is not replaced by a substituent.

13. The anti-CTLA4 conjugate or a pharmaceutically acceptable salt thereof of claim 1, wherein -L¹- is -L¹- is of formula (XIIIa) 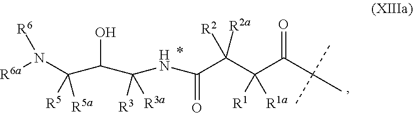
wherein
the dashed line indicates attachment to the nitrogen of the primary or secondary amine of -D;
—R¹, —R^{1a}, —R², —R^{2a}, —R³, —R^{3a}, —R⁵, —R^{5a}, —R⁶ and —R^{6a} are used as defined in claim 12; and
-L¹- is substituted with at least one moiety -L²- and is optionally further substituted, provided that the hydrogen marked with the asterisk in formula (XIIIa) is not replaced by a substituent.

14. The anti-CTLA4 conjugate or a pharmaceutically acceptable salt thereof of claim 1, wherein -L¹- is of formula (XIIIb) 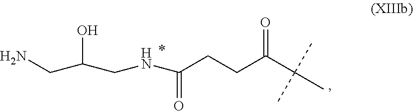
wherein
the dashed line indicates attachment to the nitrogen of the primary or secondary amine of -D; and
-L¹- is substituted with at least one moiety -L²- and is optionally further substituted, provided that the hydrogen marked with the asterisk in formula (XIIIb) is not replaced by a substituent.

15. The anti-CTLA4 conjugate or a pharmaceutically acceptable salt thereof of claim 1, wherein -L¹- is of formula (XIIIc) 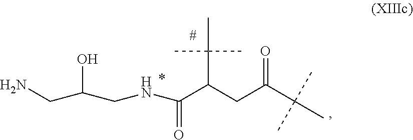
wherein
the unmarked dashed line indicates attachment to the nitrogen of the primary or secondary amine of -D, and
the dashed line marked with # indicates attachment to -L²-.

16. The anti-CTLA4 conjugate or a pharmaceutically acceptable salt thereof of claim 1, wherein -L²- is a spacer moiety.

17. The anti-CTLA4 conjugate or a pharmaceutically acceptable salt thereof of claim 1, wherein -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl;
wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R⁵, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3}a —R^{y4}, —R^{y4a}, —R^{y5}, —R^{y5a} and —R^{y5}b is independently selected from the group consisting of —H, and C₁₋₆ alkyl, wherein C₁₋₆ alkyl is optionally substituted with one or more halogen, which are the same or different.

18. The anti-CTLA4 conjugate or a pharmaceutically acceptable salt thereof of claim 1, wherein L²- comprises a moiety

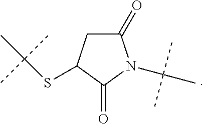

19. The anti-CTLA4 conjugate or a pharmaceutically acceptable salt thereof of claim 1, wherein -L²- has a chain length of 1 to 20 atoms.

20. The anti-CTLA4 conjugate or a pharmaceutically acceptable salt thereof of claim 1, wherein -L²- comprises a moiety of formula (XIV) 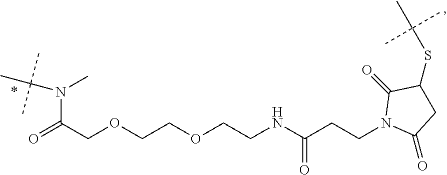
wherein
the dashed line marked with the asterisk indicates attachment to -L¹- and the unmarked dashed line indicates attachment to the remainder of -L²- or to Z.

21. A pharmaceutical composition comprising the anti-CTLA4 conjugate or a pharmaceutically acceptable salt thereof of claim 1.

22-26. (canceled)

27. A method of treating in a mammalian patient in need of the treatment of one or more diseases which can be treated with an anti-CTLA4 drug, comprising the step of administering to said patient in need thereof a therapeutically effective amount of the anti-CTLA4 conjugate or a pharmaceutically acceptable salt thereof of claim 1.

28. The method of claim 27, wherein the disease which can be treated with an anti-CTLA4 drug is a cell proliferation disorder.

29. The method of claim 28, wherein the cell proliferation disorder is cancer.

30. The method of claim 27, wherein the anti-CTLA4 conjugate or a pharmaceutically acceptable salt thereof is administered together with one or more further drug molecules or treatments.

31. The method of claim 30, wherein the one or more further drug molecules or treatments are administered to said patient prior to, together with or after administration of the anti-CTLA4 conjugate or a pharmaceutically acceptable salt thereof.


SEQUENCE LISTING (3 sequences)

SEQ ID NO: 1 - protein, 132 aa, Artificial Sequence
  aldesleukin mutein
PTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 247 aa, Artificial Sequence
  nanobody
EVQLVESGGGLVQAGGSLRLSCAASGGTFSFYGMGWFRQAPGKEQEFVADIRTSAGRTYYADSVKGRFTISRDNAKNTVYLQMNSLKPEDTAVYYCAAEMSGISGWDYWGQGTQVTVSSGGGGSGGGSEVQLVESGGGLVQAGGSLRLSCAASGGTFSFYGMGWFRQAPGKEQEFVADIRTSAGRTYYADSVKGRFTISRDNAKNTVYLQMNSLKPEDTAVYYCAAEMSGISGWDYWGQGTQVTVSS

SEQ ID NO: 3 - protein, 133 aa, Artificial Sequence
  aldesleukin mutein
APTSSSTKKTQLQLEHLLLDLQMILNGINNYKNPKLTCMLTFKFYMPKKATELKHLQCLEEELKPLEEVLNLAQSKNFHLRPRDLISNINVIVLELKGSETTFMCEYADETATIVEFLNRWITFSQSIISTLT